(19) United States
(12) Patent Application Publication
(10) Pub. No.: US 2026/0049352 A1
(43) Pub. Date: Feb. 19, 2026

(54) ADAPTERED-TAG BLOCKING OLIGONUCLEOTIDES

(71) Applicant: INTEGRATED DNA TECHNOLOGIES, INC., Coralville, IA (US)

(72) Inventors: Kyle KINNEY, North Liberty, IA (US); Rolf TURK, Iowa City, IA (US); Garrett RETTIG, Coralville, IA (US)

(21) Appl. No.: 19/298,672

(22) Filed: Aug. 13, 2025

Related U.S. Application Data
(60) Provisional application No. 63/683,028, filed on Aug. 14, 2024.
(60) Provisional application No. 63/799,154, filed on May 2, 2025.

(51) Int. Cl. C12Q 1/6853; C12Q 1/34; C12Q 1/6813; C12Q 1/686; C12Q 1/6869; G16B 30/10

(52) U.S. Cl. CPC C12Q 1/6853; C12Q 1/34; C12Q 1/6813; C12Q 1/686; C12Q 1/6869; C12Y 301/26004; G16B 30/10; C12Q 2600/16

(57) ABSTRACT

Described herein are compositions and methods for reducing adaptered-tag sequencing reads during the identification and nomination of on- and off-target CRISPR edited sites. One embodiment is a method for reducing adaptered-tag sequencing reads during the identification and nomination of on- and off-target CRISPR edited sites, the method comprising: contacting in an amplification reaction one or more adaptered-tag blocking oligonucleotides with an isolated genomic DNA having one or more tag sequences and adapter sequences; wherein the adaptered-tag blocking oligonucleotides comprise one or more blocking moieties and hybridize to adaptered-tag sequences at a junction region between the adapter and tag sequences to reduce amplification of the adaptered-tag sequences.

### CROSS-REFERENCE TO RELATED APPLICATIONS

This application claims priority to U.S. Provisional Patent Application No. 63/799,154, filed May 2, 2025, and U.S. Provisional Patent Application No. 63/683,028, filed Aug. 14, 2024, each of which is incorporated by reference herein in its entirety.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “013670-0033-US03_sequence_listing_xml_12 Aug. 2025.xml,” was created on Aug. 12, 2025, contains 916 sequences, has a file size of 832.0 kilobytes (851,968 bytes) and is incorporated by reference in its entirety into the specification.

### BACKGROUND

The CRISPR-Cas9 system is comprised of both a nuclease (Cas9) and a guideRNA and allows for the generation of targeted breaks in double-stranded DNA. The guideRNA (gRNA) consists of a constant region that allows for binding to the nuclease, as well as a variable region known as the spacer sequence which is 20 nucleotides long. The complementary region to the spacer in the targeted double-stranded DNA is referred to as the protospacer sequence. The nuclease will create a double-stranded break (DSB) in the DNA when sufficient homology exists between the spacer and protospacer. Furthermore, the double-stranded break can only occur when a nuclease-specific protospacer-adjacent motif (PAM) is present. For Cas9, the PAM sequence is NGG.

The CRISPR-Cas9 system is classified as a genome editing tool. Other examples of genome editing tools include Meganucleases, Zinc Finger Nucleases (ZNF), or transcription activator-like effector-based nucleases (TALEN). CRISPR-Cas9 falls under the clustered regularly interspaced short palindromic repeats (CRISPR) family of genome editing tools. Genome editing tools facilitate the insertion, deletion, or replacement of DNA within the genome of a living organism. As such, genome editing tools can be used to create animal models for monogenic diseases by knocking out of specific genes. Furthermore, genome editing tools can be used to repair genetic mutations to potentially cure diseases or alter cellular function by introducing genetic elements, for instance to generate CAR T-cells. The success of these applications relies on the specificity of the genome editing tools.

The specificity of the CRISPR-Cas9 system depends on the creation of a double-stranded break when sufficient homology exists between the guideRNA spacer and the DNA protospacer, as well as the presence of the PAM. Nuclease activity is optimal when complete hybridization occurs between the guideRNA and the targeting strand. Therefore, the guideRNA spacer sequence is designed to match the double-stranded DNA sequence where the double-stranded break is intended to be made, which is called the on-target site. However, double-stranded breaks can also occur at sites other than the on-target sites where incomplete homology exists between the spacer and protospacer. These locations are called off-target sites. When genome editing is performed in living organisms, off-target editing is undesired as this can affect the function of the edited cells, and thereby create a safety risk. Monitoring of the specificity of the genome editing tool is therefore necessary to be able to assess the safety of the application.

Several approaches can result in increased specificity of CRISPR-Cas9. Mutations in wild type Cas9 can lead to a decrease in off-target editing while maintaining on-target potency. Blocking of potential off-target sites by an inactive Cas9-guideRNA complex, either by using a dCas9 or truncated guideRNA (CRISPR-GUARD) can also prevent off-target editing. Introduction of deoxyribonucleic acids in the ribonucleic guideRNA can lead to a decrease in off-target editing (chRDNA). To be able to assess the efficacy of these approaches together with overall safety levels, a large number of methods have been developed to determine off-target editing. Generally, these can be classified in 3 systems: (1) in silico methods which rely on computational determination of homology between spacer and protospacer sequences, (2) in cellulo methods which determine off target editing in living cells, and (3) in vitro methods which determine off-target editing using genomic DNA as input material.

Various in cellulo methodologies (GUIDE-Seq, iGUIDE, TEG-seq) rely on the integration of a double stranded oligodeoxynucleotide tag (dsODN-tag) via the NHEJ pathway at the site where a double-stranded break occurs, thereby breaking up the protospacer/PAM sequence which prevents re-cutting of the on/off target site. Typically, the dsODN-tag is introduced in the cell together with the CRISPR-Cas9 ribonucleoprotein, or RNP, complex (Cas9 and guideRNA) and genomic isolation is performed 48-72 hours after transfection. Alternatively, CRISPR reagents can be delivered as mRNA or via an expression plasmid. After fragmentation and adapter ligation, an amplification step enriches for the adaptered fragments that contain a dsODN-tag. NGS is then applied to identify the genomic sequence surrounding the tag and thereby the genomic location where the double-stranded break occurs. The efficiency of this method relies on several factors. First, the efficiency of the nuclease-induced double-stranded break controls the tag insertion rate. As a result, off-target sites which have low levels of editing (most likely due to a larger number of mismatches between the spacer and protospacer) have a relatively smaller abundance of the inserted tag and are less likely to lead to a statistically significant outcome. Second, the sequence of the dsODN-tag can influence the likelihood of integration. See U.S. Pat. App. Pub. No. US 2022/0025365 A1, which is incorporated by reference herein in its entirety for such teachings. Therefore, some off-target sites might be more or less prone to be detected. Third, editing and therefore dsODN-tag integration depends on the epigenetic state of the genome. This can differ from cell type to cell type, and therefore the use of model systems can create different outcomes. Fourth, the repair mechanism can vary between NHEJ and MMEJ, and is dependent on the flanking sequence of the DSB. As a result, sites that favor NHEJ are more likely to incorporate the dsODN compared to sites that favor repair through the MMEJ pathway. Lastly, read loss with tag-based nomination methods is substantial, which can potentially lead to loss in assay sensitivity. The reason for this large loss of read depth is due to adaptered-tag (a dsODN tag with an adapter ligated directly to the end) read sequences. Adaptered-tag sequences are present in the reaction due to leftover dsODN-tag that does not get incorporated into the genome and gets purified along with the rest of the genomic DNA (gDNA). To selectively remove the naked dsODN-tag (tag not inserted into gDNA) is problematic because of the homologous sequence between naked and gDNA inserted dsODN-tags. Though this issue is common to all the in cellulo tag-based nomination methods mentioned (GUIDE-Seq, iGUIDE, TEG-seq), none have addressed the issue of adaptered-tag related read loss.

What is needed are methods and reagents for blocking amplification of adaptered-tag sequences while retaining tag-based amplification from genomic loci.

### SUMMARY

One embodiment described herein is a method for reducing adaptered-tag sequencing reads during the identification and nomination of on- and off-target CRISPR edited sites, the method comprising: contacting in an amplification reaction one or more adaptered-tag blocking oligonucleotides with an isolated genomic DNA having one or more tag sequences and adapter sequences; wherein the adaptered-tag blocking oligonucleotides comprise one or more blocking moieties and hybridize to adaptered-tag sequences at a junction region between the adapter and tag sequences to reduce amplification of the adaptered-tag sequences. In one aspect, the amplification reaction comprises one or more adapter-specific primers and one or more tag-specific primers to produce a first set of amplified sequences, the method further comprising: amplifying the first set of amplified sequences using universal sequencing primers targeting the tails of the tag-specific primers to produce a second set of amplified sequences; sequencing the second set of amplified sequences and obtaining sequencing data; and identifying on-/off-target CRISPR editing loci. In another aspect, the one or more tag-specific primers comprise a plurality of staggered primers, each staggered primer comprising a number of random nucleotides positioned between a tag-specific sequence portion and a universal tail sequence portion. In another aspect, the number of random nucleotides positioned between the tag-specific sequence portion and the universal tail sequence portion for each staggered primer ranges from 0 to 6. In another aspect, the one or more tag sequences comprises DNA, RNA, xeno nucleic acids, or combinations thereof. In another aspect, the one or more tag sequences comprises a double-stranded oligodeoxynucleotide tag (dsODN-tag) sequence. In another aspect, the one or more tag sequences comprises one or more modifications comprising a 5′-terminal phosphate, phosphorothioate linkages, methylphosphonate linkages, boranophosphate linkages, phosphonoacetate linkages, or combinations thereof. In another aspect, the one or more tag sequences comprises at least three phosphorothioate linkages at the 5′-terminus, 3′-terminus, or a combination thereof. In another aspect, the one or more blocking moieties of the adaptered-tag blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof. In another aspect, the adaptered-tag blocking oligonucleotides hybridize to top and bottom strands of the adaptered-tag sequences at a junction region between the adapter and tag sequences. In another aspect, the adaptered-tag blocking oligonucleotides have a sequence length of about 15 nucleotides to about 35 nucleotides. In another aspect, the adaptered-tag sequences have a sequence length of about 150 nucleotides to about 200 nucleotides. In another aspect, about 40-60% of the adaptered-tag blocking oligonucleotides hybridizes to the adapter sequence portion of the adaptered-tag sequences and about 40-60% of the adaptered-tag blocking oligonucleotides hybridizes to the tag sequence portion of the adaptered-tag sequences. In another aspect, the adaptered-tag blocking oligonucleotides reduce adaptered-tag sequencing reads by at least about 25% relative to a method without the adaptered-tag blocking oligonucleotides. In another aspect, the adaptered-tag blocking oligonucleotides increase the amount of sequencing reads at unique nominated off-target effect (OTE) sites as compared to a method without the adaptered-tag blocking oligonucleotides.

Another embodiment described herein is method for identifying and nominating on- and off-target CRISPR edited sites with improved accuracy and sensitivity, the method comprising: (a) performing a multiplex PCR reaction comprising: (i) one or more tag-specific oligonucleotide primers, each having a cleavage region comprising a ribonucleotide (rN) positioned 5′ of a blocking group and a complementary region flanking one or more tag sequences, wherein the blocking group prevents primer extension and/or inhibits the oligonucleotide primer from serving as a template for DNA synthesis; (ii) one or more adapter-specific oligonucleotide primers, each having a cleavage region comprising a rN positioned 5′ of a blocking group and a complementary region flanking the 5′ end of a universal adapter sequence; (iii) one or more adaptered-tag blocking oligonucleotides corresponding to each strand of the tag sequences and comprising one or more blocking moieties, wherein the adaptered-tag blocking oligonucleotides hybridize to top and bottom strands of adaptered-tag sequences at a junction region between the universal adapter and tag sequences and inhibit annealing of the tag-specific oligonucleotide primers to the top and bottom strands of the adaptered-tag sequences, thereby reducing amplification of the adaptered-tag sequences; and (iv) a cleaving enzyme; (b) hybridizing the tag-specific oligonucleotide primers to one or more incorporated tag sequences to form a tag sequence double stranded substrate and hybridizing one or more adapter-specific oligonucleotide primers to the 5′ end of the universal adapter sequence; (c) cleaving at a point within or adjacent to the cleavage regions with the cleaving enzyme to remove the blocking groups from the one or more tag-specific oligonucleotide primers and the one or more adapter-specific oligonucleotide primers; (d) amplifying a portion of isolated genomic DNA comprising the one or more incorporated tag sequences and the universal adapter sequence; and (e) sequencing the amplified portion of the isolated genomic DNA, thereby identifying on- and off-target CRISPR edited sites. In one aspect, the cleaving enzyme is an RNase H2 enzyme. In another aspect, the isolated genomic DNA comprising the one or more incorporated tag sequences and the universal adapter sequence is generated by: isolating genomic DNA from a cell having one or more tag sequences incorporated into a target site within a genome of the cell; and integrating a universal adapter sequence into the isolated genomic DNA. In another aspect, the universal adapter sequence comprises a unique molecular index (UMI). In another aspect, the sequencing of step (e) further comprises executing on a processor: (i) aligning sequence data to a reference genome; and (ii) outputting the alignment, analysis, and results data as custom-formatted files, tables, or graphics.

Another embodiment described herein is a method for reducing adaptered-tag sequencing reads during the identification and nomination of on- and off-target CRISPR edited sites, the method comprising: (a) co-delivering a guide sequence RNA (sgRNA) or a two-part CRISPR RNA:trans-activating crRNA (crRNA:tracrRNA) duplex, one or more tag sequences, and an RNA-guided endonuclease to cells; (b) incubating the cells for a period of time sufficient for double strand breaks to occur, and for the cells to repair the double strand breaks; (c) isolating genomic DNA from the cells, fragmenting the genomic DNA, and ligating the fragmented genomic DNA to a universal adapter sequence; (d) amplifying the ligated DNA fragments using tag-specific primers, adapter-specific primers, and blocking oligonucleotides comprising one or more blocking moieties, to produce a first set of amplified sequences; wherein the blocking oligonucleotides hybridize to top and bottom strands of adaptered-tag sequences at a junction region between the ligated adapter and tag sequences and inhibit annealing of the tag-specific primers to the top and bottom strands of the adaptered-tag sequences, thereby preventing amplification of the adaptered-tag sequences; (e) amplifying the first set of amplified sequences using universal sequencing primers targeting the tails of the tag-specific primers to produce a second set of amplified sequences; (f) sequencing the second set of amplified sequences and obtaining sequencing data; and (g) identifying on-/off-target CRISPR editing loci. In one aspect, the one or more tag sequences comprises DNA, RNA, xeno nucleic acids, or combinations thereof. In another aspect, the one or more tag sequences comprises a double-stranded oligodeoxynucleotide tag (dsODN-tag) sequence. In another aspect, the one or more tag sequences comprises one or more modifications comprising a 5′-terminal phosphate, phosphorothioate linkages, methylphosphonate linkages, boranophosphate linkages, phosphonoacetate linkages, or combinations thereof. In another aspect, the one or more tag sequences comprises at least three phosphorothioate linkages at the 5′-terminus, 3′-terminus, or a combination thereof. In another aspect, the one or more tag sequences comprises an adenine (A)-thymine (T) content of less than about 70%. In another aspect, the one or more tag sequences comprises an A-T content of less than about 50%. In another aspect, the one or more tag sequences comprises a guanine (G)-cytosine (C) content of about 30% to about 60%. In another aspect, the one or more blocking moieties of the blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof. In another aspect, the blocking oligonucleotides comprise DNA, locked nucleic acids (LNA), or combinations thereof. In another aspect, the blocking oligonucleotides have a sequence length of about 15 nucleotides to about 35 nucleotides. In another aspect, about 40-60% of the sequence of the blocking oligonucleotides hybridizes to the ligated adapter sequence portion of the adaptered-tag sequences and about 40-60% of the sequence of the blocking oligonucleotides hybridizes to the ligated tag sequence portion of the adaptered-tag sequences. In another aspect, the blocking oligonucleotides are present at a concentration of about 250 nM to about 2500 nM. In another aspect, the adaptered-tag sequences have a sequence length of about 150 nucleotides to about 200 nucleotides. In another aspect, the blocking oligonucleotides reduce adaptered-tag sequencing reads by at least about 25% as compared to a method without the blocking oligonucleotides. In another aspect, the blocking oligonucleotides increase the amount of sequencing reads at unique nominated off-target effect (OTE) sites as compared to a method without the blocking oligonucleotides. In another aspect, the blocking oligonucleotides do not inhibit the amplification of ligated tag sequences inserted in the genomic DNA. In another aspect, step (g) comprises executing on a processor: (i) aligning the sequence data to a reference genome; (ii) identifying on-/off-target CRISPR editing loci; and (iii) outputting the alignment, analysis, and results data as files, tables, or graphics. In another aspect, the method further comprises a step following step (e) comprising: (e1) normalizing the second set of amplified sequences to produce concentration normalized libraries, pooling the normalized libraries with other samples to produce pooled libraries; and continuing with steps (f)-(g). In another aspect, the sgRNA or crRNA comprises one or more modifications comprising phosphorothioate linkages, 2′-O-methyl (2′-OME) nucleotides, 2′-O-methoxy-ethyl (2′-MOE) nucleotides, 2′-F nucleotides, locked nucleic acids (LNA), or combinations thereof. In another aspect, the RNA-guided endonuclease comprises an endogenously-expressed Cas enzyme, a Cas expression vector, a Cas protein or RNP complex, or a Cas mRNA. In another aspect, the cells comprise mammalian cells. In another aspect, the cells comprise human cells or mouse cells. In another aspect, the period of time is about 24 hours to about 96 hours. In another aspect, multiple tag sequences are co-delivered.

### DESCRIPTION OF THE DRAWINGS

FIG. 1 shows a schematic for an exemplary method for reducing adaptered-tag reads during CTL-seq library prep by designing DNA/LNA blocking oligos (with a 3′-C3 spacer, dideoxy nucleotide, or alternative blocking moiety) that span the junction between the dsODN-tag and the SP1 sequence on the P5 adapter.

FIG. 2 shows an exemplary schematic overview of blocking adaptered-tag amplification through anneal inhibition of dsODN-tag specific primers with DNA/LNA blockings oligos during the CTL-seq workflow. The figure shows top and bottom strands (SEQ ID NO: 1-2); dsODN Primers (SEQ ID NO: 15-16); Adapter Primer (SEQ ID NO: 17); and Blocking Oligos (SEQ ID NO: 28, 36).

FIG. 3A-B show blocking adaptered-tag amplification with DNA/LNA blocking oligos. FIG. 3A shows plots of the top strand amplification ΔCₜ of the control sample (without blocker) minus the experimental sample (with blocker) for each indicated blocker. FIG. 3B shows plots of the bottom strand amplification ΔCₜ of the control sample (without blocker) minus the experimental sample (with blocker) for each indicated blocker. Negative ΔCₜ=decreased adaptered-tag amplification.

FIG. 4 shows a schematic overview of a 3-color probe qPCR assay to assess blocking of adaptered-tag amplification.

FIG. 5A-B show blocking adaptered-tag amplification with DNA/LNA blocking oligos. FIG. 5A shows plots of the dsODN-tag 216 top and bottom strand amplification ΔCₜ of the control sample (without blocker) minus the experimental sample (with blocker) for each indicated blocker. FIG. 5B shows plots of the dsODN-tag 064 top and bottom strand amplification ΔCₜ of the control sample (without blocker) minus the experimental sample (with blocker) for each indicated blocker. Negative ΔCₜ=decreased adaptered-tag amplification or gDNA control amplification.

FIG. 6A-D show blocking adaptered-tag amplification with DNA/LNA blocking oligos during CTL-seq NGS library preparation. FIG. 6A-B show representative images of electropherograms run on the Agilent Fragment Analyzer of CTL-seq libraries prepared with and without indicated blocking oligos for two dsODN-tags: CTL216 (FIG. 6A) and CTL064 (FIG. 6B). The adaptered-tag fragment peaks are the peaks shown around 150-200 bp. FIG. 6C-D show quantitative ratio of the concentration (ng/μL) of the adaptered-tag peak divided by the ratio of the concentration of usable NGS fragments (200-2000 bp) for various DNA/LNA blocking oligos CTL216 (FIG. 6C) and CTL064 (FIG. 6D). Negative slope indicates blocking of adaptered-tag fragment.

FIG. 7A-F show blocking adaptered-tag amplification with LNA blocking oligos during CTL-seq NGS library preparation. FIG. 7A-D show quantitative ratio of the concentration (ng/μL) of the adaptered-tag peak divided by the ratio of the concentration of usable NGS fragments (200-2000 bp) for single tube amplification of the top and bottom strand with either matched blockers (i.e., top strand amplification with top strand blocker) or mismatched blockers (i.e., top strand amplification with bottom strand blocker) for AR (FIG. 7A), EMX1 (FIG. 7B), AAVS1 (FIG. 7C), and LAG3 (FIG. 7D). FIG. 7E shows a representative electropherogram for CTL216 run on the Agilent Fragment Analyzer of CTL-seq libraries prepared with and without indicated blocking oligos in a dual strand/single tube amplification format. The adaptered-tag fragment peak is the black peak shown around 150-200 bp. FIG. 7F shows quantitative ratio of the concentration of the adaptered-tag peak divided by the ratio of the concentration of usable NGS fragments. Negative slope indicates blocking of adaptered-tag fragment.

FIG. 8A-B show NGS run metrics with (FIG. 8A) and without (FIG. 8B) adaptered-tag oligo blockers. % Q30 quality metric and % base pair composition on a per cycle basis throughout the sequencing run. NGS libraries were sequenced on a standard MiSeq flow cell with v2 chemistry.

FIG. 9A-K show OTE nomination comparison of the CTL-seq workflow with and without adaptered-tag oligo blockers. FIG. 9A-C show NGS read metrics showing percent of usable reads, percent of short reads filtered out, and percent reads mapped to the genome. FIG. 9D-E show plots of the combined number of OTE sites nominated and FIG. 9F-G show percent CTL-seq UMI read counts of sites nominated in triplicate, duplicate, or by single replicate, and UMI counts of unique sites found for samples with and without adaptered-tag blockers. FIG. 9H-K show scatterplots of the UMI read counts for samples with and without adaptered-tag blockers. The dotted line indicates OTE sites with UMI read counts≤10. OTE sites were nominated with the CTL-seq Analysis Pipeline and generated from three biological replicates per guide. Each replicate's top 500 OTE sites as determined by the position specific scoring matrix was intersected for OTE site overlap using BedtoolsIntersectLOJ and merged into unique site list for each guide.

FIG. 10A-B shows comparisons of one embodiment of the method described herein compared with GUIDE-Seq (described by Tsa et al., Nature Biotech. 33(2): 187-197 (2015) and Int. Pat. App. Pub. No. WO 2015/200378 A1). FIG. 10A shows dsODN gDNA integration comparison. Tukey box plots show the percentage of dsODN integration at matched integrated OTEs for dsODNs with either 2 or 3 phosphorothioate linkages at the 5′- and 3′-end of the dsODN. Statistical significance was determined using Wilcoxon matched-pairs signed rank test. ****P<0.0001. FIG. 10B shows a comparison of the method described herein (CTL-seq) and the GUIDE-Seq off-target analysis method. Tukey box plots show the total nominated OTE sites for each indicated methodology. All data is representative of 48 gRNAs (Targets: PDCD1, LAG3, CTLA4, NRP1, IL2RA, and TIGIT; 8 gRNAs per target). GUIDE-Seq NGS libraries were processed through the GUIDE-Seq analysis package. CTL-seq NGS libraries were processed with IDT's proprietary OTE analysis pipeline. Statistical significance was determined using Wilcoxon matched-pairs signed rank test. ****P<0.0001.

FIG. 11A-D shows a comparison of UNCOVERseq and the GUIDE-Seq off-target nomination workflows. FIG. 11A shows an overview of UNCOVERseq workflow demonstrates that cells with a genomically integrated dsDNA tag have gDNA extracted and amplified with rhPCR in a single reaction, with dsDNA tag: adapter byproducts being blocked by a targeted oligo before being sequenced and analyzed using the workflow described herein. FIG. 11B shows a depiction of the kind of events are targeted by the blocking oligo, with the usable reads (non-dsDNA: adapter reads) measured across nominating off-targets for 4 gRNAs in K562 (n=3 per gRNA) with (light blue) or without (dark blue) the blocking oligo. FIG. 11C shows a comparison of different alignment methods used in publicly available nomination packages was performed to determine differences in levenshtein distance<7 sites from a glocal alignment (Glocal), Smith-Waterman (SW; same parameters as version “original” GUIDE-Seq pipeline) or string-match method (Regex; same as version “GUIDE-Seq” pipeline). FIG. 11D shows Tukey box plots show the end-to-end differences in total nominated OTE sites for the original GUIDE-Seq method (wet lab protocol and alignment) compared with UNCOVERseq. Data is representative of 48 gRNAs (Targets: PDCD1, LAG3, CTLA4, NRP1, IL2RA, and TIGIT; 8 gRNAs per target). Statistical significance was determined for pairwise comparisons using Wilcoxon matched-pairs signed rank test and for multiple comparisons using a Friedman test with a post-hoc Dunn's test with Bonferroni correction. ****p<0.0001.

FIG. 12A-D show a comparison of UNCOVERseq nomination frequencies with and without dsDNA tag: adapter blocker. Comparison of average nomination frequencies (normalized to the UMI-corrected on-target frequency) in K562 (n=3 per gRNA) with and without the dsDNA tag: adapter blocker (Blocker) from shared sites for the following gRNAs: AR (FIG. 12A); EMX1 FIG. 12B); AAVS1 (FIG. 12C); LAG3 (FIG. 12D).

FIG. 13A-G show analysis of performance of a promiscuous cell system and the ability to translate to other cellular systems. FIG. 13A shows an exemplary diagram demonstrating promiscuous nomination systems can help sensitively represent off-target lists by increasing the frequency that off-targets are detected. FIG. 13B shows a comparison of total frequencies (represented as the cumulative % significant UMI reads) of nominated sites captured in a single replicate of HEK293-Cas9 vs wildtype Cas9 RNP transfection using three different cell types: K562 (10 gRNAs; biological duplicates per gRNA), iPSCs (6 gRNAs; one biological replicate per gRNA; wildtype and HiFi Cas9), and primary T-cells (4 gRNAs; 2 biological replicates per gRNA as two different donors). Total UMI-corrected reads, total number of nominated sites, and Spearman correlation are shown for shared nominated sites between HEK293-Cas9 and K562 (FIG. 13C); T-cells (FIG. 13D); and iPSCs (FIG. 13E). FIG. 13F shows the total number of nominated sites between iPSCs displayed in comparison to HEK293-Cas9. FIG. 13G shows the total number of nominated sites between primary T-cells displayed in comparison to HEK293-Cas9.

FIG. 14A-D show UNCOVERseq nomination reproducibility for high frequency and high priority off-targets. To measure the ability to reproducibly nominate off-targets at consistent frequencies. FIG. 14A shows a combinatorial comparison of replicates was performed of targets nominated (by frequency, measured as cumulative UMI reads on overlapping targets/UMI reads on all nominated targets) using UNCOVERseq across 5 gRNAs in HEK293-Cas9 (n=3 to 15 biological replicates per gRNA) and FIG. 14B shows results compared the target nomination frequency between replicates. To determine the reproducibility to capture high priority off-targets (defined as Tier 1 to Tier 3) 46 gRNAs across a broad specificity score spectrum were nominated in triplicate in HEK293-Cas9 (FIG. 14C) and the high priority panel content missed and the total number of off-targets for interrogation are shown (FIG. 14D) for each individual replicate (all nominated sites) compared to the total # of high priority sites (Tier 1 to Tier 3) nominated as a biological triplicate.

FIG. 15A-B show site selection for LAG3 process control panel. UNCOVERseq was used to nominate targets of the LAG3 site 9 gRNA in HEK293-Cas9 (n=12 biological replicates) and FIG. 15A the average nomination frequency (normalized to the on-target frequency) of each site was binned into 5 frequency bins (0.10-0.49%; 0.50-0.99%; 1-9.9%; 10-49.9%; >50% UMI reads relative to the on-target). FIG. 15B shows selected sites per frequency bin for interrogation as a part of process control 60-plex.

FIG. 16A-H show quality control procedures for confirming assay sensitivity and read requirements. To create a positive control for UNCOVERseq process quality control, a promiscuous LAG3 (site 9) gRNA was extensively characterized in HEK293-Cas9 using 12 biological replicate transfections with paired controls (no gRNA). Following transfection, FIG. 16A shows indel frequency and tag integration frequencies were measured at the LAG3 on-target site via NGS and FIG. 16B shows the frequency of unique sites relative to the cumulative total reaching each reproducibility frequency. FIG. 16C shows twelve sites were selected per frequency bin (Bin 5≤0.49%; Bin 4=0.50-0.99%; Bin 3=1-9.9%; Bin 2=10-49.9%; Bin 1≥50% UMI reads relative to the on-target) for routine targeted sequencing with average nomination frequency shown. FIG. 16D shows, to test application of this, paired confirmation was performed in, a highly promiscuous condition (HEK293-Cas9; n=3 biological replicates) and FIG. 16E shows high specificity condition (K562 nucleofected SpyFi RNP; n=3 biological replicates) and the status of each of the 60 measured sites meeting coverage criteria (>1,000×) recorded. FIG. 16F shows quantification of HEK293-Cas9 confirmation: nomination frequencies is plotted with final status shown as either Nominated and Confirmed (blue circle), Nominated and Not Confirmed (light blue triangle), or Not Nominated and Confirmed (orange square) are shown in FIG. 16G, and confirmed indel frequencies per bin shown. FIG. 16H shows downsampling of the HEK293-Cas9 LAG3 nomination samples (n=15) was performed and sensitivity per bin calculated for recovering all 60 LAG3 positive control confirmation loci.

FIG. 17A-H shows comparative analysis of UNCOVERseq to other nomination technologies. FIG. 17A shows a comparison of CHANGE-seq and GUIDE-Seq sensitivity and FIG. 17B shows the relative nomination technology frequency across 60 confirmed off-targets derived from the LAG3 site 9 gRNA in addition to sensitivity (FIG. 17C) and relative nomination frequency (FIG. 17D) across the full 723 UNCOVERseq derived targets that had 100% reproducibility (n=12 biological replicates). FIG. 17E shows a comparison of INDUCE-seq and GUIDE-Seq sensitivity and FIG. 17F shows relative nomination technology frequency across 81 fully reproducible UNCOVERseq off-targets (n=6) derived from the EMX1 gRNA. FIG. 17G shows a comparison of SITE-seq and GUIDE-Seq sensitivity and FIG. 17H shows relative nomination technology frequency across 46 fully reproducible UNCOVERseq off-targets (n=6) derived from the FANCF gRNA.

FIG. 18A-D show UNCOVERseq gRNA specificity scores for nominated gRNAs and ABE/CBE compatible gRNAs. 192 gRNAs were individually transfected into HEK293-Cas9 to perform UNCOVERseq. FIG. 18A shows read depth per sample of each gRNA and FIG. 18B shows the rank order specificity score were quantified. Specificity scores were binned from 0 to 1 in 0.2 increments and the # of gRNAs per binned counted for (FIG. 18C), all gRNAs and FIG. 18D shows gRNAs that met ABE criteria (at least one “A” in 5′ position 4-7) and CBE criteria (at least one “C” in 5′ position 4-8).

FIG. 19A-J show on-target and off-target editing in HEK293-Cas9 and HSPCs. FIG. 19A shows six gRNAs with a single targeted ABE or CBE base were selected with specificity scores shown. HEK293-Cas9 (n=1) and HSPCs (n=3 donors), with simultaneous delivery of S.p. Cas9 mRNA, were delivered each gRNA and had on-target indel editing quantified by NGS (FIG. 19B). HSPCs were also delivered mRNA for each gRNA of either the S.p. Cas9-ABE8 or S.p. Cas9-CBE fusion and had indel editing (FIG. 19C) and base editing quantified using NGS (FIG. 19D). FIG. 19E shows multiplexed amplicon sequencing (rhAmpSeq) panels were created for each gRNA for off-target quantification based on origin from UNCOVERseq or in silico nomination (in silico) with the number of targets interrogated per gRNA shown. After sequencing/confirmation of off-targets in all conditions, the frequency that UNCOVERseq nominated sites converted to true positives was measured for S.p. Cas9 indel editing (FIG. 19F) and S.p. Cas9 base editing conditions (FIG. 19G). Confirmed base editing sites were categorized by their respective indel confirmation status (DSB=wildtype S.p. Cas9; SSB=ABE/CBE nickase) with cumulative base editing plotted for ABE (FIG. 19H) and CBE editing conditions (FIG. 19I). FIG. 19J shows frequencies were plotted for all confirmed ABE/CBE sites with both SSB and DSB confirmation with the respective DSB indel frequency and Spearman r calculated.

FIG. 20A-C show sequencing performance per gRNA off-target panel. FIG. 20A shows the average targeted sequencing coverage for each assay within the multiplexed rhAmp Seq panel created for confirmation of off-targets is plotted using paired treatment/controls for the HSPC wildtype S.p. Cas9 editing condition (n=3 per treatment) with the >1,000× coverage requirement depicted (dotted red line). FIG. 20B shows the frequency of all replicates reaching >1,000× for each target in the panel, and FIG. 20C shows the number of targets failing to meet this threshold quantified per gRNA.

FIG. 21A-F show confirmation in HEK293-Cas9 (indels). Editing quantification at assays with >1,000× coverage. Each dot represents a gRNA on/off-target with the average raw frequency of indels of the control (x-axis) and treatment (y-axis) plotted. Blue dots indicate sites with no statistical significance while orange dots indicate significant sites (p adj<0.05) for the gRNAs: PDCD1 site 8 (FIG. 21A); CYP2C18 (FIG. 21B); RNF2 (FIG. 21C); TRAC site 7 (FIG. 21D); B2M site 1 (FIG. 21E); TIGIT site 7 (FIG. 21F). Text at the bottom right displays the total number of confirmed sites out of all interrogated.

FIG. 22A-F show confirmation in HSPCs with wildtype S.p. Cas9 delivered as mRNA (indels). Editing quantification at assays with >1,000× coverage. Each dot represents a gRNA on/off-target with the average raw frequency of indels of the control (x-axis) and treatment (y-axis) plotted. Blue dots indicate sites with no statistical significance while orange dots indicate significant sites (p adj<0.05) for the gRNAs: PDCD1 site 8 (FIG. 22A); CYP2C18 (FIG. 22B); RNF2 (FIG. 22C); TRAC site 7 (FIG. 22D); B2M site 1 (FIG. 22E); TIGIT site 7 (FIG. 22F). Text at the bottom right displays the total number of confirmed sites out of all interrogated.

FIG. 23A-B show confirmable editing sites that can be missed with Regex alignment approach. Confirmed off-targets (p-value<0.05) were intersected with those missed using the Regex method for determining off-target alignment distance<7 and frequencies of these sites determined per gRNA in HEK293-Cas9 (FIG. 23A) and per condition, between HEK293-Cas9 and HSPCs (wildtype Cas9, CBE, and ABE) (FIG. 23B).

FIG. 24A-F show confirmation in HSPCs with wildtype S.p. Cas9-ABE delivered as mRNA (ABE). Editing quantification at assays with >1,000× coverage. Each dot represents a gRNA on/off-target with the average raw frequency of cumulative ABE transition events of the control (x-axis) and treatment (y-axis) plotted. Blue dots indicate sites with no statistical significance while orange dots indicate significant sites (p adj<0.05) for the gRNAs: PDCD1 site 8 (FIG. 24A); CYP2C18 (FIG. 24B); RNF2 (FIG. 24C); TRAC site 7 (FIG. 24D); B2M site 1 (FIG. 24E); TIGIT site 7 (FIG. 24F). Text at the bottom right displays the total number of confirmed sites out of all interrogated.

FIG. 25A-F show confirmation in HSPCs with wildtype S.p. Cas9-CBE delivered as mRNA (CBE). Editing quantification at assays with >1,000× coverage. Each dot represents a gRNA on/off-target with the average raw frequency of cumulative CBE transition events of the control (x-axis) and treatment (y-axis) plotted. Blue dots indicate sites with no statistical significance while orange dots indicate significant sites (p adj<0.05) for the gRNAs: PDCD1 site 8 (FIG. 25A); CYP2C18 (FIG. 25B); RNF2 (FIG. 25C); TRAC site 7 (FIG. 25D); B2M site 1 (FIG. 25E); TIGIT site 7 (FIG. 25F). Text at the bottom right displays the total number of confirmed sites out of all interrogated.

FIG. 26A-F show confirmation in HSPCs with wildtype S.p. Cas9-ABE delivered as mRNA (indels). Editing quantification at assays with >1,000× coverage. Each dot represents a gRNA on/off-target with the average raw frequency of indel events of the control (x-axis) and treatment (y-axis) plotted. Blue dots indicate sites with no statistical significance while orange dots indicate significant sites (p adj<0.05) for the gRNAs: PDCD1 site 8 (FIG. 26A); CYP2C18 (FIG. 26B); RNF2 (FIG. 26C); TRAC site 7 (FIG. 26D); B2M site 1 (FIG. 26E); TIGIT site 7 (FIG. 26F). Text at the bottom right displays the total number of confirmed sites out of all interrogated.

FIG. 27A-F show confirmation in HSPCs with wildtype S.p. Cas9-CBE delivered as mRNA (indels). Editing quantification at assays with >1,000× coverage. Each dot represents a gRNA on/off-target with the average raw frequency of indels of the control (x-axis) and treatment (y-axis) plotted. Blue dots indicate sites with no statistical significance while orange dots indicate significant sites (p adj<0.05) for the gRNAs: PDCD1 site 8 (FIG. 27A); CYP2C18 (FIG. 27B); RNF2 (FIG. 27C); TRAC site 7 (FIG. 27D); B2M site 1 (FIG. 27E); TIGIT site 7 (FIG. 27F). Text at the bottom right displays the total number of confirmed sites out of all interrogated.

FIG. 28 shows on-target indel and base editing correlations in HSPCs. Frequencies were plotted for all on-target ABE/CBE sites with both SSB and DSB along with the respective DSB indel frequency (wildtype Cas9) and Spearman r calculated.

FIG. 29A-B show translocation and cumulative off-target risk. Targeted sequencing data also allows translocation detection between on-target: off-target and off-target: off-target assays in the same pool. FIG. 29A shows translocation quantification (FDR<0.01) at the PDCD1 site 8 gRNA across different editor modalities in HSPCs and FIG. 29B shows the cumulative off-target ratio (% cumulative off-target events/% on-target) were calculated for each gRNA in HSPCs with mRNA delivery. Cumulative off-target events for all modalities considers both base editing and indel editing.

FIG. 30A-B show that adding three phosphorothioate (PS) linkages at the 5′- and 3′-ends increase dsODN integration at CRISPR-induced double-stranded breaks. FIG. 30A shows the total number of dsODN-integrated OTEs for three gRNAs. FIG. 30B shows dsODN integration rate for matched dsODN-integrated OTEs for gRNAs: AR, EMX1, and AAVS1. Statistical significance was determined using paired t test. ****P<0.0001.

FIG. 31 shows a schematic overview of the staggered rhPCR primers.

FIG. 32A-B show base composition of the beginning of Read 2 during Illumina sequencing with (FIG. 32A) and without (FIG. 32B) staggered rhPCR1 primers.

FIG. 33A-D show dsODN identification at the beginning of Read2 with and without staggered rhPCR1 primers. dsODN identification (FIG. 33A), CRISPR read specificity (FIG. 33B), loading concentration (FIG. 33C), and Q30 (FIG. 33D).

$$CRISPR Read Specificity = \frac{UMI Reads at CRISPR Edited Sites}{Total UMI Reads} .$$

FIG. 34A-D show dsODN identification at the beginning of Read2 with and without staggered rhPCR1 primers: dsODN identification (FIG. 34A), CRISPR read specificity (FIG. 34B), loading concentration (FIG. 34C), and Q30 (FIG. 34D).

$$CRISPR Read Specificity = \frac{UMI Reads at CRISPR Edited Sites}{Total UMI Reads} .$$

FIG. 35A-D show comparisons of UNCOVERseq nomination frequencies with and without staggered PCR1 primers. Comparison of average nomination frequencies (normalized to the UMI-corrected on-target frequency) in HEK293-Cas9 (n=3 per gRNA) from shared sites for the following gRNAs: PCSK9 (FIG. 35A), FANCF (FIG. 35B), EMX1 (FIG. 35C), and LAG3 (FIG. 35D).

FIG. 36A-H show comparison of UNCOVERseq nominated off-targets from libraries made with and without staggered PCR1 primers. FIG. 36A-D show a combinatorial comparison of replicates from targets nominated (by frequency, measured as cumulative UMI reads on overlapping targets/UMI reads on all nominated targets): PCSK9 (FIG. 36A), FANCF (FIG. 36B), EMX1 (FIG. 36C), and LAG3 (FIG. 36D). FIG. 36E-H show average nomination frequencies (normalized to the UMI-corrected on-target frequency) of unique off-targets nominated from libraries made with and without staggered PCR1 primers: PCSK9 (FIG. 36E), FANCF (FIG. 36F), EMX1 (FIG. 36G), and LAG3 (FIG. 36H). Data is representative across 4 gRNAs in HEK293-Cas9 (n=3 replicates per gRNA).

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, nucleic acids may contain the following abbreviations in addition to the standard nucleotides (A, C, G, T, U), where R indicates A or G; Y indicates C or T; S indicates G or C; W indicates A or T; K indicates G or T; M indicates A or C; B indicates C or G or T; D indicates A or G or T; H indicates A or C or T; V indicates A or C or G; and N indicates any base (A, C, G, T, or U as applicable)

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points, or as described above in the definition of “about.”

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30° C., including all integers and endpoints within the specified range. In another aspect, “room temperature” refers a temperature of about 15-30° C.; about 20-30° C.; about 22-30° C.; about 25-30° C.; about 27-30° C.; about 15-22° C.; about 15-25° C.; about 15-27° C.; about 20-22° C.; about 20-25° C.; about 20-27° C.; about 22-25° C.; about 22-27° C.; about 25-27° C.; about 15° C.±10%; about 20° C.±10%; about 22° C.±10%; about 25° C.±10%; about 27° C.±10%; ˜ 20° C., ˜22° C., ˜25° C., or ˜27° C., at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein “mN” indicates 2′-O-methylation of the N nucleotide that is preceeded by the “m.”

As used herein “rN” indicates a ribonucleotide, where N is the nucleotide preceeded by the “r.”

As used herein, “/5Phos/” indicates a 5′-terminal phosphate.

As used herein “*” indicates a phosphorothioate linkage between the two nucleotides.

As used herein, “+N” indicates a locked nucleotide (LNA), where N is the nucleotide preceeded by the “+.” As used herein “locked nucleic acid” or “LNA” refers to a modified ribonucleotide comprising a methylene bridge bond linking the 2′ oxygen to the 4′ carbon of the ribose pentose ring:

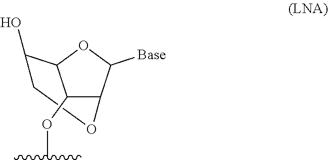

LNAs impart structural stability, including increased hybridization Tₘ and resistance to nucleases.

As used herein, “/3SpC3/” indicates a 3′-terminal C₃ spacer.

As used herein, “/56-FAM/” indicates a 5′-terminal 6-FAM (Fluorescein) fluorophore.

As used herein, “/3IABKFQ/” indicates a 3′-terminal Iowa Black® FQ fluorescence quencher.

As used herein, “/5HEX/” indicates a 5′-terminal HEX fluorophore (hexachlorofluorescein).

As used herein, “/5Cy5/” indicates a 5′-terminal Cy5™ (Cyanine 5) fluorophore.

As used herein, “/ZEN/” indicates an internal ZEN™ fluorescence quencher.

As used herein, “/TAO/” indicates an internal TAO™ fluorescence quencher.

As used herein, “/3IAbRQSp/” indicates a 3′-terminal Iowa Black® RQ fluorescence quencher.

As used herein, “/3ddC/” indicates a 3′-terminal dideoxycytidine.

As used herein, 2′-fluorine” or “2′-F” refers to a 2′-fluorine moiety.

As used herein, “2′-O-methyl” refers to a 2′-O-methyl moiety.

As used herein, “2′-O-methoxy-ethyl” or “2′-MOE” refers to a 2′-O-methoxy-ethyl moiety.

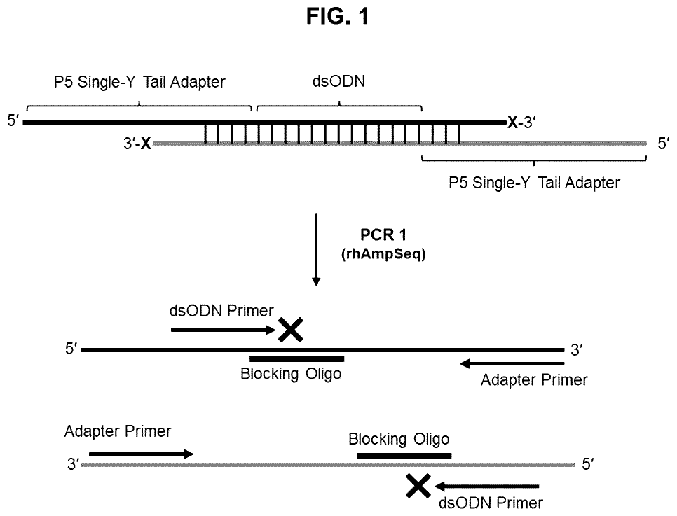

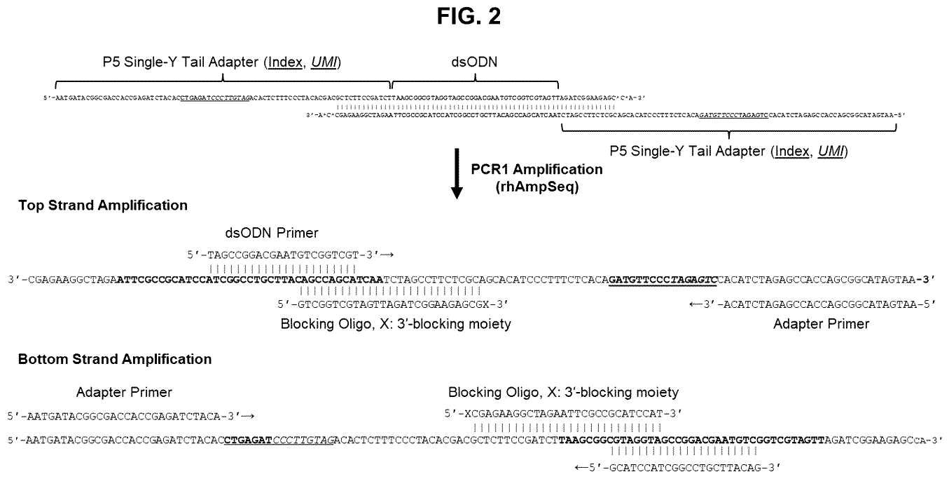

Described herein are reagents and methods for selectively blocking the amplification of adaptered-tag sequences while retaining tag-based amplification from genomic loci in in cellulo dsODN-tag based nomination workflows (e.g., “CTL-seq” as described in U.S. Pat. App. Pub. No. US 2022/0025365 A1, which is incorporated by reference herein in its entirety for such teachings) (FIG. 1-2). CTL-seq comprises co-delivering a guide sequence RNA or two-part CRISPR RNA:transactivating crRNA (crRNA:tracrRNA) duplex, one or more tag sequences, and an RNA-guided endonuclease into cells. Cells are incubated for a period of time sufficient for DSBs and subsequent repair to occur. Genomic DNA is then isolated from cells, followed by gDNA fragmentation, end-repair, A-tailing, and ligation of a unique molecular index containing a universal adapter sequence. Fragmented gDNA libraries are amplified in a 1^{st} round of PCR using primers targeting the tag and universal adapter sequences to produce a first set of amplified sequences; followed by a 2^{nd} round of PCR targeting the SP1 and SP2 sequences (or other sequences with similar functionality) embedded in the PCR1 primers. The amplified library is then sequenced to identify on-/off-target CRISPR editing loci.

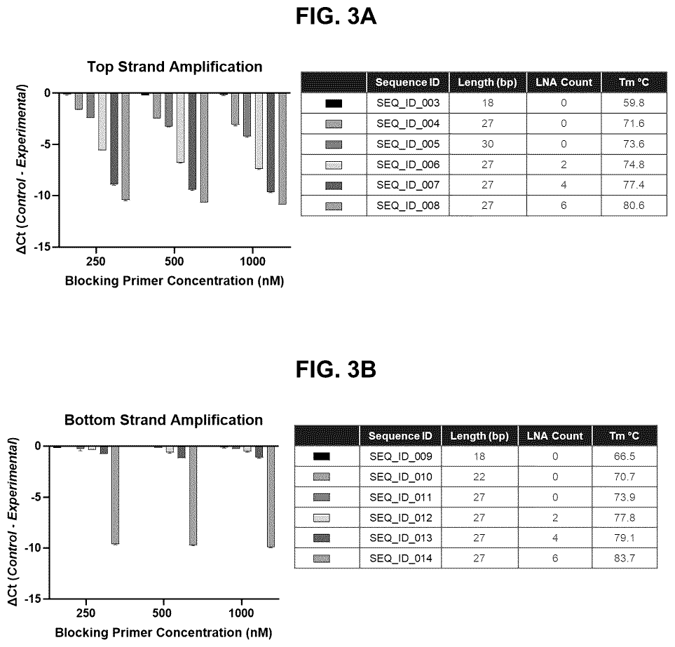

To selectively block the amplification of adaptered-tag sequences, the CTL-seq amplification protocol described above was modified to perform the 1^{st} round of PCR in the presence of DNA/LNA blocking oligos with a 3′-polymerase extension blocking moiety (C3 spacer, dideoxy, and/or inverted dideoxy nucleotides, etc.) that span the junction of dsODN-tag and SP1 region on the P5 adapter preventing adaptered-tag amplification while permitting amplification of dsODN-tag inserted into genomic loci (FIG. 3). To mitigate the occurrence of blocking genomic inserted dsODN-tags, around half of the blocking oligo spans the dsODN-tag region while the other half covers the SP1 region of the P5 adapter. In doing so, neither half of the blocking oligo should have a high enough Tₘ ° C. to bind dsODN-tag or SP1 to block genomic loci nonspecifically.

The polynucleotides described herein include variants that have substitutions, deletions, and/or additions that can involve one or more nucleotides. The variants can be altered in coding regions, non-coding regions, or both. Alterations in the coding regions can produce conservative or non-conservative amino acid substitutions, deletions, or additions. Especially preferred among these are silent substitutions, additions, and deletions, which do not alter the properties and activities of the binding.

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to nucleotide sequences, or degenerate, homologous, or codon-optimized variants thereof described herein, or nucleotide sequences capable of hybridizing to the complement of any of the nucleotide sequences described herein.

By a polynucleotide having a nucleotide sequence at least, for example, 90-99% “identical” to a reference nucleotide sequence is intended that the nucleotide sequence of the polynucleotide be identical to the reference sequence except that the polynucleotide sequence can include up to about 10 to 1 point mutations, additions, or deletions per each 100 nucleotides of the reference nucleotide sequence.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at the 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6): 6745-6763 (1986).

The polynucleotides described herein include those encoding mutations, variations, substitutions, additions, deletions, and particular examples of the polypeptides described herein. For example, guidance concerning how to make phenotypically silent amino acid substitutions is provided in Bowie, J. U. et al., “Deciphering the Message in Protein Sequences: Tolerance to Amino Acid Substitutions,” Science 247: 1306-1310 (1990), wherein the authors indicate that proteins are surprisingly tolerant of amino acid substitutions.

Another embodiment described herein is a polynucleotide vector comprising one or more nucleotide sequences described herein.

Another embodiment described herein is a cell comprising one or more nucleotide sequences described herein or a polynucleotide vector described herein.

Another embodiment described herein is a process for manufacturing one or more of the nucleotide sequence described herein or a polypeptide encoded by the nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a means for manufacturing one or more of the nucleotide sequences described herein or a polypeptide encoded by a nucleotide sequence described herein, the process comprising: transforming or transfecting a cell with a nucleic acid comprising a nucleotide sequence described herein; growing the cells; optionally isolating additional quantities of a nucleotide sequence described herein; inducing expression of a polypeptide encoded by a nucleotide sequence of described herein; isolating the polypeptide encoded by a nucleotide described herein.

Another embodiment described herein is a nucleotide sequence produced by the method or the means described herein

Another embodiment described herein is the use of an effective amount of a polypeptide encoded by one or more of the nucleotide sequences described herein.

Another embodiment described herein is a research tool comprising a nucleotide sequence described herein.

Another embodiment described herein is a reagent comprising a nucleotide sequence described herein.

rhAmpSeq™ (Integrated DNA Technology (IDT), Coralville, IA) is an RNase H2-dependent targeted amplicon sequencing technology that provides a more efficient and less error-prone method for detecting mutations in DNA, such as SNPs and insertions and deletions (indels). rhAmpSeq also provides a method for detection of DNA sequences that are altered after cleavage by a targetable endonuclease, such as CRISPR/Cas9. In the context of CRISPR/Cas9 genome editing analysis, rhAmpSeq enables precise and high accuracy quantification of on- and off-target edits, including low-frequency indels.

The rhAmpSeq technology specifically utilizes modified PCR primers containing a single RNA base and a 3′ blocking moiety (e.g., three-carbon chains (C₃ spacers)). These modified primers are activated by RNase H2, which cleaves the single RNA base within the hybridized DNA: RNA duplex, removing the disposable 3′ blocking group and allowing amplification of a target sequence using the functional/activated primer and a DNA polymerase to generate an rhAmp PCR amplicon. This mechanism enhances specificity by reducing or eliminating primer-dimer formation and non-specific amplification, even in complex multiplex reactions. A second round of PCR amplification can then be performed on the rhAmp PCR amplicons using indexing primers to generate a rhAmpSeq library. This indexing step can thus add sequencing adapters and sample-specific indexes (e.g., barcodes) to the amplicons.

In certain aspects of rhAmpSeq, the modified RNase H2-activated primers may contain greater than 10 DNA bases that are 5′ to the single RNA base and that match the target sequence, where these 5′ DNA bases ultimately form the functional/activated primer after RNase H2 cleavage. In some instances, the disposable blocking portion of the primer that is 3′ of the RNA base may contain two DNA bases that match the target sequence and flank one or more blocking groups (e.g., C3 spacers), as well as a mismatched DNA base at the terminal 3′ end that is a mismatch to the target sequence.

The rhAmpSeq technology is further described in U.S. Pat. No. 11,926,866, which is incorporated by reference herein in its entirety for such teachings.

When performing CRISPR editing for potential therapeutic or research applications, the safety and accuracy are extremely important. Also described herein are compositions and methods that provide accurate and safe CRISPR editing. For off-target nomination, an optimized method incorporates rhAmpSeq technology coupled with a data analysis pipeline. This nomination process also includes several quality control checks, like quantifying the integration rate of the tag at the intended on-target site, as well as editing a positive control site to ensure there will be appropriate sensitivity to qualify results. For off-target verification, rhAmpSeq technology coupled to proprietary analysis algorithms for classification of off-target editing and translocations with sensitivity as low as 0.1% editing frequencies. These algorithms are an improved version of the rhAmpSeq CRISPR Analysis Tool, with the addition of multiple new algorithms for statistical classification of verified off-target sites and characterization of translocation events.

S.p. Cas9 is a Cas9 variant with highly reduced off-target editing that maintains the efficiency of on-target editing. New enhancers also improve the efficiency of desired DNA repair events, like homology directed repair, without increasing off-target editing.

Most of the quality assurance challenges with CRISPR are due to the novelty of the whole system as a therapeutic modality. For instance, CRISPR is unique in the sense that mutations introduced into both gRNAs and DNA donors used for homology directed repair could have detrimental effects without proper quality control procedures. For gRNAs, mutations in the molecule could result in decreased activity, or even worse, a novel gRNA targeting new putative regions in the genome in the case of mutations within the spacer region of the molecule. For homology directed repair DNA donors, similar issues can arise, and mutations in a DNA donor can result in incorporation of unintended mutations after CRISPR gene editing in the genome of interest. To navigate this, a number of quality control assays are used for CRISPR-related oligonucleotides, spanning different analytical platforms such as ESI-MS and direct sequencing of the molecules.

Some of the most important considerations for accurately assessing the safety of CRISPR editing are standards and process controls to describe the analytical sensitivity and specificity of a method. Standards have been created to benchmark off-target nomination and validation technologies. While these standards and process controls are improved upon, it is critical to use multiple orthogonal assays to ensure that safety is being accurately assessed.

Technologies like AI and machine learning are continuing to play an important role in the genomics space including gene editing. The S.p. Cas9 on-target model implements AI technology to make sure that gRNAs chosen for experiments have high on-target editing efficiency. These types of models are also used in production processes for gene editing reagents, for example, identifying problematic motifs for oligo synthesis, providing quantitative estimation of different synthesis by-products, estimating the effects of any unintended oligo species, and more.

One embodiment described herein is a method for reducing adaptered-tag sequencing reads during the identification and nomination of on- and off-target CRISPR edited sites, the method comprising: contacting in an amplification reaction one or more adaptered-tag blocking oligonucleotides with an isolated genomic DNA having one or more tag sequences and adapter sequences; wherein the adaptered-tag blocking oligonucleotides comprise one or more blocking moieties and hybridize to adaptered-tag sequences at a junction region between the adapter and tag sequences to reduce amplification of the adaptered-tag sequences. In one aspect, the amplification reaction comprises one or more adapter-specific primers and one or more tag-specific primers to produce a first set of amplified sequences, the method further comprising: amplifying the first set of amplified sequences using universal sequencing primers targeting the tails of the tag-specific primers to produce a second set of amplified sequences; sequencing the second set of amplified sequences and obtaining sequencing data; and identifying on-/off-target CRISPR editing loci. In another aspect, the one or more tag-specific primers comprise a plurality of staggered primers, each staggered primer comprising a number of random nucleotides positioned between a tag-specific sequence portion and a universal tail sequence portion. In another aspect, the number of random nucleotides positioned between the tag-specific sequence portion and the universal tail sequence portion for each staggered primer ranges from 0 to 6. In another aspect, the one or more tag sequences comprises DNA, RNA, xeno nucleic acids, or combinations thereof. In another aspect, the one or more tag sequences comprises a double-stranded oligodeoxynucleotide tag (dsODN-tag) sequence. In another aspect, the one or more tag sequences comprises one or more modifications comprising a 5′-terminal phosphate, phosphorothioate linkages, methylphosphonate linkages, boranophosphate linkages, phosphonoacetate linkages, or combinations thereof. In another aspect, the one or more tag sequences comprises at least three phosphorothioate linkages at the 5′-terminus, 3′-terminus, or a combination thereof. In another aspect, the one or more blocking moieties of the adaptered-tag blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof. In another aspect, the adaptered-tag blocking oligonucleotides hybridize to top and bottom strands of the adaptered-tag sequences at a junction region between the adapter and tag sequences. In another aspect, the adaptered-tag blocking oligonucleotides have a sequence length of about 15 nucleotides to about 35 nucleotides. In another aspect, the adaptered-tag sequences have a sequence length of about 150 nucleotides to about 200 nucleotides. In another aspect, about 40-60% of the adaptered-tag blocking oligonucleotides hybridizes to the adapter sequence portion of the adaptered-tag sequences and about 40-60% of the adaptered-tag blocking oligonucleotides hybridizes to the tag sequence portion of the adaptered-tag sequences. In another aspect, the adaptered-tag blocking oligonucleotides reduce adaptered-tag sequencing reads by at least about 25% relative to a method without the adaptered-tag blocking oligonucleotides. In another aspect, the adaptered-tag blocking oligonucleotides increase the amount of sequencing reads at unique nominated off-target effect (OTE) sites as compared to a method without the adaptered-tag blocking oligonucleotides.

Another embodiment described herein is method for identifying and nominating on- and off-target CRISPR edited sites with improved accuracy and sensitivity, the method comprising: (a) performing a multiplex PCR reaction comprising: (i) one or more tag-specific oligonucleotide primers, each having a cleavage region comprising a ribonucleotide (rN) positioned 5′ of a blocking group and a complementary region flanking one or more tag sequences, wherein the blocking group prevents primer extension and/or inhibits the oligonucleotide primer from serving as a template for DNA synthesis; (ii) one or more adapter-specific oligonucleotide primers, each having a cleavage region comprising a rN positioned 5′ of a blocking group and a complementary region flanking the 5′ end of a universal adapter sequence; (iii) one or more adaptered-tag blocking oligonucleotides corresponding to each strand of the tag sequences and comprising one or more blocking moieties, wherein the adaptered-tag blocking oligonucleotides hybridize to top and bottom strands of adaptered-tag sequences at a junction region between the universal adapter and tag sequences and inhibit annealing of the tag-specific oligonucleotide primers to the top and bottom strands of the adaptered-tag sequences, thereby reducing amplification of the adaptered-tag sequences; and (iv) a cleaving enzyme; (b) hybridizing the tag-specific oligonucleotide primers to one or more incorporated tag sequences to form a tag sequence double stranded substrate and hybridizing one or more adapter-specific oligonucleotide primers to the 5′ end of the universal adapter sequence; (c) cleaving at a point within or adjacent to the cleavage regions with the cleaving enzyme to remove the blocking groups from the one or more tag-specific oligonucleotide primers and the one or more adapter-specific oligonucleotide primers; (d) amplifying a portion of isolated genomic DNA comprising the one or more incorporated tag sequences and the universal adapter sequence; and (e) sequencing the amplified portion of the isolated genomic DNA, thereby identifying on- and off-target CRISPR edited sites. In one aspect, the cleaving enzyme is an RNase H2 enzyme. In another aspect, the isolated genomic DNA comprising the one or more incorporated tag sequences and the universal adapter sequence is generated by: isolating genomic DNA from a cell having one or more tag sequences incorporated into a target site within a genome of the cell; and integrating a universal adapter sequence into the isolated genomic DNA. In another aspect, the universal adapter sequence comprises a unique molecular index (UMI). In another aspect, the sequencing of step (e) further comprises executing on a processor: (i) aligning sequence data to a reference genome; and (ii) outputting the alignment, analysis, and results data as custom-formatted files, tables, or graphics.

Another embodiment described herein is a method for reducing adaptered-tag sequencing reads during the identification and nomination of on- and off-target CRISPR edited sites, the method comprising: (a) co-delivering a guide sequence RNA (sgRNA) or a two-part CRISPR RNA:trans-activating crRNA (crRNA:tracrRNA) duplex, one or more tag sequences, and an RNA-guided endonuclease to cells; (b) incubating the cells for a period of time sufficient for double strand breaks to occur, and for the cells to repair the double strand breaks; (c) isolating genomic DNA from the cells, fragmenting the genomic DNA, and ligating the fragmented genomic DNA to a universal adapter sequence; (d) amplifying the ligated DNA fragments using tag-specific primers, adapter-specific primers, and blocking oligonucleotides comprising one or more blocking moieties, to produce a first set of amplified sequences; wherein the blocking oligonucleotides hybridize to top and bottom strands of adaptered-tag sequences at a junction region between the ligated adapter and tag sequences and inhibit annealing of the tag-specific primers to the top and bottom strands of the adaptered-tag sequences, thereby preventing amplification of the adaptered-tag sequences; (e) amplifying the first set of amplified sequences using universal sequencing primers targeting the tails of the tag-specific primers to produce a second set of amplified sequences; (f) sequencing the second set of amplified sequences and obtaining sequencing data; and (g) identifying on-/off-target CRISPR editing loci. In one aspect, the one or more tag sequences comprises DNA, RNA, xeno nucleic acids, or combinations thereof. In another aspect, the one or more tag sequences comprises a double-stranded oligodeoxynucleotide tag (dsODN-tag) sequence. In another aspect, the one or more tag sequences comprises one or more modifications comprising a 5′-terminal phosphate, phosphorothioate linkages, methylphosphonate linkages, boranophosphate linkages, phosphonoacetate linkages, or combinations thereof. In another aspect, the one or more tag sequences comprises at least three phosphorothioate linkages at the 5′-terminus, 3′-terminus, or a combination thereof. In another aspect, the one or more tag sequences comprises an adenine (A)-thymine (T) content of less than about 70%. In another aspect, the one or more tag sequences comprises an A-T content of less than about 50%. In another aspect, the one or more tag sequences comprises a guanine (G)-cytosine (C) content of about 30% to about 60%. In another aspect, the one or more blocking moieties of the blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof. In another aspect, the blocking oligonucleotides comprise DNA, locked nucleic acids (LNA), or combinations thereof. In another aspect, the blocking oligonucleotides have a sequence length of about 15 nucleotides to about 35 nucleotides. In another aspect, about 40-60% of the sequence of the blocking oligonucleotides hybridizes to the ligated adapter sequence portion of the adaptered-tag sequences and about 40-60% of the sequence of the blocking oligonucleotides hybridizes to the ligated tag sequence portion of the adaptered-tag sequences. In another aspect, the blocking oligonucleotides are present at a concentration of about 250 nM to about 2500 nM. In another aspect, the adaptered-tag sequences have a sequence length of about 150 nucleotides to about 200 nucleotides. In another aspect, the blocking oligonucleotides reduce adaptered-tag sequencing reads by at least about 25% as compared to a method without the blocking oligonucleotides. In another aspect, the blocking oligonucleotides increase the amount of sequencing reads at unique nominated off-target effect (OTE) sites as compared to a method without the blocking oligonucleotides. In another aspect, the blocking oligonucleotides do not inhibit the amplification of ligated tag sequences inserted in the genomic DNA. In another aspect, step (g) comprises executing on a processor: (i) aligning the sequence data to a reference genome; (ii) identifying on-/off-target CRISPR editing loci; and (iii) outputting the alignment, analysis, and results data as files, tables, or graphics. In another aspect, the method further comprises a step following step (e) comprising: (e1) normalizing the second set of amplified sequences to produce concentration normalized libraries, pooling the normalized libraries with other samples to produce pooled libraries; and continuing with steps (f)-(g). In another aspect, the sgRNA or crRNA comprises one or more modifications comprising phosphorothioate linkages, 2′-O-methyl (2′-OME) nucleotides, 2′-O-methoxy-ethyl (2′-MOE) nucleotides, 2′-F nucleotides, locked nucleic acids (LNA), or combinations thereof. In another aspect, the RNA-guided endonuclease comprises an endogenously-expressed Cas enzyme, a Cas expression vector, a Cas protein or RNP complex, or a Cas mRNA. In another aspect, the cells comprise mammalian cells. In another aspect, the cells comprise human cells or mouse cells. In another aspect, the period of time is about 24 hours to about 96 hours. In another aspect, multiple tag sequences are co-delivered.

Another embodiment described herein is a method for identifying and nominating on- and off-target CRISPR edited sites with improved accuracy and sensitivity, the process comprising the steps of: (a) co-delivering a guide sequence RNA (sgRNA) or a two-part CRISPR RNA:trans-activating crRNA (crRNA:tracrRNA) duplex, one or more double-stranded oligodeoxyribonucleotide tag sequences comprising less two or more phosphorothioates at the 3′-termini and less than 50% adenine (A) and thymine (T) content, and an RNA-guided endonuclease to cells; (b) incubating the cells for a period of time sufficient for double strand breaks to occur; (c) isolating genomic DNA from the cells, fragmenting the genomic DNA, and ligating the fragmented genomic DNA to a unique molecular index containing a universal adapter sequence; (d) amplifying the ligated DNA fragments using a tag-specific primer with a universal adapter-specific primer to produce a first set of amplified sequences, wherein the tag-specific primer comprises a 5′-universal tail sequence, a locus specific segment, a ribonucleotide 6-nucleotides from the 3′-end, a 3′-end mismatch, and a 3′-end blocker such that treatment with RNase H2 cleaves the 3′-blocker to reduce non-specific hybridization and primer dimerization; (e) amplifying the first set of amplified sequences using universal sequencing primers targeting the tails of the primers targeting the tag and universal adapter sequences to produce a second set of amplified sequences, wherein the second set of amplified sequences comprise sample indexes for sequencing, and (f) sequencing the pooled sequences and obtaining sequencing data; and (g) identifying on-/off-target CRISPR editing loci.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A method for reducing adaptered-tag sequencing reads during the identification and nomination of on- and off-target CRISPR edited sites, the method comprising:
   contacting in an amplification reaction one or more adaptered-tag blocking oligonucleotides with an isolated genomic DNA having one or more tag sequences and adapter sequences;
   wherein the adaptered-tag blocking oligonucleotides comprise one or more blocking moieties and hybridize to adaptered-tag sequences at a junction region between the adapter and tag sequences to reduce amplification of the adaptered-tag sequences.
Clause 2. The method of clause 1, wherein the amplification reaction comprises one or more adapter-specific primers and one or more tag-specific primers to produce a first set of amplified sequences, the method further comprising:
   amplifying the first set of amplified sequences using universal sequencing primers targeting the tails of the tag-specific primers to produce a second set of amplified sequences;
   sequencing the second set of amplified sequences and obtaining sequencing data; and
   identifying on-/off-target CRISPR editing loci.
Clause 3. The method of clause 1 or 2, wherein the one or more tag-specific primers comprise a plurality of staggered primers, each staggered primer comprising a number of random nucleotides positioned between a tag-specific sequence portion and a universal tail sequence portion.
Clause 4. The method of any one of clauses 1-3, wherein the number of random nucleotides positioned between the tag-specific sequence portion and the universal tail sequence portion for each staggered primer ranges from 0 to 6.
Clause 5. The method of any one of clauses 1-4, wherein the one or more tag sequences comprises DNA, RNA, xeno nucleic acids, or combinations thereof.
Clause 6. The method of any one of clauses 1-5, wherein the one or more tag sequences comprises a double-stranded oligodeoxynucleotide tag (dsODN-tag) sequence.
Clause 7. The method of any one of clauses 1-6, wherein the one or more tag sequences comprises one or more modifications comprising a 5′-terminal phosphate, phosphorothioate linkages, methylphosphonate linkages, boranophosphate linkages, phosphonoacetate linkages, or combinations thereof.
Clause 8. The method of any one of clauses 1-7, wherein the one or more tag sequences comprises at least three phosphorothioate linkages at the 5′-terminus, 3′-terminus, or a combination thereof.
Clause 9. The method of any one of clauses 1-8, wherein the one or more blocking moieties of the adaptered-tag blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof.
Clause 10. The method of any one of clauses 1-9, wherein the adaptered-tag blocking oligonucleotides hybridize to top and bottom strands of the adaptered-tag sequences at a junction region between the adapter and tag sequences.
Clause 11. The method of any one of clauses 1-10, wherein the adaptered-tag blocking oligonucleotides have a sequence length of about 15 nucleotides to about 35 nucleotides.
Clause 12. The method of any one of clauses 1-11, wherein the adaptered-tag sequences have a sequence length of about 150 nucleotides to about 200 nucleotides.
Clause 13. The method of any one of clauses 1-12, wherein about 40-60% of the adaptered-tag blocking oligonucleotides hybridizes to the adapter sequence portion of the adaptered-tag sequences and about 40-60% of the adaptered-tag blocking oligonucleotides hybridizes to the tag sequence portion of the adaptered-tag sequences.
Clause 14. The method of any one of clauses 1-13, wherein the adaptered-tag blocking oligonucleotides reduce adaptered-tag sequencing reads by at least about 25% relative to a method without the adaptered-tag blocking oligonucleotides.
Clause 15. The method of any one of clauses 1-14, wherein the adaptered-tag blocking oligonucleotides increase the amount of sequencing reads at unique nominated off-target effect (OTE) sites as compared to a method without the adaptered-tag blocking oligonucleotides.
Clause 16. A method for identifying and nominating on- and off-target CRISPR edited sites with improved accuracy and sensitivity, the method comprising:
   (a) performing a multiplex PCR reaction comprising:
      (i) one or more tag-specific oligonucleotide primers, each having a cleavage region comprising a ribonucleotide (rN) positioned 5′ of a blocking group and a complementary region flanking one or more tag sequences, wherein the blocking group prevents primer extension and/or inhibits the oligonucleotide primer from serving as a template for DNA synthesis;
      (ii) one or more adapter-specific oligonucleotide primers, each having a cleavage region comprising a rN positioned 5′ of a blocking group and a complementary region flanking the 5′ end of a universal adapter sequence;
      (iii) one or more adaptered-tag blocking oligonucleotides corresponding to each strand of the tag sequences and comprising one or more blocking moieties, wherein the adaptered-tag blocking oligonucleotides hybridize to top and bottom strands of adaptered-tag sequences at a junction region between the universal adapter and tag sequences and inhibit annealing of the tag-specific oligonucleotide primers to the top and bottom strands of the adaptered-tag sequences, thereby reducing amplification of the adaptered-tag sequences; and
      (iv) a cleaving enzyme;
   (b) hybridizing the tag-specific oligonucleotide primers to one or more incorporated tag sequences to form a tag sequence double stranded substrate and hybridizing one or more adapter-specific oligonucleotide primers to the 5′ end of the universal adapter sequence;
   (c) cleaving at a point within or adjacent to the cleavage regions with the cleaving enzyme to remove the blocking groups from the one or more tag-specific oligonucleotide primers and the one or more adapter-specific oligonucleotide primers;
   (d) amplifying a portion of isolated genomic DNA comprising the one or more incorporated tag sequences and the universal adapter sequence; and
   (e) sequencing the amplified portion of the isolated genomic DNA, thereby identifying on- and off-target CRISPR edited sites.
Clause 17. The method of clause 16, wherein the cleaving enzyme is an RNase H2 enzyme.
Clause 18. The method of clause 16 or 17, wherein the isolated genomic DNA comprising the one or more incorporated tag sequences and the universal adapter sequence is generated by:
   isolating genomic DNA from a cell having one or more tag sequences incorporated into a target site within a genome of the cell; and
   integrating a universal adapter sequence into the isolated genomic DNA.
Clause 19. The method of any one of clauses 16-18, wherein the universal adapter sequence comprises a unique molecular index (UMI).
Clause 20. The method of any one of clauses 16-19, wherein the sequencing of step (e) further comprises executing on a processor:
   (i) aligning sequence data to a reference genome; and
   (ii) outputting the alignment, analysis, and results data as custom-formatted files, tables, or graphics.
Clause 21. A method for reducing adaptered-tag sequencing reads during the identification and nomination of on- and off-target CRISPR edited sites, the method comprising:
   (a) co-delivering a guide sequence RNA (sgRNA) or a two-part CRISPR RNA:trans-activating crRNA (crRNA:tracrRNA) duplex, one or more tag sequences, and an RNA-guided endonuclease to cells;
   (b) incubating the cells for a period of time sufficient for double strand breaks to occur, and for the cells to repair the double strand breaks;
   (c) isolating genomic DNA from the cells, fragmenting the genomic DNA, and ligating the fragmented genomic DNA to a universal adapter sequence;
   (d) amplifying the ligated DNA fragments using tag-specific primers, adapter-specific primers, and blocking oligonucleotides comprising one or more blocking moieties, to produce a first set of amplified sequences;
      wherein the blocking oligonucleotides hybridize to top and bottom strands of adaptered-tag sequences at a junction region between the ligated adapter and tag sequences and inhibit annealing of the tag-specific primers to the top and bottom strands of the adaptered-tag sequences, thereby preventing amplification of the adaptered-tag sequences;
   (e) amplifying the first set of amplified sequences using universal sequencing primers targeting the tails of the tag-specific primers to produce a second set of amplified sequences;
   (f) sequencing the second set of amplified sequences and obtaining sequencing data; and
   (g) identifying on-/off-target CRISPR editing loci.
Clause 22. The method of clause 21, wherein the one or more tag sequences comprises DNA, RNA, xeno nucleic acids, or combinations thereof.
Clause 23. The method of clause 21 or 22, wherein the one or more tag sequences comprises a double-stranded oligodeoxynucleotide tag (dsODN-tag) sequence.
Clause 24. The method of any one of clauses 21-23, wherein the one or more tag sequences comprises one or more modifications comprising a 5′-terminal phosphate, phosphorothioate linkages, methylphosphonate linkages, boranophosphate linkages, phosphonoacetate linkages, or combinations thereof.
Clause 25. The method of any one of clauses 21-24, wherein the one or more tag sequences comprises at least three phosphorothioate linkages at the 5′-terminus, 3′-terminus, or a combination thereof.
Clause 26. The method of any one of clauses 21-25, wherein the one or more tag sequences comprises an adenine (A)-thymine (T) content of less than about 70%.
Clause 27. The method of any one of clauses 21-26, wherein the one or more tag sequences comprises an A-T content of less than about 50%.
Clause 28. The method of any one of clauses 21-27, wherein the one or more tag sequences comprises a guanine (G)-cytosine (C) content of about 30% to about 60%.
Clause 29. The method of any one of clauses 21-28, wherein the one or more blocking moieties of the blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof.
Clause 30. The method of any one of clauses 21-29, wherein the blocking oligonucleotides comprise DNA, locked nucleic acids (LNA), or combinations thereof.
Clause 31. The method of any one of clauses 21-30, wherein the blocking oligonucleotides have a sequence length of about 15 nucleotides to about 35 nucleotides.
Clause 32. The method of any one of clauses 21-31, wherein about 40-60% of the sequence of the blocking oligonucleotides hybridizes to the ligated adapter sequence portion of the adaptered-tag sequences and about 40-60% of the sequence of the blocking oligonucleotides hybridizes to the ligated tag sequence portion of the adaptered-tag sequences.
Clause 33. The method of any one of clauses 21-32, wherein the blocking oligonucleotides are present at a concentration of about 250 nM to about 2500 nM.
Clause 34. The method of any one of clauses 21-33, wherein the adaptered-tag sequences have a sequence length of about 150 nucleotides to about 200 nucleotides.
Clause 35. The method of any one of clauses 21-34, wherein the blocking oligonucleotides reduce adaptered-tag sequencing reads by at least about 25% as compared to a method without the blocking oligonucleotides.
Clause 36. The method of any one of clauses 21-35, wherein the blocking oligonucleotides increase the amount of sequencing reads at unique nominated off-target effect (OTE) sites as compared to a method without the blocking oligonucleotides.
Clause 37. The method of any one of clauses 21-36, wherein the blocking oligonucleotides do not inhibit the amplification of ligated tag sequences inserted in the genomic DNA.
Clause 38. The method of any one of clauses 21-37, wherein step (g) comprises executing on a processor:
   (i) aligning the sequence data to a reference genome;
   (ii) identifying on-/off-target CRISPR editing loci; and
   (iii) outputting the alignment, analysis, and results data as files, tables, or graphics.
Clause 39. The method of any one of clauses 21-38, further comprising a step following step (e) comprising:
   (e1) normalizing the second set of amplified sequences to produce concentration normalized libraries, pooling the normalized libraries with other samples to produce pooled libraries; and continuing with steps (f)-(g).
Clause 40. The method of any one of clauses 21-39, wherein the sgRNA or crRNA comprises one or more modifications comprising phosphorothioate linkages, 2′-O-methyl (2′-OME) nucleotides, 2′-O-methoxy-ethyl (2′-MOE) nucleotides, 2′-F nucleotides, locked nucleic acids (LNA), or combinations thereof.
Clause 41. The method of any one of clauses 21-40, wherein the RNA-guided endonuclease comprises an endogenously-expressed Cas enzyme, a Cas expression vector, a Cas protein or RNP complex, or a Cas mRNA.
Clause 42. The method of any one of clauses 21-41, wherein the cells comprise mammalian cells.
Clause 43. The method of any one of clauses 21-42, wherein the cells comprise human cells or mouse cells.
Clause 44. The method of any one of clauses 21-43, wherein the period of time is about 24 hours to about 96 hours.
Clause 45. The method of any one of clauses 21-44, wherein multiple tag sequences are co-delivered.

### EXAMPLES

### Example 1

Assessment of 1^{st} Generation Adaptered-Tag Blocking Oligos Via qPCR

Adaptered-tag sequences for dsODN CTL216 were ordered as Ultramers for qPCR (SEQ ID NO: 1-2) to mimic the adaptered-tag sequence generated during CTL-seq library preparation. DNA/LNA blocking oligos with 3′-C₃ spacers were designed to test three variables: oligo length, Tₘ ° C., and LNA placement (SEQ ID NO: 3-14), see Table 1. Inhibition of adaptered-tag amplification was tested in a qPCR EvaGreen assay using the IDT 2× PrimeTime Mastermix (Catalog #1055772), adaptered-tag Ultramer sequences as template with ˜1×10⁶ copies/reaction (SEQ ID NO: 1-2), top and bottom dsODN-tag specific primers (SEQ ID NO: 15-16), P5 adapter primer (SEQ ID NO: 17), and included reactions with and without blocking oligos with a dose titration (SEQ ID NO: 3-14). Reactions were run on the QuantStudio 7 Flex and blocking activity measured by ΔCₜ=Cₜ (Control, without blocker)−Cₜ (Control, with blocker) (FIG. 3). For the top strand adaptered-tag amplification blocking, all blocking oligos aside from SEQ ID NO: 3 led to decreased adaptered-tag amplification in a dose dependent manner. Increasing the Tₘ ° C. and LNA count significantly increased the blocking activity to as low as ΔCₜ=−10, or ˜1000-fold reduction in adaptered-tag copy number. The bottom strand adaptered-tag amplification did not show the same level of blocking activity as the top strand. Only SEQ ID NO: 14 with the highest Tₘ ° C. and LNA count had a significant impact on adaptered-tag amplification with ΔCₜ>−9. Overall, this shows that adaptered-tag amplification can be blocked and the interplay between Tₘ ° C. and LNA count plays a significant role in the effectiveness of the blocker.

TABLE 1
Oligonucleotide Sequences

| SEQ ID | | |
|---|---|---|
| NO: | Name | Sequence (5′→3′) |
| 1 | CTL216_Adapter_Tag_ | AATGATACGGCGACCACCGAGATCTACACCTGAGATCCCTTGTAGACAC |
| | Top | TCTTTCCCTACACGACGCTCTTCCGATCTTAAGCGGCGTAGGTAGCCGG |
| | | ACGAATGTCGGTCGTAGTTAGATCGGAAGAGC*C*A |
| 2 | CTL216_Adapter_Tag_ | AATGATACGGCGACCACCGAGATCTACACCTGAGATCCCTTGTAGACAC |
| | Bot | TCTTTCCCTACACGACGCTCTTCCGATCTAACTACGACCGACATTCGTC |
| | | CGGCTACCTACGCCGCTTAAGATCGGAAGAGC*C*A |
| 3 | negTopBlock_CTL216 | GTCGTAGTTAGATCGGAA/3SpC3/ |
| 4 | TopBlockL_CTL216 | GTCGGTCGTAGTTAGATCGGAAGAGCG/3SpC3/ |
| 5 | TopBlock_CTL216v3 | ATGTCGGTCGTAGTTAGATCGGAAGAGCGT/3SpC3/ |
| 6 | TopBlock_CTL216v2L1 | G+TCGGTCGTAGTTAGATCGGAAGAG+CG/3SpC3/ |
| 7 | TopBlock_CTL216v2L2 | G+T+CGGTCGTAGTTAGATCGGAAGA+G+CG/3SpC3/ |
| 8 | TopBlock_CTL216v2L3 | G+T+C+GGTCGTAGTTAGATCGGAAG+A+G+CG/3SpC3/ |
| 9 | negBotBlock_CTL216 | CGCCGCTTAAGATCGGAA/3SpC3/ |
| 10 | BotBlockL_CTL216 | CGCCGCTTAAGATCGGAAGAGC/3SpC3/ |
| 11 | BotBlockL_CTL216v2 | CCTACGCCGCTTAAGATCGGAAGAGCG/3SpC3/ |
| 12 | BotBlock_CTL216v2L1 | C+CTACGCCGCTTAAGATCGGAAGAG+CG/3SpC3/ |
| 13 | BotBlock_CTL216v2L2 | C+C+TACGCCGCTTAAGATCGGAAGA+G+CG/3SpC3/ |
| 14 | BotBlock_CTL216v2L3 | C+C+T+A+CGCCGCTTAAGATCGGAAG+A+G+CG/3SpC3/ |
| 15 | CTL216_For_dna | TAGCCGGACGAATGTCGGTCGT |
| 16 | CTL216_Rev_dna | GACATTCGTCCGGCTACCTACG |
| 17 | P5_2 | AATGATACGGCGACCACCGAGATCTACAC |
| 18 | AR_CTL216_Pos_Con | AATGATACGGCGACCACCGAGATCTACACCTGAGATCNNWNNWNNACAC |
| | | TCTTTCCCTACACGACGCTCTTCCGATCTACTCAGCAGTATCTTCAGTG |
| | | CTCTTGCCTGCGCTGTCGTCTAGCAGAGAACCTTTGCATTCGGCCAATG |
| | | GGGCACAAGGAGTGGGACGCACAGCGGGTGGAACTCCCAAAAGTGGGGC |
| | | GTACATGCAATCCCCCCGAAGCTGTTCCCCTGAACTACGACCGACATTC |
| | | GTCCGGCTACCTACGCCGCTTAGACTCAGATGCTCCAACGCCTCCACAC |
| | | CCAGGCCCATGGACACCGACACTGCCTTACACAACTCCTTGGCGTTGTC |
| | | AGAAATGGTCGAAGTGCCCCCTAAGTAATTGTCCTTGGAGGAAGTGGGA |
| | | GCCCCCGAGGCCTCCCTCGCTCTCCAGATCGGAAGAGCGTCGTGTAGGG |
| | | AAAGAGTGTNNWNNWNNGATCTCAGGTGTAGATCTCGGTGGTCGCCGTA |
| | | TCATT |
| 19 | AR_CTL064_Pos_Con | AATGATACGGCGACCACCGAGATCTACACCTGAGATCNNWNNWNNACAC |
| | | TCTTTCCCTACACGACGCTCTTCCGATCTGGAGAGCGAGGGAGGCCTCG |
| | | GGGGCTCCCACTTCCTCCAAGGACAATTACTTAGGGGGCACTTCGACCA |
| | | TTTCTGACAACGCCAAGGAGTTGTGTAAGGCAGTGTCGGTGTCCATGGG |
| | | CCTGGGTGTGGAGGCGTTGGAGCATCTGAGTCAGCACGCCCGACAAGTA |
| | | CGCCGGTTAGTGGTCCGTCGGCCAGGGGAACAGCTTCGGGGGGATTGCA |
| | | TGTACGCCCCACTTTTGGGAGTTCCACCCGCTGTGCGTCCCACTCCTTG |
| | | TGCCCCATTGGCCGAATGCAAAGGTTCTCTGCTAGACGACAGCGCAGGC |
| | | AAGAGCACTGAAGATACTGCTGAGTAGATCGGAAGAGCGTCGTGTAGGG |
| | | AAAGAGTGTNNWNNWNNGATCTCAGGTGTAGATCTCGGTGGTCGCCGTA |
| | | TCATT |
| 20 | Con_Probe_CTL216_ | /56-FAM/TGAGATCCC/ZEN/TTGTAGACACTCTTTCCCTAC/ |
| | P5_v2 | 3IABkFQ/ |
| 21 | CTL216_Top_Probe | /5HEX/TTTGGGAGT/ZEN/TCCACCCGCTGT/3IABkFQ/ |
| | Set 2 PRB | |
| 22 | CTL216_Bot_Probe_ | /5Cy5/ACACCGACA/TAO/CTGCCTTACACAACT/3IAbRQSp/ |
| | Cy5 | |
| 23 | P5_rh | AATGATACGGCGACCACCGAGATrCTACAT/3SpC3/ |
| 24 | CTLc216_FWD | CATAGCGGTATTACGCGAGATTACGATAGCCGGACGAATGTCGrGTCGT |
| | | T/3SpC3/ |
| 25 | CTL216_REV_v3 | CATAGCGGTATTACGCGAGATTACGAACATTCGTCCGGCTACCTrACGC |
| | | CC/3SpC3/ |
| 26 | CTL064_Top_rhPCR1 | CATAGCGGTATTACGCGAGATTACGATACGCCGGTTAGTGGTrCCGTCC |
| | | /3SpC3/ |
| 27 | CTL064_Bot_rhPCR1 | CATAGCGGTATTACGCGAGATTACGATAACCGGCGTACTTGTCGrGGCG |
| | | TC/3SpC3/ |
| 28 | CTL216T_v1 | GTCGGTCGTAGTTAGATCGGAAGAGC/3SpC3/ |
| 29 | CTL216T_v2 | G+TCGGTCGTAGTTAGATCGGAAGA+GC/3SpC3/ |
| 30 | CTL216T_v3 | G+TCGGTC+GTAGTTAGATCGGAAG+A+GC/3SpC3/ |
| 31 | CTL216T_v4 | G+TCGGTC+G+TAGTTAGATCGGAA+G+A+GC/3SpC3/ |
| 32 | CTL216T_v5 | G+TCGGTC+G+T+AGTTAGATCGGA+A+G+A+GC/3SpC3/ |
| 33 | CTL216T_v6 | G+TCGGTC+G+T+A+GTTAGATCGG+A+A+G+A+GC/3SpC3/ |
| 34 | CTL216T_v7 | G+TCGGTC+G+T+A+G+TTAGATCG+G+A+A+G+A+GC/3SpC3/ |
| 35 | CTL216T_v8 | G+TCGGTC+G+T+A+G+T+T+AGATCG+G+A+A+G+A+G+C/3SpC3/ |
| 36 | CTL216B_v1 | TACCTACGCCGCTTAAGATCGGAAGAGC/3SpC3/ |
| 37 | CTL216B_v2 | T+ACCTACGCCGCTTAAGATCGGAAGA+GC/3SpC3/ |
| 38 | CTL216B_v3 | T+A+CCTACGCCGCTTAAGATCGGAAG+A+GC/3SpC3/ |
| 39 | CTL216B_v4 | T+A+C+CTACGCCGCTTAAGATCGGAA+G+A+GC/3SpC3/ |
| 40 | CTL216B_v5 | T+A+C+C+TACGCCGCTTAAGATCGGA+A+G+A+GC/3SpC3/ |
| 41 | CTL216B_v6 | T+A+C+C+T+ACGCCGCTTAAGATCGG+A+A+G+A+GC/3SpC3/ |
| 42 | CTL216B_v7 | T+A+C+C+T+A+CGCCGCTTAAG+ATCGG+A+A+G+A+GC/3SpC3/ |
| 43 | CTL216B_v8 | T+A+C+C+T+A+CGCCGCT+TAAG+A+TCGG+A+A+G+A+GC/3SpC3/ |
| 44 | CTL064T_v1 | AGTGGTCCGTCGGCAGATCGGAAGAGCG/3SpC3/ |
| 45 | CTL064T_v2 | A+GTGGTCCGTCGGCAGATCGGAAGAG+CG/3SpC3/ |
| 46 | CTL064T_v3 | A+G+TGGTCCGTCGGCAGATCGGAAGA+G+CG/3SpC3/ |
| 47 | CTL064T_v4 | A+G+TGGTCCGTCGGCAGATCGGAAG+A+G+CG/3SpC3/ |
| 48 | CTL064T_v5 | A+G+T+GGTCCGTCGGCAGATCGGAAG+A+G+CG/3SpC3/ |
| 49 | CTL064T_v6 | A+G+T+G+GTCCGTCGGCAGATCGGAAG+A+G+CG/3SpC3/ |
| 50 | CTL064T_v7 | A+G+T+G+GTCCGTCGGCAGATCGGAA+G+A+G+CG/3SpC3/ |
| 51 | CTL064T_v8 | A+G+T+G+GTCCGTCGGCAGATCGGA+A+G+A+G+CG/3SpC3/ |
| 52 | CTL064B_v1 | TGTCGGGCGTGCTAGATCGGAAGAGC/3SpC3/ |
| 53 | CTL064B_v2 | T+GTCGGGCGTGCTAGATCGGA+AGAGC/3SpC3/ |
| 54 | CTL064B_v3 | T+G+TCGGGCGTGCTAGATCGG+A+AGAGC/3SpC3/ |
| 55 | CTL064B_v4 | T+G+T+CGGGCGTGCTAGATCG+G+A+AGAGC/3SpC3/ |
| 56 | CTL064B_v5 | T+G+T+C+GGGCGTGCTAGATC+G+G+A+AGAGC/3SpC3/ |
| 57 | CTL064B_v6 | T+G+T+C+GGGCGTGCTAG+ATC+G+G+A+AGAGC/3SpC3/ |
| 58 | CTL064B_v7 | T+G+T+C+G+GGCGTGCTAG+ATC+G+G+A+AGAGC/3SpC3/ |
| 59 | CTL064B_v8 | T+G+T+C+G+GGCGTGCTA+G+ATC+G+G+A+AGAGC/3SpC3/ |
| 60 | TopBlock_CTL064 | TAGTGGTCCGTCGGCAGATCGGAAGAGCGT/3ddC/ |
| 61 | BottomBlock_CTL064 | CTTGTCGGGCGTGCTAGATCGGAAGAGCGT/3ddC/ |
| 62 | CTL064_Adapter_Tag_ | AATGATACGGCGACCACCGAGATCTACACCTGAGATCCCTTGTAGACAC |
| | Top | TCTTTCCCTACACGACGCTCTTCCGATCTAGCACGCCCGACAAGTACGC |
| | | CGGTTAGTGGTCCGTCGGCAGATCGGAAGAGC*C*A |
| 63 | CTL064_Adapter_Tag_ | AATGATACGGCGACCACCGAGATCTACACCTGAGATCCCTTGTAGACAC |
| | Bot | TCTTTCCCTACACGACGCTCTTCCGATCTGCCGACGGACCACTAACCGG |
| | | CGTACTTGTCGGGCGTGCTAGATCGGAAGAGC*C*A |
| 64 | CTL39_216T_sHPLC | /5Phos/T*A*A*GCGGCGTAGGTAGCCGGACGAATGTCGGTCGTA*G* |
| | | T*T |
| 65 | CTL39_216B_sHPLC | /5Phos/A*A*C*TACGACCGACATTCGTCCGGCTACCTACGCCGC*T* |
| | | T*A |
| 66 | CTL064_Top | /5Phos/A*G*C*ACGCCCGACAAGTACGCCGGTTAGTGGTCCGTC*G* |
| | | G*C |
| 67 | CTL064_Bottom | /5Phos/G*C*C*GACGGACCACTAACCGGCGTACTTGTCGGGCGT*G* |
| | | C*T |
| 68 | AR_sgRNA_XT | rGrUrUrGrGrArGrCrArUrCrUrGrArGrUrCrCrArGrGrUrUrUr |
| | | UrArGrArGrCrUrArGrArArArUrArGrCrArArGrUrUrArArArA |
| | | rUrArArGrGrCrUrArGrUrCrCrGrUrUrArUrCrArArCrUrUrGr |
| | | ArArArArArGrUrGrGrCrArCrCrGrArGrUrCrGrGrUrGrCrUrU |
| | | rUrU |
| 69 | EMX1_sgRNA_XT | rGrArGrUrCrCrGrArGrCrArGrArArGrArArGrArArGrUrUrUr |
| | | UrArGrArGrCrUrArGrArArArUrArGrCrArArGrUrUrArArArA |
| | | rUrArArGrGrCrUrArGrUrCrCrGrUrUrArUrCrArArCrUrUrGr |
| | | ArArArArArGrUrGrGrCrArCrCrGrArGrUrCrGrGrUrGrCrUrU |
| | | rUrU |
| 70 | AVS1_sgRNA_XT | rGrGrGrGrCrCrArCrUrArGrGrGrArCrArGrGrArUrGrUrUrUr |
| | | UrArGrArGrCrUrArGrArArArUrArGrCrArArGrUrUrArArArA |
| | | rUrArArGrGrCrUrArGrUrCrCrGrUrUrArUrCrArArCrUrUrGr |
| | | ArArArArArGrUrGrGrCrArCrCrGrArGrUrCrGrGrUrGrCrUrU |
| | | rUrU |
| 71 | LAG3_9_sgRNA_XT4 | rGrArArGrGrCrUrGrArGrArUrCrCrUrGrGrArGrGrGrUrUrUr |
| | | UrArGrArGrCrUrArGrArArArUrArGrCrArArGrUrUrArArArA |
| | | rUrArArGrGrCrUrArGrUrCrCrGrUrUrArUrCrArArCrUrUrGr |
| | | ArArArArArGrUrGrGrCrArCrCrGrArGrUrCrGrGrUrGrCrUrU |
| | | rUrU |
| 72 | P5 Adapter | AATGATACGGCGACCACCGAGATCTACACNNNNNNNNNNWNNWNNACAC |
| | | TCTTTCCCTACACGACGCTCTTCCGATC*T |
| 73 | P5 Common Adapter | /5Phos/GATCGGAAGAGC*C*A |
| 74 | i7_H3 | CAAGCAGAAGACGGCATACGAGATNNNNNNNNGGCAGTCGGTGATCATA |
| | | GCGGTATTACGCGAGATTACGA |
| 75 | CTLH3_Index1_v2 | TCGTAATCTCGCGTAATACCGCTATGATCACCGACTGCC |
| 76 | CTLH3_Read2_v2 | GGCAGTCGGTGATCATAGCGGTATTACGCGAGATTACGA |

All sequences are shown 5′→3′. All oligonucleotides were synthesized by IDT (Coralville, IA). Abbreviations used in the sequences above are: N indicates any nucleotide - A, C, G, T; W indicates A or T; “rN” indicates a ribonucleotide, where N is the nucleotide preceeded by the “r”; /5Phos/indicates a 5′-terminal phosphate; * indicates a phosphorothioate linkage between the two nucleotides; +N indicates a locked nucleotide having a methylene bond between the 2′ oxygen and the 4′ carbon of the pentose ring, where N is the nucleotide preceeded by the “+”; /3SpC3/indicates a 3′-terminal C3 spacer; /56-FAM/indicates a 5′-terminal 6-FAM (Fluorescein) fluorophore; /ZEN/indicates an internal ZEN™ fluorescence
quencher; /3IABkFQ/indicates a 3′-terminal Iowa Black® FQ fluorescence quencher; /5HEX/indicates a 5′-terminal HEX fluorophore (Hexachlorofluorescein); /5Cy5/indicates a 5′-terminal Cy5™ (Cyanine 5) fluorophore; /TAO/indicates an internal TAO™ fluorescence quencher; /31AbRQSp/indicates a 3′-terminal Iowa Black® RQ fluorescence quencher; /3ddC/indicates a 3′-terminal dideoxycytidine.

Assessment of 2^{nd} Generation Adaptered-Tag Blocking Oligos Via qPCR

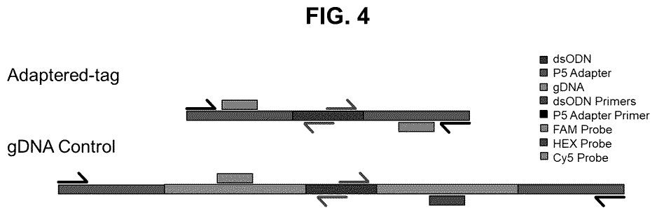

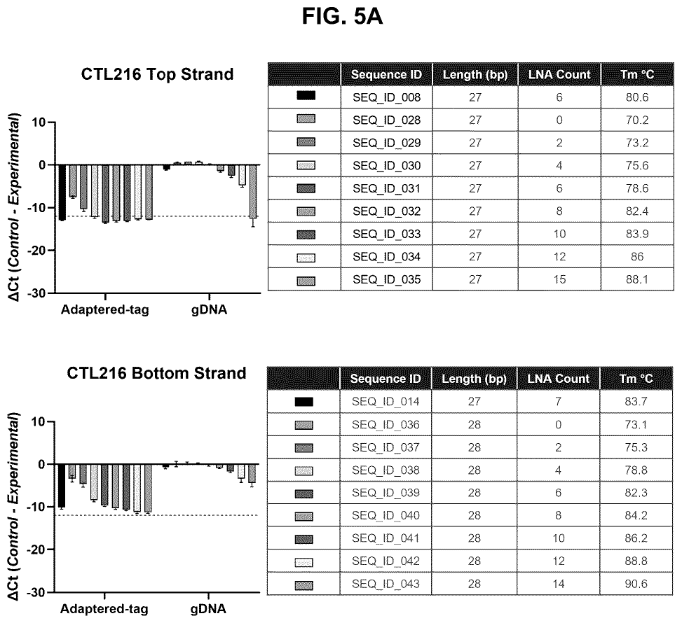

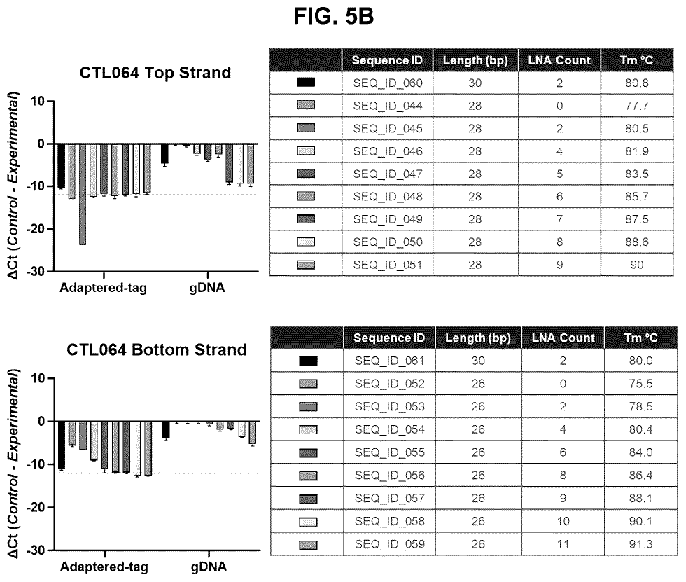

To depict experimental conditions more accurately and to ensure blocking oligos do not disrupt amplification from actual genomic sites during CTL-seq NGS library preparation, a synthetic gDNA control was constructed with a dsODN-tag inserted into the AR on-target site locus (SEQ ID NO: 18-19) (FIG. 4). Three different colored probes were designed to distinguish between adaptered-tag amplification (FAM), gDNA top strand amplification (HEX), and gDNA bottom strand amplification (Cy5) (SEQ ID NO: 20-22). In addition to the use of a gDNA control, an additional dsODN-tag, and its corresponding blocking oligos with a larger LNA count and Tₘ° C. range were tested to obtain maximum adaptered-tag blocking while retaining the ability to amplify the gDNA control. Inhibition of adaptered-tag amplification was tested with 4× rhAmpSeq PCR1 Mastermix, adaptered-tag Ultramer sequences as template with ˜1×10⁶ copies/reaction (SEQ ID NO: 1, SEQ ID NO: 2, SEQ ID NO: 62-63), gDNA control sequence as a template with ˜1×10⁵ copies/reaction (SEQ ID NO: 18-19), top and bottom rhAmpSeq dsODN-tag specific primers (SEQ ID NO: 24-27), rhAmpSeq P5 adapter primer (SEQ ID NO: 23), and included reactions with and without blocking oligos (SEQ ID NO: 8, SEQ ID NO: 14, SEQ ID NO: 28-61). Reactions were run on the QuantStudio 7 Flex and blocking activity measured for both adaptered-tag amplification and the gDNA control by ΔCₜ=Cₜ (Control, without blocker)−Cₜ (Control, with blocker). For both dsODN-tags, each successive addition to the LNA count and corresponding Tₘ ° C. increase to the blocking oligos led to decreased adaptered-tag amplification with both the top and bottom strands (FIG. 5). Moreover, the blocking oligos with Tₘ ° C. above 82° C. start to decrease amplification of the gDNA control. This highlights the need to balance the Tₘ ° C. of the blocking oligos to ensure the specificity of the blocking oligo to inhibit adaptered-tag amplification and not dsODN-tags inserted into genomic loci.

Assessment of 2^{nd} Generation Adaptered-Tag Blocking Oligos with CTL-Seq NGS Library Preparation

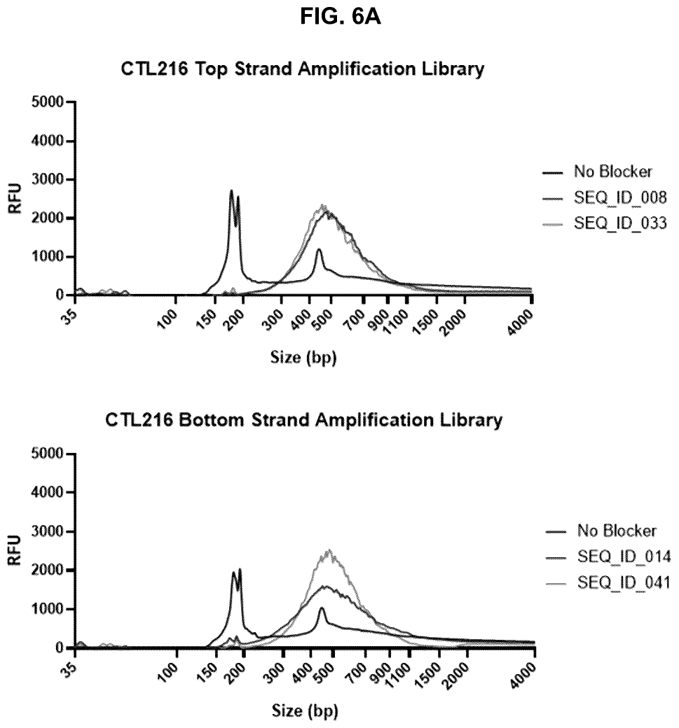

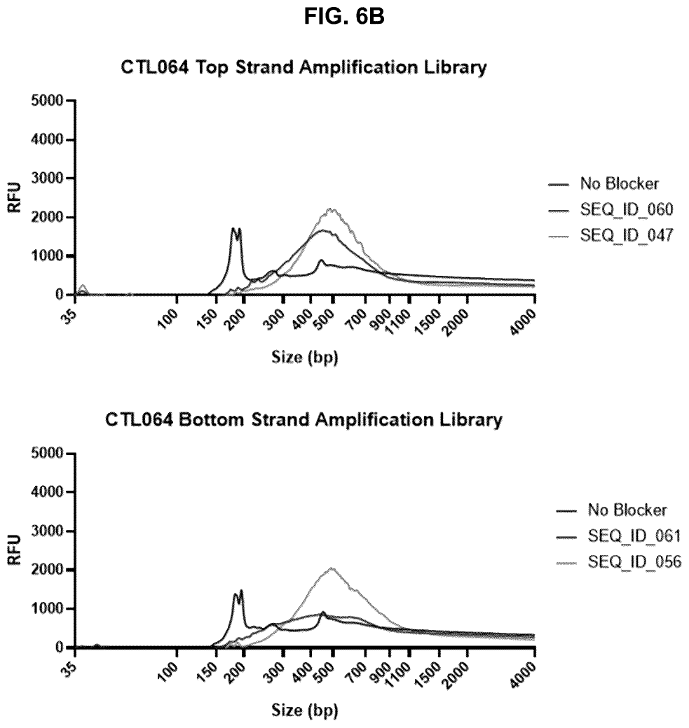

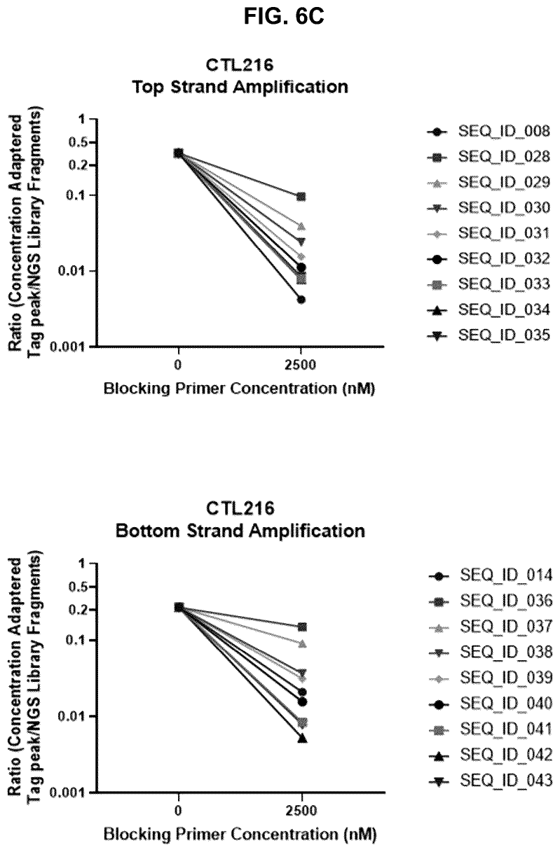

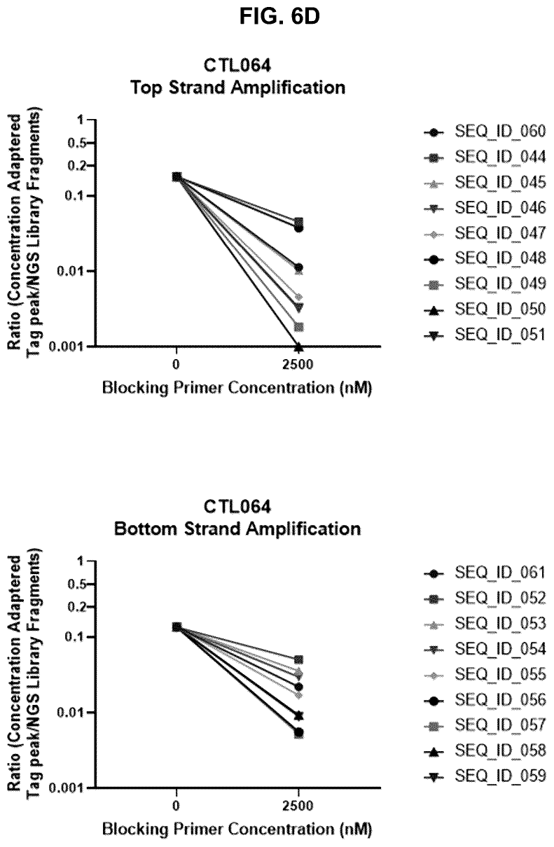

U2OS (HTB-96) were nucleofected with a single dsODN (100 μmol, 4 μM) (SEQ ID NO: 64-67) along with 4 μM RNP (WT-Cas9 V3 complexed with indicated guide) (SEQ ID NO: 68-70) using the Lonza 4D-Nucleofector System. Cellular gDNA was extracted after 72 hr and libraries were then fragmented and adaptered (SEQ ID NO: 72-73) using the xGen™ DNA Library Prep EZ UNI kit and xGen™ Deceleration Module to an average length of ˜500 bp. Tag specific amplification for PCR enrichment was achieved using the rhAmpSeq™ Library kit with PCR1 master mix (SEQ ID NO: 23-27) and RNaseH2 dependent PCR with and without adaptered-tag blocking oligos and separate strand amplification (SEQ ID NO: 8, SEQ ID NO: 14, SEQ ID NO: 28-61) followed by indexing PCR2 amplification (SEQ ID NO: 74, SEQ ID NO: 17). NGS libraries were then run on the Agilent Fragment Analyzer and the ratio of the concentration of adaptered-tag peak divided by the concentration of usable NGS fragments calculated (FIG. 6). For both dsODN-tags, addition of the blocking oligos significantly decreased adaptered-tag amplification while increasing the concentration of usable NGS fragments (200-2000 bp). Importantly, blocking oligos with LNA bases showed better adaptered-tag blocking ability than those without, further highlighting the importance of balancing LNA count and Tₘ ° C. of the blocking oligo. Lastly, the ability of the blocking oligos to block adaptered-tag amplification adhered to a similar trend as seen in the qPCR assay (FIG. 5). This strengthens the utility of the qPCR assay as an effective measure to screen additional dsODN-tag blocking oligos in the future, and reduces the cost, time and experimental complexity associated with NGS library preparation, sequencing, and analysis.

### CTL-Seq Dual Strand Library Amplification in the Presence of Blocking Oligos

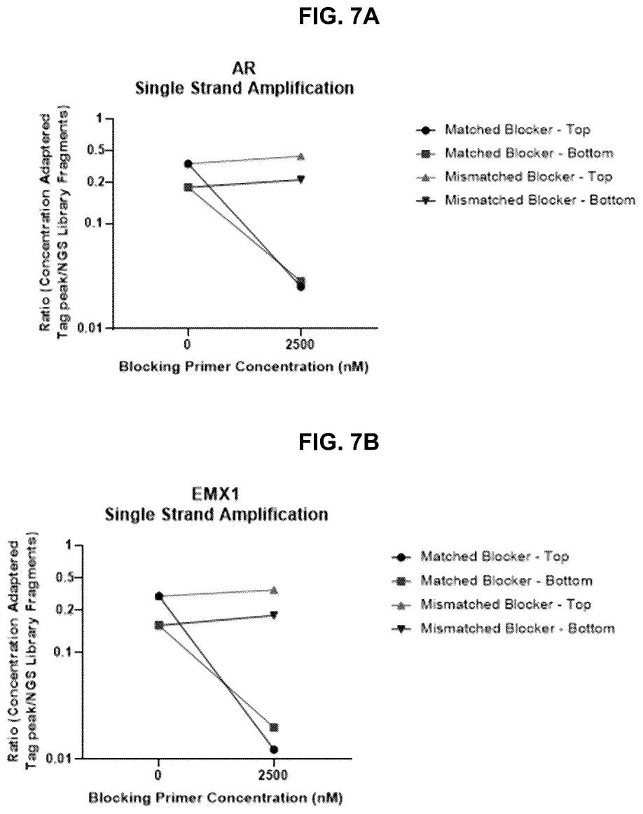

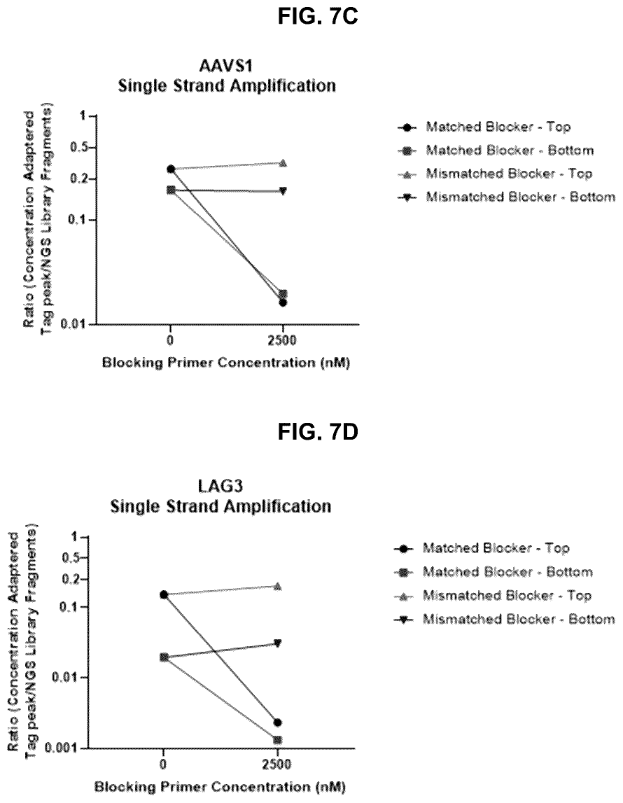

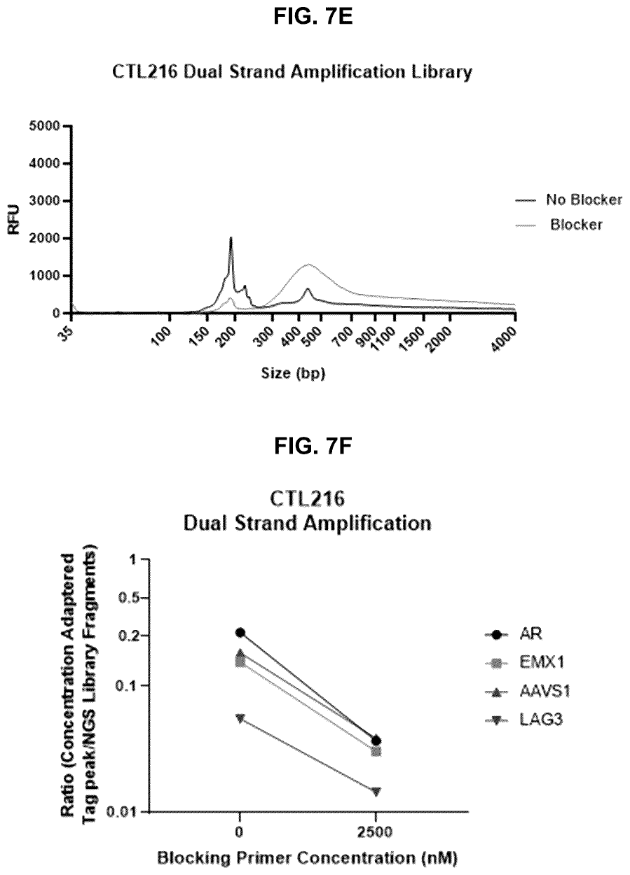

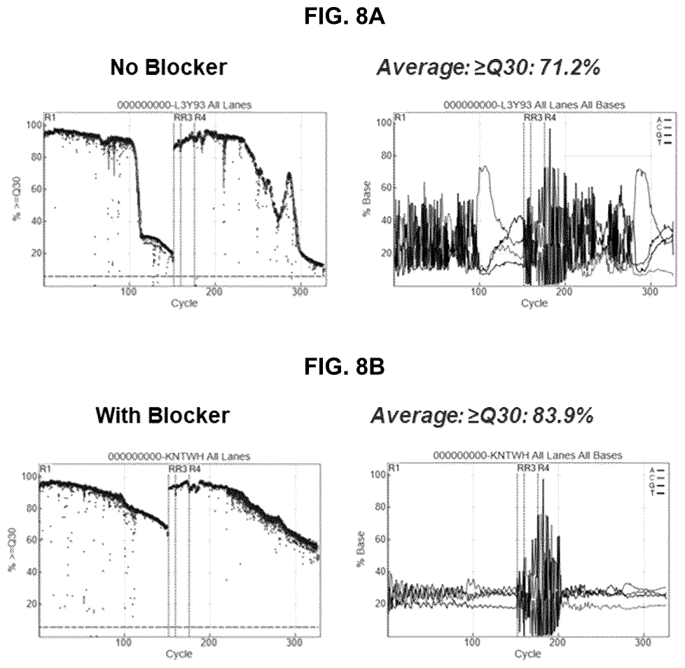

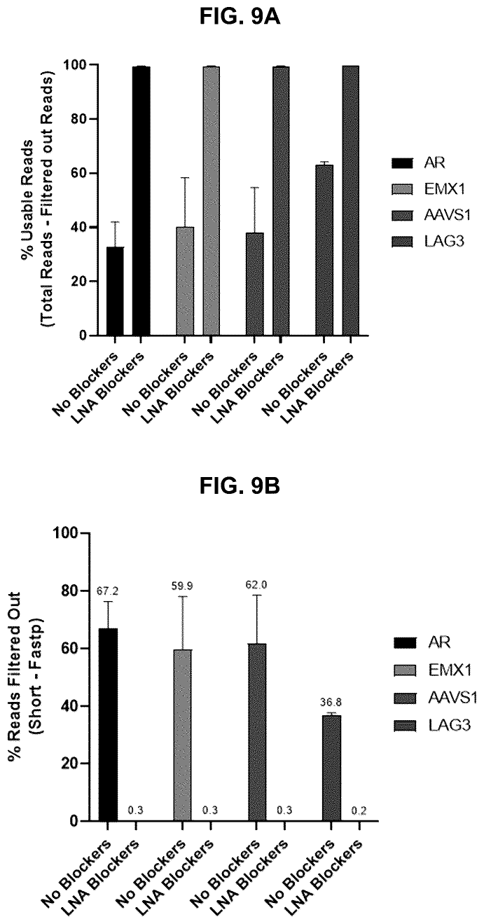

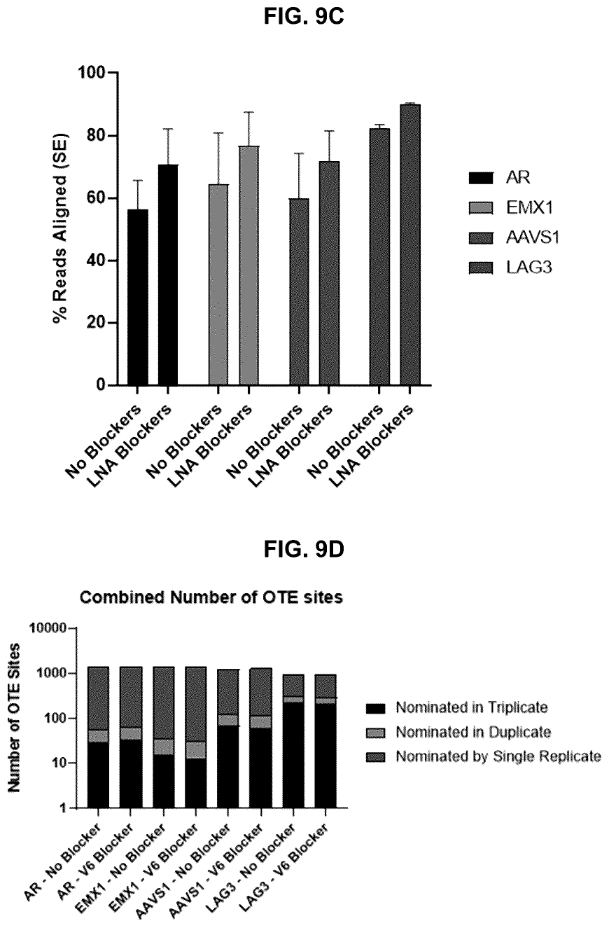

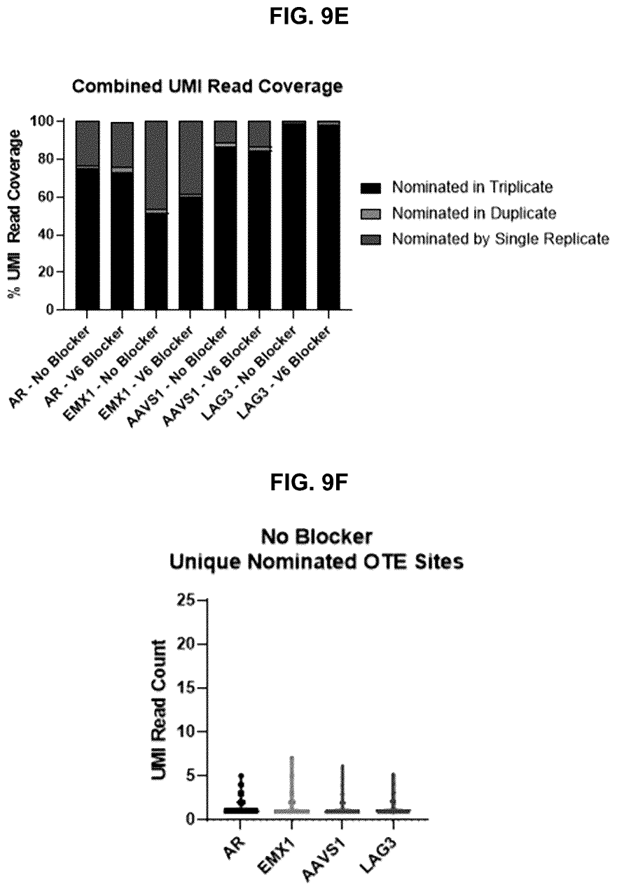

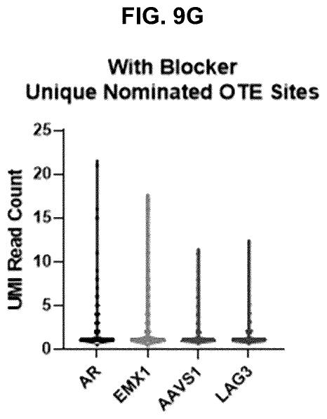

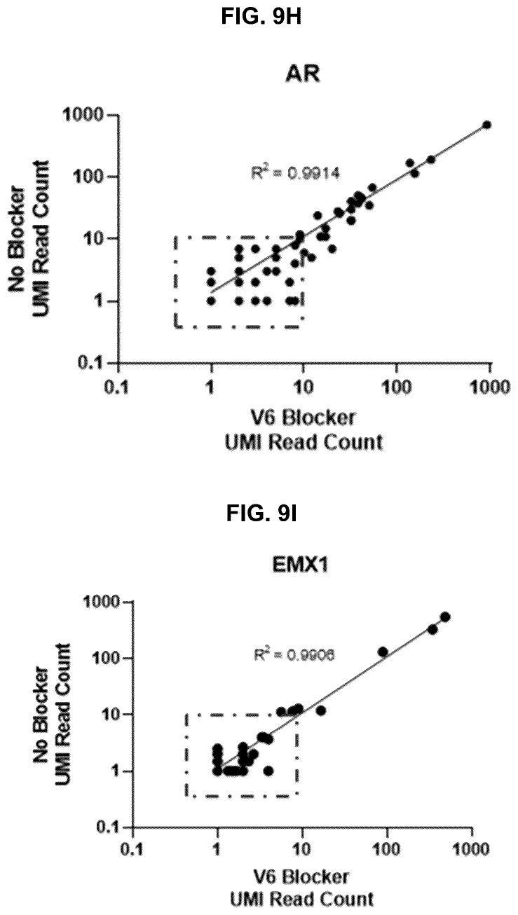

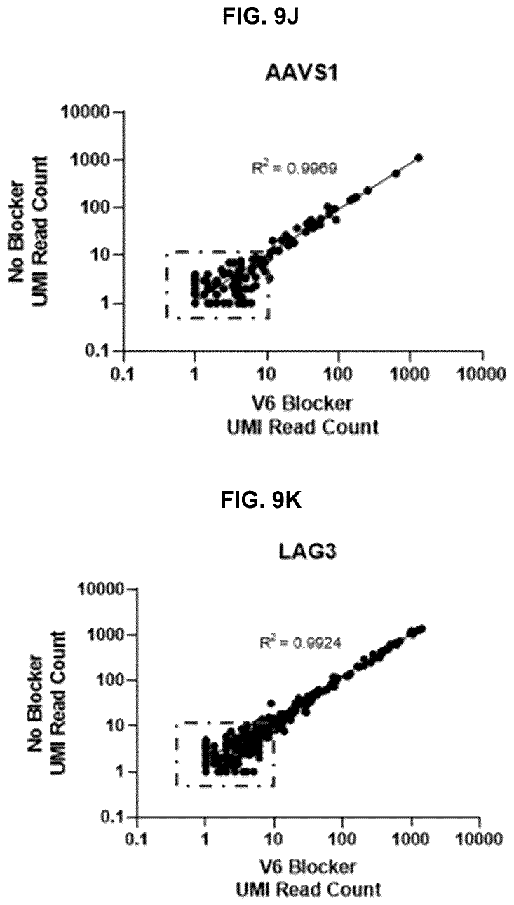

K562 (CCL-243) were nucleofected with a single dsODN (100 μmol, 4 μM) (SEQ ID NO: 64-65) along with 4 μM RNP (WT-Cas9 V3 complexed with indicated guide) (SEQ ID NO: 68-71) using the Lonza 4D-Nucleofector System. CTL-seq NGS library preparation was carried out as shown above with a few modifications: (1) single strand and dual strand (single tube) amplification was carried out with and without blockers (2) single strand amplification was carried out with matched blockers (i.e., top strand amplification with top strand blocker) and mismatched blockers (i.e., top strand amplification with bottom strand blocker) (SEQ ID NO: 33, SEQ ID NO: 41) (3) all gRNAs were done in biological replicate. NGS libraries were then run on the Agilent Fragment Analyzer and the ratio of the concentration of adaptered-tag peak divided by the concentration of usable NGS fragments was calculated (FIG. 7). The dual strand NGS libraries with and without adaptered-tag blockers were sequenced on a standard MiSeq flow cell with v2 chemistry (SEQ ID NO: 75-76) (FIG. 8) and processed through the CTL-seq Analysis Pipeline for OTE nomination (FIG. 9).

Blocking oligos significantly reduced adaptered-tag fragments with both single strand and dual strand (single tube) amplification libraries (FIG. 7). Importantly, having a matched blocker (top strand amplification with top strand blocker) is required for reduction in adaptered-tag amplification, which supports the specificity of adaptered-tag blockers to a particular fragment. When performing NGS on the dual strand amplification libraries, libraries without added blockers show substantial reduction in Q30 scores around cycle 100 and non-diverse percent base pair composition on a per cycle basis, which corresponds to the length of the adaptered-tag fragment. Conversely, libraries prepared with adaptered-tag blocking oligos, did not show steep Q30 score drops and exhibited a higher base pair diversity on a per cycle basis. Furthermore, libraries prepared with adaptered-tag blocking oligos significantly improved the number of usable reads (>99%) and improved number of reads mapped to the genome for each indicated guide (FIG. 9). Following nomination through the CTL-seq analysis pipeline, nominated sites from each per guide replicate was organized by the position specific scoring matrix into the top 500 OTE sites, intersected for OTE site overlap between replicates, and merged into unique site list for each guide. Blocker and no blocker samples exhibited similar reproducibility of OTE sites nominated with similar numbers of duplicate and triplicate nominated sites along with similar UMI read coverage (FIG. 9). Additionally, all OTE sites with UMI read counts>10 in the no blocker samples were nominated by the adaptered-tag blocking samples showing that there is no loss in nomination when adaptered-tag blockers are used. Lastly, all similar nominated sites showed highly correlative UMI reads indicating that amplification from tags inserted into genomic loci is not impacted by the addition of adaptered-tag blocking oligos.

CTL-Seq with Three Phosphorothioate Linkages

The GUIDE-Seq method inserts a dsODN into a nuclease-induced DSB and using the dsODN as an anchor for PCR amplification of the surrounding gDNA to elucidate the DSB location. GUIDE-Seq uses a dsODN that either has a single phosphorothioate linkage on the 5′- and 3′-terminus or two phosphorothioate linkages on each terminus. Typically, a single dsODN sequence that is 5′-phosphorylated and contains 2 phosphorothioates on the 5′ and 3′ end of each strand is used. See Table 2 (SEQ ID NO: 77-78).

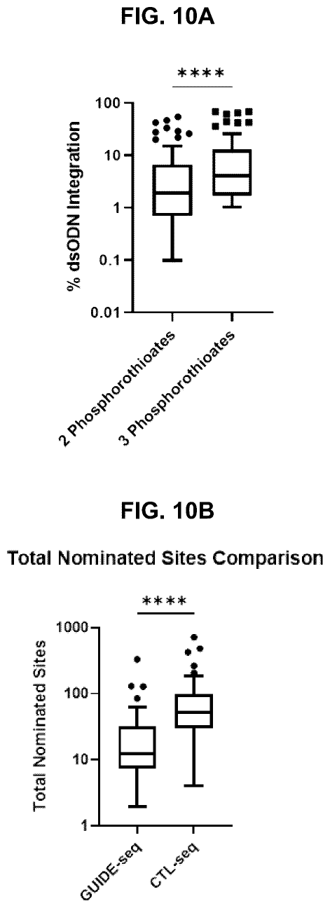

Unlike the static GUIDE-Seq dsODN sequence, CTL-seq utilizes a dynamic set of dsODN sequences that that are designed to be multiplexed (top and bottoms strand reactions as well as multiple dsODN primer sets in a single tube). Using multiple dsODN sequences increases the dsODN end base-pair diversity, which can increase integration into DSBs that repair via the microhomology-mediated end joining pathway thus increasing the potential sensitivity of the nomination assay. Pooled CTL dsODNs lead to increased number of OTEs with a dsODN integrated. In addition, CTL-seq uses an optimized phosphorothioate pattern where an additional phosphorothioate linkage was added to each strand's 5′- and 3′-terminus, for a total of 6 phosphorothioates per strand. This led to increased dsODN integration across multiple OTEs for 3 gRNAs: AR, EMX1, and AAVS1 that were assessed with targeted amplicon sequencing. See Table 3, and FIG. 10A-B.

TABLE 2
Sequences for GUIDE-Seq Comparison (5′→3′)

| | | SEQ |
|---|---|---|
| Name | Sequence | ID |
| CTLSeq | /5Phos/T*A*A*GCGGCGTAGGTAGCCGGACG | 64 |
| Top | AATGTCGGTCGTA*G*T*T | |
| CTLSeq | /5Phos/A*A*C*TACGACCGACATTCGTCCGG | 65 |
| Bot | CTACCTACGCCGC*T*T*A | |
| GUIDESeq_ | /5Phos/G*T*TTAATTGAGTTGTCATATGTTA | 77 |
| Top | ATAACGGT*A*T | |
| GUIDESeq_ | /5Phos/A*T*ACCGTTATTAACATATGACAAC | 78 |
| Bot | TCAATTAA*A*C | |

/5Phos/indicates a 5′-terminal phosphate; * indicates a phosphorothioate linkage between the two nucleotides.

TABLE 3
Total dsODN integrated sites for each corresponding
dsODN using three gRNAs AR, EMX1, and AAVS1

| | |
|---|---|
| dsODN Phosphorothioate | Total dsODN Integration Sites |

| Number (5′/3′ both strands) | AR | EM1 | AAVS1 |
|---|---|---|---|
| 2 | 117 | 35 | 134 |
| 3 | 168 | 42 | 155 |

GUIDE-Seq uses two rounds of dsODN-specific PCR with nested primers for the second round of amplification along with a P7 adapter primer that will extend off of the 5′-terminus of the nested dsODN-specific PCR2 primer. GUIDE-Seq primers are DNA only and amplify from the ends of the dsODN. Thus, mispriming events are not distinguishable from actual dsODNs inserted into the gDNA. In designing the primers this way, the positive and negative strand primers must be separated in order to prevent exponential amplification of primer dimers. In contrast, CTL-seq uses rhPCR primers with the format rDDDDx. These primers are only partially overlapping on the 5′-termini and do not anneal to the ends of the dsODN sequence. The CTL-seq primer design overcomes both issues of the GUIDE-Seq primer design. The CTL-seq primers allowing for positive and negative strand primers to be utilized in the same reaction for multiplexing and they permit distinguishing mispriming events through interrogation of the sequence adjacent to the primer after sequencing (i.e., if an amplification event is from a dsODN then the sequence should align with the dsODN sequence). Furthermore, CTL-seq can amplify multiple dsODNs in a single tube to increase the sensitivity of OTE nomination as the rh design should prevent primer dimers from forming between multiple dsODN primer pairs.

A fundamental challenge with GUIDE-Seq arises from the dsODN sequence and subsequent primer design. The GUIDE-Seq dsODN is 73.5% AT-rich, which creates challenges when designing primers that have high enough Tₘ's for efficient PCR. In order to increase the primer Tₘ, GUIDE-Seq uses long primers that increase the overlap between the positive and negative strand primer. The overlap on the 3′-ends of each primer leads to primer dimer formation followed by exponential amplification if both primers used in the same reaction. Therefore, GUIDE-Seq cannot be multiplexed and requires two reactions per sample, which decreases efficiency and increases hands-on-time and costs. In addition, a high AT-rich dsODN sequence can create large amounts of non-specific amplification of AT-rich regions in the genome. The GUIDE-Seq primers amplify from the very ends of the GUIDE-Seq dsODN. This creates the issue of not being able to distinguish between a properly amplified dsODN inserted into a DSB from a mispriming events. Therefore, GUIDE-Seq has high levels of noise and reduces specificity.

### Example 2

### Human Cell Culture and Transfection (K562 and HEK293-Cas9)

K562 (ATCC) and HEK293-Cas9 (ATCC) cells were cultured in Iscove's Modified Dulbecco's Medium (IMDM; ATCC) and Eagle's Minimum Essential Medium (EMEM; ATCC) supplemented with 10% FBS at 37° C. with 5% CO₂. RNPs were formed by the addition of Alt-RT Sp. Cas9 Nuclease V3 (IDT) and incubating for 20 minutes at room temperature (Molar Ratio: 1:1.2, Cas9:sgRNA). For each transfection, 8.0×10⁵ cells were washed with 1× phosphate-buffered saline, resuspended in 20 μL of solution SF (Lonza). For K562 cells, RNP complexes at 4 μM were combined with 4 μM of the dsODN into the SF solution, while for the HEK293-Cas9 cells, 5 μM sgRNA and 0.5 μM dsODN were added to the SF solution. This mixture was transferred into 1 well of a 96-well Nucleocuvette plate (Lonza) and electroporated using program FF-120 (K562) or DS150 (HEK293-Cas9). Two nucleofections per replicate were performed and each treatment done in triplicate. Following electroporation, cells were transferred to a 6-well plate preheated with either IMDM or EMEM and were incubated at 37° C. with 5% CO₂ for 72 hours. After incubation, gDNA was extracted using either the Purelink™ Pro 96 Genomic DNA Purification kit or the Monarch™ Spin gDNA Extraction Kit (New England Biolabs) according to the manufacturer's instructions, eluted in low-EDTA TE buffer (IDT, 11-05-01-05), and quantified using a NanoDrop 8000 UV-Vis Spectrophotometer (ND-8000-GL).

### Primary T-Cell Culture and Transfection

Frozen human primary pan-T cells (STEMCELL Technologies) from 2 unique human donors were thawed in ImmunoCult-XF T Cell Expansion Medium including 300IU IL-2 (Cytiva) and activated with 10 μL/mL TransAct, human, T cell activator (Miltenyi Biotec) for 48 hours. To prepare for transfection using Lonza 96-well plate 4-D Nucleofector system, cells were counted, pelleted using centrifugation (300×g, 10 minutes at room temperature), and washed gently with 10 mL 1×PBS. Cells were again pelleted and resuspended in Lonza Nucleofection Solution P3 at 2.5×10⁶ cells/mL. For each electroporation, 5 μL of RNP complex and 3 UL ds Tag was added to 20 μL of cells in P3 (5×10⁵ cells/nucleofection) for a final concentration of 4 μM RNP (1:1.2 ratio of Cas9 to gRNA) and 1-4 μM dsODN. Where tag was not included, 3 μL of IDT Alt-R Cas9 Electroporation Enhancer was added for 3 μM final concentration to achieve a fixed final nucleofection reaction volume of 28 μL. Each reaction was mixed by pipetting and 25 μL was transferred to an electroporation cuvette plate. The cells were electroporated according to the manufacturer's protocol using the Amaxa 96-well Shuttle and nucleofection protocol 96-EH-140. After electroporation, the cells were resuspended in 75 μL pre-warmed IL-2 culture media in the electroporation cuvette. Triplicate aliquots of 25 μL of recovered cells were further cultured in 175 μL pre-warmed IL-2 media with TransAct. Cells were incubated for 72 hours, after which gDNA was isolated and quantified.

iPSC Culture and Transfection

iPSCs from fibroblasts (Coriell Institute, GM23338) were cultured in mTeSR™ Plus media (Stemcell Technologies) at 37° C. with 5% CO₂. RNPs were formed by mixing Alt-R S.p. Cas9 Nuclease V3 (IDT) and Alt-R CRISPR-Cas9 sgRNA (IDT) incubating for 20 minutes at room temperature (Molar Ratio: 1:1.2, Cas9:sgRNA). For transfection using Lonza 96-well plate 4-D Nucleofector system, cells were detached using ReLeSR™ (Stemcell Technologies) and washed with 1× phosphate-buffered saline. CRISPR reagents at required concentrations (4 μM RNP; 0.5 μM dsODN) were added to the mix to make a final volume up of 25 μL, and of which 20 μL was transferred to the nucleocuvette for electroporation. The nucleovette plate was electroporated using code CA-137. After the nucleofection, cells were recovered and plated in complete mTeSR Plus medium with 1× CloneR™ 2 supplement (Stemcell Technologies). Recovery media was added to the zapped transfected cells to make up a final volume of 100 μL, and 25 μL of this was added to 175 μL media per replicate well for final plating in a vitronectin coated 96-well plate. During recovery and growth at 37° C. with 5% CO₂ for up to 96 to 120 hours, media changes were performed as desired and/or following manufacturer's protocols for media and CloneR 2 supplement. gDNA extraction and quantification occurred as described above.

Off-Target Nomination with UNCOVERseq

500 ng of purified gDNA was enzymatically fragmented and adapter-ligated using the xGen™ DNA Library Prep EZ UNI kit along with the xGen Deceleration Module (IDT, xGen DNA Library Prep EZ UNI 96 rxn, 10009822; xGen Deceleration Module 96 rxn, 10009823) according to the manufacturer's instructions and cleaned with AMPure XP beads (Beckman). Following fragmentation and adapter ligation, rhPCR was performed using rhAmpSeq™ Library Mix 1 (IDT) to amplify the DNA in a single tube using a forward primer specific to the P5 adapter, a reverse primer specific for top and bottoms strand of the integrated dsODN tag, and an adaptered-tag blocking oligo corresponding to each strand of the dsODN. Following PCR, samples were diluted 1:40 with nuclease-free water and used in a second PCR with rhAmpSeq™ Library Mix 2 (IDT) that added a unique P7 adapter to each library. Libraries were then cleaned with AMPure XP beads and run on an Agilent Fragment Analyzer for library quality assessment. All libraries were quantified with the Qubit 1× dsDNA HS Assay kit (Invitrogen) and pooled in equimolar amounts. All libraries were run on an Illumina MiSeq or NextSeq2000 instrument with 150-bp paired-end reads.

### Computational Analysis-Nomination

Following next-generation sequencing, Illumina adapters and UMIs were identified and annotated using Picard MarkIlluminaAdapters. Tag sequences were identified and trimmed using Cutadapt v4.2. Sequencing reads were aligned to hg38 (GRCh38.p12) reference genome using BWA mem v0.7.15 and UMI consensus reads were generated based on consensus from a single-strand (minimum UMI consensus size=1) using fgbio v0.7.0 (github.com/fulcrumgenomics/fgbio). Nomination of candidate off-target sites began by using mapped UMI consensus reads to create a flanked search space (+40 bp) to perform alignment between the guide and empirical target region using a glocal implementation of the Needleman-Wunsch alignment. After a candidate match to the gRNA spacer region was identified in the sequencing data, nominated off-target sites were identified using a hypergeometric test with multiple testing correction (Benjamini & Hochberg; FDR<0.05) by comparing individual treatment samples and pooled control samples for significant differences in representation between the two. The following criteria were used to nominate off-target sites from this analysis for verification: (1) at least one sample nominated a given site with NGS evidence on both sides of the cut site (2) Levenshtein distance<7 as determined post-alignment and 3) significant adjusted p-value when comparing the frequency of the event to the pooled control(s). Nominated on/off-target sites had additional meta-data added based on alignment/genomic context and were placed into described Tiers based on this meta-data.

### Library Preparation—Confirmation

Genomic DNA was extracted from control and genome-edited cells as described above. Libraries for amplicon NGS were prepared using a previously described rhAmpSeq amplification-based method (IDT) using 100 ng of gDNA input. Libraries were purified using Agencourt AMPure XP system (Beckman Coulter, Brea, CA, USA) and quantified by qPCR before being sequencing on the Illumina MiSeq platform (v.2 chemistry, 150-bp paired end reads; Illumina). Read demultiplexing was performed on the resulting BCL files using Picard v2.18.9 IlluminaBasecallsToFastq.

### Computational Analysis—Confirmation

Analysis of the sequencing data to identify confirmed off-target editing at the nominated sites was performed using CRISPAltRations v1.2.1, see U.S. Pat. No. 12,254,959, which is incorporated by reference herein for such teachings. This analysis comprised two parallel workflows: identification of indels at the position of the DSB/SSB, and identification of base-editor induced A→G (ABE) or C→T (CBE) transversions in the relevant base-editing window.

For identifying indels, the window for event quantification was centered on the canonical cut site and events quantified utilizing the default window size for Cas9 (8 bp). To determine whether indels found in the sequencing data could result from bona fide off-target cutting, indels were grouped by location relative to the cut site (prioritizing minimum distance to cut site) followed by fitting counts of events to a negative binominal model with a Wald test for significance in each location bin per off-target using the DESeq2 package within IDT's OTEasy tool. For classification of indel off-target editing, the tool requires (1) sufficient read coverage for the site (>1000×) in all replicates; (2) significant edits to occur at or adjacent to the cut site after optimal alignment; (3) the classified cumulative significant edits to exceed 0.01%; (4) the comparison of treatment/control samples at the site to have a significant adjusted p-value (p<0.05); and (5) an average coverage frequency of at least 5× the ascribed cumulative frequency observed (e.g., for 0.1% editing, at least 5,000× coverage).

For identifying base-editing generated off-target effects, the window for event quantification was centered in the middle of canonical base-editing window between position +5/+6 of the spacer (5′ to 3′) with a 5 bp window for quantification. To determine significant base-editing transitions resulting in off-target editing, all individual events that contained an ABE (A→G or T→C) or CBE (C→T or G→A) transition were grouped according to unique base editing events in the window and fitting counts of events to a negative binominal model with a Wald test for significance in each location bin per off-target using the DESeq2 package within IDT's OTEasy tool. For classification of adenine base editing at off-targets, the tool requires (1) sufficient read coverage for the site (>1000×) in all replicates; (2) the classified cumulative significant edits to exceed 0.5%; (3) the comparison of treatment/control samples at the site to have a significant adjusted p-value (p<0.05); and (4) an average coverage frequency of at least 5× the ascribed cumulative frequency observed.

### Computational Analysis—Translocations

To quantify translocations from editing, Primer Anchored Statistical Translocation Analysis (PASTA) was used. This analysis was only performed on the amplicon sequencing pools containing the on-target because multiplexed amplification is a requirement for event detection using the method, and reactions not containing the on-target are unlikely to have any significant translocation events. To quantify translocations, expected primers were identified in reads using fg-idprimer (github.com/fulcrumgenomics/fg-idprimer; -k=6, -K=8, -S=5, -max-mismatch-rate=0.07). Following this, treatment/control pairs had their counts paired and primer count frequencies subjected to a one-tailed hypergeometric test with Benjamini-Hochberg correction (statsmodel v0.15.0; default settings) to calculate an adjusted p-value (p-adj). Unexpected primer pairs with padj<0.01 with no flags were classified as a translocation and had the translocation frequency (P) calculated using the following equation:

$${P}_{t} = \frac{{n}_{t}}{{f}_{total}} + \frac{{n}_{t}}{{r}_{total}}$$

where n is equal to the count of the unexpected primer pair of interest, t is the significant translocation being interpreted, f is the total count of the shared forward primer events excluding the count participating in the n translocation event, and r is the total count of shared reverse primer events excluding the count participating in the n translocation event. The translocation frequency is then adjusted by the background level frequency in the control by subtracting any translocation frequency observed in the control sample from the treatment frequency. Total translocation burden (B) was calculated using the following equation:

$$b = 1 - {\prod }_{t}^{{t}_{n}} ( 1 - {P}_{t} )$$

where t is equal to a significant translocation, and tₙ is equal to the last significant translocation of all translocations. All translocations for the purposes of this equation are assumed to be occurring independently. Using the method, translocations are quantified if (1) the estimated frequency exceeds 0.1% of editing; (2) if the translocation has a significant p-value (p<0.01); and (3) if the translocation is found to meet these criteria in all replicates.

### Results

### Optimization of UNCOVERseq

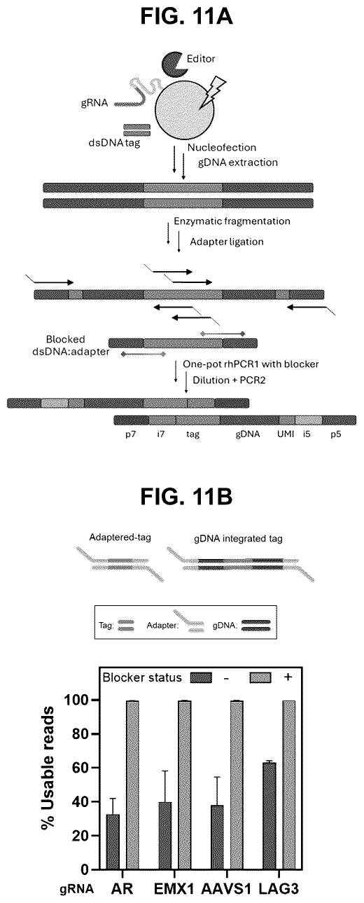

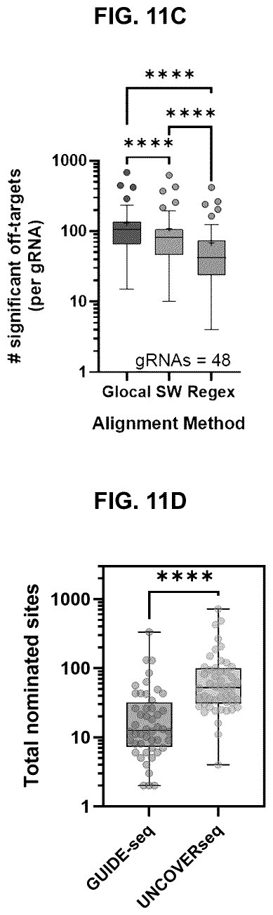

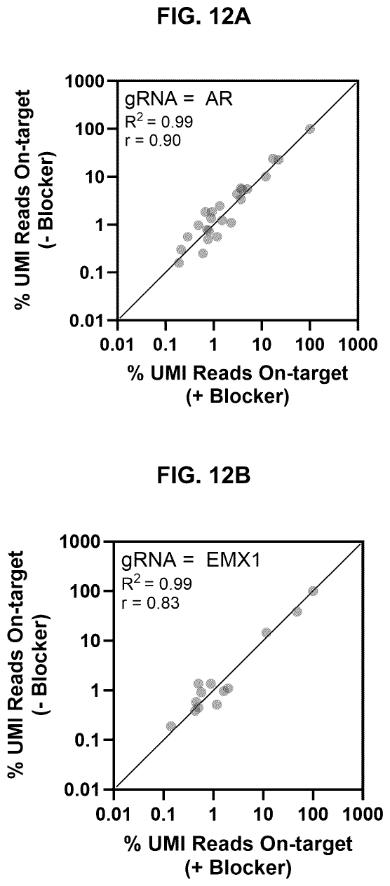

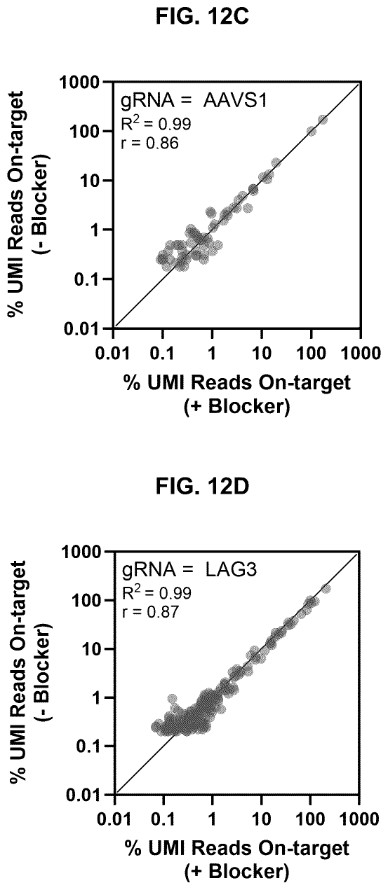

To create the nomination method, the original GUIDE-Seq protocol was used and a novel orthogonal dsDNA sequence was designed with sufficient length to perform a modified rhPCR to multiplex primers in close proximity within a single reaction while avoiding primer-dimers. To streamline the process for preparing the nomination gDNA libraries, conversion was done from a mechanical to enzymatic fragmentation. Upon analyzing data, it was observed that freely adaptered dsDNA tag was allocated an average range of 37% to 67% of reads, varying across 4 gRNAs (FIG. 11B). This same artifact was also observed with the original GUIDE-Seq protocol. To improve usable reads resulting from NGS, a blocking oligo was introduced into the PCR1 preparation designed to target the adapter: dsDNA junction (FIG. 11A). Introduction of this blocker reduced reads belonging to the adapter: dsDNA artifact to an average range of 0.3% to 0.5%, meaning >99% of reads were now belonging to gDNA: dsDNA junctions (FIG. 11B). Nomination frequencies were found to be conserved for all gRNAs (R²=0.99) with and without the blocking oligo (FIG. 12).

In parallel to creation of the wet-lab protocol, an analysis pipeline was created with features such as heuristic nomination criteria (Levenshtein distance<7; read-evidence from both sides of a prospective off-target), statistical comparison of treatment: control samples as nomination criteria (FDR<0.05) and integrated genomic annotations. Optimizations in the computational pipeline were then investigated for nomination of gRNAs using a set of 48 gRNAs spread across the PDCD1, LAG3, CTLA4, NRP1, IL2RA, and TIGIT genes. In off-target nomination, off-target loci are generally determined to be trustworthy based on (1) frequency, (2) reproducibility, and/or (3) similarity to the intended target sequence (gRNA), with Levenshtein distance>6 often being used to disqualify an off-target.

To investigate the effect of alignment method used for determination of an off-target list, existing GUIDE-Seq pipeline methods (github.com/aryeelab/guideseq; commit: 997b892; fuzzy regular expression based; Regex) and historical GUIDE-Seq pipeline methods (github.com/aryeelab/guideseq; tag: v1.0; Smith-Waterman alignment with −100/−100 gap open/extension penalty) were tested as compared to a glocal implementation of the Needleman-Wunsch algorithm. Investigation of 48 different gRNAs found a significant difference in the number of Levenshtein distance<7 loci nominated using each approach, and that the glocal Needleman-Wunsch alignment approach yielded a median of 30% and 150% more qualified off-target locations than the current and historical GUIDE-Seq analysis approaches (FIG. 11C). To do an end-to-end comparison of the current GUIDE-Seq method to UNCOVERseq, an end-to-end comparison was performed across gRNAs and it was found that the method disclosed herein nominated a range of 15% to 883% more off-targets per gRNA, with an average of 95 off-targets as compared to 30 using the GUIDE-Seq protocol (FIG. 11D). The final instantiation of the end-to-end method was termed UNCOVERseq (Unbiased Nomination of CRISPR Off-target Variants using Enhanced RhPCR; v1.0).

### Promiscuous Cell Systems as Sensitive UNCOVERseq Proxy Nomination Models

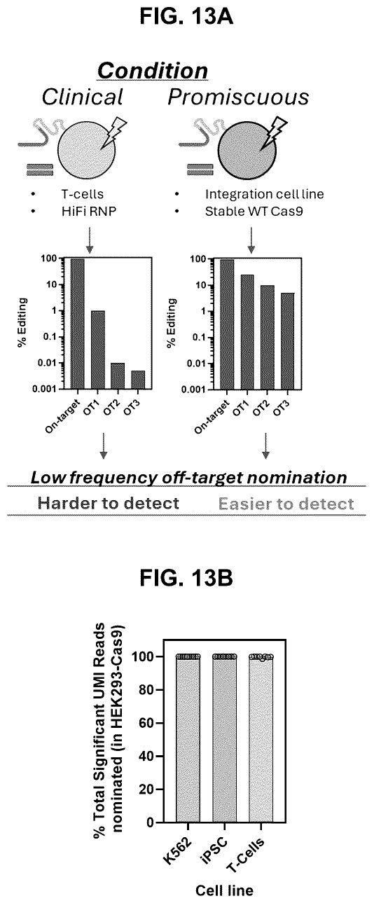

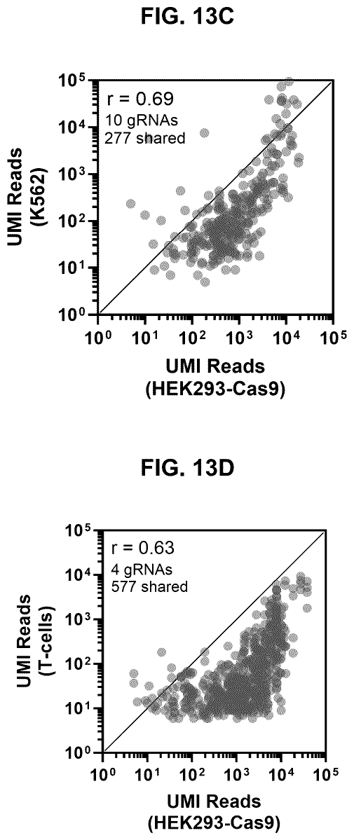

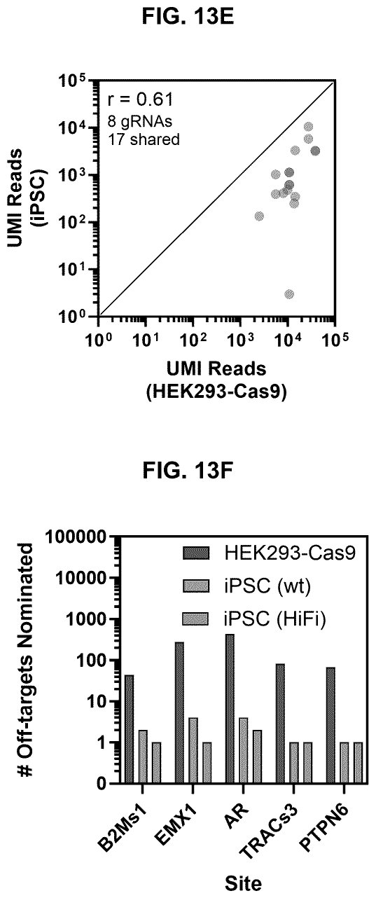

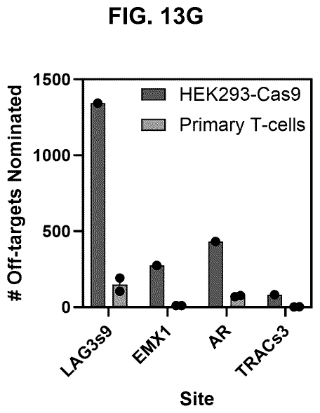

To identify ideal biological operating conditions, biological variables were explored with potential workflow impacts on nomination performance. Promiscuous editing conditions are known to increase editing frequencies at off-targets, which is hypothesized to increase the sensitivity of in cellulo methods like UNCOVERseq (FIG. 13A). To test this, 4-10 gRNAs per cell line were selected and off-targets were nominated using UNCOVERseq in K562, iPSCs (wildtype S.p. Cas9 or HiFi Cas9), Primary T-cells, or a promiscuous HEK293 cell line stably expressing S.p. Cas9 (HEK293-Cas9). Investigation of the overlap of off-target frequencies between these cell lines and HEK293-Cas9 found an average of 99.7% to 100% of total UMI corrected events (corresponding to frequency) in each cell line in the off-targets of just a single replicate of HEK293-Cas9 (FIG. 13B). Comparison of nomination frequencies showed a high rank order correlation for HEK293-Cas9 nominated off-targets between K562 (r=0.63), iPSC (r=0.61), and Primary T-cells (r=0.69), demonstrating that the frequency-based importance of different off-targets for prioritization was still largely conserved (FIG. 13). It was also observed that the overall nominated off-targets number for the same gRNAs could vary significantly in different primary cell lines for the same gRNAs, with low numbers of off-targets nominated in iPSCs, further demonstrating why a promiscuous system can be used to remove this variable (FIG. 13F). Overall nomination in a promiscuous cell line like HEK293-Cas9 was capable of generating an average of 196% to 1,560% more candidate targets per gRNA compared to an efficient primary cell type for nomination, like Primary T-cells, supporting that this is a more sensitive model for off-target nomination even in translational contexts (FIG. 13G).

### Off-Target Reproducibility Using UNCOVERseq

To determine ideal experimental conditions for off-target nomination, factors affecting reproducibility were characterized in a functional context. When making decisions about nominated off-targets, ideally off-targets are prioritized based on (1) frequency, (2) reproducibility, and (3) genomic impact. To this end, a tiering system was developed based on UNCOVERseq data to prioritize off-targets for confirmation (Tier 1 to 3) from less important ones.

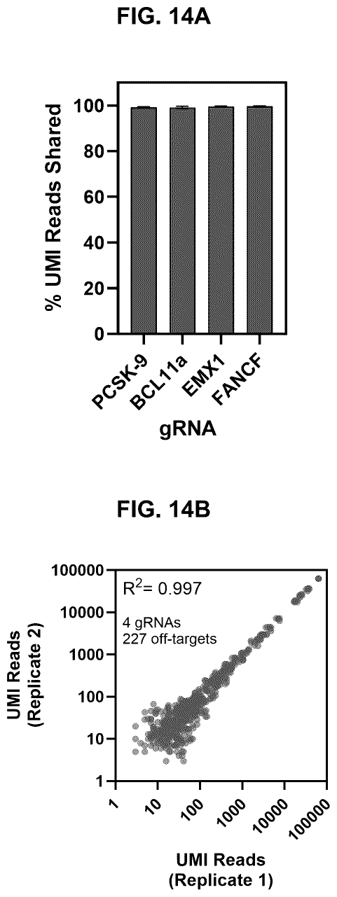

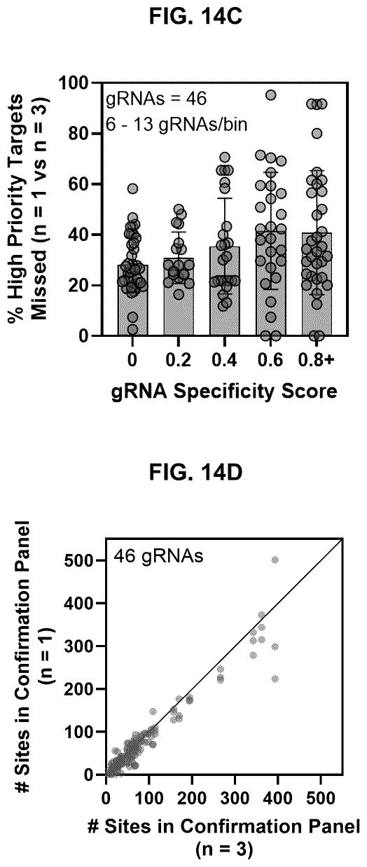

To assess sample-to-sample reproducibility, biological triplicates of UNCOVERseq were compared in HEK293-Cas9 across four gRNAs. An average of 99.2% to 99.7% of instances based on frequency were shared between any two biological replicates, indicating high frequency sites were consistently captured with a single replicate (FIG. 14A). Frequency rank order of off-targets was highly conserved (R²=0.997) across replicates as well (FIG. 14B). This indicated that both frequencies and sites containing the majority of reads were highly reproducible between UNCOVERseq replicates.

To assess reproducibility for prioritizing important off-targets (Tier 1-3), biological triplicates were compared to single replicates of 48 gRNAs in HEK293-Cas9. Without biological triplicates, 30% to 40% of high priority off-targets were not captured or prioritized (FIG. 14C). The average frequency of missed high priority targets increased with gRNA specificity, though this is partially because the denominator (total off-targets) is smaller (FIG. 13C). While high frequency events were reproducible (FIG. 14A), low frequency events lacking full reproducibility or those in important genomic contexts (e.g., exonic) were not appropriately identified without replicates (FIG. 14C). Replication additionally theoretically allows more appropriate tiering of off-targets. To investigate the effect of tiering on panel size, the possibility of creating a confirmation panel for appropriately tiered off-targets of biological triplicates (Tier 1 to 3) was compared versus all off-targets of a single replicate. It can be seen that even though replicates lead to more off-targets nominated, panel sizes are roughly equivalent (FIG. 14D). This provides evidence that tiering off-targets in replicate nominations leads to more impactful sites being nominated and interrogated without significantly increasing the size of confirmation panels.

### Determination of UNCOVERseq Sensitivity and Process Controls

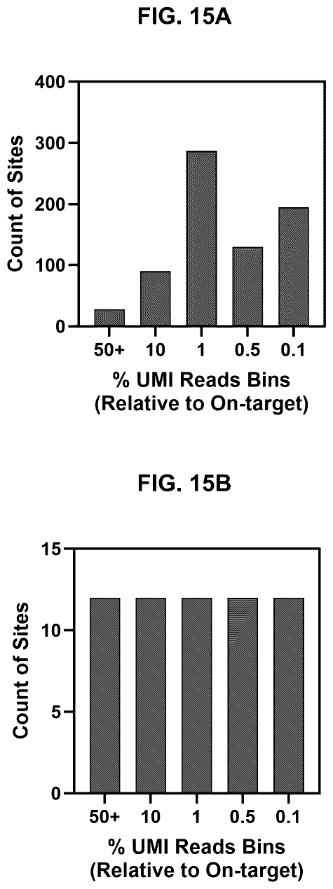

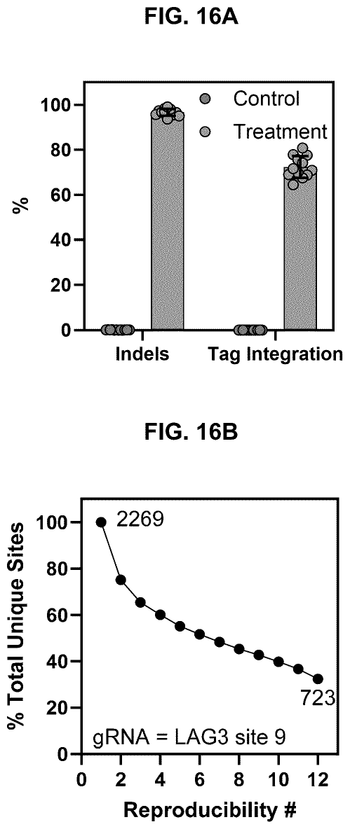

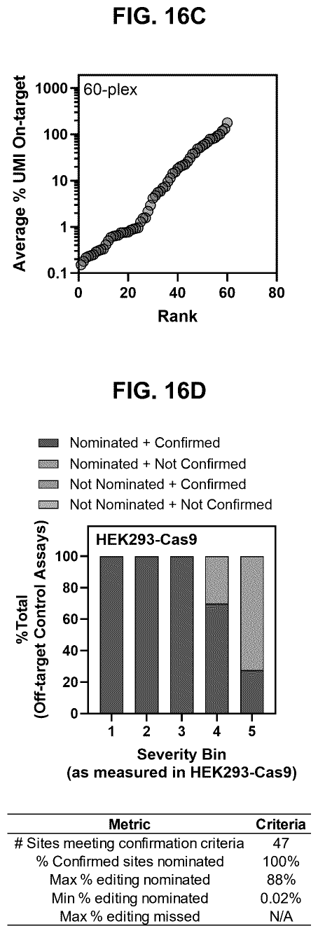

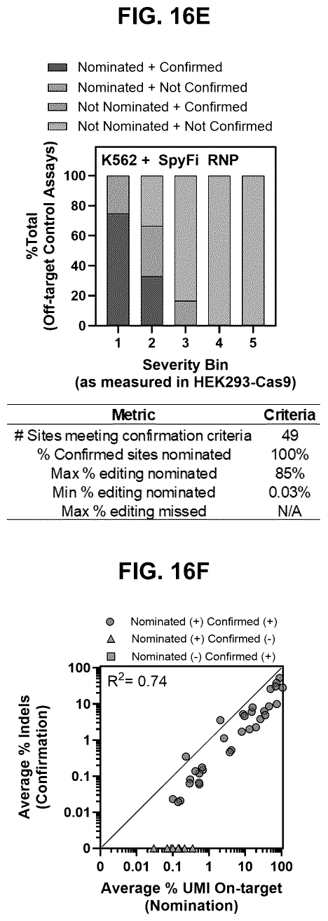

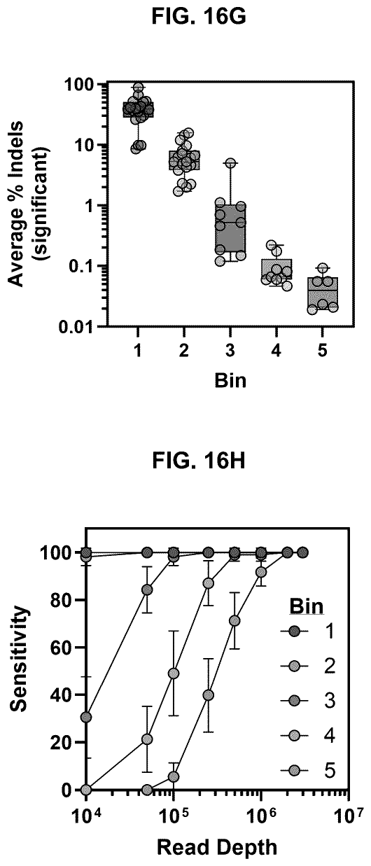

The sensitivity of UNCOVERseq was characterized, considering variable conditions like different cell lines and culture environments, given the known impacts of these conditions on total off-targets nominated (FIG. 13). Using a promiscuous LAG3 targeting gRNA (LAG3 site 9), off-targets were nominated in 12 biological replicates using HEK293-Cas9. All replicates showed high editing (>90%) with tag integration frequencies between 64% and 81% (FIG. 16A). 2269 unique nominated sites were identified across all replicates, with 723 consistently reproduced across all replicates (FIG. 16B). The relative rate at which off-targets could not be reproduced between each subsequent replicate rapidly dropped below <5% after 3 replicates and decreased linearly afterwards (FIG. 16B). Although only off-targets reproduced in all replicates were pursued for downstream confirmation, this suggests that after three replicates real off-targets that were harder to reproduce due to low frequencies and random sampling differences may be lost from high replication requirements. To monitor editing frequencies, 12 sites from each frequency bin were selected, creating a subset of 60 for sequencing and confirmation (FIG. 16C; FIG. 15). Confirmation in HEK293-Cas9 showed significant indels ranging from 88% to 0.02%, approaching the sensitivity limit (0.01%) (FIG. 16D).

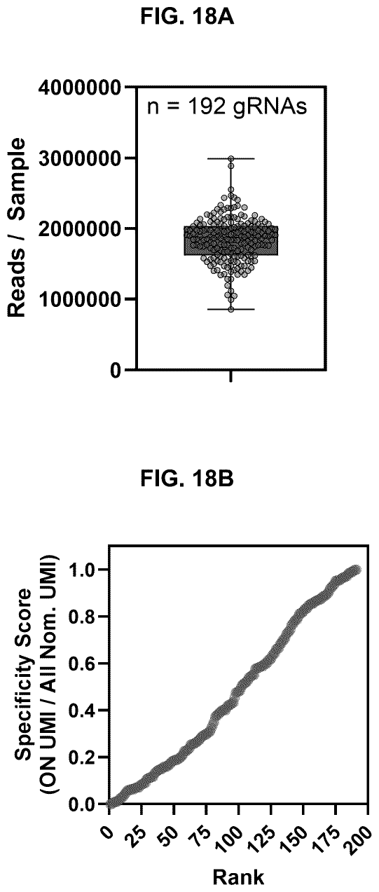

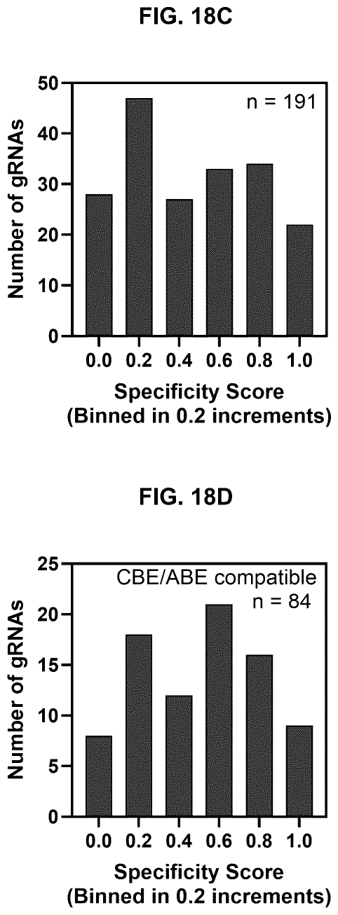

Of the interrogated panel, 30% of sites in the <0.5% bin (Bin 5) and 72.3% in the 0.5 to 1% bin (Bin 4) could not be confirmed down to 0.01% indels, suggesting UNCOVERseq nominates sites with frequencies below 0.01% indels (FIG. 18). This may be due in part to the higher genomic DNA input in UNCOVERseq (500 ng) as compared to the subsequent confirmation library preparation (100 ng). In high specificity conditions (SpyFi Cas9+ribonucleoprotein nucleofection), fewer off-targets were edited, but a similar dynamic range of editing was retained (0.03% to 85% indels), with 100% of confirmed sites successfully nominated (FIG. 16E). This indicated that the approach can be used as a process control to ensure high sensitivity with reportable metrics. UNCOVERseq nomination frequencies were highly correlated (R²=0.74) with confirmation frequencies (FIG. 16F). The high true positive rate and consistent decrease in confirmation success in bins approaching the detection limit suggest these sites represent ˜100% true positives.

### Determination of UNCOVERseq Input and Sequencing Requirements

The number of genomes in an amplification reaction and the number of sequencing reads allocated are key limiters for NGS assay performance. To maximize sensitivity, all UNCOVERseq experiments use ˜150,000 genome equivalents. While gDNA input could potentially be increased, it was rationalized that this amount of gDNA is attainable by most experimental conditions and represents the ability to potentially detect down to 0.001%, which is below the limit of detection for any currently published confirmation techniques for CRISPR gene editing.

Read depth requirements were characterized for reproducible off-target nomination by downsampling the LAG3 site 9 UNCOVERseq dataset (n=12) to frequencies ranging from 3 million to 10,000 reads per sample. Significant editing across confirmable sites showed interquartile range (IQR) frequencies as follows: Bin1, 26-48%; Bin2, 2.6-7.6%; Bin3, 0.13-0.82%; Bin4, 0.06-0.13%; Bin5, 0.01-0.02% (FIG. 16G). Thus, this dataset represented the full dynamic range of what can currently be detected by confirmation (>0.01% indels). High frequency sites in Bin1 and Bin2 were nominated with 100% sensitivity using 50,000 reads per sample (FIG. 16H). To nominate down to 0.01-0.02% indel frequencies with 100% sensitivity, at least 2 million reads per sample were required (FIG. 16H). Given HEK293-Cas9's tendency to over-nominate off-targets, performing UNCOVERseq with >500,000 reads per sample is recommended to aim for >50% analytical sensitivity in the lowest frequency bins, and >2 million reads per sample for maximum sensitivity in assessing candidate off-target sites (FIG. 16H). It is possible that read depth requirements may vary with off-target number. However, by using a promiscuous gRNA (specificity score=0.013) to determine this value, it was proposed that this represents the number of reads to successfully nominate sites even with gRNAs with very poor specificity.

### Comparative Analysis of UNCOVERseq to Other Nomination Methods

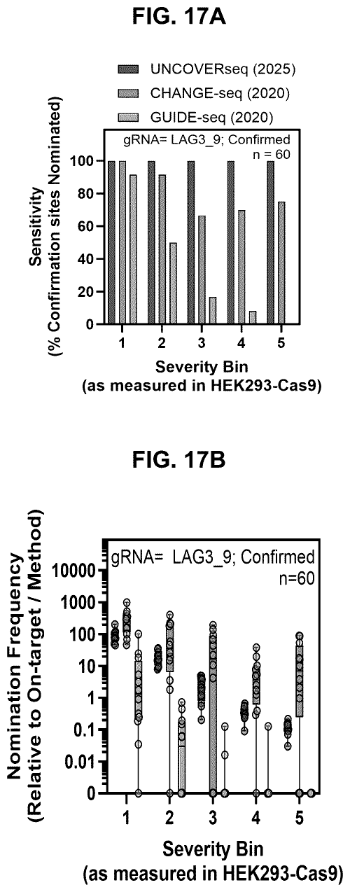

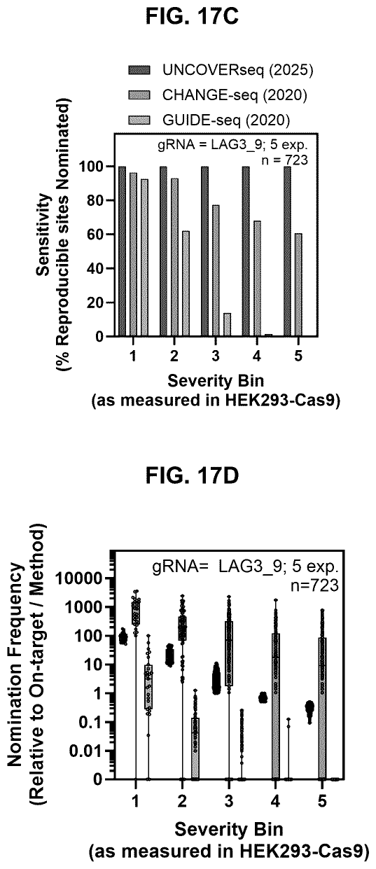

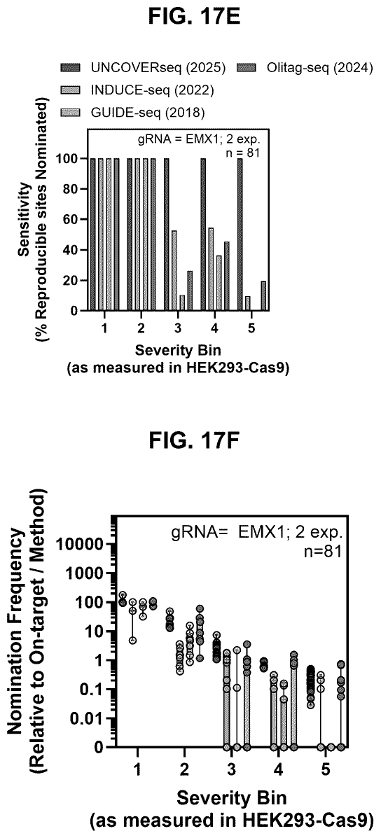

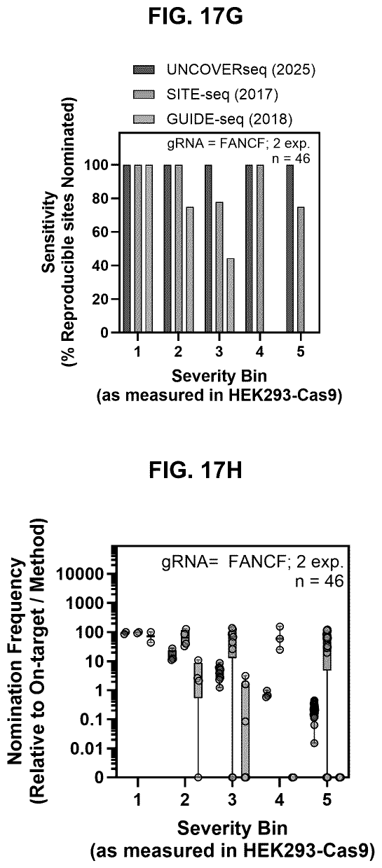

A comparative analysis of UNCOVERseq to published accounts of other nominations methods was performed to better understand how the sensitivity and nomination frequencies of diverse methods compare. Due to variable operational conditions, false positive rates, and total nomination list sizes reported of different methods, it is postulated that sensitivity is most appropriately measured using either confirmed or methods with high true positive rates. Interrogation of the 60 LAG3 site 9 gRNA off-targets confirmed to CHANGE-seq and GUIDE-Seq showed that both methods could nominate the most frequent group of confirmed off-targets (Bin 1) with 91-100% sensitivity, but sensitivity rapidly decreased in the lower frequency off-target bins. CHANGE-seq was demonstrated to have a sensitivity between 66-75% for recovering Bin 3 to Bin 5 off-targets, while GUIDE-Seq had a linear decrease from 16% to 0% for these same bins (FIG. 17A). Normalized frequencies for GUIDE-Seq roughly correlated with expected editing in the different bins, while CHANGE-seq frequencies were more uniformly distributed across bins (FIG. 17B).

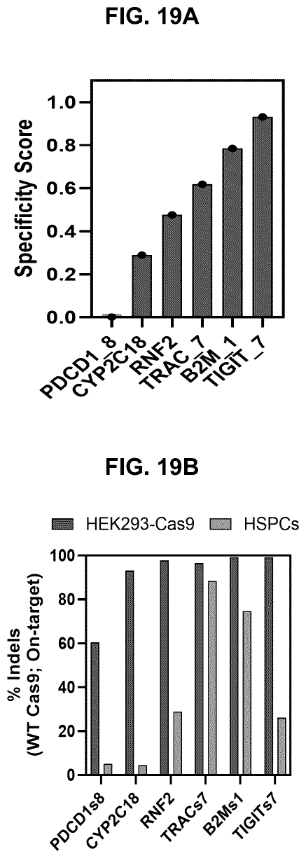

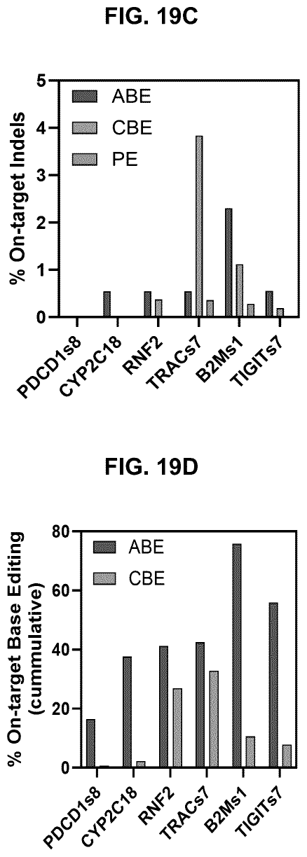

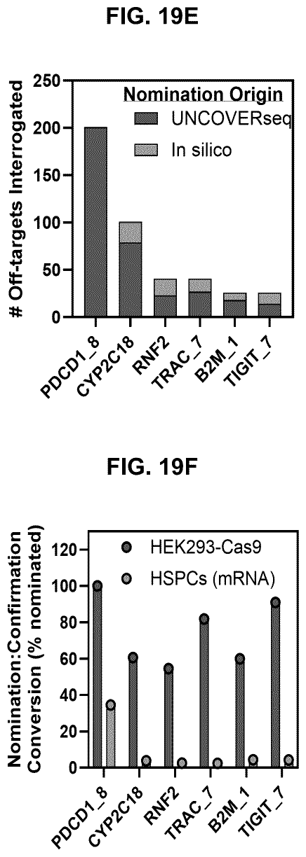

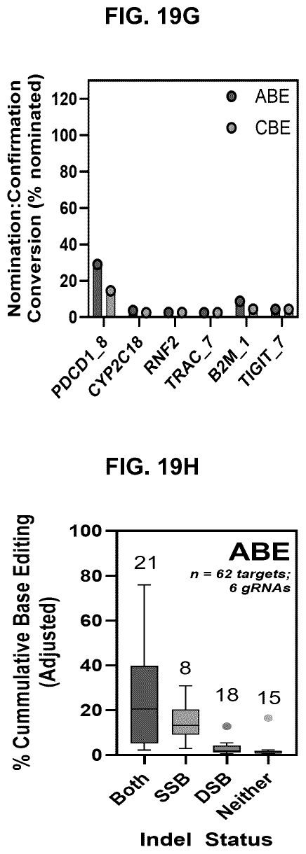

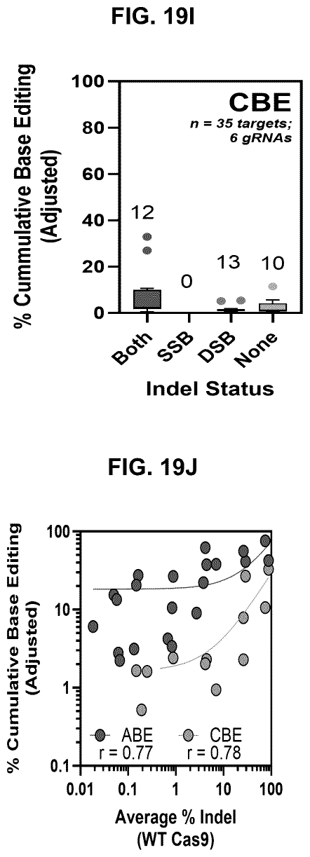

Random sampling of the LAG3 site 9 dataset with 100% reproducibility showed UNCOVERseq nominated sites had ˜100% true positive rate, with confirmation frequencies correlating to average nomination frequencies (FIG. 16F). Using this logic, it was postulated that the full 723 sites in this fully reproducible set are also likely to represent true positives. Investigation of sensitivity and frequencies of previous accounts of CHANGE-seq and GUIDE-Seq for this gRNA yielded similar trends in sensitivity and the frequency of the sites per bin, further supporting this idea (FIG. 19). This provides evidence that using sites with high likelihood of true positive rate may serve as an appropriate proxy for measuring sensitivity.

Using the previous finding that off-targets with >3 replicates reproducing a site is likely indicative of true positives (FIG. 16B), fully reproduced off-targets from 6 replicate UNCOVERseq experiments for three gRNAs (EMX1, FANCF, PCSK9) were compared to previous accounts of off-targets from GUIDE-Seq, INDUCE-seq, OliTag-seq, and SITE-seq nomination methods. For EMX1, GUIDE-Seq and INDUCE-seq sensitivity dropped after Bin 2 (10-50% frequency), ranging from 54.5-0% for frequencies below 10% in UNCOVERseq (FIG. 17E). However, nomination frequencies for GUIDE-Seq and INDUCE-seq correlated well with expected frequencies if detected (FIG. 17F). For FANCF, GUIDE-Seq sensitivity quickly decreased below 50% after Bin 2 to 0% below Bin 3 (FIG. 17G). SITE-seq showed 75-100% sensitivity across bins, but poor correlation with expected nomination frequencies (FIG. 17H). Furthermore, it can be seen that frequencies derived from in cellulo and in situ methods (UNCOVERseq, GUIDE-Seq, OliTag-seq, INDUCE-seq) better correlate to observed or predicted frequencies (FIG. 17). These findings demonstrate that UNCOVERseq improves upon existing in cellulo methods such as GUIDE-Seq, in addition to improvements upon it such as OliTag-seq. It also demonstrates in vitro methods are not inherently more sensitive than in cellulo methods for discovering true off-targets, and UNCOVERseq nominates confirmable off-targets not present in other methods.

Screening gRNAs of Variable Specificity

To identify the specificity of a broad set of gRNAs for future experimental design, 192 gRNAs were selected and UNCOVERseq was performed in HEK293-Cas9. Samples were sequenced to a median of 1.8 million reads, in line with previous recommendations for maximizing sensitivity (FIG. 18A). Following this, a specificity score was calculated for each gRNA as previously defined. It was rationalized that only a single replicate per gRNA was needed for this experimentation since higher frequency off-targets that would be recovered from a single replicate in the promiscuous system were of most interest (FIG. 14A). From this, a relatively uniform distribution of gRNA specificity scores was recovered (binned in increments of 0.2) with each bin containing a range between 22 to 47 gRNAs each and representing a continuous range from 0 to 1 (FIG. 18). These gRNAs were further subset by those that were ABE and CBE compatible as defined as having either an “A” in the +4 to +7 positions (5′ to 3′) or a “C” in the +4 to +8 positions (5′ to 3′). This resulted in a less uniform distribution of gRNAs across the specificity spectrum with a range of 8 to 21 gRNAs per specificity score bin (FIG. 18D). From this, six gRNAs were selected for further experimentation as representing a continuous range from 0 to 1 supporting all editor modalities: PDCD1 site 8, CYP2C18, RNF2, TRAC site 7, B2M site 1 and TIGIT site 7 (FIG. 19A).

### Comparative Analysis of Editors in HEK293-Cas9 and HSPCs (On-Target)

Next, it was sought to determine the translation of UNCOVERseq off-targets across a broad range of specificities in a translational ex-vivo system (HSPCs with mRNA editor nucleofection) across different editing modalities (Cas9, Base Editors, Prime Editors). To do this, HSPCs were edited with one of six gRNAs along with mRNA of either (a) wildtype S.p. Cas9; (b) S.p. Cas9 fused to a Cytosine Base Editor; (c) S.p. Cas9 fused to Adenine Base Editor version 8 (ABE8); or (d) S.p. Cas9 fused to the PE2 system with a pegRNA intended to introduce a single SNP. HEK293-Cas9 was also edited in parallel with just the wildtype S.p. Cas9 nuclease. Evaluation of on-target S.p. Cas9 editing found that editing in HEK293-Cas9 was highly efficient at all sites, ranging from 60.4-99.4% indel editing, but with a trend of decreased frequencies at lower specificity gRNAs (FIG. 19B). Evaluation of on-target indel editing in HSPCs had a range of 4.4-88.4% indel editing for the DSB-based S.p. Cas9 editor, 0.0-2.3% indel editing for ABE editors, and 0.0-3.8% for CBE editors, and 0.0-0.36% for Prime Editors (FIG. 19C). Intended on-target cumulative base editing ranged from 16.5-75.9% for ABE editors, and from 0.78-32.9% for CBE editors (FIG. 19D). No significant base editing was observed for either the DSB-based S.p. Cas9 editor or prime editor, as would be expected. No significant frequencies were observed for the intended mutation to be introduced via prime editing at any sites. For this reason, further evaluation of the prime editor was excluded. Since evidence of on-target indel editing with the PE construct was shown, it was hypothesized that the lack of intended activity was due to the need for substantial pegRNA optimization to achieve successful prime editing, as has been previously reported. For all editors in HSPCs, similar trends were observed to HEK293-Cas9 that the lower specificity gRNAs PDCD1 site 8 and CYP2C18 had trends with decreased editing, suggesting that these sites may be overall less effective at the on-target, potentially due to competing off-targets (FIG. 19). It was concluded from this that on-target editing was successful in all conditions except prime editing, with highly variable frequencies as may be expected without substantial optimization.

### Comparative Analysis of DSB Editors in HEK293-Cas9 and HSPCs (Off-Target)

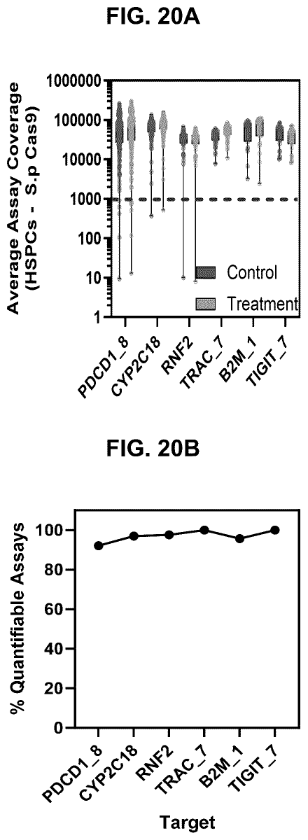

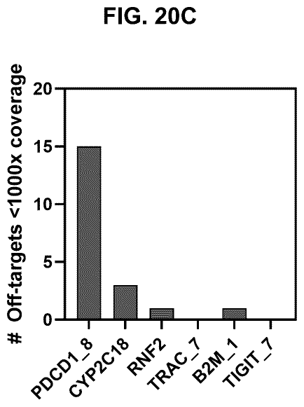

A range of nominated off-targets were selected from two orthogonal methods for downstream confirmation: UNCOVERseq nominations and in silico nominations. A range of 26 to 201 putative editing sites were interrogated per multiplexed amplicon rhAmpSeq panel with an UNCOVERseq: in silico nomination origin split ranging from 53.8% to 100% for the interrogated target lists (FIG. 19E). Panels were sequenced with a goal of at least reaching 1,000× coverage per off-target to ensure adequate sensitivity for calling significantly edited off-targets. Sequencing the six panels demonstrated the ability to hit a median read coverage ranging between 34,000× to 76,000×, with consistency in coverage between edited and corresponding control samples (FIG. 20A). The number of off-targets reaching sufficient coverage (>1,000×) ranged from 92% to 100% per panel, with a range of 0 to 15 targets not being at sufficient coverage per panel (FIG. 20B).

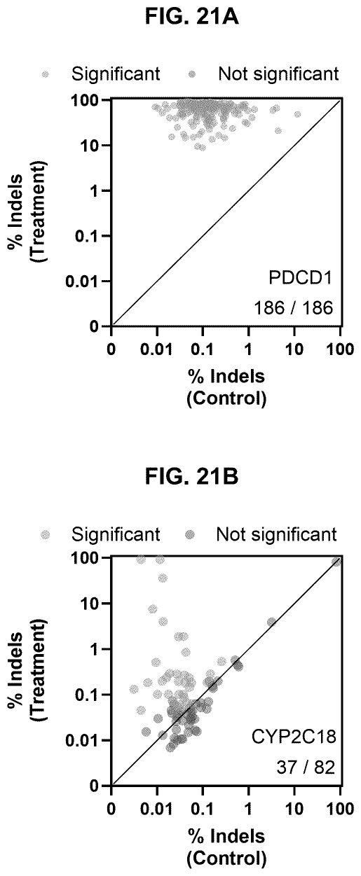

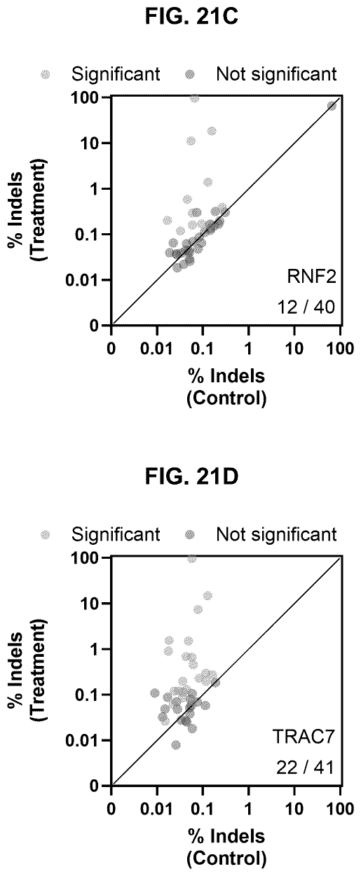

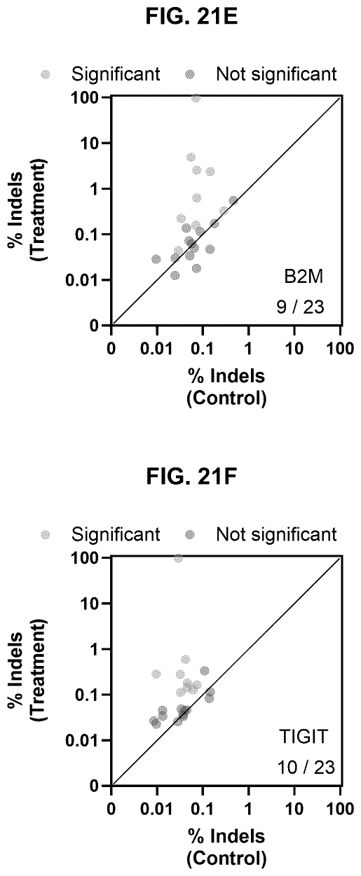

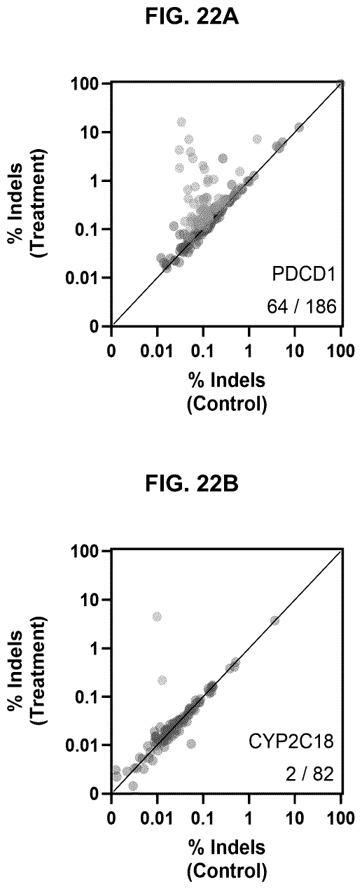

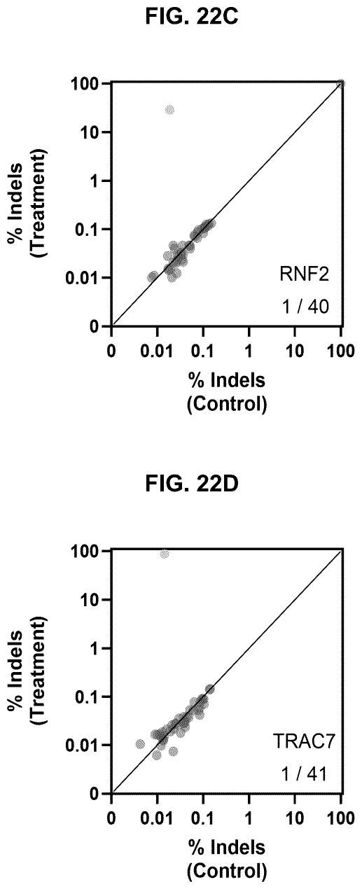

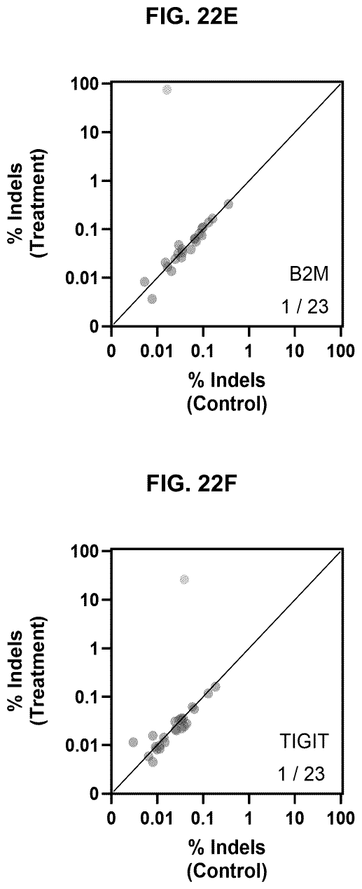

To determine the frequency of UNCOVERseq HEK293-Cas9 nominations that convert to empirically edited sites in variable DSB editing contexts, this frequency for S.p. Cas9 was compared both in HEK293-Cas9 and HSPCs. For HEK293-Cas9, a range of 54.5% of nominated off-targets all the way to 100% of off-targets had confirmed editing ranging from to 0.02-95% indel editing, demonstrating the true positive rate for UNCOVERseq nominated sites remains high even with only a single replicate in the appropriately paired confirmation context (FIG. 19F; FIG. 21). For HSPCs, a range of 2.4-34.5% of nominated targets were successfully confirmed per gRNA, with confirmed indel editing ranging from 0.06-88% (FIG. 19F; FIG. 22). Nomination: confirmation frequencies trended to increase as gRNA specificity decreased, suggesting that the method is still successfully nominating relevant off-targets, but that these sites likely no longer exceed detectable frequencies or are no longer edited in the higher genome editing specificity context of HSPCs delivered an mRNA editor (FIG. 19). Furthermore, at higher gRNA specificities the only nominated target being confirmed is the on-target site in HSPCs (FIG. 22).

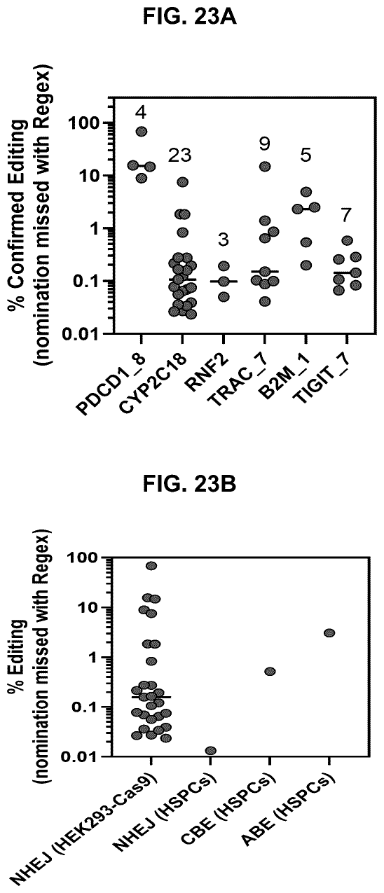

Off-targets that were confirmed were compared to the list of those that would have been dropped given a different previously evaluated alignment method (Regex method; FIG. 11). A range of 3 to 23 bona fide off-targets per gRNA in HEK293-Cas9 were successfully nominated using the alignment method that were missed using GUIDE-Seq analysis Regex method, with a range of observed indel editing from 0.02-68% (FIG. 23A). In HSPCs, 1 bona fide off-target was identified with a frequency of 0.01% with the alignment method that was missed using the Regex alignment method (FIG. 23B). This demonstrates that DSB off-target sites that were nominated due to differences in alignment criteria can result in bona fide off-target indel editing in both HEK293-Cas9 and HSPCs.

### Comparative Analysis of Non-DSB Editors in HSPCs (Off-Target)

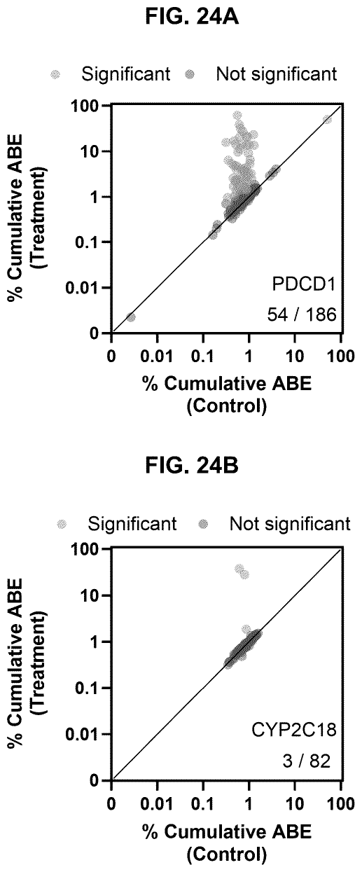

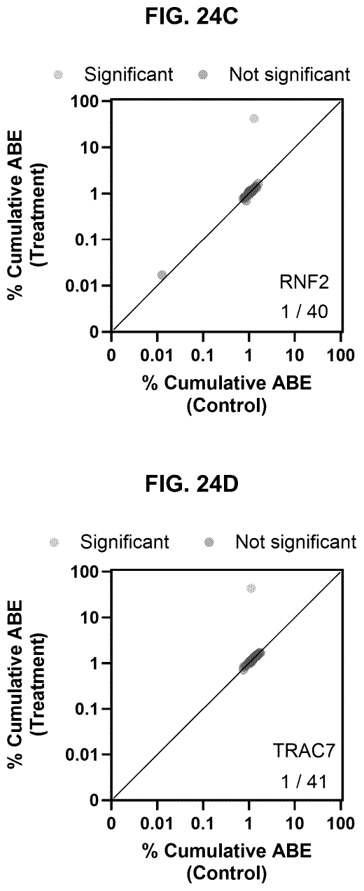

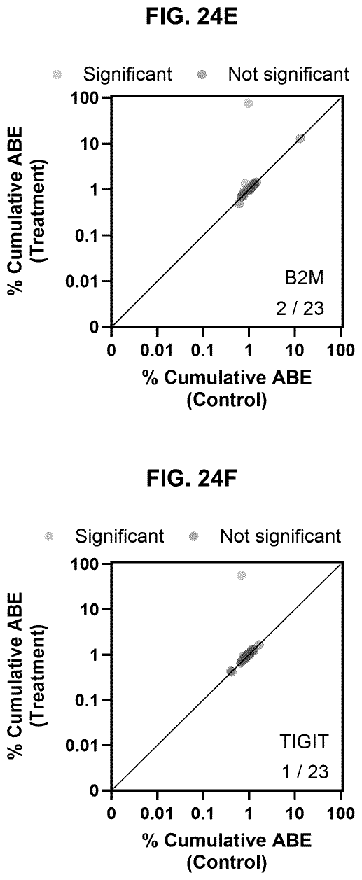

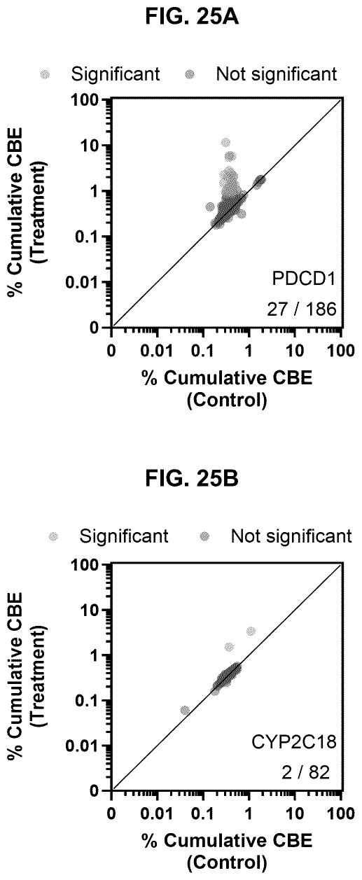

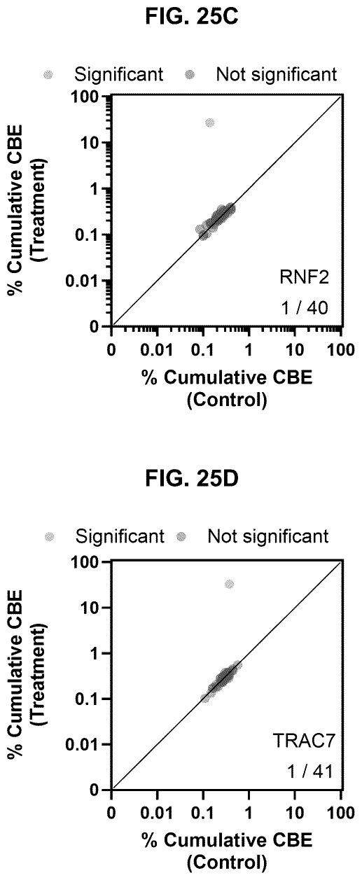

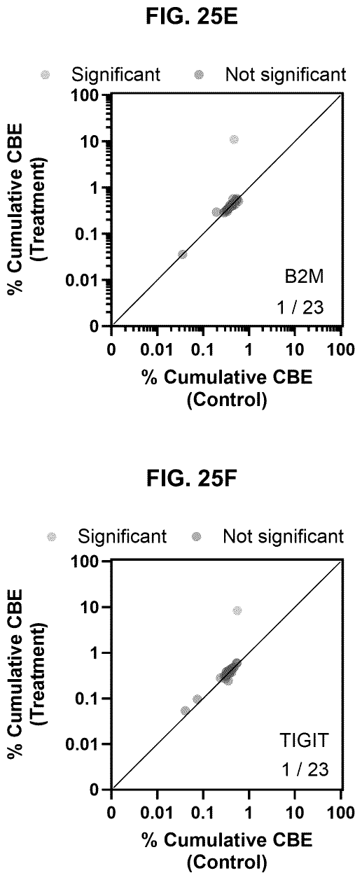

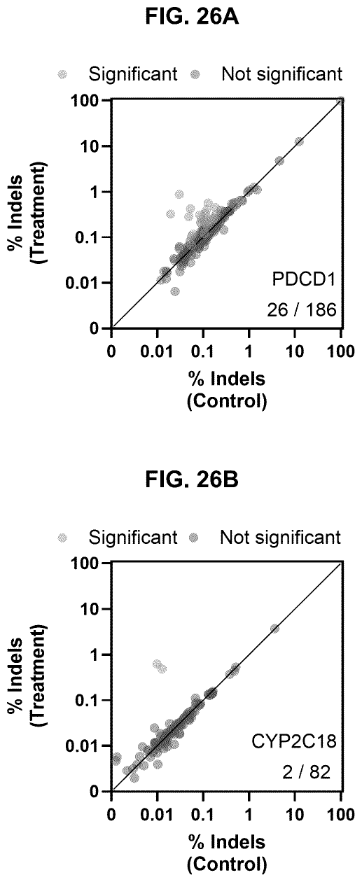

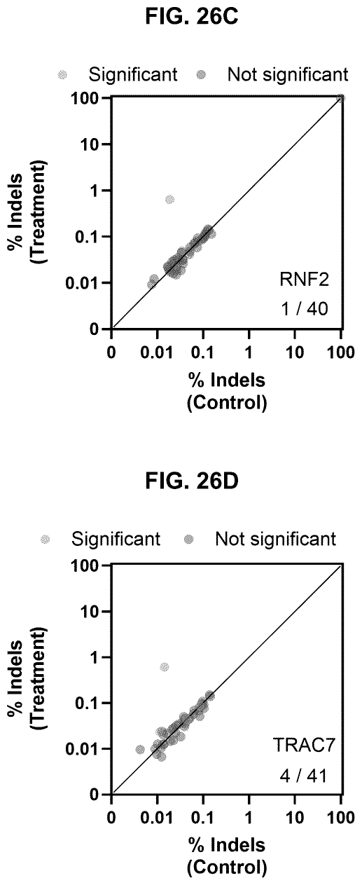

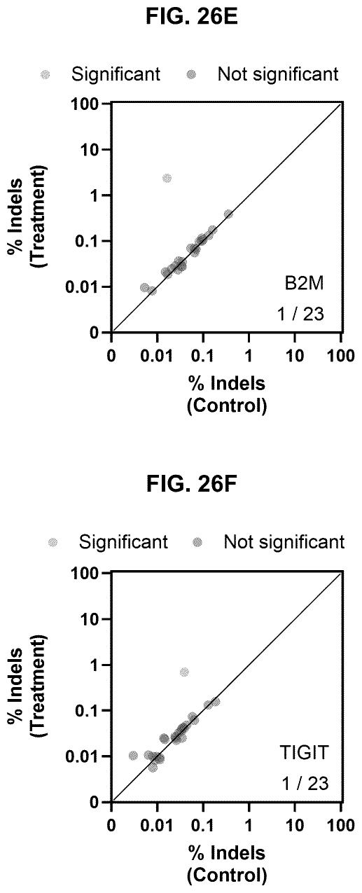

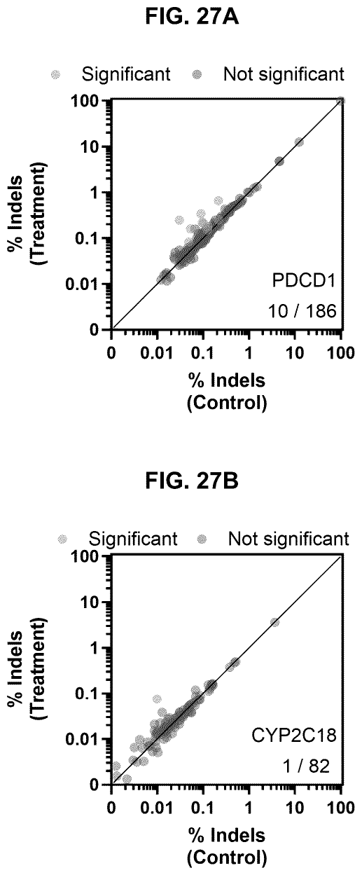

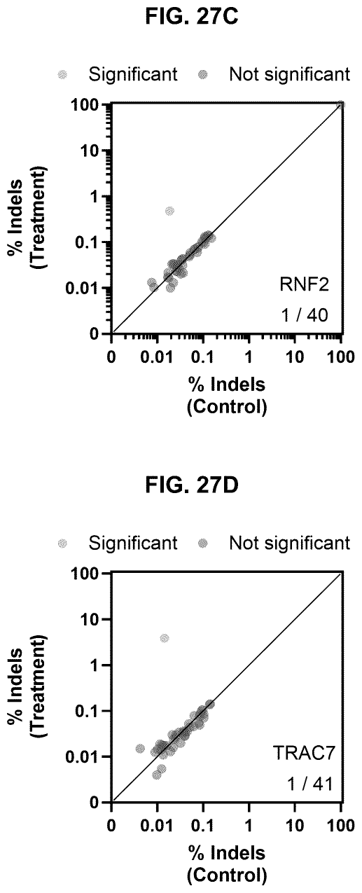

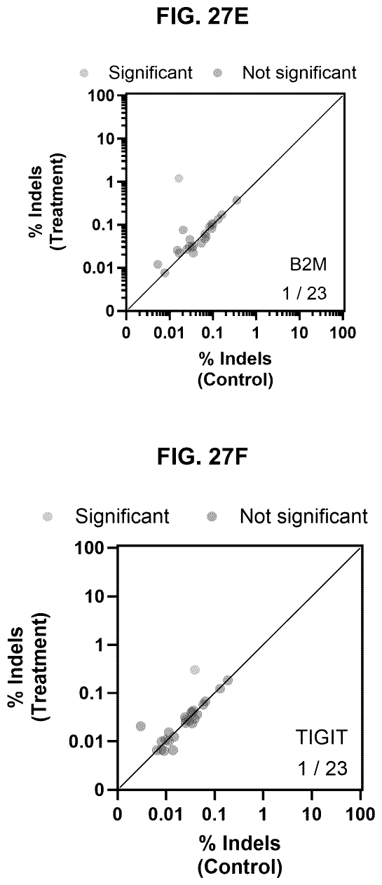

Off-targets were simultaneously confirmed for both indel and base editing in the non-DSB treatments for HSPCs (ABE and CBE). Similarly, the frequency that UNCOVERseq HEK293-Cas9 nominations convert to empirically edited sites in HSPCs being delivered a base-editor was interrogated. For ABE treatments, a range of 2.4% of nominated targets to 29% of nominated targets had confirmed editing ranging from 0.53-75.9% cumulative ABE editing (FIG. 19G; FIG. 24). For CBE treatments, a range of 2.4% of nominated targets to 14.5% of targets had confirmed editing ranging from 0.51-32.9% CBE editing (FIG. 19G; FIG. 25). Significant indel editing was observed for all ABE and CBE editing treatments, with largely only the on-target gRNA containing indels at higher specificity gRNAs (FIG. 19C; FIG. 26; FIG. 27). Off-target indel frequencies for ABE treatments ranged from 0.02-0.88% indels across different gRNAs (FIG. 26). Interestingly, three off-target sites were found to generate indel events at the higher specificity TRAC7 gRNA under ABE treatment conditions, which lacked any significant off-targets in paired wildtype S.p. Cas9 treatment (FIG. 26). Off-target indel frequencies for CBE treatments ranged from 0.08-0.66% indels across different gRNAs (FIG. 27). Generally, it was observed that indel and base editing frequencies were lower in CBE treated samples in comparison to ABE treated samples (FIG. 19; FIG. 24; FIG. 25), although this could be a result of lower overall activity instead of off-target propensity.

When comparing the list of confirmed ABE/CBE off-targets to those that would have been excluded given a different alignment method during nomination, 1 bona fide off-target of the PDCD1 gRNA was found that was identified for both ABE and CBE treatments with a frequency range of 0.5-3.1% base editing that was missed using the Regex alignment method (FIG. 23B). This demonstrated that off-target sites that were nominated due to differences in alignment criteria can also result in bona fide off-target base editing activity for both ABE and CBE editors in HSPCs.

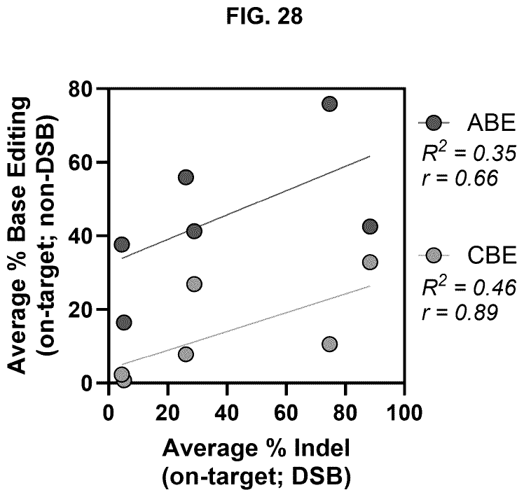

To investigate relationships between DSB indels, SSB indels, and base editing, confirmed base editing off-targets were binned based on their presence of indels in either DSB or SSB systems. Base editing with the highest frequencies (median 20.6% and 2.3% for ABE and CBE, respectively), were found to coincide with indel editing for both DSB and SSB systems (FIG. 19H). Interestingly, only ABE treatments were found to have an increased frequency of base editing at SSB only sites, with eight detected SSB-only off-targets with a median 13.1% cumulative base editing compared to zero sites for CBE (FIG. 19). This may coincide to activity differences, as both ABE editing/indel activity was generally higher than CBE editing/indel activity across the different sites (FIG. 24-27). DSB-only and sites with no evidence of significant indel editing were present in confirmed sites for both ABE and CBE treatments, albeit with lower median cumulative base editing frequencies (FIG. 19). The on-target indel and base editing activity of the different gRNAs were rank order correlated (r=0.66-0.89), suggesting that indel editing frequencies may be predictive of base editing frequencies (FIG. 28). Similarly, off-target DSB indel editing frequencies from S.p. Cas9 demonstrated rank-order correlation with off-target base editing frequencies (r=0.77-0.78) at sites that had significant DSB indel editing frequencies in HSPCs (FIG. 19J). This provided evidence that DSB editing may be indicative of base editing activity, meaning that DSB nominated sites are meaningful for interrogation in the context of both indel and base editing off-target assessment for both ABE and CBE modalities.

### Comparative Translocation Analysis and Overall Editing Burden Across Editing Modalities

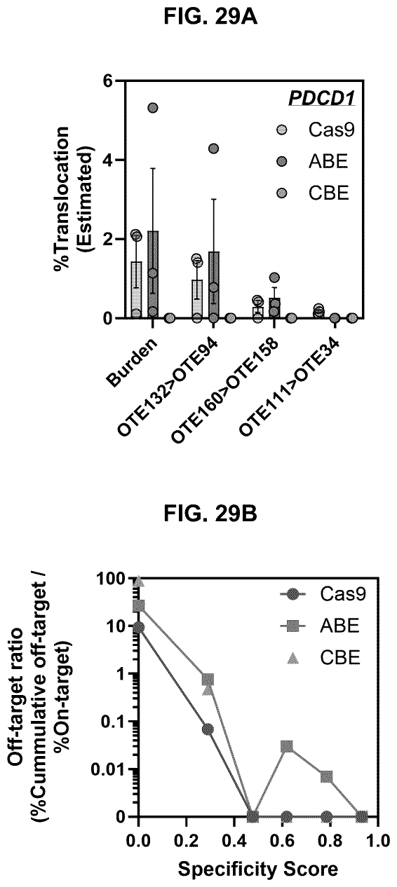

To investigate differential frequencies of editor modalities to generate large structural variants (>0.1% frequencies) in HSPCs, the previously described six sites were investigated for on-target: off-target and off-target: off-target translocations using amplicon sequencing. Only the PDCD1 gRNA had detectable translocations, with two out of three of the translocations being shared between the S.p. Cas9 and S.p. Cas9-ABE conditions (FIG. 29A). Shared translocations included a fusion of OTE132 to OTE94 and OTE160 to OTE158, with comparable average frequencies ranging between 1.0-1.7% and 0.3-0.5%, respectively (FIG. 29A). The overall estimated translocation burden for this gRNA was estimated to be an average of 1.4% translocations for S.p. Cas9 and an average of 2.2% translocations for S.p. Cas9-ABE (FIG. 29A). This suggests that translocations are either below 0.1% or not occurring in healthy HSPC donors across higher gRNA specificities. However, its noteworthy that they are still occurring for both SSB and DSB modalities.

When calculating the normalized risk of cumulative off-target frequencies (indels, base edits, and translocations) across editor modalities throughout the spectrum of gRNA specificities, off-target ratios were observed for the PDCD1 gRNA over a range 9.4-89.0 off-target events per 1 on-target event (FIG. 29B). Off-target ratios for the CYP2C18 gRNA ranged from 0.07-0.76 off-target events per 1 on-target event (FIG. 29B). Trends consistently showed that overall off-target burden of DSB editors was actually decreased in comparison to SSB base editors for the cumulative frequency of event types monitored using this strategy (FIG. 29B). Even though the B2M gRNA was considered higher specificity, a single significant ABE off-target was observed for this treatment contributing to a higher ratio (FIG. 29). This highlighted that even higher specificity gRNAs may generate observable off-targets in clinically relevant cell types.

### DISCUSSION

This study presents a versioned, end-to-end characterized in cellulo method for the nomination of off-target sites in CRISPR experiments that are collectively referred to as UNCOVERseq (v1.0). This method leverages several technological improvements to collectively streamline the in cellulo nomination process, improve NGS data quality, and increase the number of high confidence nominated sites compared to other previously published methods. By demonstrating recommended operational conditions that can allow the experiments to be performed independent of cell context with controls grounded in empirical data, a framework to ensure translation to different treatment modalities with quantifiable levels of performance from experiment to experiment is provided. Furthermore, it is demonstrated that the workflow is capable of nominating relevant unique and shared off-targets for both DSB-based and SSB-based CRISPR editing systems and demonstrate correlations between DSB formation and the frequency of a site to be edited by ABE/CBE editors.

To ensure all relevant off-targets are assessed, high analytical sensitivity is a critical off-target nomination metric. However, accurate calculations of false-negative rates from nomination methods have been challenged by technical difficulties in obtaining an empirically defined gold-standard of all true-positive off-targets. Previous work has led to a mentality that in vitro biochemical methods are inherently more sensitive that in cellulo ones as evidenced by (1) true positive sites captured by in vitro methods like CHANGE-seq that are missed with GUIDE-Seq and (2) multiple accounts of in cellulo methods being largely a subset of in vitro results. To reduce risk of false negatives, a strategy for in cellulo off-target nomination using UNCOVERseq was demonstrated where high gDNA input and promiscuous editing conditions are used to greatly amplify nomination signal to reproducibly detect sub-0.05% editing events while still retaining sites derived from higher fidelity and primary cell lines. Using UNCOVERseq, it was demonstrated that previously published accounts of in cellulo methods were not very sensitive as compared to UNCOVERseq. However, it is not clear whether this is due to insufficient operational conditions (read depth, library complexity, etc.) to maximize capabilities of the assay as opposed to the technical improvements that confer enhanced nomination capabilities to UNCOVERseq. It was also found that in vitro biochemical assays do not sensitively cover the full range of true positive lists generated from UNCOVERseq nomination. This provides evidence refuting claims that in vitro methods are inherently more sensitive for off-target nomination. Future work should look to further expand gRNAs nominated/confirmed and provide empirical knowledge on the optimal operating conditions for different assays.

High analytical specificity is another important metric for off-target nomination methods to appropriately select sites for downstream confirmation. Some methods for off-target nomination can lead to thousands of sites being nominated which is cost prohibitive for downstream interrogation given the high coverage depth required for sensitive off-target confirmation. Using UNCOVERseq with sufficient replication, it was demonstrated that true positive rates can be obtained from nomination that approach 100% specificity using replication as an indication, enabling rapid identification of true positive sites for benchmarking. It was additionally demonstrated that logic suggesting that sites with ≥3 replicates nominating a site from UNCOVERseq are highly likely to be true positives. However, future work should look to better confirm this logic by performing targeted sequencing on sites with different levels of reproducibility.

Using a simple prioritization method based on frequency, replication, and high level indicators of risk (exonic regions vs. intergenic, etc.), it was demonstrated that UNCOVERseq nominations with recommended experimental structures can result in manageable panel sizes for downstream confirmation (e.g., <300 sites across a range of specificities). However, to better understand risk after off-target nomination and confirmation across methods, a more standardized scoring system to prioritize off-targets is needed in the future. The fields of oncology and heritable diseases have encountered similar issues and derived guidelines including tiered scoring systems from the American College of Medical Genetics (ACMG) and Association of Molecular Pathology (AMP) and modifications leveraging these criteria. Gene editing may be able to leverage some of these learnings, but will face unique challenges in categorization of off-target risk since even off-targets in intergenic space during the nomination phase can be at risk for known structural variations derived from DSBs and SSBs. This includes events such as translocations, loss of heterozygosity (LoH), aneuploidy, and other large variants like multi-kilobase deletions. Given the possibility that even intergenic off-targets can result in large pathogenic rearrangements, it seems likely that probability, frequency, and even potentially proximity to other coding regions will have to be important criteria for triaging off-targets for assessment. In agreement with previous findings, it was found that in cellulo methods provide a much stronger relationship between frequency of off-targets nominated and observed compared to in vitro methods. This highlights that in cellulo methods like UNCOVERseq may have additional utility in future risk scoring criteria given their ability to be predictive of observable frequencies.

Translational contexts for nomination and confirmation already need to support both DSB and SSB-based editing modalities. By selecting a variable range of gRNA specificities, it was demonstrated that even in popular ex vivo models like HSPCs with mRNA delivery, high specificity gRNAs are still sensitive to both SSB indels and base editing off-target effects at frequencies>0.01% and 0.5%, respectively. Furthermore, it was demonstrated that indel editing and base editing are rank order correlated across 34 base editing on/off-targets, supporting the idea that DSB-based nomination methods are effective tools for nominating both indel and base editing activity. Base editing specific nomination methods, such as SELICT-seq and CHANGE-seq BE, have been developed to target base editing events, while demonstrating unique off-target confirmation findings. It is believed that orthogonal methods for nominating both base editing events and indel events may be necessary for future studies, especially given some of the findings that some UNCOVERseq nominated sites generate confirmable indels only in conditions using the SSB base editing modalities in translational cellular contexts.

It is envisioned that UNCOVERseq coupled with promiscuous conditions provides a powerful tool to help sensitively identify CRISPR-Cas off-targets for interrogation during pre-clinical development phases.

TABLE 4
dsODN Sequences

| | | SEQ |
|---|---|---|
| | | ID |
| Name | DNA Sequence (5′→3′) | NO |
| Top_ | /5Phos/T*A*A*GCGGCGTAGGTAGCCGGACGAAT | 79 |
| Strand | GTCGGTCGTA*G*T*T | |
| Bottom_ | /5Phos/A*A*C*TACGACCGACATTCGTCCGGCTA | 80 |
| Strand | CCTACGCCGC*T*T*A | |

/5Phos/indicates a 5′-terminal phosphate; * indicates a phosphorothioate linkage between the two nucleotides.

TABLE 5
Spacers and gRNAs
Spacers

| | | SEQ ID |
|---|---|---|
| Name | DNA Sequence (5′→3′) | NO: |
| AR sgRNA | GTTGGAGCATCTGAGTCCAG | 81 |
| AAVS1 | GGGGCCACTAGGGACAGGAT | 82 |
| sgRNA | | |
| LAG3 sgRNA | GAAGGCTGAGATCCTGGAGG | 83 |
| PCSK9-1 | CCCGCACCTTGGCGCAGCGG | 84 |
| BCL11a | CTAACAGTTGCTTTTATCAC | 85 |
| sgRNA | | |
| EMX1 sgRNA | GAGTCCGAGCAGAAGAAGAA | 86 |
| FANCF | GGAATCCCTTCTGCAGCACC | 87 |
| sgRNA | | |
| PDCD1s8 | GAGCAGGGCTGGGGAGAAGG | 88 |
| sgRNA | | |
| CYP2C18 | ACGAGCACCACTCTGAGATA | 89 |
| sgRNA | | |
| RNF2 sgRNA | GTCATCTTAGTCATTACCTG | 90 |
| TRACs7 | CGTCATGAGCAGATTAAACC | 91 |
| sgRNA | | |
| B2Ms1 sgRNA | GGCCGAGATGTCTCGCTCCG | 92 |
| TIGITs7 | CGCTGACCGTGAACGATACA | 93 |
| sgRNA | | |
| PDCD1_1 | CGTCTGGGCGGTGCTACAAC | 94 |
| PDCD1_2 | TGTAGCACCGCCCAGACGAC | 95 |
| PDCD1_3 | GTCTGGGCGGTGCTACAACT | 96 |
| PDCD1_4 | GAGAAGGCGGCACTCTGGTG | 97 |
| PDCD1_5 | CCCCTTCGGTCACCACGAGC | 98 |
| PDCD1_6 | CCCTTCGGTCACCACGAGCA | 99 |
| PDCD1_7 | GTGTCACACAACTGCCCAAC | 100 |
| PDCD1_8 | CGTGTCACACAACTGCCCAA | 101 |
| LAG3_1 | ACAGAGCAAAGTGGCCGTCG | 102 |
| LAG3_2 | AGCCTCCCACATCTCTCCTA | 103 |
| LAG3_3 | GAACGGCATCCCAGCCACGA | 104 |
| LAG3_4 | CCCACATCTCTCCTATGGTC | 105 |
| LAG3_5 | GCGCTGAGCCCTCCAAAAGG | 106 |
| LAG3_6 | CCACATCTCTCCTATGGTCT | 107 |
| LAG3_7 | GCAGCGCTGAGCCCTCCAAA | 108 |
| LAG3_8 | GACCAGAGGCCGGAATCCAG | 109 |
| CTLA4_1 | GTGCGGCAACCTACATGATG | 110 |
| CTLA4_2 | CCTCACTATCCAAGGACTGA | 111 |
| CTLA4_3 | CAAGTGAACCTCACTATCCA | 112 |
| CTLA4_4 | GGGACTCTACATCTGCAAGG | 113 |
| CTLA4_5 | CACGGGACTCTACATCTGCA | 114 |
| CTLA4_6 | TGTGCGGCAACCTACATGAT | 115 |
| CTLA4_7 | GATGTAGAGTCCCGTGTCCA | 116 |
| CTLA4_8 | CCGCACAGACTTCAGTCACC | 117 |
| NRP1_1 | TGGCACAAATAGCTGGCCAA | 118 |
| NRP1_2 | GGCACAAATAGCTGGCCAAA | 119 |
| NRP1_3 | CGGCTTGTTTCTGGACCCGT | 120 |
| NRP1_4 | CAACGGGTCCAGAAACAAGC | 121 |
| NRP1_5 | CTTTTCTCCAAGACGGGCTG | 122 |
| NRP1_6 | AGGCAATGCCTGGATCCGAG | 123 |
| NRP1_7 | TGCATCCTGTCATTTAGCTC | 124 |
| NRP1_8 | GAAAGCAGCGAGGCAATGCC | 125 |
| IL2RA_1 | GGGACTGCTCACGTTCATCA | 126 |
| IL2RA_2 | GGATTCATACCTGCTGATGT | 127 |
| IL2RA_3 | AAAAGAGGCTGACGGCAACT | 128 |
| IL2RA_4 | AAAAAGAGGCTGACGGCAAC | 129 |
| IL2RA_5 | ACTGCCCCGGCTGGTCCCAA | 130 |
| IL2RA_6 | CGATGCCAAAAAGAGGCTGA | 131 |
| IL2RA_7 | GAAACTCTAGCCACTCGTCC | 132 |
| IL2RA_8 | AAACTCTAGCCACTCGTCCT | 133 |
| TIGIT_1 | ACCCTGATGGGACGTACACT | 134 |
| TIGIT_2 | TACCCTGATGGGACGTACAC | 135 |
| TIGIT_3 | CACCACGGCACAAGTGACCC | 136 |
| TIGIT_4 | GCTGACCGTGAACGATACAG | 137 |
| TIGIT_5 | CTCCCAGTGTACGTCCCATC | 138 |
| TIGIT_6 | TGGGGCCACTCGATCCTTGA | 139 |
| TIGIT_7 | CGCTGACCGTGAACGATACA | 140 |
| TIGIT_8 | TCGCTGACCGTGAACGATAC | 141 |
| FOXO1_1 | GGGTCGATCTCCACCACCTG | 142 |
| FOXO1_2 | GGAGTTTAGCCAGTCCAACT | 143 |
| FOXO1_3 | GAGTTGGACTGGCTAAACTC | 144 |
| FOXO1_4 | CACCAAGGCCATCGAGAGCT | 145 |
| FOXO1_5 | ATCCACATCGAGGCTCCTCG | 146 |
| FOXO1_6 | GAGCCCAGAACTTAACTTCG | 147 |
| FOXO1_7 | CATCCACATCGAGGCTCCTC | 148 |
| FOXO1_8 | CTACGCCGACCTCATCACCA | 149 |
| FOXP3_1 | GCTCCCTGGACACCCATTCC | 150 |
| FOXP3_2 | TCCCAAATCCCAGTGCACCC | 151 |
| FOXP3_3 | TTCGAAGACCTTCTCACATC | 152 |
| FOXP3_4 | TCGAAGACCTTCTCACATCC | 153 |
| FOXP3_5 | CAAGTGGCCCGGATGTGAGA | 154 |
| FOXP3_6 | GAAGGTCTTCGAAGAGCCAG | 155 |
| FOXP3_7 | ACTGTACCATCTCTCTCTGG | 156 |
| FOXP3_8 | GGACCATCTTCTGGATGAGA | 157 |
| TRAC_1 | TCTCTCAGCTGGTACACGGC | 158 |
| TRAC_2 | CTCGACCAGCTTGACATCAC | 159 |
| TRAC_3 | AAGTTCCTGTGATGTCAAGC | 160 |
| TRAC_4 | TTCGGAACCCAATCACTGAC | 161 |
| TRAC_5 | GATTAAACCCGGCCACTTTC | 162 |
| TRAC_6 | ACCCGGCCACTTTCAGGAGG | 163 |
| TRAC_7 | CGTCATGAGCAGATTAAACC | 164 |
| TRAC_8 | TAAACCCGGCCACTTTCAGG | 165 |
| TRBC1_1 | GAACAAGGTGTTCCCACCCG | 166 |
| TRBC1_2 | CGGGTGGGAACACCTTGTTC | 167 |
| TRBC1_3 | TCAAACACAGCGACCTCGGG | 168 |
| TRBC1_4 | CGTAGAACTGGACTTGACAG | 169 |
| TRBC1_5 | ATGACGAGTGGACCCAGGAT | 170 |
| TRBC1_6 | GCTGTCAAGTCCAGTTCTAC | 171 |
| TRBC1_7 | TGACGAGTGGACCCAGGATA | 172 |
| TRBC1_8 | CTTGACAGCGGAAGTGGTTG | 173 |
| MAP4K1_1 | ACCACTATGACCTGCTACAG | 174 |
| MAP4K1_2 | CATTTTCAATAGAGACCCCC | 175 |
| MAP4K1_3 | GGGTCCACGACGTCCATCCC | 176 |
| MAP4K1_4 | GGTCCACGACGTCCATCCCT | 177 |
| MAP4K1_5 | GTCCACGACGTCCATCCCTG | 178 |
| MAP4K1_6 | TCCACGACGTCCATCCCTGG | 179 |
| MAP4K1_7 | CCAACATCGTGGCCTACCAT | 180 |
| MAP4K1_8 | CCCATGGTAGGCCACGATGT | 181 |
| CD52_1 | TAGGATCTTCGTGGCTGTCT | 182 |
| CD52_2 | ACCAGGTTGTAGAAGTTGAC | 183 |
| CD52_3 | AAGTTGACAGGCAGTGCCAT | 184 |
| CD52_4 | GCATCCAGCAACATAAGCGG | 185 |
| CD52_5 | TAACTTTATTGACCCCCAGC | 186 |
| CD52_6 | CAACCCCTCCCAAAGATGGA | 187 |
| CD52_7 | TTCTACAACCTGGTGATGTC | 188 |
| CD52_8 | GCCTGTCAACTTCTACAACC | 189 |
| B2M_1 | AAGTCAACTTCAATGTCGGA | 190 |
| B2M_2 | CGTGAGTAAACCTGAATCTT | 191 |
| B2M_3 | ACAGCCCAAGATAGTTAAGT | 192 |
| B2M_4 | ATTGTTTAGAGCTACCCAGC | 193 |
| B2M_5 | CTTACCCCACTTAACTATCT | 194 |
| B2M_6 | CGAACATCTCAAGAAGGTAT | 195 |
| B2M_7 | TTACCCCACTTAACTATCTT | 196 |
| B2M_8 | CCAATCCAGCCAGAAAGTAC | 197 |
| TRAC_June | TGTGCTAGACATGAGGTCTA | 198 |
| TRBC_June | GGAGAATGACGAGTGGACCC | 199 |
| PD1_June | GGCGCCCTGGCCAGTCGTCT | 200 |
| TRAC_Eyquem | CAGGGTTCTGGATATCTGTG | 201 |
| B2M_Eyquem | GGCCACGGAGCGAGACATCT | 202 |
| HEK1_ | GGGAAAGACCCAGCATCCGT | 203 |
| Chaudhari | | |
| HEK3_ | GGCCCAGACTGAGCACGTGA | 204 |
| Chaudhari | | |
| RNF2_ | GTCATCTTAGTCATTACCTG | 205 |
| Chaudhari | | |
| FANCF_ | GGAATCCCTTCTGCAGCACC | 206 |
| Chaudhari | | |
| VEGFA1_ | GGGTGGGGGGAGTTTGCTCC | 207 |
| Chaudhari | | |
| IL2RG_ | TGGTAATGATGGCTTCAACA | 208 |
| Chaudhari | | |
| HEK2_ | GAACACAAAGCATAGACTGC | 209 |
| Chaudhari | | |
| CCR5_ | GTGTTCATCTTTGGTTTTGT | 210 |
| Chaudhari | | |
| ALKAL1 | TGTCCCCGCACGGAGCCCAC | 211 |
| C19orf84 | GGGGGCCTACACCTTCCAAC | 212 |
| ATP6V0A2 | TGTTTGGATAGGGGTACACG | 213 |
| ADPGK | AGCCCAAGGGAAGTCACCGC | 214 |
| C17orf99 | GCGGGCCAACTTCACTCTGC | 215 |
| ACAT1 | TCAAGCTTTACCCCACCATA | 216 |
| AR | GTTGGAGCATCTGAGTCCAG | 217 |
| EMX1 | GAGTCCGAGCAGAAGAAGAA | 218 |
| LAG3 | GAAGGCTGAGATCCTGGAGG | 219 |
| AAVS1_site_ | GGGAACCCAGCGAGTGAAGA | 220 |
| 10 | | |
| AAVS1_site_3 | GAGCCACATTAACCGGCCCT | 221 |
| AAVS1_site_ | GGTGAGGGAGGAGAGATGCC | 222 |
| 11 | | |
| B2M_site_1 | GGCCGAGATGTCTCGCTCCG | 223 |
| B2M_site_5 | GAAGTTGACTTACTGAAGAA | 224 |
| B2M_site_2 | GCTACTCTCTCTTTCTGGCC | 225 |
| CBLB_site_4 | GGCAGAAACCCTGGTGGTCG | 226 |
| CBLB_site_6 | GGATTTCCTCCTCGACCACC | 227 |
| CBLB_site_8 | GGGTATTATTGATGCTATTC | 228 |
| CCR5_site_9 | GGTACCTATCGATTGTCAGG | 229 |
| CCR5_site_13 | GACATTAAAGATAGTCATCT | 230 |
| CCR5_site_4 | GTAGAGCGGAGGCAGGAGGC | 231 |
| CTLA4_site_ | GAGGTTCACTTGATTTCCAC | 232 |
| 10 | | |
| CTLA4_site_6 | GTGCGGCAACCTACATGATG | 233 |
| CTLA4_site_ | GCACAAGGCTCAGCTGAACC | 234 |
| 12 | | |
| CXCR4_site_ | GATAACTACACCGAGGAAAT | 235 |
| 1 | | |
| CXCR4_site_ | GCCGTGGCAAACTGGTACTT | 236 |
| 10 | | |
| CXCR4_site_ | GAAGATGATGGAGTAGATGG | 237 |
| 3 | | |
| FAS_site_3 | GGGGCAGCTCCGGCGCTCCT | 238 |
| FAS_site_2 | GCTGACCCCGCTGGGCAGGC | 239 |
| FAS_site_1 | GAGGGCTCACCAGAGGTAGG | 240 |
| LAG3_site_2 | GCTGTTTCTGCAGCCGCTTT | 241 |
| LAG3_site_5 | GGTCCCGGTGGTGTGGGCCC | 242 |
| LAG3_site_6 | GGTGGTGTGGGCCCAGGAGG | 243 |
| PDCD1_site_ | GCGTGACTTCCACATGAGCG | 244 |
| 13 | | |
| PDCD1_site_ | GTCTGGGCGGTGCTACAACT | 245 |
| 3 | | |
| PDCD1_site_ | GAGCAGGGCTGGGGAGAAGG | 246 |
| 8 | | |
| PTPN2_site_1 | GGAAACTTGGCCACTCTATG | 247 |
| PTPN2_site_2 | GGCACCAACTGGATGGATCA | 248 |
| PTPN2_site_3 | GTCTCCCTGATCCATCCAGT | 249 |
| PTPN6_site_8 | GTTTGCGACTCTGACAGAGC | 250 |
| PTPN6_site_4 | GGTTTCACCGAGACCTCAGT | 251 |
| PTPN6_site_3 | GATTTCTATGACCTGTATGG | 252 |
| TRAC_site_3 | GAGAATCAAAATCGGTGAAT | 253 |
| TRAC_site_4 | GACACCTTCTTCCCCAGCCC | 254 |
| TRAC_site_2 | GCTGGTACACGGCAGGGTCA | 255 |
| TRBC1_site_1 | GAACAAGGTGTTCCCACCCG | 256 |
| TRBC1_site_2 | GGTGCACAGTGGGGTCAGCA | 257 |
| RAB6B | GACGTCGTCGATCCACTTAG | 258 |
| ZFX | TCACCCGTCAAGACGTGTTC | 259 |
| EPM2A | TGTACCAGAACGTGTCCACG | 260 |
| CPXM2 | ACGGACACTGTGATCATCGT | 261 |
| SYNGAP1 | CCAACCAGGACGATCATACG | 262 |
| GPR141 | TGTCACTATAGGATCGCAAG | 263 |
| KRTAP13-2 | CCTTGCAAGACGACTTACTC | 264 |
| RNF10 | GTGTCCACAACGGGTTATCT | 265 |
| DMXL2 | GGAGACAACTGCTACTCCGT | 266 |
| ADGRV1 | TTGTCCTTTCCACGAACTAC | 267 |
| PTP4A3 | GAAGTACGGGGCTACCACTG | 268 |
| CYP2C18 | ACGAGCACCACTCTGAGATA | 269 |
| OR4A15 | TGTCGGAGCCTACAAACAAA | 270 |
| PCBP2 | ATGGACACCGGTGTGATTGA | 271 |
| PAPSS1 | GCAACCACGAAAGCCACCTC | 272 |
| DPY19L3 | GCTTGTAGTAGGAGTAATAC | 273 |
| SLFN12 | TCATGGAGCTTGAACACCTC | 274 |
| NKX2-8 | AACCAGATCTTGACCTGCGT | 275 |
| RIPPLY2 | CAGGAAAGCTTTACCAATTC | 276 |
| PKLR | CGGCACGACCCGGACAATAT | 277 |
| XRCC5 | GGTGGACAAGCGGCAGATAG | 278 |
| CD34 | ATAGGAGAAGATGATGTATA | 279 |
| PAPSS2_tgt_ | GCATACAGTGATTTGATGAA | 280 |
| 1 | | |
| CD151 | GCTGATGTAGTCACTCTTGA | 281 |
| PTPRC_tgt_2 | GCAAAACTCAACCCTACCCC | 282 |
| PTPRC_tgt_5 | CTCGTCTGATAAGACAACAG | 283 |
| HBB | CTTGCCCCACAGGGCAGTAA | 284 |

gRNAS

| | | SEQ ID |
|---|---|---|
| Name | RNA Sequence (5′→3′) | NO |
| AR sgRNA | /5XT/GUUGGAGCAUCUGAGUCCAGGUUUUAGAGCUAGAAAUAGCAAGUUAAAAU | 285 |
| | AAGGCUAGUCCGUUAUCAACUUGAAAAAGUGGCACCGAGUCGGUGCUUUU/3XT/ | |
| AAVS1 | /5XT/GGGGCCACUAGGGACAGGAUGUUUUAGAGCUAGAAAUAGCAAGUUAAAAU | 286 |
| sgRNA | AAGGCUAGUCCGUUAUCAACUUGAAAAAGUGGCACCGAGUCGGUGCUUUU/3XT/ | |
| LAG3 sgRNA | /5XT/GAAGGCUGAGAUCCUGGAGGGUUUUAGAGCUAGAAAUAGCAAGUUAAAAU | 287 |
| | AAGGCUAGUCCGUUAUCAACUUGAAAAAGUGGCACCGAGUCGGUGCUUUU/3XT/ | |
| PCSK9-1 | /5XT/CCCGCACCUUGGCGCAGCGGGUUUUAGAGCUAGAAAUAGCAAGUUAAAAU | 288 |
| | AAGGCUAGUCCGUUAUCAACUUGAAAAAGUGGCACCGAGUCGGUGCUUUU/3XT/ | |
| BCL11a | /5XT/CUAACAGUUGCUUUUAUCACGUUUUAGAGCUAGAAAUAGCAAGUUAAAAU | 289 |
| sgRNA | AAGGCUAGUCCGUUAUCAACUUGAAAAAGUGGCACCGAGUCGGUGCUUUU/3XT/ | |
| EMX1 sgRNA | /5XT/GAGUCCGAGCAGAAGAAGAAGUUUUAGAGCUAGAAAUAGCAAGUUAAAAU | 290 |
| | AAGGCUAGUCCGUUAUCAACUUGAAAAAGUGGCACCGAGUCGGUGCUUUU/3XT/ | |
| FANCF | /5XT/GGAAUCCCUUCUGCAGCACCGUUUUAGAGCUAGAAAUAGCAAGUUAAAAU | 291 |
| sgRNA | AAGGCUAGUCCGUUAUCAACUUGAAAAAGUGGCACCGAGUCGGUGCUUUU/3XT/ | |
| PDCD1s8 | /5XT/GAGCAGGGCUGGGGAGAAGGGUUUUAGAGCUAGAAAUAGCAAGUUAAAAU | 292 |
| sgRNA | AAGGCUAGUCCGUUAUCAACUUGAAAAAGUGGCACCGAGUCGGUGCUUUU/3XT/ | |
| CYP2C18 | /5XT/ACGAGCACCACUCUGAGAUAGUUUUAGAGCUAGAAAUAGCAAGUUAAAAU | 293 |
| sgRNA | AAGGCUAGUCCGUUAUCAACUUGAAAAAGUGGCACCGAGUCGGUGCUUUU/3XT/ | |
| RNF2 sgRNA | /5XT/GUCAUCUUAGUCAUUACCUGGUUUUAGAGCUAGAAAUAGCAAGUUAAAAU | 294 |
| | AAGGCUAGUCCGUUAUCAACUUGAAAAAGUGGCACCGAGUCGGUGCUUUU/3XT/ | |
| TRACs7 | /5XT/CGUCAUGAGCAGAUUAAACCGUUUUAGAGCUAGAAAUAGCAAGUUAAAAU | 295 |
| sgRNA | AAGGCUAGUCCGUUAUCAACUUGAAAAAGUGGCACCGAGUCGGUGCUUUU/3XT/ | |
| B2Ms1 sgRNA | /5XT/GGCCGAGAUGUCUCGCUCCGGUUUUAGAGCUAGAAAUAGCAAGUUAAAAU | 296 |
| | AAGGCUAGUCCGUUAUCAACUUGAAAAAGUGGCACCGAGUCGGUGCUUUU/3XT/ | |
| TIGITs7 | /5XT/CGCUGACCGUGAACGAUACAGUUUUAGAGCUAGAAAUAGCAAGUUAAAAU | 297 |
| sgRNA | AAGGCUAGUCCGUUAUCAACUUGAAAAAGUGGCACCGAGUCGGUGCUUUU/3XT/ | |
| PDCD1_1 | /5XT/CGUCUGGGCGGUGCUACAACGUUUUAGAGCUAGAAAUAGCAAGUUAAAAU | 298 |
| | AAGGCUAGUCCGUUAUCAACUUGAAAAAGUGGCACCGAGUCGGUGCUUUU/3XT/ | |
| PDCD1_2 | /5XT/UGUAGCACCGCCCAGACGACGUUUUAGAGCUAGAAAUAGCAAGUUAAAAU | 299 |
| | AAGGCUAGUCCGUUAUCAACUUGAAAAAGUGGCACCGAGUCGGUGCUUUU/3XT/ | |
| PDCD1_3 | /5XT/GUCUGGGCGGUGCUACAACUGUUUUAGAGCUAGAAAUAGCAAGUUAAAAU | 300 |
| | AAGGCUAGUCCGUUAUCAACUUGAAAAAGUGGCACCGAGUCGGUGCUUUU/3XT/ | |
| PDCD1_4 | /5XT/GAGAAGGCGGCACUCUGGUGGUUUUAGAGCUAGAAAUAGCAAGUUAAAAU | 301 |
| | AAGGCUAGUCCGUUAUCAACUUGAAAAAGUGGCACCGAGUCGGUGCUUUU/3XT/ | |
| PDCD1_5 | /5XT/CCCCUUCGGUCACCACGAGCGUUUUAGAGCUAGAAAUAGCAAGUUAAAAU | 302 |
| | AAGGCUAGUCCGUUAUCAACUUGAAAAAGUGGCACCGAGUCGGUGCUUUU/3XT/ | |
| PDCD1_6 | /5XT/CCCUUCGGUCACCACGAGCAGUUUUAGAGCUAGAAAUAGCAAGUUAAAAU | 303 |
| | AAGGCUAGUCCGUUAUCAACUUGAAAAAGUGGCACCGAGUCGGUGCUUUU/3XT/ | |
| PDCD1_7 | /5XT/GUGUCACACAACUGCCCAACGUUUUAGAGCUAGAAAUAGCAAGUUAAAAU | 304 |
| | AAGGCUAGUCCGUUAUCAACUUGAAAAAGUGGCACCGAGUCGGUGCUUUU/3XT/ | |
| PDCD1_8 | /5XT/CGUGUCACACAACUGCCCAAGUUUUAGAGCUAGAAAUAGCAAGUUAAAAU | 305 |
| | AAGGCUAGUCCGUUAUCAACUUGAAAAAGUGGCACCGAGUCGGUGCUUUU/3XT/ | |
| LAG3_1 | /5XT/ACAGAGCAAAGUGGCCGUCGGUUUUAGAGCUAGAAAUAGCAAGUUAAAAU | 306 |
| | AAGGCUAGUCCGUUAUCAACUUGAAAAAGUGGCACCGAGUCGGUGCUUUU/3XT/ | |
| LAG3_2 | /5XT/AGCCUCCCACAUCUCUCCUAGUUUUAGAGCUAGAAAUAGCAAGUUAAAAU | 307 |
| | AAGGCUAGUCCGUUAUCAACUUGAAAAAGUGGCACCGAGUCGGUGCUUUU/3XT/ | |
| LAG3_3 | /5XT/GAACGGCAUCCCAGCCACGAGUUUUAGAGCUAGAAAUAGCAAGUUAAAAU | 308 |
| | AAGGCUAGUCCGUUAUCAACUUGAAAAAGUGGCACCGAGUCGGUGCUUUU/3XT/ | |
| LAG3_4 | /5XT/CCCACAUCUCUCCUAUGGUCGUUUUAGAGCUAGAAAUAGCAAGUUAAAAU | 309 |
| | AAGGCUAGUCCGUUAUCAACUUGAAAAAGUGGCACCGAGUCGGUGCUUUU/3XT/ | |
| LAG3_5 | /5XT/GCGCUGAGCCCUCCAAAAGGGUUUUAGAGCUAGAAAUAGCAAGUUAAAAU | 310 |
| | AAGGCUAGUCCGUUAUCAACUUGAAAAAGUGGCACCGAGUCGGUGCUUUU/3XT/ | |
| LAG3_6 | /5XT/CCACAUCUCUCCUAUGGUCUGUUUUAGAGCUAGAAAUAGCAAGUUAAAAU | 311 |
| | AAGGCUAGUCCGUUAUCAACUUGAAAAAGUGGCACCGAGUCGGUGCUUUU/3XT/ | |
| LAG3_7 | /5XT/GCAGCGCUGAGCCCUCCAAAGUUUUAGAGCUAGAAAUAGCAAGUUAAAAU | 312 |
| | AAGGCUAGUCCGUUAUCAACUUGAAAAAGUGGCACCGAGUCGGUGCUUUU/3XT/ | |
| LAG3_8 | /5XT/GACCAGAGGCCGGAAUCCAGGUUUUAGAGCUAGAAAUAGCAAGUUAAAAU | 313 |
| | AAGGCUAGUCCGUUAUCAACUUGAAAAAGUGGCACCGAGUCGGUGCUUUU/3XT/ | |
| CTLA4_1 | /5XT/GUGCGGCAACCUACAUGAUGGUUUUAGAGCUAGAAAUAGCAAGUUAAAAU | 314 |
| | AAGGCUAGUCCGUUAUCAACUUGAAAAAGUGGCACCGAGUCGGUGCUUUU/3XT/ | |
| CTLA4_2 | /5XT/CCUCACUAUCCAAGGACUGAGUUUUAGAGCUAGAAAUAGCAAGUUAAAAU | 315 |
| | AAGGCUAGUCCGUUAUCAACUUGAAAAAGUGGCACCGAGUCGGUGCUUUU/3XT/ | |
| CTLA4_3 | /5XT/CAAGUGAACCUCACUAUCCAGUUUUAGAGCUAGAAAUAGCAAGUUAAAAU | 316 |
| | AAGGCUAGUCCGUUAUCAACUUGAAAAAGUGGCACCGAGUCGGUGCUUUU/3XT/ | |
| CTLA4_4 | /5XT/GGGACUCUACAUCUGCAAGGGUUUUAGAGCUAGAAAUAGCAAGUUAAAAU | 317 |
| | AAGGCUAGUCCGUUAUCAACUUGAAAAAGUGGCACCGAGUCGGUGCUUUU/3XT/ | |
| CTLA4_5 | /5XT/CACGGGACUCUACAUCUGCAGUUUUAGAGCUAGAAAUAGCAAGUUAAAAU | 318 |
| | AAGGCUAGUCCGUUAUCAACUUGAAAAAGUGGCACCGAGUCGGUGCUUUU/3XT/ | |
| CTLA4_6 | /5XT/UGUGCGGCAACCUACAUGAUGUUUUAGAGCUAGAAAUAGCAAGUUAAAAU | 319 |
| | AAGGCUAGUCCGUUAUCAACUUGAAAAAGUGGCACCGAGUCGGUGCUUUU/3XT/ | |
| CTLA4_7 | /5XT/GAUGUAGAGUCCCGUGUCCAGUUUUAGAGCUAGAAAUAGCAAGUUAAAAU | 320 |
| | AAGGCUAGUCCGUUAUCAACUUGAAAAAGUGGCACCGAGUCGGUGCUUUU/3XT/ | |
| CTLA4_8 | /5XT/CCGCACAGACUUCAGUCACCGUUUUAGAGCUAGAAAUAGCAAGUUAAAAU | 321 |
| | AAGGCUAGUCCGUUAUCAACUUGAAAAAGUGGCACCGAGUCGGUGCUUUU/3XT/ | |
| NRP1_1 | /5XT/UGGCACAAAUAGCUGGCCAAGUUUUAGAGCUAGAAAUAGCAAGUUAAAAU | 322 |
| | AAGGCUAGUCCGUUAUCAACUUGAAAAAGUGGCACCGAGUCGGUGCUUUU/3XT/ | |
| NRP1_2 | /5XT/GGCACAAAUAGCUGGCCAAAGUUUUAGAGCUAGAAAUAGCAAGUUAAAAU | 323 |
| | AAGGCUAGUCCGUUAUCAACUUGAAAAAGUGGCACCGAGUCGGUGCUUUU/3XT/ | |
| NRP1_3 | /5XT/CGGCUUGUUUCUGGACCCGUGUUUUAGAGCUAGAAAUAGCAAGUUAAAAU | 324 |
| | AAGGCUAGUCCGUUAUCAACUUGAAAAAGUGGCACCGAGUCGGUGCUUUU/3XT/ | |
| NRP1_4 | /5XT/CAACGGGUCCAGAAACAAGCGUUUUAGAGCUAGAAAUAGCAAGUUAAAAU | 325 |
| | AAGGCUAGUCCGUUAUCAACUUGAAAAAGUGGCACCGAGUCGGUGCUUUU/3XT/ | |
| NRP1_5 | /5XT/CUUUUCUCCAAGACGGGCUGGUUUUAGAGCUAGAAAUAGCAAGUUAAAAU | 326 |
| | AAGGCUAGUCCGUUAUCAACUUGAAAAAGUGGCACCGAGUCGGUGCUUUU/3XT/ | |
| NRP1_6 | /5XT/AGGCAAUGCCUGGAUCCGAGGUUUUAGAGCUAGAAAUAGCAAGUUAAAAU | 327 |
| | AAGGCUAGUCCGUUAUCAACUUGAAAAAGUGGCACCGAGUCGGUGCUUUU/3XT/ | |
| NRP1_7 | /5XT/UGCAUCCUGUCAUUUAGCUCGUUUUAGAGCUAGAAAUAGCAAGUUAAAAU | 328 |
| | AAGGCUAGUCCGUUAUCAACUUGAAAAAGUGGCACCGAGUCGGUGCUUUU/3XT/ | |
| NRP1_8 | /5XT/GAAAGCAGCGAGGCAAUGCCGUUUUAGAGCUAGAAAUAGCAAGUUAAAAU | 329 |
| | AAGGCUAGUCCGUUAUCAACUUGAAAAAGUGGCACCGAGUCGGUGCUUUU/3XT/ | |
| IL2RA_1 | /5XT/GGGACUGCUCACGUUCAUCAGUUUUAGAGCUAGAAAUAGCAAGUUAAAAU | 330 |
| | AAGGCUAGUCCGUUAUCAACUUGAAAAAGUGGCACCGAGUCGGUGCUUUU/3XT/ | |
| IL2RA_2 | /5XT/GGAUUCAUACCUGCUGAUGUGUUUUAGAGCUAGAAAUAGCAAGUUAAAAU | 331 |
| | AAGGCUAGUCCGUUAUCAACUUGAAAAAGUGGCACCGAGUCGGUGCUUUU/3XT/ | |
| IL2RA_3 | /5XT/AAAAGAGGCUGACGGCAACUGUUUUAGAGCUAGAAAUAGCAAGUUAAAAU | 332 |
| | AAGGCUAGUCCGUUAUCAACUUGAAAAAGUGGCACCGAGUCGGUGCUUUU/3XT/ | |
| IL2RA_4 | /5XT/AAAAAGAGGCUGACGGCAACGUUUUAGAGCUAGAAAUAGCAAGUUAAAAU | 333 |
| | AAGGCUAGUCCGUUAUCAACUUGAAAAAGUGGCACCGAGUCGGUGCUUUU/3XT/ | |
| IL2RA_5 | /5XT/ACUGCCCCGGCUGGUCCCAAGUUUUAGAGCUAGAAAUAGCAAGUUAAAAU | 334 |
| | AAGGCUAGUCCGUUAUCAACUUGAAAAAGUGGCACCGAGUCGGUGCUUUU/3XT/ | |
| IL2RA_6 | /5XT/CGAUGCCAAAAAGAGGCUGAGUUUUAGAGCUAGAAAUAGCAAGUUAAAAU | 335 |
| | AAGGCUAGUCCGUUAUCAACUUGAAAAAGUGGCACCGAGUCGGUGCUUUU/3XT/ | |
| IL2RA_7 | /5XT/GAAACUCUAGCCACUCGUCCGUUUUAGAGCUAGAAAUAGCAAGUUAAAAU | 336 |
| | AAGGCUAGUCCGUUAUCAACUUGAAAAAGUGGCACCGAGUCGGUGCUUUU/3XT/ | |
| IL2RA_8 | /5XT/AAACUCUAGCCACUCGUCCUGUUUUAGAGCUAGAAAUAGCAAGUUAAAAU | 337 |
| | AAGGCUAGUCCGUUAUCAACUUGAAAAAGUGGCACCGAGUCGGUGCUUUU/3XT/ | |
| TIGIT_1 | /5XT/ACCCUGAUGGGACGUACACUGUUUUAGAGCUAGAAAUAGCAAGUUAAAAU | 338 |
| | AAGGCUAGUCCGUUAUCAACUUGAAAAAGUGGCACCGAGUCGGUGCUUUU/3XT/ | |
| TIGIT_2 | /5XT/UACCCUGAUGGGACGUACACGUUUUAGAGCUAGAAAUAGCAAGUUAAAAU | 339 |
| | AAGGCUAGUCCGUUAUCAACUUGAAAAAGUGGCACCGAGUCGGUGCUUUU/3XT/ | |
| TIGIT_3 | /5XT/CACCACGGCACAAGUGACCCGUUUUAGAGCUAGAAAUAGCAAGUUAAAAU | 340 |
| | AAGGCUAGUCCGUUAUCAACUUGAAAAAGUGGCACCGAGUCGGUGCUUUU/3XT/ | |
| TIGIT_4 | /5XT/GCUGACCGUGAACGAUACAGGUUUUAGAGCUAGAAAUAGCAAGUUAAAAU | 341 |
| | AAGGCUAGUCCGUUAUCAACUUGAAAAAGUGGCACCGAGUCGGUGCUUUU/3XT/ | |
| TIGIT_5 | /5XT/CUCCCAGUGUACGUCCCAUCGUUUUAGAGCUAGAAAUAGCAAGUUAAAAU | 342 |
| | AAGGCUAGUCCGUUAUCAACUUGAAAAAGUGGCACCGAGUCGGUGCUUUU/3XT/ | |
| TIGIT_6 | /5XT/UGGGGCCACUCGAUCCUUGAGUUUUAGAGCUAGAAAUAGCAAGUUAAAAU | 343 |
| | AAGGCUAGUCCGUUAUCAACUUGAAAAAGUGGCACCGAGUCGGUGCUUUU/3XT/ | |
| TIGIT_7 | /5XT/CGCUGACCGUGAACGAUACAGUUUUAGAGCUAGAAAUAGCAAGUUAAAAU | 344 |
| | AAGGCUAGUCCGUUAUCAACUUGAAAAAGUGGCACCGAGUCGGUGCUUUU/3XT/ | |
| TIGIT_8 | /5XT/UCGCUGACCGUGAACGAUACGUUUUAGAGCUAGAAAUAGCAAGUUAAAAU | 345 |
| | AAGGCUAGUCCGUUAUCAACUUGAAAAAGUGGCACCGAGUCGGUGCUUUU/3XT/ | |
| FOXO1_1 | /5XT/GGGUCGAUCUCCACCACCUGGUUUUAGAGCUAGAAAUAGCAAGUUAAAAU | 346 |
| | AAGGCUAGUCCGUUAUCAACUUGAAAAAGUGGCACCGAGUCGGUGCUUUU/3XT/ | |
| FOXO1_2 | /5XT/GGAGUUUAGCCAGUCCAACUGUUUUAGAGCUAGAAAUAGCAAGUUAAAAU | 347 |
| | AAGGCUAGUCCGUUAUCAACUUGAAAAAGUGGCACCGAGUCGGUGCUUUU/3XT/ | |
| FOXO1_3 | /5XT/GAGUUGGACUGGCUAAACUCGUUUUAGAGCUAGAAAUAGCAAGUUAAAAU | 348 |
| | AAGGCUAGUCCGUUAUCAACUUGAAAAAGUGGCACCGAGUCGGUGCUUUU/3XT/ | |
| FOXO1_4 | /5XT/CACCAAGGCCAUCGAGAGCUGUUUUAGAGCUAGAAAUAGCAAGUUAAAAU | 349 |
| | AAGGCUAGUCCGUUAUCAACUUGAAAAAGUGGCACCGAGUCGGUGCUUUU/3XT/ | |
| FOXO1_5 | /5XT/AUCCACAUCGAGGCUCCUCGGUUUUAGAGCUAGAAAUAGCAAGUUAAAAU | 350 |
| | AAGGCUAGUCCGUUAUCAACUUGAAAAAGUGGCACCGAGUCGGUGCUUUU/3XT/ | |
| FOXO1_6 | /5XT/GAGCCCAGAACUUAACUUCGGUUUUAGAGCUAGAAAUAGCAAGUUAAAAU | 351 |
| | AAGGCUAGUCCGUUAUCAACUUGAAAAAGUGGCACCGAGUCGGUGCUUUU/3XT/ | |
| FOXO1_7 | /5XT/CAUCCACAUCGAGGCUCCUCGUUUUAGAGCUAGAAAUAGCAAGUUAAAAU | 352 |
| | AAGGCUAGUCCGUUAUCAACUUGAAAAAGUGGCACCGAGUCGGUGCUUUU/3XT/ | |
| FOXO1_8 | /5XT/CUACGCCGACCUCAUCACCAGUUUUAGAGCUAGAAAUAGCAAGUUAAAAU | 353 |
| | AAGGCUAGUCCGUUAUCAACUUGAAAAAGUGGCACCGAGUCGGUGCUUUU/3XT/ | |
| FOXP3_1 | /5XT/GCUCCCUGGACACCCAUUCCGUUUUAGAGCUAGAAAUAGCAAGUUAAAAU | 354 |
| | AAGGCUAGUCCGUUAUCAACUUGAAAAAGUGGCACCGAGUCGGUGCUUUU/3XT/ | |
| FOXP3_2 | /5XT/UCCCAAAUCCCAGUGCACCCGUUUUAGAGCUAGAAAUAGCAAGUUAAAAU | 355 |
| | AAGGCUAGUCCGUUAUCAACUUGAAAAAGUGGCACCGAGUCGGUGCUUUU/3XT/ | |
| FOXP3_3 | /5XT/UUCGAAGACCUUCUCACAUCGUUUUAGAGCUAGAAAUAGCAAGUUAAAAU | 356 |
| | AAGGCUAGUCCGUUAUCAACUUGAAAAAGUGGCACCGAGUCGGUGCUUUU/3XT/ | |
| FOXP3_4 | /5XT/UCGAAGACCUUCUCACAUCCGUUUUAGAGCUAGAAAUAGCAAGUUAAAAU | 357 |
| | AAGGCUAGUCCGUUAUCAACUUGAAAAAGUGGCACCGAGUCGGUGCUUUU/3XT/ | |
| FOXP3_5 | /5XT/CAAGUGGCCCGGAUGUGAGAGUUUUAGAGCUAGAAAUAGCAAGUUAAAAU | 358 |
| | AAGGCUAGUCCGUUAUCAACUUGAAAAAGUGGCACCGAGUCGGUGCUUUU/3XT/ | |
| FOXP3_6 | /5XT/GAAGGUCUUCGAAGAGCCAGGUUUUAGAGCUAGAAAUAGCAAGUUAAAAU | 359 |
| | AAGGCUAGUCCGUUAUCAACUUGAAAAAGUGGCACCGAGUCGGUGCUUUU/3XT/ | |
| FOXP3_7 | /5XT/ACUGUACCAUCUCUCUCUGGGUUUUAGAGCUAGAAAUAGCAAGUUAAAAU | 360 |
| | AAGGCUAGUCCGUUAUCAACUUGAAAAAGUGGCACCGAGUCGGUGCUUUU/3XT/ | |
| FOXP3_8 | /5XT/GGACCAUCUUCUGGAUGAGAGUUUUAGAGCUAGAAAUAGCAAGUUAAAAU | 361 |
| | AAGGCUAGUCCGUUAUCAACUUGAAAAAGUGGCACCGAGUCGGUGCUUUU/3XT/ | |
| TRAC_1 | /5XT/UCUCUCAGCUGGUACACGGCGUUUUAGAGCUAGAAAUAGCAAGUUAAAAU | 362 |
| | AAGGCUAGUCCGUUAUCAACUUGAAAAAGUGGCACCGAGUCGGUGCUUUU/3XT/ | |
| TRAC_2 | /5XT/CUCGACCAGCUUGACAUCACGUUUUAGAGCUAGAAAUAGCAAGUUAAAAU | 363 |
| | AAGGCUAGUCCGUUAUCAACUUGAAAAAGUGGCACCGAGUCGGUGCUUUU/3XT/ | |
| TRAC_3 | /5XT/AAGUUCCUGUGAUGUCAAGCGUUUUAGAGCUAGAAAUAGCAAGUUAAAAU | 364 |
| | AAGGCUAGUCCGUUAUCAACUUGAAAAAGUGGCACCGAGUCGGUGCUUUU/3XT/ | |
| TRAC_4 | /5XT/UUCGGAACCCAAUCACUGACGUUUUAGAGCUAGAAAUAGCAAGUUAAAAU | 365 |
| | AAGGCUAGUCCGUUAUCAACUUGAAAAAGUGGCACCGAGUCGGUGCUUUU/3XT/ | |
| TRAC_5 | /5XT/GAUUAAACCCGGCCACUUUCGUUUUAGAGCUAGAAAUAGCAAGUUAAAAU | 366 |
| | AAGGCUAGUCCGUUAUCAACUUGAAAAAGUGGCACCGAGUCGGUGCUUUU/3XT/ | |
| TRAC_6 | /5XT/ACCCGGCCACUUUCAGGAGGGUUUUAGAGCUAGAAAUAGCAAGUUAAAAU | 367 |
| | AAGGCUAGUCCGUUAUCAACUUGAAAAAGUGGCACCGAGUCGGUGCUUUU/3XT/ | |
| TRAC_7 | /5XT/CGUCAUGAGCAGAUUAAACCGUUUUAGAGCUAGAAAUAGCAAGUUAAAAU | 368 |
| | AAGGCUAGUCCGUUAUCAACUUGAAAAAGUGGCACCGAGUCGGUGCUUUU/3XT/ | |
| TRAC_8 | /5XT/UAAACCCGGCCACUUUCAGGGUUUUAGAGCUAGAAAUAGCAAGUUAAAAU | 369 |
| | AAGGCUAGUCCGUUAUCAACUUGAAAAAGUGGCACCGAGUCGGUGCUUUU/3XT/ | |
| TRBC1_1 | /5XT/GAACAAGGUGUUCCCACCCGGUUUUAGAGCUAGAAAUAGCAAGUUAAAAU | 370 |
| | AAGGCUAGUCCGUUAUCAACUUGAAAAAGUGGCACCGAGUCGGUGCUUUU/3XT/ | |
| TRBC1_2 | /5XT/CGGGUGGGAACACCUUGUUCGUUUUAGAGCUAGAAAUAGCAAGUUAAAAU | 371 |
| | AAGGCUAGUCCGUUAUCAACUUGAAAAAGUGGCACCGAGUCGGUGCUUUU/3XT/ | |
| TRBC1_3 | /5XT/UCAAACACAGCGACCUCGGGGUUUUAGAGCUAGAAAUAGCAAGUUAAAAU | 372 |
| | AAGGCUAGUCCGUUAUCAACUUGAAAAAGUGGCACCGAGUCGGUGCUUUU/3XT/ | |
| TRBC1_4 | /5XT/CGUAGAACUGGACUUGACAGGUUUUAGAGCUAGAAAUAGCAAGUUAAAAU | 373 |
| | AAGGCUAGUCCGUUAUCAACUUGAAAAAGUGGCACCGAGUCGGUGCUUUU/3XT/ | |
| TRBC1_5 | /5XT/AUGACGAGUGGACCCAGGAUGUUUUAGAGCUAGAAAUAGCAAGUUAAAAU | 374 |
| | AAGGCUAGUCCGUUAUCAACUUGAAAAAGUGGCACCGAGUCGGUGCUUUU/3XT/ | |
| TRBC1_6 | /5XT/GCUGUCAAGUCCAGUUCUACGUUUUAGAGCUAGAAAUAGCAAGUUAAAAU | 375 |
| | AAGGCUAGUCCGUUAUCAACUUGAAAAAGUGGCACCGAGUCGGUGCUUUU/3XT/ | |
| TRBC1_7 | /5XT/UGACGAGUGGACCCAGGAUAGUUUUAGAGCUAGAAAUAGCAAGUUAAAAU | 376 |
| | AAGGCUAGUCCGUUAUCAACUUGAAAAAGUGGCACCGAGUCGGUGCUUUU/3XT/ | |
| TRBC1_8 | /5XT/CUUGACAGCGGAAGUGGUUGGUUUUAGAGCUAGAAAUAGCAAGUUAAAAU | 377 |
| | AAGGCUAGUCCGUUAUCAACUUGAAAAAGUGGCACCGAGUCGGUGCUUUU/3XT/ | |
| MAP4K1_1 | /5XT/ACCACUAUGACCUGCUACAGGUUUUAGAGCUAGAAAUAGCAAGUUAAAAU | 378 |
| | AAGGCUAGUCCGUUAUCAACUUGAAAAAGUGGCACCGAGUCGGUGCUUUU/3XT/ | |
| MAP4K1_2 | /5XT/CAUUUUCAAUAGAGACCCCCGUUUUAGAGCUAGAAAUAGCAAGUUAAAAU | 379 |
| | AAGGCUAGUCCGUUAUCAACUUGAAAAAGUGGCACCGAGUCGGUGCUUUU/3XT/ | |
| MAP4K1_3 | /5XT/GGGUCCACGACGUCCAUCCCGUUUUAGAGCUAGAAAUAGCAAGUUAAAAU | 380 |
| | AAGGCUAGUCCGUUAUCAACUUGAAAAAGUGGCACCGAGUCGGUGCUUUU/3XT/ | |
| MAP4K1_4 | /5XT/GGUCCACGACGUCCAUCCCUGUUUUAGAGCUAGAAAUAGCAAGUUAAAAU | 381 |
| | AAGGCUAGUCCGUUAUCAACUUGAAAAAGUGGCACCGAGUCGGUGCUUUU/3XT/ | |
| MAP4K1_5 | /5XT/GUCCACGACGUCCAUCCCUGGUUUUAGAGCUAGAAAUAGCAAGUUAAAAU | 382 |
| | AAGGCUAGUCCGUUAUCAACUUGAAAAAGUGGCACCGAGUCGGUGCUUUU/3XT/ | |
| MAP4K1_6 | /5XT/UCCACGACGUCCAUCCCUGGGUUUUAGAGCUAGAAAUAGCAAGUUAAAAU | 383 |
| | AAGGCUAGUCCGUUAUCAACUUGAAAAAGUGGCACCGAGUCGGUGCUUUU/3XT/ | |
| MAP4K1_7 | /5XT/CCAACAUCGUGGCCUACCAUGUUUUAGAGCUAGAAAUAGCAAGUUAAAAU | 384 |
| | AAGGCUAGUCCGUUAUCAACUUGAAAAAGUGGCACCGAGUCGGUGCUUUU/3XT/ | |
| MAP4K1_8 | /5XT/CCCAUGGUAGGCCACGAUGUGUUUUAGAGCUAGAAAUAGCAAGUUAAAAU | 385 |
| | AAGGCUAGUCCGUUAUCAACUUGAAAAAGUGGCACCGAGUCGGUGCUUUU/3XT/ | |
| CD52_1 | /5XT/UAGGAUCUUCGUGGCUGUCUGUUUUAGAGCUAGAAAUAGCAAGUUAAAAU | 386 |
| | AAGGCUAGUCCGUUAUCAACUUGAAAAAGUGGCACCGAGUCGGUGCUUUU/3XT/ | |
| CD52_2 | /5XT/ACCAGGUUGUAGAAGUUGACGUUUUAGAGCUAGAAAUAGCAAGUUAAAAU | 387 |
| | AAGGCUAGUCCGUUAUCAACUUGAAAAAGUGGCACCGAGUCGGUGCUUUU/3XT/ | |
| CD52_3 | /5XT/AAGUUGACAGGCAGUGCCAUGUUUUAGAGCUAGAAAUAGCAAGUUAAAAU | 388 |
| | AAGGCUAGUCCGUUAUCAACUUGAAAAAGUGGCACCGAGUCGGUGCUUUU/3XT/ | |
| CD52_4 | /5XT/GCAUCCAGCAACAUAAGCGGGUUUUAGAGCUAGAAAUAGCAAGUUAAAAU | 389 |
| | AAGGCUAGUCCGUUAUCAACUUGAAAAAGUGGCACCGAGUCGGUGCUUUU/3XT/ | |
| CD52_5 | /5XT/UAACUUUAUUGACCCCCAGCGUUUUAGAGCUAGAAAUAGCAAGUUAAAAU | 390 |
| | AAGGCUAGUCCGUUAUCAACUUGAAAAAGUGGCACCGAGUCGGUGCUUUU/3XT/ | |
| CD52_6 | /5XT/CAACCCCUCCCAAAGAUGGAGUUUUAGAGCUAGAAAUAGCAAGUUAAAAU | 391 |
| | AAGGCUAGUCCGUUAUCAACUUGAAAAAGUGGCACCGAGUCGGUGCUUUU/3XT/ | |
| CD52_7 | /5XT/UUCUACAACCUGGUGAUGUCGUUUUAGAGCUAGAAAUAGCAAGUUAAAAU | 392 |
| | AAGGCUAGUCCGUUAUCAACUUGAAAAAGUGGCACCGAGUCGGUGCUUUU/3XT/ | |
| CD52_8 | /5XT/GCCUGUCAACUUCUACAACCGUUUUAGAGCUAGAAAUAGCAAGUUAAAAU | 393 |
| | AAGGCUAGUCCGUUAUCAACUUGAAAAAGUGGCACCGAGUCGGUGCUUUU/3XT/ | |
| B2M_1 | /5XT/AAGUCAACUUCAAUGUCGGAGUUUUAGAGCUAGAAAUAGCAAGUUAAAAU | 394 |
| | AAGGCUAGUCCGUUAUCAACUUGAAAAAGUGGCACCGAGUCGGUGCUUUU/3XT/ | |
| B2M_2 | /5XT/CGUGAGUAAACCUGAAUCUUGUUUUAGAGCUAGAAAUAGCAAGUUAAAAU | 395 |
| | AAGGCUAGUCCGUUAUCAACUUGAAAAAGUGGCACCGAGUCGGUGCUUUU/3XT/ | |
| B2M_3 | /5XT/ACAGCCCAAGAUAGUUAAGUGUUUUAGAGCUAGAAAUAGCAAGUUAAAAU | 396 |
| | AAGGCUAGUCCGUUAUCAACUUGAAAAAGUGGCACCGAGUCGGUGCUUUU/3XT/ | |
| B2M_4 | /5XT/AUUGUUUAGAGCUACCCAGCGUUUUAGAGCUAGAAAUAGCAAGUUAAAAU | 397 |
| | AAGGCUAGUCCGUUAUCAACUUGAAAAAGUGGCACCGAGUCGGUGCUUUU/3XT/ | |
| B2M_5 | /5XT/CUUACCCCACUUAACUAUCUGUUUUAGAGCUAGAAAUAGCAAGUUAAAAU | 398 |
| | AAGGCUAGUCCGUUAUCAACUUGAAAAAGUGGCACCGAGUCGGUGCUUUU/3XT/ | |
| B2M_6 | /5XT/CGAACAUCUCAAGAAGGUAUGUUUUAGAGCUAGAAAUAGCAAGUUAAAAU | 399 |
| | AAGGCUAGUCCGUUAUCAACUUGAAAAAGUGGCACCGAGUCGGUGCUUUU/3XT/ | |
| B2M_7 | /5XT/UUACCCCACUUAACUAUCUUGUUUUAGAGCUAGAAAUAGCAAGUUAAAAU | 400 |
| | AAGGCUAGUCCGUUAUCAACUUGAAAAAGUGGCACCGAGUCGGUGCUUUU/3XT/ | |
| B2M_8 | /5XT/CCAAUCCAGCCAGAAAGUACGUUUUAGAGCUAGAAAUAGCAAGUUAAAAU | 401 |
| | AAGGCUAGUCCGUUAUCAACUUGAAAAAGUGGCACCGAGUCGGUGCUUUU/3XT/ | |
| TRAC_June | /5XT/UGUGCUAGACAUGAGGUCUAGUUUUAGAGCUAGAAAUAGCAAGUUAAAAU | 402 |
| | AAGGCUAGUCCGUUAUCAACUUGAAAAAGUGGCACCGAGUCGGUGCUUUU/3XT/ | |
| TRBC_June | /5XT/GGAGAAUGACGAGUGGACCCGUUUUAGAGCUAGAAAUAGCAAGUUAAAAU | 403 |
| | AAGGCUAGUCCGUUAUCAACUUGAAAAAGUGGCACCGAGUCGGUGCUUUU/3XT/ | |
| PD1_June | /5XT/GGCGCCCUGGCCAGUCGUCUGUUUUAGAGCUAGAAAUAGCAAGUUAAAAU | 404 |
| | AAGGCUAGUCCGUUAUCAACUUGAAAAAGUGGCACCGAGUCGGUGCUUUU/3XT/ | |
| TRAC_Eyque | /5XT/CAGGGUUCUGGAUAUCUGUGGUUUUAGAGCUAGAAAUAGCAAGUUAAAAU | 405 |
| | AAGGCUAGUCCGUUAUCAACUUGAAAAAGUGGCACCGAGUCGGUGCUUUU/3XT/ | |
| B2M_Eyquem | /5XT/GGCCACGGAGCGAGACAUCUGUUUUAGAGCUAGAAAUAGCAAGUUAAAAU | 406 |
| | AAGGCUAGUCCGUUAUCAACUUGAAAAAGUGGCACCGAGUCGGUGCUUUU/3XT/ | |
| HEK1_ | /5XT/GGGAAAGACCCAGCAUCCGUGUUUUAGAGCUAGAAAUAGCAAGUUAAAAU | 407 |
| Chaudhari | AAGGCUAGUCCGUUAUCAACUUGAAAAAGUGGCACCGAGUCGGUGCUUUU/3XT/ | |
| HEK3_ | /5XT/GGCCCAGACUGAGCACGUGAGUUUUAGAGCUAGAAAUAGCAAGUUAAAAU | 408 |
| Chaudhari | AAGGCUAGUCCGUUAUCAACUUGAAAAAGUGGCACCGAGUCGGUGCUUUU/3XT/ | |
| RNF2_ | /5XT/GUCAUCUUAGUCAUUACCUGGUUUUAGAGCUAGAAAUAGCAAGUUAAAAU | 409 |
| Chaudhari | AAGGCUAGUCCGUUAUCAACUUGAAAAAGUGGCACCGAGUCGGUGCUUUU/3XT/ | |
| FANCF_ | /5XT/GGAAUCCCUUCUGCAGCACCGUUUUAGAGCUAGAAAUAGCAAGUUAAAAU | 410 |
| Chaudhari | AAGGCUAGUCCGUUAUCAACUUGAAAAAGUGGCACCGAGUCGGUGCUUUU/3XT/ | |
| VEGFA1_ | /5XT/GGGUGGGGGGAGUUUGCUCCGUUUUAGAGCUAGAAAUAGCAAGUUAAAAU | 411 |
| Chaudhari | AAGGCUAGUCCGUUAUCAACUUGAAAAAGUGGCACCGAGUCGGUGCUUUU/3XT/ | |
| IL2RG_ | /5XT/UGGUAAUGAUGGCUUCAACAGUUUUAGAGCUAGAAAUAGCAAGUUAAAAU | 412 |
| Chaudhari | AAGGCUAGUCCGUUAUCAACUUGAAAAAGUGGCACCGAGUCGGUGCUUUU/3XT/ | |
| HEK2_ | /5XT/GAACACAAAGCAUAGACUGCGUUUUAGAGCUAGAAAUAGCAAGUUAAAAU | 413 |
| Chaudhari | AAGGCUAGUCCGUUAUCAACUUGAAAAAGUGGCACCGAGUCGGUGCUUUU/3XT/ | |
| CCR5_ | /5XT/GUGUUCAUCUUUGGUUUUGUGUUUUAGAGCUAGAAAUAGCAAGUUAAAAU | 414 |
| Chaudhari | AAGGCUAGUCCGUUAUCAACUUGAAAAAGUGGCACCGAGUCGGUGCUUUU/3XT/ | |
| ALKAL1 | /5XT/UGUCCCCGCACGGAGCCCACGUUUUAGAGCUAGAAAUAGCAAGUUAAAAU | 415 |
| | AAGGCUAGUCCGUUAUCAACUUGAAAAAGUGGCACCGAGUCGGUGCUUUU/3XT/ | |
| C19orf84 | /5XT/GGGGGCCUACACCUUCCAACGUUUUAGAGCUAGAAAUAGCAAGUUAAAAU | 416 |
| | AAGGCUAGUCCGUUAUCAACUUGAAAAAGUGGCACCGAGUCGGUGCUUUU/3XT/ | |
| ATP6V0A2 | /5XT/UGUUUGGAUAGGGGUACACGGUUUUAGAGCUAGAAAUAGCAAGUUAAAAU | 417 |
| | AAGGCUAGUCCGUUAUCAACUUGAAAAAGUGGCACCGAGUCGGUGCUUUU/3XT/ | |
| ADPGK | /5XT/AGCCCAAGGGAAGUCACCGCGUUUUAGAGCUAGAAAUAGCAAGUUAAAAU | 418 |
| | AAGGCUAGUCCGUUAUCAACUUGAAAAAGUGGCACCGAGUCGGUGCUUUU/3XT/ | |
| C17orf99 | /5XT/GCGGGCCAACUUCACUCUGCGUUUUAGAGCUAGAAAUAGCAAGUUAAAAU | 419 |
| | AAGGCUAGUCCGUUAUCAACUUGAAAAAGUGGCACCGAGUCGGUGCUUUU/3XT/ | |
| ACAT1 | /5XT/UCAAGCUUUACCCCACCAUAGUUUUAGAGCUAGAAAUAGCAAGUUAAAAU | 420 |
| | AAGGCUAGUCCGUUAUCAACUUGAAAAAGUGGCACCGAGUCGGUGCUUUU/3XT/ | |
| AR | /5XT/GUUGGAGCAUCUGAGUCCAGGUUUUAGAGCUAGAAAUAGCAAGUUAAAAU | 421 |
| | AAGGCUAGUCCGUUAUCAACUUGAAAAAGUGGCACCGAGUCGGUGCUUUU/3XT/ | |
| EMX1 | /5XT/GAGUCCGAGCAGAAGAAGAAGUUUUAGAGCUAGAAAUAGCAAGUUAAAAU | 422 |
| | AAGGCUAGUCCGUUAUCAACUUGAAAAAGUGGCACCGAGUCGGUGCUUUU/3XT/ | |
| LAG3 | /5XT/GAAGGCUGAGAUCCUGGAGGGUUUUAGAGCUAGAAAUAGCAAGUUAAAAU | 423 |
| | AAGGCUAGUCCGUUAUCAACUUGAAAAAGUGGCACCGAGUCGGUGCUUUU/3XT/ | |
| AAVS1_site_ | /5XT/GGGAACCCAGCGAGUGAAGAGUUUUAGAGCUAGAAAUAGCAAGUUAAAAU | 424 |
| 10 | AAGGCUAGUCCGUUAUCAACUUGAAAAAGUGGCACCGAGUCGGUGCUUUU/3XT/ | |
| AAVS1_site_3 | /5XT/GAGCCACAUUAACCGGCCCUGUUUUAGAGCUAGAAAUAGCAAGUUAAAAU | 425 |
| | AAGGCUAGUCCGUUAUCAACUUGAAAAAGUGGCACCGAGUCGGUGCUUUU/3XT/ | |
| AAVS1_site_ | /5XT/GGUGAGGGAGGAGAGAUGCCGUUUUAGAGCUAGAAAUAGCAAGUUAAAAU | 426 |
| 11 | AAGGCUAGUCCGUUAUCAACUUGAAAAAGUGGCACCGAGUCGGUGCUUUU/3XT/ | |
| B2M_site_1 | /5XT/GGCCGAGAUGUCUCGCUCCGGUUUUAGAGCUAGAAAUAGCAAGUUAAAAU | 427 |
| | AAGGCUAGUCCGUUAUCAACUUGAAAAAGUGGCACCGAGUCGGUGCUUUU/3XT/ | |
| B2M_site_5 | /5XT/GAAGUUGACUUACUGAAGAAGUUUUAGAGCUAGAAAUAGCAAGUUAAAAU | 428 |
| | AAGGCUAGUCCGUUAUCAACUUGAAAAAGUGGCACCGAGUCGGUGCUUUU/3XT/ | |
| B2M_site_2 | /5XT/GCUACUCUCUCUUUCUGGCCGUUUUAGAGCUAGAAAUAGCAAGUUAAAAU | 429 |
| | AAGGCUAGUCCGUUAUCAACUUGAAAAAGUGGCACCGAGUCGGUGCUUUU/3XT/ | |
| CBLB_site_4 | /5XT/GGCAGAAACCCUGGUGGUCGGUUUUAGAGCUAGAAAUAGCAAGUUAAAAU | 430 |
| | AAGGCUAGUCCGUUAUCAACUUGAAAAAGUGGCACCGAGUCGGUGCUUUU/3XT/ | |
| CBLB_site_6 | /5XT/GGAUUUCCUCCUCGACCACCGUUUUAGAGCUAGAAAUAGCAAGUUAAAAU | 431 |
| | AAGGCUAGUCCGUUAUCAACUUGAAAAAGUGGCACCGAGUCGGUGCUUUU/3XT/ | |
| CBLB_site_8 | /5XT/GGGUAUUAUUGAUGCUAUUCGUUUUAGAGCUAGAAAUAGCAAGUUAAAAU | 432 |
| | AAGGCUAGUCCGUUAUCAACUUGAAAAAGUGGCACCGAGUCGGUGCUUUU/3XT/ | |
| CCR5_site_9 | /5XT/GGUACCUAUCGAUUGUCAGGGUUUUAGAGCUAGAAAUAGCAAGUUAAAAU | 433 |
| | AAGGCUAGUCCGUUAUCAACUUGAAAAAGUGGCACCGAGUCGGUGCUUUU/3XT/ | |
| CCR5_site_13 | /5XT/GACAUUAAAGAUAGUCAUCUGUUUUAGAGCUAGAAAUAGCAAGUUAAAAU | 434 |
| | AAGGCUAGUCCGUUAUCAACUUGAAAAAGUGGCACCGAGUCGGUGCUUUU/3XT/ | |
| CCR5_site_4 | /5XT/GUAGAGCGGAGGCAGGAGGCGUUUUAGAGCUAGAAAUAGCAAGUUAAAAU | 435 |
| | AAGGCUAGUCCGUUAUCAACUUGAAAAAGUGGCACCGAGUCGGUGCUUUU/3XT/ | |
| CTLA4_site_ | /5XT/GAGGUUCACUUGAUUUCCACGUUUUAGAGCUAGAAAUAGCAAGUUAAAAU | 436 |
| 10 | AAGGCUAGUCCGUUAUCAACUUGAAAAAGUGGCACCGAGUCGGUGCUUUU/3XT/ | |
| CTLA4_site_6 | /5XT/GUGCGGCAACCUACAUGAUGGUUUUAGAGCUAGAAAUAGCAAGUUAAAAU | 437 |
| | AAGGCUAGUCCGUUAUCAACUUGAAAAAGUGGCACCGAGUCGGUGCUUUU/3XT/ | |
| CTLA4_site_ | /5XT/GCACAAGGCUCAGCUGAACCGUUUUAGAGCUAGAAAUAGCAAGUUAAAAU | 438 |
| 12 | AAGGCUAGUCCGUUAUCAACUUGAAAAAGUGGCACCGAGUCGGUGCUUUU/3XT/ | |
| CXCR4_site_ | /5XT/GAUAACUACACCGAGGAAAUGUUUUAGAGCUAGAAAUAGCAAGUUAAAAU | 439 |
| 1 | AAGGCUAGUCCGUUAUCAACUUGAAAAAGUGGCACCGAGUCGGUGCUUUU/3XT/ | |
| CXCR4_site | /5XT/GCCGUGGCAAACUGGUACUUGUUUUAGAGCUAGAAAUAGCAAGUUAAAAU | 440 |
| 10 | AAGGCUAGUCCGUUAUCAACUUGAAAAAGUGGCACCGAGUCGGUGCUUUU/3XT/ | |
| CXCR4_site | /5XT/GAAGAUGAUGGAGUAGAUGGGUUUUAGAGCUAGAAAUAGCAAGUUAAAAU | 441 |
| 3 | AAGGCUAGUCCGUUAUCAACUUGAAAAAGUGGCACCGAGUCGGUGCUUUU/3XT/ | |
| FAS_site_3 | /5XT/GGGGCAGCUCCGGCGCUCCUGUUUUAGAGCUAGAAAUAGCAAGUUAAAAU | 442 |
| | AAGGCUAGUCCGUUAUCAACUUGAAAAAGUGGCACCGAGUCGGUGCUUUU/3XT/ | |
| FAS_site_2 | /5XT/GCUGACCCCGCUGGGCAGGCGUUUUAGAGCUAGAAAUAGCAAGUUAAAAU | 443 |
| | AAGGCUAGUCCGUUAUCAACUUGAAAAAGUGGCACCGAGUCGGUGCUUUU/3XT/ | |
| FAS_site_1 | /5XT/GAGGGCUCACCAGAGGUAGGGUUUUAGAGCUAGAAAUAGCAAGUUAAAAU | 444 |
| | AAGGCUAGUCCGUUAUCAACUUGAAAAAGUGGCACCGAGUCGGUGCUUUU/3XT/ | |
| LAG3_site_2 | /5XT/GCUGUUUCUGCAGCCGCUUUGUUUUAGAGCUAGAAAUAGCAAGUUAAAAU | 445 |
| | AAGGCUAGUCCGUUAUCAACUUGAAAAAGUGGCACCGAGUCGGUGCUUUU/3XT/ | |
| LAG3_site_5 | /5XT/GGUCCCGGUGGUGUGGGCCCGUUUUAGAGCUAGAAAUAGCAAGUUAAAAU | 446 |
| | AAGGCUAGUCCGUUAUCAACUUGAAAAAGUGGCACCGAGUCGGUGCUUUU/3XT/ | |
| LAG3_site_6 | /5XT/GGUGGUGUGGGCCCAGGAGGGUUUUAGAGCUAGAAAUAGCAAGUUAAAAU | 447 |
| | AAGGCUAGUCCGUUAUCAACUUGAAAAAGUGGCACCGAGUCGGUGCUUUU/3XT/ | |
| PDCD1_site | /5XT/GCGUGACUUCCACAUGAGCGGUUUUAGAGCUAGAAAUAGCAAGUUAAAAU | 448 |
| 13 | AAGGCUAGUCCGUUAUCAACUUGAAAAAGUGGCACCGAGUCGGUGCUUUU/3XT/ | |
| PDCD1_site_ | /5XT/GUCUGGGCGGUGCUACAACUGUUUUAGAGCUAGAAAUAGCAAGUUAAAAU | 449 |
| 3 | AAGGCUAGUCCGUUAUCAACUUGAAAAAGUGGCACCGAGUCGGUGCUUUU/3XT/ | |
| PDCD1_site_ | /5XT/GAGCAGGGCUGGGGAGAAGGGUUUUAGAGCUAGAAAUAGCAAGUUAAAAU | 450 |
| 8 | AAGGCUAGUCCGUUAUCAACUUGAAAAAGUGGCACCGAGUCGGUGCUUUU/3XT/ | |
| PTPN2_site_1 | /5XT/GGAAACUUGGCCACUCUAUGGUUUUAGAGCUAGAAAUAGCAAGUUAAAAU | 451 |
| | AAGGCUAGUCCGUUAUCAACUUGAAAAAGUGGCACCGAGUCGGUGCUUUU/3XT/ | |
| PTPN2_site_2 | /5XT/GGCACCAACUGGAUGGAUCAGUUUUAGAGCUAGAAAUAGCAAGUUAAAAU | 452 |
| | AAGGCUAGUCCGUUAUCAACUUGAAAAAGUGGCACCGAGUCGGUGCUUUU/3XT/ | |
| PTPN2_site_3 | /5XT/GUCUCCCUGAUCCAUCCAGUGUUUUAGAGCUAGAAAUAGCAAGUUAAAAU | 453 |
| | AAGGCUAGUCCGUUAUCAACUUGAAAAAGUGGCACCGAGUCGGUGCUUUU/3XT/ | |
| PTPN6_site_8 | /5XT/GUUUGCGACUCUGACAGAGCGUUUUAGAGCUAGAAAUAGCAAGUUAAAAU | 454 |
| | AAGGCUAGUCCGUUAUCAACUUGAAAAAGUGGCACCGAGUCGGUGCUUUU/3XT/ | |
| PTPN6_site_4 | /5XT/GGUUUCACCGAGACCUCAGUGUUUUAGAGCUAGAAAUAGCAAGUUAAAAU | 455 |
| | AAGGCUAGUCCGUUAUCAACUUGAAAAAGUGGCACCGAGUCGGUGCUUUU/3XT/ | |
| PTPN6_site_3 | /5XT/GAUUUCUAUGACCUGUAUGGGUUUUAGAGCUAGAAAUAGCAAGUUAAAAU | 456 |
| | AAGGCUAGUCCGUUAUCAACUUGAAAAAGUGGCACCGAGUCGGUGCUUUU/3XT/ | |
| TRAC_site_3 | /5XT/GAGAAUCAAAAUCGGUGAAUGUUUUAGAGCUAGAAAUAGCAAGUUAAAAU | 457 |
| | AAGGCUAGUCCGUUAUCAACUUGAAAAAGUGGCACCGAGUCGGUGCUUUU/3XT/ | |
| TRAC_site_4 | /5XT/GACACCUUCUUCCCCAGCCCGUUUUAGAGCUAGAAAUAGCAAGUUAAAAU | 458 |
| | AAGGCUAGUCCGUUAUCAACUUGAAAAAGUGGCACCGAGUCGGUGCUUUU/3XT/ | |
| TRAC_site_2 | /5XT/GCUGGUACACGGCAGGGUCAGUUUUAGAGCUAGAAAUAGCAAGUUAAAAU | 459 |
| | AAGGCUAGUCCGUUAUCAACUUGAAAAAGUGGCACCGAGUCGGUGCUUUU/3XT/ | |
| TRBC1_site_1 | /5XT/GAACAAGGUGUUCCCACCCGGUUUUAGAGCUAGAAAUAGCAAGUUAAAAU | 460 |
| | AAGGCUAGUCCGUUAUCAACUUGAAAAAGUGGCACCGAGUCGGUGCUUUU/3XT/ | |
| TRBC1_site_2 | /5XT/GGUGCACAGUGGGGUCAGCAGUUUUAGAGCUAGAAAUAGCAAGUUAAAAU | 461 |
| | AAGGCUAGUCCGUUAUCAACUUGAAAAAGUGGCACCGAGUCGGUGCUUUU/3XT/ | |
| RAB6B | /5XT/GACGUCGUCGAUCCACUUAGGUUUUAGAGCUAGAAAUAGCAAGUUAAAAU | 462 |
| | AAGGCUAGUCCGUUAUCAACUUGAAAAAGUGGCACCGAGUCGGUGCUUUU/3XT/ | |
| ZFX | /5XT/UCACCCGUCAAGACGUGUUCGUUUUAGAGCUAGAAAUAGCAAGUUAAAAU | 463 |
| | AAGGCUAGUCCGUUAUCAACUUGAAAAAGUGGCACCGAGUCGGUGCUUUU/3XT/ | |
| EPM2A | /5XT/UGUACCAGAACGUGUCCACGGUUUUAGAGCUAGAAAUAGCAAGUUAAAAU | 464 |
| | AAGGCUAGUCCGUUAUCAACUUGAAAAAGUGGCACCGAGUCGGUGCUUUU/3XT/ | |
| CPXM2 | /5XT/ACGGACACUGUGAUCAUCGUGUUUUAGAGCUAGAAAUAGCAAGUUAAAAU | 465 |
| | AAGGCUAGUCCGUUAUCAACUUGAAAAAGUGGCACCGAGUCGGUGCUUUU/3XT/ | |
| SYNGAP1 | /5XT/CCAACCAGGACGAUCAUACGGUUUUAGAGCUAGAAAUAGCAAGUUAAAAU | 466 |
| | AAGGCUAGUCCGUUAUCAACUUGAAAAAGUGGCACCGAGUCGGUGCUUUU/3XT/ | |
| GPR141 | /5XT/UGUCACUAUAGGAUCGCAAGGUUUUAGAGCUAGAAAUAGCAAGUUAAAAU | 467 |
| | AAGGCUAGUCCGUUAUCAACUUGAAAAAGUGGCACCGAGUCGGUGCUUUU/3XT/ | |
| KRTAP13-2 | /5XT/CCUUGCAAGACGACUUACUCGUUUUAGAGCUAGAAAUAGCAAGUUAAAAU | 468 |
| | AAGGCUAGUCCGUUAUCAACUUGAAAAAGUGGCACCGAGUCGGUGCUUUU/3XT/ | |
| RNF10 | /5XT/GUGUCCACAACGGGUUAUCUGUUUUAGAGCUAGAAAUAGCAAGUUAAAAU | 469 |
| | AAGGCUAGUCCGUUAUCAACUUGAAAAAGUGGCACCGAGUCGGUGCUUUU/3XT/ | |
| DMXL2 | /5XT/GGAGACAACUGCUACUCCGUGUUUUAGAGCUAGAAAUAGCAAGUUAAAAU | 470 |
| | AAGGCUAGUCCGUUAUCAACUUGAAAAAGUGGCACCGAGUCGGUGCUUUU/3XT/ | |
| ADGRV1 | /5XT/UUGUCCUUUCCACGAACUACGUUUUAGAGCUAGAAAUAGCAAGUUAAAAU | 471 |
| | AAGGCUAGUCCGUUAUCAACUUGAAAAAGUGGCACCGAGUCGGUGCUUUU/3XT/ | |
| PTP4A3 | /5XT/GAAGUACGGGGCUACCACUGGUUUUAGAGCUAGAAAUAGCAAGUUAAAAU | 472 |
| | AAGGCUAGUCCGUUAUCAACUUGAAAAAGUGGCACCGAGUCGGUGCUUUU/3XT/ | |
| CYP2C18 | /5XT/ACGAGCACCACUCUGAGAUAGUUUUAGAGCUAGAAAUAGCAAGUUAAAAU | 473 |
| | AAGGCUAGUCCGUUAUCAACUUGAAAAAGUGGCACCGAGUCGGUGCUUUU/3XT/ | |
| OR4A15 | /5XT/UGUCGGAGCCUACAAACAAAGUUUUAGAGCUAGAAAUAGCAAGUUAAAAU | 474 |
| | AAGGCUAGUCCGUUAUCAACUUGAAAAAGUGGCACCGAGUCGGUGCUUUU/3XT/ | |
| PCBP2 | /5XT/AUGGACACCGGUGUGAUUGAGUUUUAGAGCUAGAAAUAGCAAGUUAAAAU | 475 |
| | AAGGCUAGUCCGUUAUCAACUUGAAAAAGUGGCACCGAGUCGGUGCUUUU/3XT/ | |
| PAPSS1 | /5XT/GCAACCACGAAAGCCACCUCGUUUUAGAGCUAGAAAUAGCAAGUUAAAAU | 476 |
| | AAGGCUAGUCCGUUAUCAACUUGAAAAAGUGGCACCGAGUCGGUGCUUUU/3XT/ | |
| DPY19L3 | /5XT/GCUUGUAGUAGGAGUAAUACGUUUUAGAGCUAGAAAUAGCAAGUUAAAAU | 477 |
| | AAGGCUAGUCCGUUAUCAACUUGAAAAAGUGGCACCGAGUCGGUGCUUUU/3XT/ | |
| SLFN12 | /5XT/UCAUGGAGCUUGAACACCUCGUUUUAGAGCUAGAAAUAGCAAGUUAAAAU | 478 |
| | AAGGCUAGUCCGUUAUCAACUUGAAAAAGUGGCACCGAGUCGGUGCUUUU/3XT/ | |
| NKX2-8 | /5XT/AACCAGAUCUUGACCUGCGUGUUUUAGAGCUAGAAAUAGCAAGUUAAAAU | 479 |
| | AAGGCUAGUCCGUUAUCAACUUGAAAAAGUGGCACCGAGUCGGUGCUUUU/3XT/ | |
| RIPPLY2 | /5XT/CAGGAAAGCUUUACCAAUUCGUUUUAGAGCUAGAAAUAGCAAGUUAAAAU | 480 |
| | AAGGCUAGUCCGUUAUCAACUUGAAAAAGUGGCACCGAGUCGGUGCUUUU/3XT/ | |
| PKLR | /5XT/CGGCACGACCCGGACAAUAUGUUUUAGAGCUAGAAAUAGCAAGUUAAAAU | 481 |
| | AAGGCUAGUCCGUUAUCAACUUGAAAAAGUGGCACCGAGUCGGUGCUUUU/3XT/ | |
| XRCC5 | /5XT/GGUGGACAAGCGGCAGAUAGGUUUUAGAGCUAGAAAUAGCAAGUUAAAAU | 482 |
| | AAGGCUAGUCCGUUAUCAACUUGAAAAAGUGGCACCGAGUCGGUGCUUUU/3XT/ | |
| CD34 | /5XT/AUAGGAGAAGAUGAUGUAUAGUUUUAGAGCUAGAAAUAGCAAGUUAAAAU | 483 |
| | AAGGCUAGUCCGUUAUCAACUUGAAAAAGUGGCACCGAGUCGGUGCUUUU/3XT/ | |
| PAPSS2_tgt_ | /5XT/GCAUACAGUGAUUUGAUGAAGUUUUAGAGCUAGAAAUAGCAAGUUAAAAU | 484 |
| 1 | AAGGCUAGUCCGUUAUCAACUUGAAAAAGUGGCACCGAGUCGGUGCUUUU/3XT/ | |
| CD151 | /5XT/GCUGAUGUAGUCACUCUUGAGUUUUAGAGCUAGAAAUAGCAAGUUAAAAU | 485 |
| | AAGGCUAGUCCGUUAUCAACUUGAAAAAGUGGCACCGAGUCGGUGCUUUU/3XT/ | |
| PTPRC_tgt_2 | /5XT/GCAAAACUCAACCCUACCCCGUUUUAGAGCUAGAAAUAGCAAGUUAAAAU | 486 |
| | AAGGCUAGUCCGUUAUCAACUUGAAAAAGUGGCACCGAGUCGGUGCUUUU/3XT/ | |
| PTPRC_tgt_5 | /5XT/CUCGUCUGAUAAGACAACAGGUUUUAGAGCUAGAAAUAGCAAGUUAAAAU | 487 |
| | AAGGCUAGUCCGUUAUCAACUUGAAAAAGUGGCACCGAGUCGGUGCUUUU/3XT/ | |
| HBB | /5XT/CUUGCCCCACAGGGCAGUAAGUUUUAGAGCUAGAAAUAGCAAGUUAAAAU | 488 |
| | AAGGCUAGUCCGUUAUCAACUUGAAAAAGUGGCACCGAGUCGGUGCUUUU/3XT/ | |

/5XT/indicates proprietary 5′-terminal modifications to enhance effectiveness; /3XT/indicates proprietary 3′-terminal modifications to enhance effectiveness.

TABLE 6
UNCOVERseq NGS Primers
PCR1_PCR2

| | | SEQ ID |
|---|---|---|
| Name | DNA Sequence (5′→3′) | NO |
| Top_PCR1_FWD | CATAGCGGTATTACGCGAGATTACGATAGCCGGACGAATGTCGrGTCGT/ | 489 |
| | 3SpC3/ | |
| Bottom_PCR1_ | CATAGCGGTATTACGCGAGATTACGAACATTCGTCCGGCTACCTrACGCC/ | 490 |
| REV | 3SpC3/ | |
| P5_PCR1 | AATGATACGGCGACCACCGAGATrCTACA/3SpC3/ | 491 |
| PCR1_T_Blocker | GTCGGTCGTAGTTAGATCGGAAGAGC/3SpC3/ | 492 |
| PCR1_B_Blocker | TACCTACGCCGCTTAAGATCGGAAGAGC/3SpC3/ | 493 |
| P5_PCR2 | AATGATACGGCGACCACCGAGATCTACAC | 494 |

Sequencing Primers

| | | SEQ ID |
|---|---|---|
| Name | DNA Sequence (5′→3′) | NO |
| CTLseq_Index1 | TCGTAATCTCGCGTAATACCGCTATGATCACCGACTGCC | 495 |
| CTLseq_Read2 | GGCAGTCGGTGATCATAGCGGTATTACGCGAGATTACGA | 496 |

“rN” indicates a ribonucleotide, where N is the nucleotide preceeded by the “r”; /3SpC3/ indicates a 3′-terminal C₃ spacer.

TABLE 7
P5-P7 Oligonucleotides

| | | SEQ ID |
|---|---|---|
| Name | DNA Sequence (5′→3′) | NO |
| P501 | AATGATACGGCGACCACCGAGATCTACACATATGCGCNNWNNWNNACACTCTTTCCCT | 497 |
| | ACACGACGCTCTTCCGATC*T | |
| P502 | AATGATACGGCGACCACCGAGATCTACACTGGTACAGNNWNNWNNACACTCTTTCCCT | 498 |
| | ACACGACGCTCTTCCGATC*T | |
| P503 | AATGATACGGCGACCACCGAGATCTACACAACCGTTCNNWNNWNNACACTCTTTCCCT | 499 |
| | ACACGACGCTCTTCCGATC*T | |
| P504 | AATGATACGGCGACCACCGAGATCTACACTAACCGGTNNWNNWNNACACTCTTTCCCT | 500 |
| | ACACGACGCTCTTCCGATC*T | |
| P505 | AATGATACGGCGACCACCGAGATCTACACGAACATCGNNWNNWNNACACTCTTTCCCT | 501 |
| | ACACGACGCTCTTCCGATC*T | |
| P506 | AATGATACGGCGACCACCGAGATCTACACCCTTGTAGNNWNNWNNACACTCTTTCCCT | 502 |
| | ACACGACGCTCTTCCGATC*T | |
| P507 | AATGATACGGCGACCACCGAGATCTACACTCAGGCTTNNWNNWNNACACTCTTTCCCT | 503 |
| | ACACGACGCTCTTCCGATC*T | |
| P508 | AATGATACGGCGACCACCGAGATCTACACGTTCTCGTNNWNNWNNACACTCTTTCCCT | 504 |
| | ACACGACGCTCTTCCGATC*T | |
| P509 | AATGATACGGCGACCACCGAGATCTACACAGAACGAGNNWNNWNNACACTCTTTCCCT | 505 |
| | ACACGACGCTCTTCCGATC*T | |
| P510 | AATGATACGGCGACCACCGAGATCTACACTGCTTCCANNWNNWNNACACTCTTTCCCT | 506 |
| | ACACGACGCTCTTCCGATC*T | |
| P511 | AATGATACGGCGACCACCGAGATCTACACCTTCGACTNNWNNWNNACACTCTTTCCCT | 507 |
| | ACACGACGCTCTTCCGATC*T | |
| P512 | AATGATACGGCGACCACCGAGATCTACACCACCTGTTNNWNNWNNACACTCTTTCCCT | 508 |
| | ACACGACGCTCTTCCGATC*T | |
| P513 | AATGATACGGCGACCACCGAGATCTACACATCACACGNNWNNWNNACACTCTTTCCCT | 509 |
| | ACACGACGCTCTTCCGATC*T | |
| P514 | AATGATACGGCGACCACCGAGATCTACACCCGTAAGANNWNNWNNACACTCTTTCCCT | 510 |
| | ACACGACGCTCTTCCGATC*T | |
| P515 | AATGATACGGCGACCACCGAGATCTACACTACGCCTTNNWNNWNNACACTCTTTCCCT | 511 |
| | ACACGACGCTCTTCCGATC*T | |
| P516 | AATGATACGGCGACCACCGAGATCTACACCGACGTTANNWNNWNNACACTCTTTCCCT | 512 |
| | ACACGACGCTCTTCCGATC*T | |
| P517 | AATGATACGGCGACCACCGAGATCTACACATGCACGANNWNNWNNACACTCTTTCCCT | 513 |
| | ACACGACGCTCTTCCGATC*T | |
| P518 | AATGATACGGCGACCACCGAGATCTACACCCTGATTGNNWNNWNNACACTCTTTCCCT | 514 |
| | ACACGACGCTCTTCCGATC*T | |
| P519 | AATGATACGGCGACCACCGAGATCTACACGTAGGAGTNNWNNWNNACACTCTTTCCCT | 515 |
| | ACACGACGCTCTTCCGATC*T | |
| P520 | AATGATACGGCGACCACCGAGATCTACACACTAGGAGNNWNNWNNACACTCTTTCCCT | 516 |
| | ACACGACGCTCTTCCGATC*T | |
| P521 | AATGATACGGCGACCACCGAGATCTACACCACTAGCTNNWNNWNNACACTCTTTCCCT | 517 |
| | ACACGACGCTCTTCCGATC*T | |
| P522 | AATGATACGGCGACCACCGAGATCTACACACGACTTGNNWNNWNNACACTCTTTCCCT | 518 |
| | ACACGACGCTCTTCCGATC*T | |
| P523 | AATGATACGGCGACCACCGAGATCTACACCGTGTGTANNWNNWNNACACTCTTTCCCT | 519 |
| | ACACGACGCTCTTCCGATC*T | |
| P524 | AATGATACGGCGACCACCGAGATCTACACGTTGACCTNNWNNWNNACACTCTTTCCCT | 520 |
| | ACACGACGCTCTTCCGATC*T | |
| P525 | AATGATACGGCGACCACCGAGATCTACACACTCCATCNNWNNWNNACACTCTTTCCCT | 521 |
| | ACACGACGCTCTTCCGATC*T | |
| P526 | AATGATACGGCGACCACCGAGATCTACACCAATGTGGNNWNNWNNACACTCTTTCCCT | 522 |
| | ACACGACGCTCTTCCGATC*T | |
| P527 | AATGATACGGCGACCACCGAGATCTACACTTGCAGACNNWNNWNNACACTCTTTCCCT | 523 |
| | ACACGACGCTCTTCCGATC*T | |
| P528 | AATGATACGGCGACCACCGAGATCTACACCAGTCCAANNWNNWNNACACTCTTTCCCT | 524 |
| | ACACGACGCTCTTCCGATC*T | |
| P529 | AATGATACGGCGACCACCGAGATCTACACACGTTCAGNNWNNWNNACACTCTTTCCCT | 525 |
| | ACACGACGCTCTTCCGATC*T | |
| P530 | AATGATACGGCGACCACCGAGATCTACACAACGTCTGNNWNNWNNACACTCTTTCCCT | 526 |
| | ACACGACGCTCTTCCGATC*T | |
| P531 | AATGATACGGCGACCACCGAGATCTACACTATCGGTCNNWNNWNNACACTCTTTCCCT | 527 |
| | ACACGACGCTCTTCCGATC*T | |
| P532 | AATGATACGGCGACCACCGAGATCTACACCGCTCTATNNWNNWNNACACTCTTTCCCT | 528 |
| | ACACGACGCTCTTCCGATC*T | |
| P533 | AATGATACGGCGACCACCGAGATCTACACGATTGCTCNNWNNWNNACACTCTTTCCCT | 529 |
| | ACACGACGCTCTTCCGATC*T | |
| P534 | AATGATACGGCGACCACCGAGATCTACACGATGTGTGNNWNNWNNACACTCTTTCCCT | 530 |
| | ACACGACGCTCTTCCGATC*T | |
| P535 | AATGATACGGCGACCACCGAGATCTACACCGCAATCTNNWNNWNNACACTCTTTCCCT | 531 |
| | ACACGACGCTCTTCCGATC*T | |
| P536 | AATGATACGGCGACCACCGAGATCTACACTGGTAGCTNNWNNWNNACACTCTTTCCCT | 532 |
| | ACACGACGCTCTTCCGATC*T | |
| P537 | AATGATACGGCGACCACCGAGATCTACACGATAGGCTNNWNNWNNACACTCTTTCCCT | 533 |
| | ACACGACGCTCTTCCGATC*T | |
| P538 | AATGATACGGCGACCACCGAGATCTACACAGTGGATCNNWNNWNNACACTCTTTCCCT | 534 |
| | ACACGACGCTCTTCCGATC*T | |
| P539 | AATGATACGGCGACCACCGAGATCTACACTTGGACGTNNWNNWNNACACTCTTTCCCT | 535 |
| | ACACGACGCTCTTCCGATC*T | |
| P540 | AATGATACGGCGACCACCGAGATCTACACATGACGTCNNWNNWNNACACTCTTTCCCT | 536 |
| | ACACGACGCTCTTCCGATC*T | |
| P541 | AATGATACGGCGACCACCGAGATCTACACGAAGTTGGNNWNNWNNACACTCTTTCCCT | 537 |
| | ACACGACGCTCTTCCGATC*T | |
| P542 | AATGATACGGCGACCACCGAGATCTACACCATACCACNNWNNWNNACACTCTTTCCCT | 538 |
| | ACACGACGCTCTTCCGATC*T | |
| P543 | AATGATACGGCGACCACCGAGATCTACACCTGTTGACNNWNNWNNACACTCTTTCCCT | 539 |
| | ACACGACGCTCTTCCGATC*T | |
| P544 | AATGATACGGCGACCACCGAGATCTACACTGGCATGTNNWNNWNNACACTCTTTCCCT | 540 |
| | ACACGACGCTCTTCCGATC*T | |
| P545 | AATGATACGGCGACCACCGAGATCTACACATCGCCATNNWNNWNNACACTCTTTCCCT | 541 |
| | ACACGACGCTCTTCCGATC*T | |
| P546 | AATGATACGGCGACCACCGAGATCTACACTTGCGAAGNNWNNWNNACACTCTTTCCCT | 542 |
| | ACACGACGCTCTTCCGATC*T | |
| P547 | AATGATACGGCGACCACCGAGATCTACACAGTTCGTCNNWNNWNNACACTCTTTCCCT | 543 |
| | ACACGACGCTCTTCCGATC*T | |
| P548 | AATGATACGGCGACCACCGAGATCTACACGAGCAGTANNWNNWNNACACTCTTTCCCT | 544 |
| | ACACGACGCTCTTCCGATC*T | |
| P549 | AATGATACGGCGACCACCGAGATCTACACACAGCTCANNWNNWNNACACTCTTTCCCT | 545 |
| | ACACGACGCTCTTCCGATC*T | |
| P550 | AATGATACGGCGACCACCGAGATCTACACGATCGAGTNNWNNWNNACACTCTTTCCCT | 546 |
| | ACACGACGCTCTTCCGATC*T | |
| P551 | AATGATACGGCGACCACCGAGATCTACACAGCGTGTTNNWNNWNNACACTCTTTCCCT | 547 |
| | ACACGACGCTCTTCCGATC*T | |
| P552 | AATGATACGGCGACCACCGAGATCTACACGTTACGCANNWNNWNNACACTCTTTCCCT | 548 |
| | ACACGACGCTCTTCCGATC*T | |
| P553 | AATGATACGGCGACCACCGAGATCTACACTGAAGACGNNWNNWNNACACTCTTTCCCT | 549 |
| | ACACGACGCTCTTCCGATC*T | |
| P554 | AATGATACGGCGACCACCGAGATCTACACACTGAGGTNNWNNWNNACACTCTTTCCCT | 550 |
| | ACACGACGCTCTTCCGATC*T | |
| P555 | AATGATACGGCGACCACCGAGATCTACACCGGTTGTTNNWNNWNNACACTCTTTCCCT | 551 |
| | ACACGACGCTCTTCCGATC*T | |
| P556 | AATGATACGGCGACCACCGAGATCTACACGTTGTTCGNNWNNWNNACACTCTTTCCCT | 552 |
| | ACACGACGCTCTTCCGATC*T | |
| P557 | AATGATACGGCGACCACCGAGATCTACACGAAGGAAGNNWNNWNNACACTCTTTCCCT | 553 |
| | ACACGACGCTCTTCCGATC*T | |
| P558 | AATGATACGGCGACCACCGAGATCTACACAGCACTTCNNWNNWNNACACTCTTTCCCT | 554 |
| | ACACGACGCTCTTCCGATC*T | |
| P559 | AATGATACGGCGACCACCGAGATCTACACGTCATCGANNWNNWNNACACTCTTTCCCT | 555 |
| | ACACGACGCTCTTCCGATC*T | |
| P560 | AATGATACGGCGACCACCGAGATCTACACTGTGACTGNNWNNWNNACACTCTTTCCCT | 556 |
| | ACACGACGCTCTTCCGATC*T | |
| P561 | AATGATACGGCGACCACCGAGATCTACACCAACACCTNNWNNWNNACACTCTTTCCCT | 557 |
| | ACACGACGCTCTTCCGATC*T | |
| P562 | AATGATACGGCGACCACCGAGATCTACACATGCCTGTNNWNNWNNACACTCTTTCCCT | 558 |
| | ACACGACGCTCTTCCGATC*T | |
| P563 | AATGATACGGCGACCACCGAGATCTACACCATGGCTANNWNNWNNACACTCTTTCCCT | 559 |
| | ACACGACGCTCTTCCGATC*T | |
| P564 | AATGATACGGCGACCACCGAGATCTACACGTGAAGTGNNWNNWNNACACTCTTTCCCT | 560 |
| | ACACGACGCTCTTCCGATC*T | |
| P565 | AATGATACGGCGACCACCGAGATCTACACCGTTGCAANNWNNWNNACACTCTTTCCCT | 561 |
| | ACACGACGCTCTTCCGATC*T | |
| P566 | AATGATACGGCGACCACCGAGATCTACACATCCGGTANNWNNWNNACACTCTTTCCCT | 562 |
| | ACACGACGCTCTTCCGATC*T | |
| P567 | AATGATACGGCGACCACCGAGATCTACACGCGTCATTNNWNNWNNACACTCTTTCCCT | 563 |
| | ACACGACGCTCTTCCGATC*T | |
| P568 | AATGATACGGCGACCACCGAGATCTACACGCACAACTNNWNNWNNACACTCTTTCCCT | 564 |
| | ACACGACGCTCTTCCGATC*T | |
| P569 | AATGATACGGCGACCACCGAGATCTACACGATTACCGNNWNNWNNACACTCTTTCCCT | 565 |
| | ACACGACGCTCTTCCGATC*T | |
| P570 | AATGATACGGCGACCACCGAGATCTACACACCACGATNNWNNWNNACACTCTTTCCCT | 566 |
| | ACACGACGCTCTTCCGATC*T | |
| P571 | AATGATACGGCGACCACCGAGATCTACACGTCGAAGANNWNNWNNACACTCTTTCCCT | 567 |
| | ACACGACGCTCTTCCGATC*T | |
| P572 | AATGATACGGCGACCACCGAGATCTACACCCTTGATCNNWNNWNNACACTCTTTCCCT | 568 |
| | ACACGACGCTCTTCCGATC*T | |
| P573 | AATGATACGGCGACCACCGAGATCTACACAAGCACTGNNWNNWNNACACTCTTTCCCT | 569 |
| | ACACGACGCTCTTCCGATC*T | |
| P574 | AATGATACGGCGACCACCGAGATCTACACTTCGTTGGNNWNNWNNACACTCTTTCCCT | 570 |
| | ACACGACGCTCTTCCGATC*T | |
| P575 | AATGATACGGCGACCACCGAGATCTACACTCGCTGTTNNWNNWNNACACTCTTTCCCT | 571 |
| | ACACGACGCTCTTCCGATC*T | |
| P576 | AATGATACGGCGACCACCGAGATCTACACGAATCCGANNWNNWNNACACTCTTTCCCT | 572 |
| | ACACGACGCTCTTCCGATC*T | |
| P577 | AATGATACGGCGACCACCGAGATCTACACGTGCCATANNWNNWNNACACTCTTTCCCT | 573 |
| | ACACGACGCTCTTCCGATC*T | |
| P578 | AATGATACGGCGACCACCGAGATCTACACCTTAGGACNNWNNWNNACACTCTTTCCCT | 574 |
| | ACACGACGCTCTTCCGATC*T | |
| P579 | AATGATACGGCGACCACCGAGATCTACACAACTGAGCNNWNNWNNACACTCTTTCCCT | 575 |
| | ACACGACGCTCTTCCGATC*T | |
| P580 | AATGATACGGCGACCACCGAGATCTACACGACGATCTNNWNNWNNACACTCTTTCCCT | 576 |
| | ACACGACGCTCTTCCGATC*T | |
| P581 | AATGATACGGCGACCACCGAGATCTACACATCCAGAGNNWNNWNNACACTCTTTCCCT | 577 |
| | ACACGACGCTCTTCCGATC*T | |
| P582 | AATGATACGGCGACCACCGAGATCTACACAGAGTAGCNNWNNWNNACACTCTTTCCCT | 578 |
| | ACACGACGCTCTTCCGATC*T | |
| P583 | AATGATACGGCGACCACCGAGATCTACACTGGACTCTNNWNNWNNACACTCTTTCCCT | 579 |
| | ACACGACGCTCTTCCGATC*T | |
| P584 | AATGATACGGCGACCACCGAGATCTACACTACGCTACNNWNNWNNACACTCTTTCCCT | 580 |
| | ACACGACGCTCTTCCGATC*T | |
| P585 | AATGATACGGCGACCACCGAGATCTACACGCTATCCTNNWNNWNNACACTCTTTCCCT | 581 |
| | ACACGACGCTCTTCCGATC*T | |
| P586 | AATGATACGGCGACCACCGAGATCTACACGCAAGATCNNWNNWNNACACTCTTTCCCT | 582 |
| | ACACGACGCTCTTCCGATC*T | |
| P587 | AATGATACGGCGACCACCGAGATCTACACATCGATCGNNWNNWNNACACTCTTTCCCT | 583 |
| | ACACGACGCTCTTCCGATC*T | |
| P588 | AATGATACGGCGACCACCGAGATCTACACCGGCTAATNNWNNWNNACACTCTTTCCCT | 584 |
| | ACACGACGCTCTTCCGATC*T | |
| P589 | AATGATACGGCGACCACCGAGATCTACACACGGAACANNWNNWNNACACTCTTTCCCT | 585 |
| | ACACGACGCTCTTCCGATC*T | |
| P590 | AATGATACGGCGACCACCGAGATCTACACCGCATGATNNWNNWNNACACTCTTTCCCT | 586 |
| | ACACGACGCTCTTCCGATC*T | |
| P591 | AATGATACGGCGACCACCGAGATCTACACTTCCAAGGNNWNNWNNACACTCTTTCCCT | 587 |
| | ACACGACGCTCTTCCGATC*T | |
| P592 | AATGATACGGCGACCACCGAGATCTACACCTTGTCGANNWNNWNNACACTCTTTCCCT | 588 |
| | ACACGACGCTCTTCCGATC*T | |
| P593 | AATGATACGGCGACCACCGAGATCTACACGAGACGATNNWNNWNNACACTCTTTCCCT | 589 |
| | ACACGACGCTCTTCCGATC*T | |
| P594 | AATGATACGGCGACCACCGAGATCTACACTGAGCTAGNNWNNWNNACACTCTTTCCCT | 590 |
| | ACACGACGCTCTTCCGATC*T | |
| P595 | AATGATACGGCGACCACCGAGATCTACACACTCTCGANNWNNWNNACACTCTTTCCCT | 591 |
| | ACACGACGCTCTTCCGATC*T | |
| P596 | AATGATACGGCGACCACCGAGATCTACACCTGATCGTNNWNNWNNACACTCTTTCCCT | 592 |
| | ACACGACGCTCTTCCGATC*T | |
| P597 | AATGATACGGCGACCACCGAGATCTACACCGACCATTNNWNNWNNACACTCTTTCCCT | 593 |
| | ACACGACGCTCTTCCGATC*T | |
| P598 | AATGATACGGCGACCACCGAGATCTACACGATAGCGANNWNNWNNACACTCTTTCCCT | 594 |
| | ACACGACGCTCTTCCGATC*T | |
| P599 | AATGATACGGCGACCACCGAGATCTACACAATGGACGNNWNNWNNACACTCTTTCCCT | 595 |
| | ACACGACGCTCTTCCGATC*T | |
| P5100 | AATGATACGGCGACCACCGAGATCTACACCGCTAGTANNWNNWNNACACTCTTTCCCT | 596 |
| | ACACGACGCTCTTCCGATC*T | |
| P5101 | AATGATACGGCGACCACCGAGATCTACACTCTCTAGGNNWNNWNNACACTCTTTCCCT | 597 |
| | ACACGACGCTCTTCCGATC*T | |
| P5102 | AATGATACGGCGACCACCGAGATCTACACACATTGCGNNWNNWNNACACTCTTTCCCT | 598 |
| | ACACGACGCTCTTCCGATC*T | |
| P5103 | AATGATACGGCGACCACCGAGATCTACACTGAGGTGTNNWNNWNNACACTCTTTCCCT | 599 |
| | ACACGACGCTCTTCCGATC*T | |
| P5104 | AATGATACGGCGACCACCGAGATCTACACAATGCCTCNNWNNWNNACACTCTTTCCCT | 600 |
| | ACACGACGCTCTTCCGATC*T | |
| P5105 | AATGATACGGCGACCACCGAGATCTACACCTGGAGTANNWNNWNNACACTCTTTCCCT | 601 |
| | ACACGACGCTCTTCCGATC*T | |
| P5106 | AATGATACGGCGACCACCGAGATCTACACGTATGCTGNNWNNWNNACACTCTTTCCCT | 602 |
| | ACACGACGCTCTTCCGATC*T | |
| P5107 | AATGATACGGCGACCACCGAGATCTACACTGGAGAGTNNWNNWNNACACTCTTTCCCT | 603 |
| | ACACGACGCTCTTCCGATC*T | |
| P5108 | AATGATACGGCGACCACCGAGATCTACACCGATAGAGNNWNNWNNACACTCTTTCCCT | 604 |
| | ACACGACGCTCTTCCGATC*T | |
| P5109 | AATGATACGGCGACCACCGAGATCTACACCTCATTGCNNWNNWNNACACTCTTTCCCT | 605 |
| | ACACGACGCTCTTCCGATC*T | |
| P5110 | AATGATACGGCGACCACCGAGATCTACACACCAGCTTNNWNNWNNACACTCTTTCCCT | 606 |
| | ACACGACGCTCTTCCGATC*T | |
| P5111 | AATGATACGGCGACCACCGAGATCTACACGAATCGTGNNWNNWNNACACTCTTTCCCT | 607 |
| | ACACGACGCTCTTCCGATC*T | |
| P5112 | AATGATACGGCGACCACCGAGATCTACACAGGCTTCTNNWNNWNNACACTCTTTCCCT | 608 |
| | ACACGACGCTCTTCCGATC*T | |
| P5113 | AATGATACGGCGACCACCGAGATCTACACCAGTTCTGNNWNNWNNACACTCTTTCCCT | 609 |
| | ACACGACGCTCTTCCGATC*T | |
| P5114 | AATGATACGGCGACCACCGAGATCTACACTTGGTGAGNNWNNWNNACACTCTTTCCCT | 610 |
| | ACACGACGCTCTTCCGATC*T | |
| P5115 | AATGATACGGCGACCACCGAGATCTACACCATTCGGTNNWNNWNNACACTCTTTCCCT | 611 |
| | ACACGACGCTCTTCCGATC*T | |
| P5116 | AATGATACGGCGACCACCGAGATCTACACTGTGAAGCNNWNNWNNACACTCTTTCCCT | 612 |
| | ACACGACGCTCTTCCGATC*T | |
| P5117 | AATGATACGGCGACCACCGAGATCTACACTAAGTGGCNNWNNWNNACACTCTTTCCCT | 613 |
| | ACACGACGCTCTTCCGATC*T | |
| P5118 | AATGATACGGCGACCACCGAGATCTACACACGTGATGNNWNNWNNACACTCTTTCCCT | 614 |
| | ACACGACGCTCTTCCGATC*T | |
| P5119 | AATGATACGGCGACCACCGAGATCTACACGTAGAGCANNWNNWNNACACTCTTTCCCT | 615 |
| | ACACGACGCTCTTCCGATC*T | |
| P5120 | AATGATACGGCGACCACCGAGATCTACACGTCAGTTGNNWNNWNNACACTCTTTCCCT | 616 |
| | ACACGACGCTCTTCCGATC*T | |
| P5121 | AATGATACGGCGACCACCGAGATCTACACATTCGAGGNNWNNWNNACACTCTTTCCCT | 617 |
| | ACACGACGCTCTTCCGATC*T | |
| P5122 | AATGATACGGCGACCACCGAGATCTACACGATACTGGNNWNNWNNACACTCTTTCCCT | 618 |
| | ACACGACGCTCTTCCGATC*T | |
| P5123 | AATGATACGGCGACCACCGAGATCTACACGCCTTGTTNNWNNWNNACACTCTTTCCCT | 619 |
| | ACACGACGCTCTTCCGATC*T | |
| P5124 | AATGATACGGCGACCACCGAGATCTACACTTGGTCTCNNWNNWNNACACTCTTTCCCT | 620 |
| | ACACGACGCTCTTCCGATC*T | |
| P5125 | AATGATACGGCGACCACCGAGATCTACACCCGACTATNNWNNWNNACACTCTTTCCCT | 621 |
| | ACACGACGCTCTTCCGATC*T | |
| P5126 | AATGATACGGCGACCACCGAGATCTACACGTCCTAAGNNWNNWNNACACTCTTTCCCT | 622 |
| | ACACGACGCTCTTCCGATC*T | |
| P5127 | AATGATACGGCGACCACCGAGATCTACACACCAATGCNNWNNWNNACACTCTTTCCCT | 623 |
| | ACACGACGCTCTTCCGATC*T | |
| P5128 | AATGATACGGCGACCACCGAGATCTACACGATGCACTNNWNNWNNACACTCTTTCCCT | 624 |
| | ACACGACGCTCTTCCGATC*T | |
| P5129 | AATGATACGGCGACCACCGAGATCTACACGCTGGATTNNWNNWNNACACTCTTTCCCT | 625 |
| | ACACGACGCTCTTCCGATC*T | |
| P5130 | AATGATACGGCGACCACCGAGATCTACACATGGTTGCNNWNNWNNACACTCTTTCCCT | 626 |
| | ACACGACGCTCTTCCGATC*T | |
| P5131 | AATGATACGGCGACCACCGAGATCTACACCAGAATCGNNWNNWNNACACTCTTTCCCT | 627 |
| | ACACGACGCTCTTCCGATC*T | |
| P5132 | AATGATACGGCGACCACCGAGATCTACACGAACGCTTNNWNNWNNACACTCTTTCCCT | 628 |
| | ACACGACGCTCTTCCGATC*T | |
| P5133 | AATGATACGGCGACCACCGAGATCTACACTCGAACCANNWNNWNNACACTCTTTCCCT | 629 |
| | ACACGACGCTCTTCCGATC*T | |
| P5134 | AATGATACGGCGACCACCGAGATCTACACCTATCGCANNWNNWNNACACTCTTTCCCT | 630 |
| | ACACGACGCTCTTCCGATC*T | |
| P5135 | AATGATACGGCGACCACCGAGATCTACACTACGGTTGNNWNNWNNACACTCTTTCCCT | 631 |
| | ACACGACGCTCTTCCGATC*T | |
| P5136 | AATGATACGGCGACCACCGAGATCTACACGAGATGTCNNWNNWNNACACTCTTTCCCT | 632 |
| | ACACGACGCTCTTCCGATC*T | |
| P5137 | AATGATACGGCGACCACCGAGATCTACACCTTACAGCNNWNNWNNACACTCTTTCCCT | 633 |
| | ACACGACGCTCTTCCGATC*T | |
| P5138 | AATGATACGGCGACCACCGAGATCTACACAGGAGGAANNWNNWNNACACTCTTTCCCT | 634 |
| | ACACGACGCTCTTCCGATC*T | |
| P5139 | AATGATACGGCGACCACCGAGATCTACACGACGAATGNNWNNWNNACACTCTTTCCCT | 635 |
| | ACACGACGCTCTTCCGATC*T | |
| P5140 | AATGATACGGCGACCACCGAGATCTACACGAAGAGGTNNWNNWNNACACTCTTTCCCT | 636 |
| | ACACGACGCTCTTCCGATC*T | |
| P5141 | AATGATACGGCGACCACCGAGATCTACACCGTCAATGNNWNNWNNACACTCTTTCCCT | 637 |
| | ACACGACGCTCTTCCGATC*T | |
| P5142 | AATGATACGGCGACCACCGAGATCTACACTACCAGGANNWNNWNNACACTCTTTCCCT | 638 |
| | ACACGACGCTCTTCCGATC*T | |
| P5143 | AATGATACGGCGACCACCGAGATCTACACCGTACGAANNWNNWNNACACTCTTTCCCT | 639 |
| | ACACGACGCTCTTCCGATC*T | |
| P5144 | AATGATACGGCGACCACCGAGATCTACACGACTTAGGNNWNNWNNACACTCTTTCCCT | 640 |
| | ACACGACGCTCTTCCGATC*T | |
| P5145 | AATGATACGGCGACCACCGAGATCTACACAGTGCAGTNNWNNWNNACACTCTTTCCCT | 641 |
| | ACACGACGCTCTTCCGATC*T | |
| P5146 | AATGATACGGCGACCACCGAGATCTACACTTGATCCGNNWNNWNNACACTCTTTCCCT | 642 |
| | ACACGACGCTCTTCCGATC*T | |
| P5147 | AATGATACGGCGACCACCGAGATCTACACTGCCATTCNNWNNWNNACACTCTTTCCCT | 643 |
| | ACACGACGCTCTTCCGATC*T | |
| P5148 | AATGATACGGCGACCACCGAGATCTACACCTTGCTGTNNWNNWNNACACTCTTTCCCT | 644 |
| | ACACGACGCTCTTCCGATC*T | |
| P5149 | AATGATACGGCGACCACCGAGATCTACACCCTACTGANNWNNWNNACACTCTTTCCCT | 645 |
| | ACACGACGCTCTTCCGATC*T | |
| P5150 | AATGATACGGCGACCACCGAGATCTACACCCAAGTTGNNWNNWNNACACTCTTTCCCT | 646 |
| | ACACGACGCTCTTCCGATC*T | |
| P5151 | AATGATACGGCGACCACCGAGATCTACACTGATCGGANNWNNWNNACACTCTTTCCCT | 647 |
| | ACACGACGCTCTTCCGATC*T | |
| P5152 | AATGATACGGCGACCACCGAGATCTACACTAGTTGCGNNWNNWNNACACTCTTTCCCT | 648 |
| | ACACGACGCTCTTCCGATC*T | |
| P5153 | AATGATACGGCGACCACCGAGATCTACACGTCTGATCNNWNNWNNACACTCTTTCCCT | 649 |
| | ACACGACGCTCTTCCGATC*T | |
| P5154 | AATGATACGGCGACCACCGAGATCTACACCGTTATGCNNWNNWNNACACTCTTTCCCT | 650 |
| | ACACGACGCTCTTCCGATC*T | |
| P5155 | AATGATACGGCGACCACCGAGATCTACACGCTCTGTANNWNNWNNACACTCTTTCCCT | 651 |
| | ACACGACGCTCTTCCGATC*T | |
| P5156 | AATGATACGGCGACCACCGAGATCTACACTTACCGAGNNWNNWNNACACTCTTTCCCT | 652 |
| | ACACGACGCTCTTCCGATC*T | |
| P5157 | AATGATACGGCGACCACCGAGATCTACACGCCATAACNNWNNWNNACACTCTTTCCCT | 653 |
| | ACACGACGCTCTTCCGATC*T | |
| P5158 | AATGATACGGCGACCACCGAGATCTACACCTCAGAGTNNWNNWNNACACTCTTTCCCT | 654 |
| | ACACGACGCTCTTCCGATC*T | |
| P5159 | AATGATACGGCGACCACCGAGATCTACACCGAGACTANNWNNWNNACACTCTTTCCCT | 655 |
| | ACACGACGCTCTTCCGATC*T | |
| P5160 | AATGATACGGCGACCACCGAGATCTACACTGTGCGTTNNWNNWNNACACTCTTTCCCT | 656 |
| | ACACGACGCTCTTCCGATC*T | |
| P5161 | AATGATACGGCGACCACCGAGATCTACACTTCAGGAGNNWNNWNNACACTCTTTCCCT | 657 |
| | ACACGACGCTCTTCCGATC*T | |
| P5162 | AATGATACGGCGACCACCGAGATCTACACGACTATGCNNWNNWNNACACTCTTTCCCT | 658 |
| | ACACGACGCTCTTCCGATC*T | |
| P5163 | AATGATACGGCGACCACCGAGATCTACACAGGTTCGANNWNNWNNACACTCTTTCCCT | 659 |
| | ACACGACGCTCTTCCGATC*T | |
| P5164 | AATGATACGGCGACCACCGAGATCTACACAGTCTGTGNNWNNWNNACACTCTTTCCCT | 660 |
| | ACACGACGCTCTTCCGATC*T | |
| P5165 | AATGATACGGCGACCACCGAGATCTACACACCTAAGGNNWNNWNNACACTCTTTCCCT | 661 |
| | ACACGACGCTCTTCCGATC*T | |
| P5166 | AATGATACGGCGACCACCGAGATCTACACTGCAGGTANNWNNWNNACACTCTTTCCCT | 662 |
| | ACACGACGCTCTTCCGATC*T | |
| P5167 | AATGATACGGCGACCACCGAGATCTACACAAGGACACNNWNNWNNACACTCTTTCCCT | 663 |
| | ACACGACGCTCTTCCGATC*T | |
| P5168 | AATGATACGGCGACCACCGAGATCTACACCAACCTAGNNWNNWNNACACTCTTTCCCT | 664 |
| | ACACGACGCTCTTCCGATC*T | |
| P5169 | AATGATACGGCGACCACCGAGATCTACACCTGACACANNWNNWNNACACTCTTTCCCT | 665 |
| | ACACGACGCTCTTCCGATC*T | |
| P5170 | AATGATACGGCGACCACCGAGATCTACACACTCGTTGNNWNNWNNACACTCTTTCCCT | 666 |
| | ACACGACGCTCTTCCGATC*T | |
| P5171 | AATGATACGGCGACCACCGAGATCTACACAGCTCCTANNWNNWNNACACTCTTTCCCT | 667 |
| | ACACGACGCTCTTCCGATC*T | |
| P5172 | AATGATACGGCGACCACCGAGATCTACACTACATCGGNNWNNWNNACACTCTTTCCCT | 668 |
| | ACACGACGCTCTTCCGATC*T | |
| P5173 | AATGATACGGCGACCACCGAGATCTACACCACAAGTCNNWNNWNNACACTCTTTCCCT | 669 |
| | ACACGACGCTCTTCCGATC*T | |
| P5174 | AATGATACGGCGACCACCGAGATCTACACCGGATTGANNWNNWNNACACTCTTTCCCT | 670 |
| | ACACGACGCTCTTCCGATC*T | |
| P5175 | AATGATACGGCGACCACCGAGATCTACACAGTCGACANNWNNWNNACACTCTTTCCCT | 671 |
| | ACACGACGCTCTTCCGATC*T | |
| P5176 | AATGATACGGCGACCACCGAGATCTACACGTCTCCTTNNWNNWNNACACTCTTTCCCT | 672 |
| | ACACGACGCTCTTCCGATC*T | |
| P5177 | AATGATACGGCGACCACCGAGATCTACACGAGATACGNNWNNWNNACACTCTTTCCCT | 673 |
| | ACACGACGCTCTTCCGATC*T | |
| P5178 | AATGATACGGCGACCACCGAGATCTACACATCGGTGTNNWNNWNNACACTCTTTCCCT | 674 |
| | ACACGACGCTCTTCCGATC*T | |
| P5179 | AATGATACGGCGACCACCGAGATCTACACTCTCGCAANNWNNWNNACACTCTTTCCCT | 675 |
| | ACACGACGCTCTTCCGATC*T | |
| P5180 | AATGATACGGCGACCACCGAGATCTACACTCTAACGCNNWNNWNNACACTCTTTCCCT | 676 |
| | ACACGACGCTCTTCCGATC*T | |
| P5181 | AATGATACGGCGACCACCGAGATCTACACCAATCGACNNWNNWNNACACTCTTTCCCT | 677 |
| | ACACGACGCTCTTCCGATC*T | |
| P5182 | AATGATACGGCGACCACCGAGATCTACACGAGGACTTNNWNNWNNACACTCTTTCCCT | 678 |
| | ACACGACGCTCTTCCGATC*T | |
| P5183 | AATGATACGGCGACCACCGAGATCTACACTGGAGTTGNNWNNWNNACACTCTTTCCCT | 679 |
| | ACACGACGCTCTTCCGATC*T | |
| P5184 | AATGATACGGCGACCACCGAGATCTACACCTAGGCATNNWNNWNNACACTCTTTCCCT | 680 |
| | ACACGACGCTCTTCCGATC*T | |
| P5185 | AATGATACGGCGACCACCGAGATCTACACCTCTACTCNNWNNWNNACACTCTTTCCCT | 681 |
| | ACACGACGCTCTTCCGATC*T | |
| P5186 | AATGATACGGCGACCACCGAGATCTACACAGAAGCGTNNWNNWNNACACTCTTTCCCT | 682 |
| | ACACGACGCTCTTCCGATC*T | |
| P5187 | AATGATACGGCGACCACCGAGATCTACACTCGAAGGTNNWNNWNNACACTCTTTCCCT | 683 |
| | ACACGACGCTCTTCCGATC*T | |
| P5188 | AATGATACGGCGACCACCGAGATCTACACGTCGGTAANNWNNWNNACACTCTTTCCCT | 684 |
| | ACACGACGCTCTTCCGATC*T | |
| P5189 | AATGATACGGCGACCACCGAGATCTACACACGATGACNNWNNWNNACACTCTTTCCCT | 685 |
| | ACACGACGCTCTTCCGATC*T | |
| P5190 | AATGATACGGCGACCACCGAGATCTACACTCCGTATGNNWNNWNNACACTCTTTCCCT | 686 |
| | ACACGACGCTCTTCCGATC*T | |
| P5191 | AATGATACGGCGACCACCGAGATCTACACCTAGGTGANNWNNWNNACACTCTTTCCCT | 687 |
| | ACACGACGCTCTTCCGATC*T | |
| P5192 | AATGATACGGCGACCACCGAGATCTACACCATTGCCTNNWNNWNNACACTCTTTCCCT | 688 |
| | ACACGACGCTCTTCCGATC*T | |
| P5 | /5Phos/GATCGGAAGAGC*C*A | 689 |
| Common | | |
| Adapter | | |
| i7_1 | CAAGCAGAAGACGGCATACGAGATACGATCAGGGCAGTCGGTGATCATAGCGGTATTA | 690 |
| | CGCGAGATTACGA | |
| i7_2 | CAAGCAGAAGACGGCATACGAGATTCGAGAGTGGCAGTCGGTGATCATAGCGGTATTA | 691 |
| | CGCGAGATTACGA | |
| i7_3 | CAAGCAGAAGACGGCATACGAGATCTAGCTCAGGCAGTCGGTGATCATAGCGGTATTA | 692 |
| | CGCGAGATTACGA | |
| i7_4 | CAAGCAGAAGACGGCATACGAGATATCGTCTCGGCAGTCGGTGATCATAGCGGTATTA | 693 |
| | CGCGAGATTACGA | |
| i7_5 | CAAGCAGAAGACGGCATACGAGATTCGACAAGGGCAGTCGGTGATCATAGCGGTATTA | 694 |
| | CGCGAGATTACGA | |
| i7_6 | CAAGCAGAAGACGGCATACGAGATCCTTGGAAGGCAGTCGGTGATCATAGCGGTATTA | 695 |
| | CGCGAGATTACGA | |
| i7_7 | CAAGCAGAAGACGGCATACGAGATATCATGCGGGCAGTCGGTGATCATAGCGGTATTA | 696 |
| | CGCGAGATTACGA | |
| i7_8 | CAAGCAGAAGACGGCATACGAGATTGTTCCGTGGCAGTCGGTGATCATAGCGGTATTA | 697 |
| | CGCGAGATTACGA | |
| i7_9 | CAAGCAGAAGACGGCATACGAGATATTAGCCGGGCAGTCGGTGATCATAGCGGTATTA | 698 |
| | CGCGAGATTACGA | |
| i7_10 | CAAGCAGAAGACGGCATACGAGATCGATCGATGGCAGTCGGTGATCATAGCGGTATTA | 699 |
| | CGCGAGATTACGA | |
| i7_11 | CAAGCAGAAGACGGCATACGAGATGATCTTGCGGCAGTCGGTGATCATAGCGGTATTA | 700 |
| | CGCGAGATTACGA | |
| i7_12 | CAAGCAGAAGACGGCATACGAGATAGGATAGCGGCAGTCGGTGATCATAGCGGTATTA | 701 |
| | CGCGAGATTACGA | |
| i7_13 | CAAGCAGAAGACGGCATACGAGATGTAGCGTAGGCAGTCGGTGATCATAGCGGTATTA | 702 |
| | CGCGAGATTACGA | |
| i7_14 | CAAGCAGAAGACGGCATACGAGATAGAGTCCAGGCAGTCGGTGATCATAGCGGTATTA | 703 |
| | CGCGAGATTACGA | |
| i7_15 | CAAGCAGAAGACGGCATACGAGATGCTACTCTGGCAGTCGGTGATCATAGCGGTATTA | 704 |
| | CGCGAGATTACGA | |
| i7_16 | CAAGCAGAAGACGGCATACGAGATCTCTGGATGGCAGTCGGTGATCATAGCGGTATTA | 705 |
| | CGCGAGATTACGA | |
| i7_17 | CAAGCAGAAGACGGCATACGAGATAGATCGTCGGCAGTCGGTGATCATAGCGGTATTA | 706 |
| | CGCGAGATTACGA | |
| i7_18 | CAAGCAGAAGACGGCATACGAGATGCTCAGTTGGCAGTCGGTGATCATAGCGGTATTA | 707 |
| | CGCGAGATTACGA | |
| i7_19 | CAAGCAGAAGACGGCATACGAGATGTCCTAAGGGCAGTCGGTGATCATAGCGGTATTA | 708 |
| | CGCGAGATTACGA | |
| i7_20 | CAAGCAGAAGACGGCATACGAGATTATGGCACGGCAGTCGGTGATCATAGCGGTATTA | 709 |
| | CGCGAGATTACGA | |
| i7_21 | CAAGCAGAAGACGGCATACGAGATTCGGATTCGGCAGTCGGTGATCATAGCGGTATTA | 710 |
| | CGCGAGATTACGA | |
| i7_22 | CAAGCAGAAGACGGCATACGAGATAACAGCGAGGCAGTCGGTGATCATAGCGGTATTA | 711 |
| | CGCGAGATTACGA | |
| i7_23 | CAAGCAGAAGACGGCATACGAGATCCAACGAAGGCAGTCGGTGATCATAGCGGTATTA | 712 |
| | CGCGAGATTACGA | |
| i7_24 | CAAGCAGAAGACGGCATACGAGATCAGTGCTTGGCAGTCGGTGATCATAGCGGTATTA | 713 |
| | CGCGAGATTACGA | |
| i7_25 | CAAGCAGAAGACGGCATACGAGATGATCAAGGGGCAGTCGGTGATCATAGCGGTATTA | 714 |
| | CGCGAGATTACGA | |
| i7_26 | CAAGCAGAAGACGGCATACGAGATTCTTCGACGGCAGTCGGTGATCATAGCGGTATTA | 715 |
| | CGCGAGATTACGA | |
| i7_27 | CAAGCAGAAGACGGCATACGAGATATCGTGGTGGCAGTCGGTGATCATAGCGGTATTA | 716 |
| | CGCGAGATTACGA | |
| i7_28 | CAAGCAGAAGACGGCATACGAGATCGGTAATCGGCAGTCGGTGATCATAGCGGTATTA | 717 |
| | CGCGAGATTACGA | |
| i7_29 | CAAGCAGAAGACGGCATACGAGATAGTTGTGCGGCAGTCGGTGATCATAGCGGTATTA | 718 |
| | CGCGAGATTACGA | |
| i7_30 | CAAGCAGAAGACGGCATACGAGATAATGACGCGGCAGTCGGTGATCATAGCGGTATTA | 719 |
| | CGCGAGATTACGA | |
| i7_31 | CAAGCAGAAGACGGCATACGAGATTACCGGATGGCAGTCGGTGATCATAGCGGTATTA | 720 |
| | CGCGAGATTACGA | |
| i7_32 | CAAGCAGAAGACGGCATACGAGATTTGCAACGGGCAGTCGGTGATCATAGCGGTATTA | 721 |
| | CGCGAGATTACGA | |
| i7_33 | CAAGCAGAAGACGGCATACGAGATCACTTCACGGCAGTCGGTGATCATAGCGGTATTA | 722 |
| | CGCGAGATTACGA | |
| i7_34 | CAAGCAGAAGACGGCATACGAGATTAGCCATGGGCAGTCGGTGATCATAGCGGTATTA | 723 |
| | CGCGAGATTACGA | |
| i7_35 | CAAGCAGAAGACGGCATACGAGATACAGGCATGGCAGTCGGTGATCATAGCGGTATTA | 724 |
| | CGCGAGATTACGA | |
| i7_36 | CAAGCAGAAGACGGCATACGAGATAGGTGTTGGGCAGTCGGTGATCATAGCGGTATTA | 725 |
| | CGCGAGATTACGA | |
| i7_37 | CAAGCAGAAGACGGCATACGAGATCAGTCACAGGCAGTCGGTGATCATAGCGGTATTA | 726 |
| | CGCGAGATTACGA | |
| i7_38 | CAAGCAGAAGACGGCATACGAGATTCGATGACGGCAGTCGGTGATCATAGCGGTATTA | 727 |
| | CGCGAGATTACGA | |
| i7_39 | CAAGCAGAAGACGGCATACGAGATGAAGTGCTGGCAGTCGGTGATCATAGCGGTATTA | 728 |
| | CGCGAGATTACGA | |
| i7_40 | CAAGCAGAAGACGGCATACGAGATCTTCCTTCGGCAGTCGGTGATCATAGCGGTATTA | 729 |
| | CGCGAGATTACGA | |
| i7_41 | CAAGCAGAAGACGGCATACGAGATCGAACAACGGCAGTCGGTGATCATAGCGGTATTA | 730 |
| | CGCGAGATTACGA | |
| i7_42 | CAAGCAGAAGACGGCATACGAGATAACAACCGGGCAGTCGGTGATCATAGCGGTATTA | 731 |
| | CGCGAGATTACGA | |
| i7_43 | CAAGCAGAAGACGGCATACGAGATACCTCAGTGGCAGTCGGTGATCATAGCGGTATTA | 732 |
| | CGCGAGATTACGA | |
| i7_44 | CAAGCAGAAGACGGCATACGAGATCGTCTTCAGGCAGTCGGTGATCATAGCGGTATTA | 733 |
| | CGCGAGATTACGA | |
| i7_45 | CAAGCAGAAGACGGCATACGAGATTGCGTAACGGCAGTCGGTGATCATAGCGGTATTA | 734 |
| | CGCGAGATTACGA | |
| i7_46 | CAAGCAGAAGACGGCATACGAGATAACACGCTGGCAGTCGGTGATCATAGCGGTATTA | 735 |
| | CGCGAGATTACGA | |
| i7_47 | CAAGCAGAAGACGGCATACGAGATACTCGATCGGCAGTCGGTGATCATAGCGGTATTA | 736 |
| | CGCGAGATTACGA | |
| i7_48 | CAAGCAGAAGACGGCATACGAGATTGAGCTGTGGCAGTCGGTGATCATAGCGGTATTA | 737 |
| | CGCGAGATTACGA | |
| i7_49 | CAAGCAGAAGACGGCATACGAGATTACTGCTCGGCAGTCGGTGATCATAGCGGTATTA | 738 |
| | CGCGAGATTACGA | |
| i7_50 | CAAGCAGAAGACGGCATACGAGATGACGAACTGGCAGTCGGTGATCATAGCGGTATTA | 739 |
| | CGCGAGATTACGA | |
| i7_51 | CAAGCAGAAGACGGCATACGAGATCTTCGCAAGGCAGTCGGTGATCATAGCGGTATTA | 740 |
| | CGCGAGATTACGA | |
| i7_52 | CAAGCAGAAGACGGCATACGAGATATGGCGATGGCAGTCGGTGATCATAGCGGTATTA | 741 |
| | CGCGAGATTACGA | |
| i7_53 | CAAGCAGAAGACGGCATACGAGATACATGCCAGGCAGTCGGTGATCATAGCGGTATTA | 742 |
| | CGCGAGATTACGA | |
| i7_54 | CAAGCAGAAGACGGCATACGAGATGTCAACAGGGCAGTCGGTGATCATAGCGGTATTA | 743 |
| | CGCGAGATTACGA | |
| i7_55 | CAAGCAGAAGACGGCATACGAGATGTGGTATGGGCAGTCGGTGATCATAGCGGTATTA | 744 |
| | CGCGAGATTACGA | |
| i7_56 | CAAGCAGAAGACGGCATACGAGATCCAACTTCGGCAGTCGGTGATCATAGCGGTATTA | 745 |
| | CGCGAGATTACGA | |
| i7_57 | CAAGCAGAAGACGGCATACGAGATGACGTCATGGCAGTCGGTGATCATAGCGGTATTA | 746 |
| | CGCGAGATTACGA | |
| i7_58 | CAAGCAGAAGACGGCATACGAGATACGTCCAAGGCAGTCGGTGATCATAGCGGTATTA | 747 |
| | CGCGAGATTACGA | |
| i7_59 | CAAGCAGAAGACGGCATACGAGATGATCCACTGGCAGTCGGTGATCATAGCGGTATTA | 748 |
| | CGCGAGATTACGA | |
| i7_60 | CAAGCAGAAGACGGCATACGAGATAGCCTATCGGCAGTCGGTGATCATAGCGGTATTA | 749 |
| | CGCGAGATTACGA | |
| i7_61 | CAAGCAGAAGACGGCATACGAGATAGCTACCAGGCAGTCGGTGATCATAGCGGTATTA | 750 |
| | CGCGAGATTACGA | |
| i7_62 | CAAGCAGAAGACGGCATACGAGATAGATTGCGGGCAGTCGGTGATCATAGCGGTATTA | 751 |
| | CGCGAGATTACGA | |
| i7_63 | CAAGCAGAAGACGGCATACGAGATCACACATCGGCAGTCGGTGATCATAGCGGTATTA | 752 |
| | CGCGAGATTACGA | |
| i7_64 | CAAGCAGAAGACGGCATACGAGATGAGCAATCGGCAGTCGGTGATCATAGCGGTATTA | 753 |
| | CGCGAGATTACGA | |
| i7_65 | CAAGCAGAAGACGGCATACGAGATATAGAGCGGGCAGTCGGTGATCATAGCGGTATTA | 754 |
| | CGCGAGATTACGA | |
| i7_66 | CAAGCAGAAGACGGCATACGAGATGACCGATAGGCAGTCGGTGATCATAGCGGTATTA | 755 |
| | CGCGAGATTACGA | |
| i7_67 | CAAGCAGAAGACGGCATACGAGATCAGACGTTGGCAGTCGGTGATCATAGCGGTATTA | 756 |
| | CGCGAGATTACGA | |
| i7_68 | CAAGCAGAAGACGGCATACGAGATCTGAACGTGGCAGTCGGTGATCATAGCGGTATTA | 757 |
| | CGCGAGATTACGA | |
| i7_69 | CAAGCAGAAGACGGCATACGAGATTTGGACTGGGCAGTCGGTGATCATAGCGGTATTA | 758 |
| | CGCGAGATTACGA | |
| i7_70 | CAAGCAGAAGACGGCATACGAGATGTCTGCAAGGCAGTCGGTGATCATAGCGGTATTA | 759 |
| | CGCGAGATTACGA | |
| i7_71 | CAAGCAGAAGACGGCATACGAGATCCACATTGGGCAGTCGGTGATCATAGCGGTATTA | 760 |
| | CGCGAGATTACGA | |
| i7_72 | CAAGCAGAAGACGGCATACGAGATGATGGAGTGGCAGTCGGTGATCATAGCGGTATTA | 761 |
| | CGCGAGATTACGA | |
| i7_73 | CAAGCAGAAGACGGCATACGAGATAGGTCAACGGCAGTCGGTGATCATAGCGGTATTA | 762 |
| | CGCGAGATTACGA | |
| i7_74 | CAAGCAGAAGACGGCATACGAGATTACACACGGGCAGTCGGTGATCATAGCGGTATTA | 763 |
| | CGCGAGATTACGA | |
| i7_75 | CAAGCAGAAGACGGCATACGAGATCAAGTCGTGGCAGTCGGTGATCATAGCGGTATTA | 764 |
| | CGCGAGATTACGA | |
| i7_76 | CAAGCAGAAGACGGCATACGAGATAGCTAGTGGGCAGTCGGTGATCATAGCGGTATTA | 765 |
| | CGCGAGATTACGA | |
| i7_77 | CAAGCAGAAGACGGCATACGAGATCTCCTAGTGGCAGTCGGTGATCATAGCGGTATTA | 766 |
| | CGCGAGATTACGA | |
| i7_78 | CAAGCAGAAGACGGCATACGAGATACTCCTACGGCAGTCGGTGATCATAGCGGTATTA | 767 |
| | CGCGAGATTACGA | |
| i7_79 | CAAGCAGAAGACGGCATACGAGATCAATCAGGGGCAGTCGGTGATCATAGCGGTATTA | 768 |
| | CGCGAGATTACGA | |
| i7_80 | CAAGCAGAAGACGGCATACGAGATTCGTGCATGGCAGTCGGTGATCATAGCGGTATTA | 769 |
| | CGCGAGATTACGA | |
| i7_81 | CAAGCAGAAGACGGCATACGAGATTAACGTCGGGCAGTCGGTGATCATAGCGGTATTA | 770 |
| | CGCGAGATTACGA | |
| i7_82 | CAAGCAGAAGACGGCATACGAGATAAGGCGTAGGCAGTCGGTGATCATAGCGGTATTA | 771 |
| | CGCGAGATTACGA | |
| i7_83 | CAAGCAGAAGACGGCATACGAGATTCTTACGGGGCAGTCGGTGATCATAGCGGTATTA | 772 |
| | CGCGAGATTACGA | |
| i7_84 | CAAGCAGAAGACGGCATACGAGATCGTGTGATGGCAGTCGGTGATCATAGCGGTATTA | 773 |
| | CGCGAGATTACGA | |
| i7_85 | CAAGCAGAAGACGGCATACGAGATAACAGGTGGGCAGTCGGTGATCATAGCGGTATTA | 774 |
| | CGCGAGATTACGA | |
| i7_86 | CAAGCAGAAGACGGCATACGAGATAGTCGAAGGGCAGTCGGTGATCATAGCGGTATTA | 775 |
| | CGCGAGATTACGA | |
| i7_87 | CAAGCAGAAGACGGCATACGAGATTGGAAGCAGGCAGTCGGTGATCATAGCGGTATTA | 776 |
| | CGCGAGATTACGA | |
| i7_88 | CAAGCAGAAGACGGCATACGAGATCTCGTTCTGGCAGTCGGTGATCATAGCGGTATTA | 777 |
| | CGCGAGATTACGA | |
| i7_89 | CAAGCAGAAGACGGCATACGAGATACGAGAACGGCAGTCGGTGATCATAGCGGTATTA | 778 |
| | CGCGAGATTACGA | |
| i7_90 | CAAGCAGAAGACGGCATACGAGATAAGCCTGAGGCAGTCGGTGATCATAGCGGTATTA | 779 |
| | CGCGAGATTACGA | |
| i7_91 | CAAGCAGAAGACGGCATACGAGATCTACAAGGGGCAGTCGGTGATCATAGCGGTATTA | 780 |
| | CGCGAGATTACGA | |
| i7_92 | CAAGCAGAAGACGGCATACGAGATCGATGTTCGGCAGTCGGTGATCATAGCGGTATTA | 781 |
| | CGCGAGATTACGA | |
| i7_93 | CAAGCAGAAGACGGCATACGAGATACCGGTTAGGCAGTCGGTGATCATAGCGGTATTA | 782 |
| | CGCGAGATTACGA | |
| i7_94 | CAAGCAGAAGACGGCATACGAGATGAACGGTTGGCAGTCGGTGATCATAGCGGTATTA | 783 |
| | CGCGAGATTACGA | |
| i7_95 | CAAGCAGAAGACGGCATACGAGATCTGTACCAGGCAGTCGGTGATCATAGCGGTATTA | 784 |
| | CGCGAGATTACGA | |
| i7_96 | CAAGCAGAAGACGGCATACGAGATGCGCATATGGCAGTCGGTGATCATAGCGGTATTA | 785 |
| | CGCGAGATTACGA | |
| i7_97 | CAAGCAGAAGACGGCATACGAGATTGATAGGCGGCAGTCGGTGATCATAGCGGTATTA | 786 |
| | CGCGAGATTACGA | |
| i7_98 | CAAGCAGAAGACGGCATACGAGATCATCCAAGGGCAGTCGGTGATCATAGCGGTATTA | 787 |
| | CGCGAGATTACGA | |
| i7_99 | CAAGCAGAAGACGGCATACGAGATGTGAGACTGGCAGTCGGTGATCATAGCGGTATTA | 788 |
| | CGCGAGATTACGA | |
| i7_100 | CAAGCAGAAGACGGCATACGAGATCTGATGAGGGCAGTCGGTGATCATAGCGGTATTA | 789 |
| | CGCGAGATTACGA | |
| i7_101 | CAAGCAGAAGACGGCATACGAGATACGGTACAGGCAGTCGGTGATCATAGCGGTATTA | 790 |
| | CGCGAGATTACGA | |
| i7_102 | CAAGCAGAAGACGGCATACGAGATCTCGACTTGGCAGTCGGTGATCATAGCGGTATTA | 791 |
| | CGCGAGATTACGA | |
| i7_103 | CAAGCAGAAGACGGCATACGAGATACAACGTGGGCAGTCGGTGATCATAGCGGTATTA | 792 |
| | CGCGAGATTACGA | |
| i7_104 | CAAGCAGAAGACGGCATACGAGATTGCTGTGAGGCAGTCGGTGATCATAGCGGTATTA | 793 |
| | CGCGAGATTACGA | |
| i7_105 | CAAGCAGAAGACGGCATACGAGATCCAAGTAGGGCAGTCGGTGATCATAGCGGTATTA | 794 |
| | CGCGAGATTACGA | |
| i7_106 | CAAGCAGAAGACGGCATACGAGATAACTGAGGGGCAGTCGGTGATCATAGCGGTATTA | 795 |
| | CGCGAGATTACGA | |
| i7_107 | CAAGCAGAAGACGGCATACGAGATAGGTAGGAGGCAGTCGGTGATCATAGCGGTATTA | 796 |
| | CGCGAGATTACGA | |
| i7_108 | CAAGCAGAAGACGGCATACGAGATTTCGCCATGGCAGTCGGTGATCATAGCGGTATTA | 797 |
| | CGCGAGATTACGA | |
| i7_109 | CAAGCAGAAGACGGCATACGAGATCAGGTAAGGGCAGTCGGTGATCATAGCGGTATTA | 798 |
| | CGCGAGATTACGA | |
| i7_110 | CAAGCAGAAGACGGCATACGAGATGTATCGAGGGCAGTCGGTGATCATAGCGGTATTA | 799 |
| | CGCGAGATTACGA | |
| i7_111 | CAAGCAGAAGACGGCATACGAGATTTCACGGAGGCAGTCGGTGATCATAGCGGTATTA | 800 |
| | CGCGAGATTACGA | |
| i7_112 | CAAGCAGAAGACGGCATACGAGATGAGCTCTAGGCAGTCGGTGATCATAGCGGTATTA | 801 |
| | CGCGAGATTACGA | |
| i7_113 | CAAGCAGAAGACGGCATACGAGATGTCAGTCAGGCAGTCGGTGATCATAGCGGTATTA | 802 |
| | CGCGAGATTACGA | |
| i7_114 | CAAGCAGAAGACGGCATACGAGATCACGTCTAGGCAGTCGGTGATCATAGCGGTATTA | 803 |
| | CGCGAGATTACGA | |
| i7_115 | CAAGCAGAAGACGGCATACGAGATAATTCCGGGGCAGTCGGTGATCATAGCGGTATTA | 804 |
| | CGCGAGATTACGA | |
| i7_116 | CAAGCAGAAGACGGCATACGAGATTCTAGGAGGGCAGTCGGTGATCATAGCGGTATTA | 805 |
| | CGCGAGATTACGA | |
| i7_117 | CAAGCAGAAGACGGCATACGAGATATCCGTTGGGCAGTCGGTGATCATAGCGGTATTA | 806 |
| | CGCGAGATTACGA | |
| i7_118 | CAAGCAGAAGACGGCATACGAGATGATAGCCAGGCAGTCGGTGATCATAGCGGTATTA | 807 |
| | CGCGAGATTACGA | |
| i7_119 | CAAGCAGAAGACGGCATACGAGATTATGACCGGGCAGTCGGTGATCATAGCGGTATTA | 808 |
| | CGCGAGATTACGA | |
| i7_120 | CAAGCAGAAGACGGCATACGAGATCGATTGGAGGCAGTCGGTGATCATAGCGGTATTA | 809 |
| | CGCGAGATTACGA | |
| i7_121 | CAAGCAGAAGACGGCATACGAGATACAAGCTCGGCAGTCGGTGATCATAGCGGTATTA | 810 |
| | CGCGAGATTACGA | |
| i7_122 | CAAGCAGAAGACGGCATACGAGATGAACCTTCGGCAGTCGGTGATCATAGCGGTATTA | 811 |
| | CGCGAGATTACGA | |
| i7_123 | CAAGCAGAAGACGGCATACGAGATAGCGAGATGGCAGTCGGTGATCATAGCGGTATTA | 812 |
| | CGCGAGATTACGA | |
| i7_124 | CAAGCAGAAGACGGCATACGAGATCCGTAACTGGCAGTCGGTGATCATAGCGGTATTA | 813 |
| | CGCGAGATTACGA | |
| i7_125 | CAAGCAGAAGACGGCATACGAGATTCAGACACGGCAGTCGGTGATCATAGCGGTATTA | 814 |
| | CGCGAGATTACGA | |
| i7_126 | CAAGCAGAAGACGGCATACGAGATCGAAGTCAGGCAGTCGGTGATCATAGCGGTATTA | 815 |
| | CGCGAGATTACGA | |
| i7_127 | CAAGCAGAAGACGGCATACGAGATGTGATCCAGGCAGTCGGTGATCATAGCGGTATTA | 816 |
| | CGCGAGATTACGA | |
| i7_128 | CAAGCAGAAGACGGCATACGAGATACTGGTGTGGCAGTCGGTGATCATAGCGGTATTA | 817 |
| | CGCGAGATTACGA | |
| i7_129 | CAAGCAGAAGACGGCATACGAGATCTAACCTGGGCAGTCGGTGATCATAGCGGTATTA | 818 |
| | CGCGAGATTACGA | |
| i7_130 | CAAGCAGAAGACGGCATACGAGATAGCCAACTGGCAGTCGGTGATCATAGCGGTATTA | 819 |
| | CGCGAGATTACGA | |
| i7_131 | CAAGCAGAAGACGGCATACGAGATCCAGTTGAGGCAGTCGGTGATCATAGCGGTATTA | 820 |
| | CGCGAGATTACGA | |
| i7_132 | CAAGCAGAAGACGGCATACGAGATAAGTGCAGGGCAGTCGGTGATCATAGCGGTATTA | 821 |
| | CGCGAGATTACGA | |
| i7_133 | CAAGCAGAAGACGGCATACGAGATAACCGTGTGGCAGTCGGTGATCATAGCGGTATTA | 822 |
| | CGCGAGATTACGA | |
| i7_134 | CAAGCAGAAGACGGCATACGAGATCGCGTATTGGCAGTCGGTGATCATAGCGGTATTA | 823 |
| | CGCGAGATTACGA | |
| i7_135 | CAAGCAGAAGACGGCATACGAGATAGTTCGCAGGCAGTCGGTGATCATAGCGGTATTA | 824 |
| | CGCGAGATTACGA | |
| i7_136 | CAAGCAGAAGACGGCATACGAGATTAGTCAGCGGCAGTCGGTGATCATAGCGGTATTA | 825 |
| | CGCGAGATTACGA | |
| i7_137 | CAAGCAGAAGACGGCATACGAGATAACACCACGGCAGTCGGTGATCATAGCGGTATTA | 826 |
| | CGCGAGATTACGA | |
| i7_138 | CAAGCAGAAGACGGCATACGAGATGTAAGCACGGCAGTCGGTGATCATAGCGGTATTA | 827 |
| | CGCGAGATTACGA | |
| i7_139 | CAAGCAGAAGACGGCATACGAGATGTCCTTGAGGCAGTCGGTGATCATAGCGGTATTA | 828 |
| | CGCGAGATTACGA | |
| i7_140 | CAAGCAGAAGACGGCATACGAGATCAGGTTCAGGCAGTCGGTGATCATAGCGGTATTA | 829 |
| | CGCGAGATTACGA | |
| i7_141 | CAAGCAGAAGACGGCATACGAGATCCAACACTGGCAGTCGGTGATCATAGCGGTATTA | 830 |
| | CGCGAGATTACGA | |
| i7_142 | CAAGCAGAAGACGGCATACGAGATGAGAGTACGGCAGTCGGTGATCATAGCGGTATTA | 831 |
| | CGCGAGATTACGA | |
| i7_143 | CAAGCAGAAGACGGCATACGAGATAGATACGGGGCAGTCGGTGATCATAGCGGTATTA | 832 |
| | CGCGAGATTACGA | |
| i7_144 | CAAGCAGAAGACGGCATACGAGATGTTCTTCGGGCAGTCGGTGATCATAGCGGTATTA | 833 |
| | CGCGAGATTACGA | |
| i7_145 | CAAGCAGAAGACGGCATACGAGATATTCCGCTGGCAGTCGGTGATCATAGCGGTATTA | 834 |
| | CGCGAGATTACGA | |
| i7_146 | CAAGCAGAAGACGGCATACGAGATAAGCTCACGGCAGTCGGTGATCATAGCGGTATTA | 835 |
| | CGCGAGATTACGA | |
| i7_147 | CAAGCAGAAGACGGCATACGAGATTGATCACGGGCAGTCGGTGATCATAGCGGTATTA | 836 |
| | CGCGAGATTACGA | |
| i7_148 | CAAGCAGAAGACGGCATACGAGATCAATGCGAGGCAGTCGGTGATCATAGCGGTATTA | 837 |
| | CGCGAGATTACGA | |
| i7_149 | CAAGCAGAAGACGGCATACGAGATATGCGTCAGGCAGTCGGTGATCATAGCGGTATTA | 838 |
| | CGCGAGATTACGA | |
| i7_150 | CAAGCAGAAGACGGCATACGAGATTACATCGGGGCAGTCGGTGATCATAGCGGTATTA | 839 |
| | CGCGAGATTACGA | |
| i7_151 | CAAGCAGAAGACGGCATACGAGATACTGCGAAGGCAGTCGGTGATCATAGCGGTATTA | 840 |
| | CGCGAGATTACGA | |
| i7_152 | CAAGCAGAAGACGGCATACGAGATTCTGTCGTGGCAGTCGGTGATCATAGCGGTATTA | 841 |
| | CGCGAGATTACGA | |
| i7_153 | CAAGCAGAAGACGGCATACGAGATCTCAAGCTGGCAGTCGGTGATCATAGCGGTATTA | 842 |
| | CGCGAGATTACGA | |
| i7_154 | CAAGCAGAAGACGGCATACGAGATAACCACTCGGCAGTCGGTGATCATAGCGGTATTA | 843 |
| | CGCGAGATTACGA | |
| i7_155 | CAAGCAGAAGACGGCATACGAGATCTTACAGCGGCAGTCGGTGATCATAGCGGTATTA | 844 |
| | CGCGAGATTACGA | |
| i7_156 | CAAGCAGAAGACGGCATACGAGATAGTCTTGGGGCAGTCGGTGATCATAGCGGTATTA | 845 |
| | CGCGAGATTACGA | |
| i7_157 | CAAGCAGAAGACGGCATACGAGATCACGCAATGGCAGTCGGTGATCATAGCGGTATTA | 846 |
| | CGCGAGATTACGA | |
| i7_158 | CAAGCAGAAGACGGCATACGAGATAGCTTCAGGGCAGTCGGTGATCATAGCGGTATTA | 847 |
| | CGCGAGATTACGA | |
| i7_159 | CAAGCAGAAGACGGCATACGAGATCCTCGTTAGGCAGTCGGTGATCATAGCGGTATTA | 848 |
| | CGCGAGATTACGA | |
| i7_160 | CAAGCAGAAGACGGCATACGAGATTGAGACGAGGCAGTCGGTGATCATAGCGGTATTA | 849 |
| | CGCGAGATTACGA | |
| i7_161 | CAAGCAGAAGACGGCATACGAGATCACAGGAAGGCAGTCGGTGATCATAGCGGTATTA | 850 |
| | CGCGAGATTACGA | |
| i7_162 | CAAGCAGAAGACGGCATACGAGATACTCAACGGGCAGTCGGTGATCATAGCGGTATTA | 851 |
| | CGCGAGATTACGA | |
| i7_163 | CAAGCAGAAGACGGCATACGAGATAAGCGACTGGCAGTCGGTGATCATAGCGGTATTA | 852 |
| | CGCGAGATTACGA | |
| i7_164 | CAAGCAGAAGACGGCATACGAGATCCTACCTAGGCAGTCGGTGATCATAGCGGTATTA | 853 |
| | CGCGAGATTACGA | |
| i7_165 | CAAGCAGAAGACGGCATACGAGATATCTCCTGGGCAGTCGGTGATCATAGCGGTATTA | 854 |
| | CGCGAGATTACGA | |
| i7_166 | CAAGCAGAAGACGGCATACGAGATTCACGATGGGCAGTCGGTGATCATAGCGGTATTA | 855 |
| | CGCGAGATTACGA | |
| i7_167 | CAAGCAGAAGACGGCATACGAGATCCACAACAGGCAGTCGGTGATCATAGCGGTATTA | 856 |
| | CGCGAGATTACGA | |
| i7_168 | CAAGCAGAAGACGGCATACGAGATAGGTCTGTGGCAGTCGGTGATCATAGCGGTATTA | 857 |
| | CGCGAGATTACGA | |
| i7_169 | CAAGCAGAAGACGGCATACGAGATAGAAGGACGGCAGTCGGTGATCATAGCGGTATTA | 858 |
| | CGCGAGATTACGA | |
| i7_170 | CAAGCAGAAGACGGCATACGAGATGCGTATCAGGCAGTCGGTGATCATAGCGGTATTA | 859 |
| | CGCGAGATTACGA | |
| i7_171 | CAAGCAGAAGACGGCATACGAGATCAACACAGGGCAGTCGGTGATCATAGCGGTATTA | 860 |
| | CGCGAGATTACGA | |
| i7_172 | CAAGCAGAAGACGGCATACGAGATTCCACGTTGGCAGTCGGTGATCATAGCGGTATTA | 861 |
| | CGCGAGATTACGA | |
| i7_173 | CAAGCAGAAGACGGCATACGAGATATCGCAACGGCAGTCGGTGATCATAGCGGTATTA | 862 |
| | CGCGAGATTACGA | |
| i7_174 | CAAGCAGAAGACGGCATACGAGATACGTCGTTGGCAGTCGGTGATCATAGCGGTATTA | 863 |
| | CGCGAGATTACGA | |
| i7_175 | CAAGCAGAAGACGGCATACGAGATCGAATACGGGCAGTCGGTGATCATAGCGGTATTA | 864 |
| | CGCGAGATTACGA | |
| i7_176 | CAAGCAGAAGACGGCATACGAGATTGCTTGCTGGCAGTCGGTGATCATAGCGGTATTA | 865 |
| | CGCGAGATTACGA | |
| i7_177 | CAAGCAGAAGACGGCATACGAGATCTCGAACAGGCAGTCGGTGATCATAGCGGTATTA | 866 |
| | CGCGAGATTACGA | |
| i7_178 | CAAGCAGAAGACGGCATACGAGATACATGGAGGGCAGTCGGTGATCATAGCGGTATTA | 867 |
| | CGCGAGATTACGA | |
| i7_179 | CAAGCAGAAGACGGCATACGAGATACAAGACGGGCAGTCGGTGATCATAGCGGTATTA | 868 |
| | CGCGAGATTACGA | |
| i7_180 | CAAGCAGAAGACGGCATACGAGATCGCCTTATGGCAGTCGGTGATCATAGCGGTATTA | 869 |
| | CGCGAGATTACGA | |
| i7_181 | CAAGCAGAAGACGGCATACGAGATAGCAGACAGGCAGTCGGTGATCATAGCGGTATTA | 870 |
| | CGCGAGATTACGA | |
| i7_182 | CAAGCAGAAGACGGCATACGAGATGTTAAGCGGGCAGTCGGTGATCATAGCGGTATTA | 871 |
| | CGCGAGATTACGA | |
| i7_183 | CAAGCAGAAGACGGCATACGAGATCATGGATCGGCAGTCGGTGATCATAGCGGTATTA | 872 |
| | CGCGAGATTACGA | |
| i7_184 | CAAGCAGAAGACGGCATACGAGATACAGAGGTGGCAGTCGGTGATCATAGCGGTATTA | 873 |
| | CGCGAGATTACGA | |
| i7_185 | CAAGCAGAAGACGGCATACGAGATTAAGTGGCGGCAGTCGGTGATCATAGCGGTATTA | 874 |
| | CGCGAGATTACGA | |
| i7_186 | CAAGCAGAAGACGGCATACGAGATAGTCAGGTGGCAGTCGGTGATCATAGCGGTATTA | 875 |
| | CGCGAGATTACGA | |
| i7_187 | CAAGCAGAAGACGGCATACGAGATGCCTTAACGGCAGTCGGTGATCATAGCGGTATTA | 876 |
| | CGCGAGATTACGA | |
| i7_188 | CAAGCAGAAGACGGCATACGAGATGTTGGCATGGCAGTCGGTGATCATAGCGGTATTA | 877 |
| | CGCGAGATTACGA | |
| i7_189 | CAAGCAGAAGACGGCATACGAGATCAACCTCTGGCAGTCGGTGATCATAGCGGTATTA | 878 |
| | CGCGAGATTACGA | |
| i7_190 | CAAGCAGAAGACGGCATACGAGATTGGATGGTGGCAGTCGGTGATCATAGCGGTATTA | 879 |
| | CGCGAGATTACGA | |
| i7_191 | CAAGCAGAAGACGGCATACGAGATCTATCCACGGCAGTCGGTGATCATAGCGGTATTA | 880 |
| | CGCGAGATTACGA | |
| i7_192 | CAAGCAGAAGACGGCATACGAGATGATCTCAGGGCAGTCGGTGATCATAGCGGTATTA | 881 |
| | CGCGAGATTACGA | |

/5Phos/ indicates a 5′-terminal phosphate; * indicates a phosphorothioate linkage between the two nucleotides; N indicates any nucleotide - A, C, G, T; W indicates A or T.

TABLE 8
rhAmpSeq Oligonucleotides

| | | SEQ |
|---|---|---|
| | | ID |
| Panel Name | DNA Sequence (5′→3′) | NO: |
| CTLA4 site | TTGTGACTGGTAGCAGGAGrCCCAT/3SpC3/ | 882 |
| 9 Fwd | | |
| CTLA4 site | TCTATCAGGCTTCAGCAGACrCCAGA/3SpC3/ | 883 |
| 9 Rev | | |

“rN” indicates a ribonucleotide, where N is the nucleotide preceeded by the “r”; /3SpC3/ indicates a 3′-terminal C₃ spacer.

### Example 3

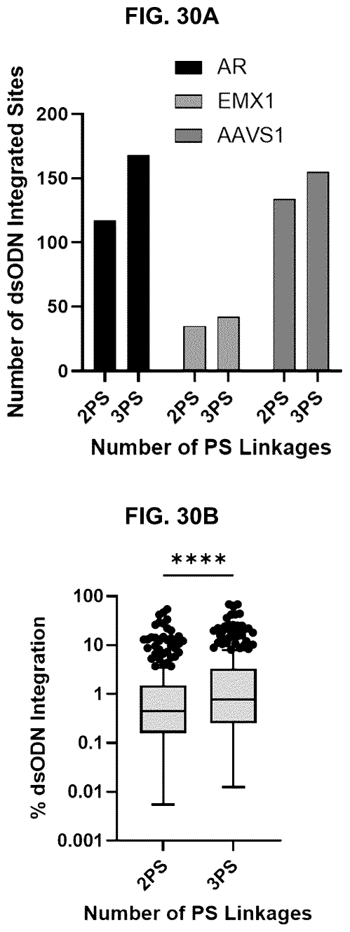

Strategically placing increased numbers of phosphorothioate linkages at the 5′- and/or 3′-termini of dsODNs provides increased protection from enzymatic cleavage of cellular exonucleases, allowing for increased ligation into CRISPR-induced double-stranded breaks. See FIG. 30A-B.

TABLE 9
dsODNs with Increased Protection and Improved Ligation into CRISPR-induced
Double-Stranded Breaks

| | | SEQ ID |
|---|---|---|
| Name | DNA Sequence (5′→3′) | NO: |
| 2PS-1 | /5Phos/A*C*TAGCGATCGGTACCTAGCGCCGAAACCTATTACCGCGACCTAGCGTT* | 884 |
| | G*C*G | |
| 2PS-2 | /5Phos/C*G*CAACGCTAGGTCGCGGTAATAGGTTTCGGCGCTAGGTACCGATCGCT* | 885 |
| | A*G*T | |
| 3PS-1 | /5Phos/A*C*T*AGCGATCGGTACCTAGCGCCGAAACCTATTACCGCGACCTAGCGT* | 886 |
| | T*G*C*G | |
| 3PS-3 | /5Phos/C*G*C*AACGCTAGGTCGCGGTAATAGGTTTCGGCGCTAGGTACCGATCGC* | 887 |
| | T*A*G*T | |

/5Phos/ indicates a 5′-terminal phosphate; * indicates a phosphorothioate linkage between the two nucleotides.

### Example 4

Improved UNCOVERseq Sequencing Quality Using Staggered rhPCR Primers

The UNCOVERseq method described herein represents a significant advancement in the sensitive and controlled nomination of CRISPR-Cas off-target editing events. Developed as an enhanced in cellulo workflow, UNCOVERseq leverages RNase H-dependent PCR (rhPCR) and a novel dsODN integration system to detect off-target sites with sub-0.01% editing frequencies. Importantly, the method demonstrates high concordance between off-target indel and base editing frequencies, supporting its utility across diverse CRISPR modalities, including DSB- and SSB-based editors. This method provides a robust framework for empirical risk assessment in translational gene editing applications, offering standardized input requirements, process controls, and analytical rigor that enhance the reliability of off-target detection across a broad spectrum of editing contexts.

UNCOVERseq and similar methodologies like GUIDE-seq can have low sequence diversity issues that pose a challenge for Illumina sequencing platforms where base diversity is important for cluster identification and color matrix calibration. This issue is largely due to the dsODN sequence that marks the CRISPR-induced edit and is used as an anchor for PCR and NGS library generation. dsODN specific portions of the primer are adjacent to the Read2 sequence which means that the first 20-30 cycles of Illumina Read2 reads all share the same sequence leading to low diversity and sequencing quality that creates downstream effects of correctly identifying and removing the dsODN sequence to mark the editing site during NGS analysis.

Traditional mitigation strategies for low diversity libraries, such as PhiX spike-in, improve sequencing quality but at the cost of reduced read economy, throughput, and increased reagent consumption. To address these limitations, spacer-linked primers—incorporating heterogeneity spacers of variable length or randomized nucleotides—have emerged as a powerful strategy to artificially introduce base diversity at the start of sequencing reads. This type of approach has been described for use with singleplex targeted amplicon sequencing to ensure adequate read diversity when looking at only a single targeted locus. A similar approach has not been used for CRISPR-Cas in cellulo dsODN-based off-target nomination where the amplified loci are not known until after NGS analysis.

In this example, a strategy was developed to mitigate Illumina read diversity issues at the beginning of Read2 during the UNCOVERseq workflow by incorporating staggered UNCOVERseq rhPCR1 primers where an increasing number of randomized nucleotides are placed in between the SP2 and dsODN specific portions of the PCR1 primer. After amplification with staggered PCR1 pooled primer sets, the random nucleotides stagger the start position of each NGS fragment, thus increasing diversity without sacrificing read economy.

Implementation of Staggered rhPCR1 Primers into the UNCOVERseq Workflow

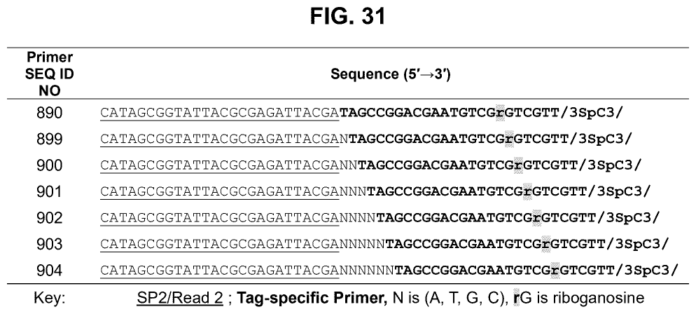

HEK293-Cas9 (CRL-1573Cas9) were nucleofected with a single dsODN (12.5 μmol, 0.5 μM) (SEQ ID NO: 888, SEQ ID NO: 889) along with 5 μM sgRNA (SEQ ID NO: 911-914) using the Lonza 4D-Nucleofector System. Cellular gDNA was extracted after 72 hr, and libraries were then fragmented and adaptered (SEQ ID NO: 895, SEQ ID NO: 896) using the xGen™ DNA Library Prep EZ UNI kit and xGen™ Deceleration Module to an average length of ˜500 bp. dsODN specific amplification for PCR enrichment was achieved using the rhAmpSeq™ Library kit with PCR1 master mix with either non-staggered rhPCR1 primers (SEQ ID NO: 890-891) or staggered rhPCR1 primers pools (SEQ ID NO: 899-910) in the presence of adaptered-tag blocking oligos (SEQ ID NO: 915, SEQ ID NO: 916). The primer pools included equimolar ratios of six staggered rhPCR1 primers with increasing number of heterogeneity spacers of random nucleotides between the 5′-SP2 sequence and the 3′-dsODN specific portion of the primer (SEQ ID NO: 899-910) (FIG. 31). NGS libraries amplified with the non-staggered rhPCR1 primers were sequenced on a Next2000 P1 flow cell with increasing concentrations of Phix to increase sequencing diversity. NGS libraries amplified with the staggered rhPCR1 primers were similarly run on a NextSeq2000 P1 flow cell but did not have PhiX spiked in. All libraries were processed through the Gambit analysis pipeline for OTE nomination.

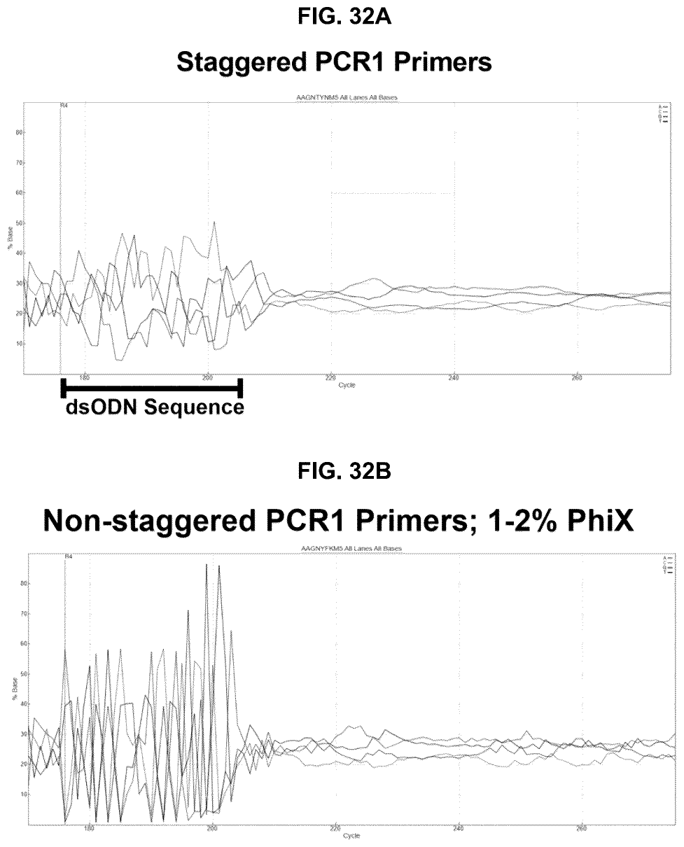

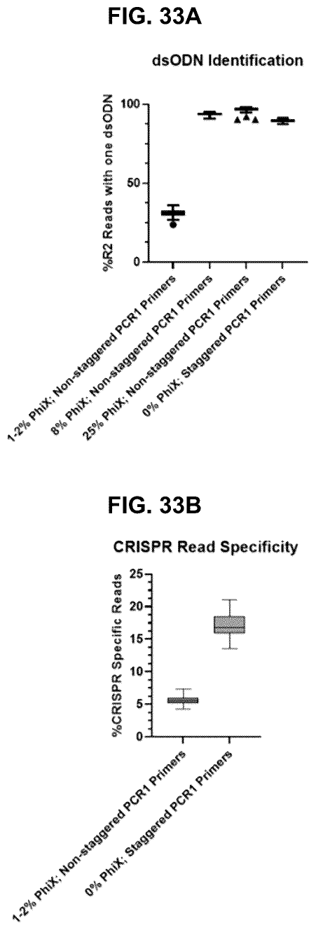

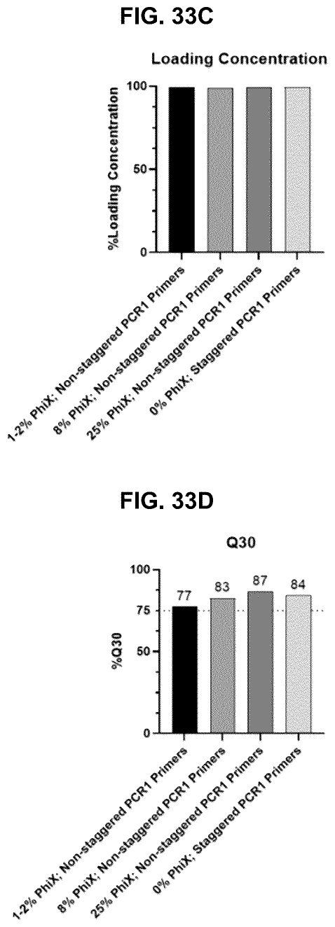

The staggered rhPCR1 primers significantly improved the base pair diversity at the beginning of read2 (FIG. 32). Nearly all bases dropped below 50% on a per cycle basis compared to libraries prepared with non-staggered primers and PhiX spike-in. An important step in processing UNCOVERseq nomination data involves correctly identifying the dsODN to mark the CRISPR-Cas cut site used for alignment. Reads without a dsODN are not processed further for off-target nomination, hence the importance of sequence diversity leading to correct Illumina base calls for downstream identification of the dsODN. Libraries amplified with staggered rhPCR1 primers improved dsODN identification to nearly equivalent levels of libraries prepared with non-staggered primers and PhiX spike-ins of 8-25% and improved 2-3-fold over 1-2% PhiX spike-in libraries (FIG. 33). In addition, libraries prepared with staggered PCR1 primers had 2-3-fold increases in CRISPR read specificity compared to non-staggered PCR1 primer prepared libraries. Percent loading concentration on the flow cell was used to ensure these differences were not due to differences in library concentrations loaded on the flow cell (FIG. 33C). Overall, these results show that introducing heterogeneity spacers of random nucleotides between the 5′-SP2 sequence and the 3′-dsODN specific portion of the primer can significantly improve sequencing quality for off-target nomination.

### Assessment of the Lower Limit of Heterogeneity Spacers and Performance During Off-Target Nomination

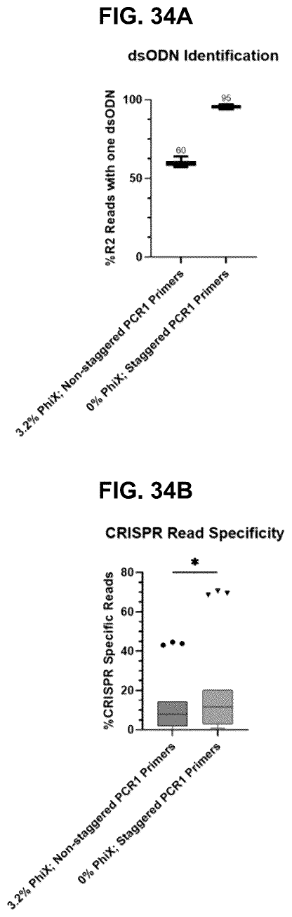

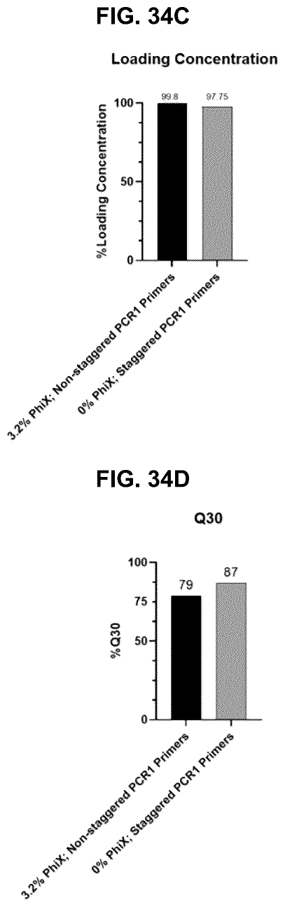

To test the lower limit of the amount of heterogeneity spacers needed to achieve improved base pair diversity and dsODN identification, libraries were prepared as described above with a pool of staggered rhPCR1 primers with a max of 3 Ns between the SP2 and dsODN specific portion of the primer (SEQ ID NO: 899, SEQ ID NO: 900, SEQ ID NO: 901, SEQ ID NO: 905, SEQ ID NO: 906, SEQ ID NO: 907). Once again, libraries prepared with the staggered primers significantly improved dsODN identification and CRISPR read specificity (FIG. 34).

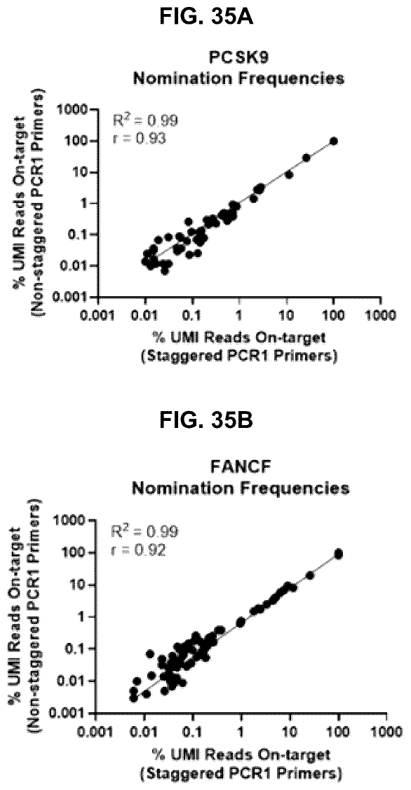

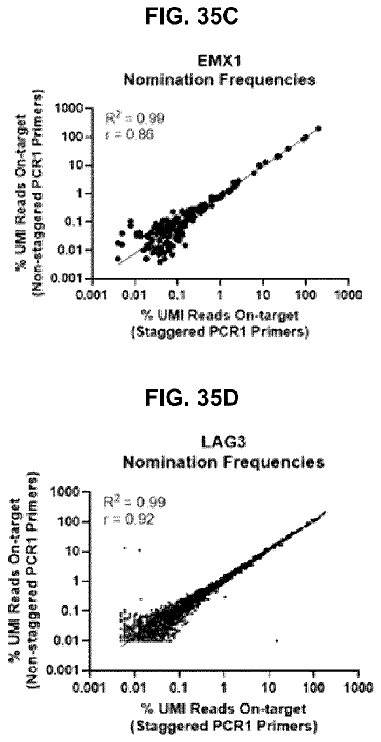

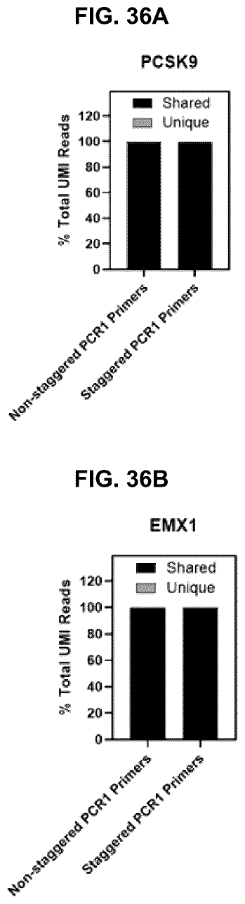

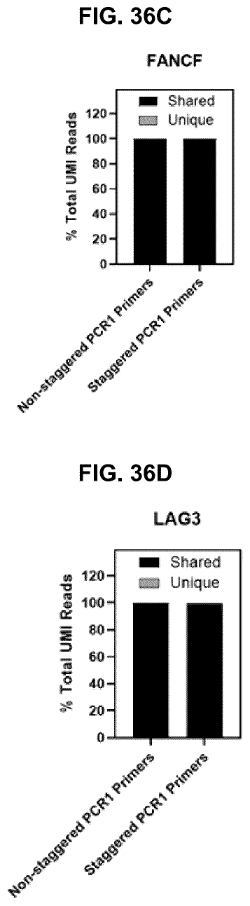

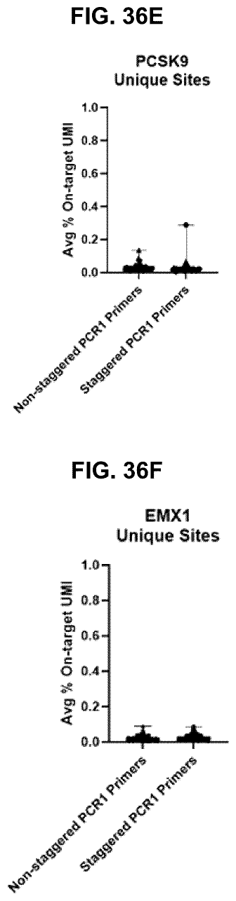

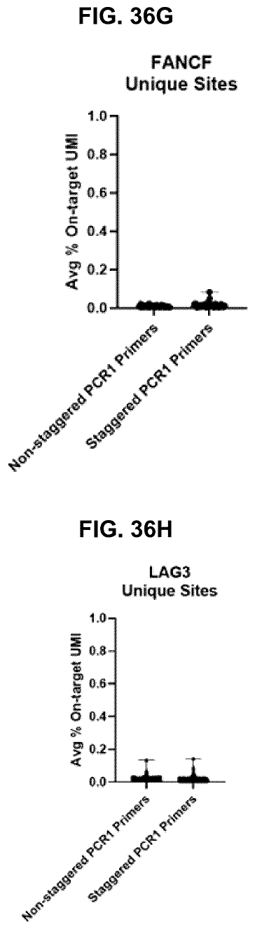

To assess reproducibility of off-target nomination between libraries prepped with and without staggered PCR1 primers, nominated sites across four gRNAs with biological triplicates were compared. For similarly nominated off-targets, the nomination frequencies were highly conserved (R²=0.99) (FIG. 35). Moreover, >99% of the total UMI reads were nominated on shared off-targets between libraries prepped with and without staggered PCR primers (FIG. 36A-D). Unique nominated sites did account for any nomination frequencies above 0.3% (median frequency=0.013%) (FIG. 36E-H). This indicates that libraries prepared with staggered PCR primers had similar off-target nomination performance while boosting the sequencing quality and NGS read economy. Additionally, heterogeneity spacers of 3-6 Ns between the SP2 and dsODN specific portion of the primer were sufficient for the improved quality.

TABLE 10
Oligonucleotide Sequences

| | | SEQ ID |
|---|---|---|
| Name | Sequence (5′→3′) | NO: |
| CTL_216T | /5Phos/T*A*A*GCGGCGTAGGTAGCCGGACGAATGTCGGTCGTA*G*T*T | 888 |
| CTL_216B | /5Phos/A*A*C*TACGACCGACATTCGTCCGGCTACCTACGCCGC*T*T*A | 889 |
| CTL216_ | CATAGCGGTATTACGCGAGATTACGATAGCCGGACGAATGTCGrGTCGTT/3SpC3/ | 890 |
| FWD | | |
| CTL216_ | CATAGCGGTATTACGCGAGATTACGAACATTCGTCCGGCTACCTrACGCCC/3SpC3/ | 891 |
| REV | | |
| P5_rh | AATGATACGGCGACCACCGAGATrCTACAT/3SpC3/ | 892 |
| P5_2 | AATGATACGGCGACCACCGAGATCTACAC | 893 |
| i7_H3 | CAAGCAGAAGACGGCATACGAGATNNNNNNNNGGCAGTCGGTGATCATAGCGGTATT | 894 |
| | ACGCGAGATTACGA | |
| P5 Adapter | AATGATACGGCGACCACCGAGATCTACACNNNNNNNNNNWNNWNNACACTCTTTCCC | 895 |
| | TACACGACGCTCTTCCGATC*T | |
| P5 | /5Phos/GATCGGAAGAGC*C*A | 896 |
| Common | | |
| Adapter | | |
| CTLH3Index1_ | TCGTAATCTCGCGTAATACCGCTATGATCACCGACTGCC | 897 |
| v2 | | |
| CTLH3_ | GGCAGTCGGTGATCATAGCGGTATTACGCGAGATTACGA | 898 |
| Read2_v2 | | |
| CTL216_ | CATAGCGGTATTACGCGAGATTACGANTAGCCGGACGAATGTCGrGTCGTT/ | 899 |
| N1PCR1_ | 3SpC3/ | |
| FWD | | |
| CTL216_N | CATAGCGGTATTACGCGAGATTACGANNTAGCCGGACGAATGTCGrGTCGTT/ | 900 |
| N2PCR1_ | 3SpC3/ | |
| FWD | | |
| CTL216_ | CATAGCGGTATTACGCGAGATTACGANNNTAGCCGGACGAATGTCGrGTCGTT/ | 901 |
| N3PCR1_ | 3SpC3/ | |
| FWD | | |
| CTL216_ | CATAGCGGTATTACGCGAGATTACGANNNNTAGCCGGACGAATGTCGrGTCGTT/ | 902 |
| N4PCR1_ | 3SpC3/ | |
| FWD | | |
| CTL216_ | CATAGCGGTATTACGCGAGATTACGANNNNNTAGCCGGACGAATGTCGrGTCGTT/ | 903 |
| N5PCR1_ | 3SpC3/ | |
| FWD | | |
| CTL216_ | CATAGCGGTATTACGCGAGATTACGANNNNNNTAGCCGGACGAATGTCGrGTCGTT/ | 904 |
| N6PCR1_ | 3SpC3/ | |
| FWD | | |
| CTL216_ | CATAGCGGTATTACGCGAGATTACGANACATTCGTCCGGCTACCTrACGCCC/ | 905 |
| N1PCR1_ | 3SpC3/ | |
| REV | | |
| CTL216_ | CATAGCGGTATTACGCGAGATTACGANNACATTCGTCCGGCTACCTrACGCCC/ | 906 |
| N2PCR1_ | 3SpC3/ | |
| REV | | |
| CTL216_ | CATAGCGGTATTACGCGAGATTACGANNNACATTCGTCCGGCTACCTrACGCCC/ | 907 |
| N3PCR1_ | 3SpC3/ | |
| REV | | |
| CTL216_ | CATAGCGGTATTACGCGAGATTACGANNNNACATTCGTCCGGCTACCTrACGCCC/ | 908 |
| N4PCR1_ | 3SpC3/ | |
| REV | | |
| CTL216_ | CATAGCGGTATTACGCGAGATTACGANNNNNACATTCGTCCGGCTACCTrACGCCC/ | 909 |
| N5PCR1_ | 3SpC3/ | |
| REV | | |
| CTL216_ | CATAGCGGTATTACGCGAGATTACGANNNNNNACATTCGTCCGGCTACCTrACGCCC/ | 910 |
| N6PCR1_ | 3SpC3/ | |
| REV | | |
| PCSK9 | mC*mC*mC*rGrCrArCrCrUrUrGrGrCrGrCrArGrCrGrGrGrUrUrUrUrArG | 911 |
| sgRNA | rArGrCrUrArGrArArArUrArGrCrArArGrUrUrArArArArUrArArGrGrCr | |
| | UrArGrUrCrCrGrUrUrArUrCrArArCrUrUrGrArArArArArGrUrGrGrCrA | |
| | rCrCrGrArGrUrCrGrGrUrGrCmU*mU*mU*rU | |
| LAG3 | mG*mA*mA*rGrGrCrUrGrArGrArUrCrCrUrGrGrArGrGrGrUrUrUrUrArG | 912 |
| sgRNA | rArGrCrUrArGrArArArUrArGrCrArArGrUrUrArArArArUrArArGrGrCr | |
| | UrArGrUrCrCrGrUrUrArUrCrArArCrUrUrGrArArArArArGrUrGrGrCrA | |
| | rCrCrGrArGrUrCrGrGrUrGrCmU*mU*mU*rU | |
| EMX1 | mG*mA*mG*rUrCrCrGrArGrCrArGrArArGrArArGrArArGrUrUrUrUrArG | 913 |
| sgRNA | rArGrCrUrArGrArArArUrArGrCrArArGrUrUrArArArArUrArArGrGrCr | |
| | UrArGrUrCrCrGrUrUrArUrCrArArCrUrUrGrArArArArArGrUrGrGrCrA | |
| | rCrCrGrArGrUrCrGrGrUrGrCmU*mU*mU*rU | |
| FANCF | mG*mG*mA*rArUrCrCrCrUrUrCrUrGrCrArGrCrArCrCrGrUrUrUrUrArG | 914 |
| sgRNA | rArGrCrUrArGrArArArUrArGrCrArArGrUrUrArArArArUrArArGrGrCr | |
| | UrArGrUrCrCrGrUrUrArUrCrArArCrUrUrGrArArArArArGrUrGrGrCrA | |
| | rCrCrGrArGrUrCrGrGrUrGrCmU*mU*mU*rU | |
| CTL216T_ | G+TCGGTC+G+T+AGTTAGATCGGA+A+G+A+GC/3SpC3/ | 915 |
| v5 | | |
| CTL216B_ | T+A+C+C+TACGCCGCTTAAGATCGGA+A+G+A+GC/3SpC3/ | 916 |
| v5 | | |

All oligonucleotides were synthesized by IDT (Coralville, IA). Abbreviations used in the sequences above are: N indicates any nucleotide - A, C, G, T; “rN” indicates a ribonucleotide, where N is the nucleotide preceeded by the “r”; /5Phos/ indicates a 5′-terminal phosphate; * indicates a phosphorothioate linkage between the two nucleotides; +N indicates a locked nucleotide having a methylene bond between the 2′ oxygen and the 4′ carbon of the pentose ring, where N is the nucleotide preceeded by the “+”; /3SpC3/ indicates a 3′-terminal C₃ spacer.

What is claimed:

1. A method for reducing adaptered-tag sequencing reads during the identification and nomination of on- and off-target CRISPR edited sites, the method comprising:
contacting in an amplification reaction one or more adaptered-tag blocking oligonucleotides with an isolated genomic DNA having one or more tag sequences and adapter sequences;
wherein the adaptered-tag blocking oligonucleotides comprise one or more blocking moieties and hybridize to adaptered-tag sequences at a junction region between the adapter and tag sequences to reduce amplification of the adaptered-tag sequences.

2. The method of claim 1, wherein the amplification reaction comprises one or more adapter-specific primers and one or more tag-specific primers to produce a first set of amplified sequences, the method further comprising:
amplifying the first set of amplified sequences using universal sequencing primers targeting the tails of the tag-specific primers to produce a second set of amplified sequences;
sequencing the second set of amplified sequences and obtaining sequencing data; and
identifying on-/off-target CRISPR editing loci.

3. The method of claim 2, wherein the one or more tag-specific primers comprise a plurality of staggered primers, each staggered primer comprising a number of random nucleotides positioned between a tag-specific sequence portion and a universal tail sequence portion.

4. The method of claim 3, wherein the number of random nucleotides positioned between the tag-specific sequence portion and the universal tail sequence portion for each staggered primer ranges from 0 to 6.

5. The method of claim 1, wherein the one or more tag sequences comprises DNA, RNA, xeno nucleic acids, or combinations thereof.

6. The method of claim 1, wherein the one or more tag sequences comprises a double-stranded oligodeoxynucleotide tag (dsODN-tag) sequence.

7. The method of claim 1, wherein the one or more tag sequences comprises one or more modifications comprising a 5′-terminal phosphate, phosphorothioate linkages, methylphosphonate linkages, boranophosphate linkages, phosphonoacetate linkages, or combinations thereof.

8. The method of claim 1, wherein the one or more tag sequences comprises at least three phosphorothioate linkages at the 5′-terminus, 3′-terminus, or a combination thereof.

9. The method of claim 1, wherein the one or more blocking moieties of the adaptered-tag blocking oligonucleotides comprises a 3′-terminal C₃ spacer, a dideoxy nucleotide, an inverted dideoxy nucleotide, 3′-terminal phosphorylation, an amino, a 2′-O-methoxy-ethyl (2′-MOE), or combinations thereof.

10. The method of claim 1, wherein the adaptered-tag blocking oligonucleotides hybridize to top and bottom strands of the adaptered-tag sequences at a junction region between the adapter and tag sequences.

11. The method of claim 1, wherein the adaptered-tag blocking oligonucleotides have a sequence length of about 15 nucleotides to about 35 nucleotides.

12. The method of claim 1, wherein the adaptered-tag sequences have a sequence length of about 150 nucleotides to about 200 nucleotides.

13. The method of claim 1, wherein about 40-60% of the adaptered-tag blocking oligonucleotides hybridizes to the adapter sequence portion of the adaptered-tag sequences and about 40-60% of the adaptered-tag blocking oligonucleotides hybridizes to the tag sequence portion of the adaptered-tag sequences.

14. The method of claim 1, wherein the adaptered-tag blocking oligonucleotides reduce adaptered-tag sequencing reads by at least about 25% relative to a method without the adaptered-tag blocking oligonucleotides.

15. The method of claim 1, wherein the adaptered-tag blocking oligonucleotides increase the amount of sequencing reads at unique nominated off-target effect (OTE) sites as compared to a method without the adaptered-tag blocking oligonucleotides.

16. A method for identifying and nominating on- and off-target CRISPR edited sites with improved accuracy and sensitivity, the method comprising:
(a) performing a multiplex PCR reaction comprising:
(i) one or more tag-specific oligonucleotide primers, each having a cleavage region comprising a ribonucleotide (rN) positioned 5′ of a blocking group and a complementary region flanking one or more tag sequences, wherein the blocking group prevents primer extension and/or inhibits the oligonucleotide primer from serving as a template for DNA synthesis;
(ii) one or more adapter-specific oligonucleotide primers, each having a cleavage region comprising a rN positioned 5′ of a blocking group and a complementary region flanking the 5′ end of a universal adapter sequence;
(iii) one or more adaptered-tag blocking oligonucleotides corresponding to each strand of the tag sequences and comprising one or more blocking moieties, wherein the adaptered-tag blocking oligonucleotides hybridize to top and bottom strands of adaptered-tag sequences at a junction region between the universal adapter and tag sequences and inhibit annealing of the tag-specific oligonucleotide primers to the top and bottom strands of the adaptered-tag sequences, thereby reducing amplification of the adaptered-tag sequences; and
(iv) a cleaving enzyme;
(b) hybridizing the tag-specific oligonucleotide primers to one or more incorporated tag sequences to form a tag sequence double stranded substrate and hybridizing one or more adapter-specific oligonucleotide primers to the 5′ end of the universal adapter sequence;
(c) cleaving at a point within or adjacent to the cleavage regions with the cleaving enzyme to remove the blocking groups from the one or more tag-specific oligonucleotide primers and the one or more adapter-specific oligonucleotide primers;
(d) amplifying a portion of isolated genomic DNA comprising the one or more incorporated tag sequences and the universal adapter sequence; and
(e) sequencing the amplified portion of the isolated genomic DNA, thereby identifying on- and off-target CRISPR edited sites.

17. The method of claim 16, wherein the cleaving enzyme is an RNase H2 enzyme.

18. The method of claim 16, wherein the isolated genomic DNA comprising the one or more incorporated tag sequences and the universal adapter sequence is generated by:
isolating genomic DNA from a cell having one or more tag sequences incorporated into a target site within a genome of the cell; and
integrating a universal adapter sequence into the isolated genomic DNA.

19. The method of claim 16, wherein the universal adapter sequence comprises a unique molecular index (UMI).

20. The method of claim 16, wherein the sequencing of step (e) further comprises executing on a processor:
(i) aligning sequence data to a reference genome; and
(ii) outputting the alignment, analysis, and results data as custom-formatted files, tables, or graphics.


SEQUENCE LISTING (916 sequences)

SEQ ID NO: 1 - DNA, 132 bp, synthetic construct
  source 1..132: mol_type = other DNA
aatgatacggcgaccaccgagatctacacctgagatcccttgtagacactctttccctacacgacgctcttccgatcttaagcggcgtaggtagccggacgaatgtcggtcgtagttagatcggaagagcca

SEQ ID NO: 2 - DNA, 132 bp, synthetic construct
  source 1..132: mol_type = other DNA
aatgatacggcgaccaccgagatctacacctgagatcccttgtagacactctttccctacacgacgctcttccgatctaactacgaccgacattcgtccggctacctacgccgcttaagatcggaagagcca

SEQ ID NO: 3 - DNA, 18 bp, synthetic construct
  source 1..18: mol_type = other DNA
gtcgtagttagatcggaa

SEQ ID NO: 4 - DNA, 27 bp, synthetic construct
  source 1..27: mol_type = other DNA
gtcggtcgtagttagatcggaagagcg

SEQ ID NO: 5 - DNA, 30 bp, synthetic construct
  source 1..30: mol_type = other DNA
atgtcggtcgtagttagatcggaagagcgt

SEQ ID NO: 6 - DNA, 27 bp, synthetic construct
  source 1..27: mol_type = other DNA
gtcggtcgtagttagatcggaagagcg

SEQ ID NO: 7 - DNA, 27 bp, synthetic construct
  source 1..27: mol_type = other DNA
gtcggtcgtagttagatcggaagagcg

SEQ ID NO: 8 - DNA, 27 bp, synthetic construct
  source 1..27: mol_type = other DNA
gtcggtcgtagttagatcggaagagcg

SEQ ID NO: 9 - DNA, 18 bp, synthetic construct
  source 1..18: mol_type = other DNA
cgccgcttaagatcggaa

SEQ ID NO: 10 - DNA, 22 bp, synthetic construct
  source 1..22: mol_type = other DNA
cgccgcttaagatcggaagagc

SEQ ID NO: 11 - DNA, 27 bp, synthetic construct
  source 1..27: mol_type = other DNA
cctacgccgcttaagatcggaagagcg

SEQ ID NO: 12 - DNA, 27 bp, synthetic construct
  source 1..27: mol_type = other DNA
cctacgccgcttaagatcggaagagcg

SEQ ID NO: 13 - DNA, 27 bp, synthetic construct
  source 1..27: mol_type = other DNA
cctacgccgcttaagatcggaagagcg

SEQ ID NO: 14 - DNA, 27 bp, synthetic construct
  source 1..27: mol_type = other DNA
cctacgccgcttaagatcggaagagcg

SEQ ID NO: 15 - DNA, 22 bp, synthetic construct
  source 1..22: mol_type = other DNA
tagccggacgaatgtcggtcgt

SEQ ID NO: 16 - DNA, 22 bp, synthetic construct
  source 1..22: mol_type = other DNA
gacattcgtccggctacctacg

SEQ ID NO: 17 - DNA, 29 bp, synthetic construct
  source 1..29: mol_type = other DNA
aatgatacggcgaccaccgagatctacac

SEQ ID NO: 18 - DNA, 495 bp, synthetic construct
  source 1..495: mol_type = other DNA
aatgatacggcgaccaccgagatctacacctgagatcnnwnnwnnacactctttccctacacgacgctcttccgatctactcagcagtatcttcagtgctcttgcctgcgctgtcgtctagcagagaacctttgcattcggccaatggggcacaaggagtgggacgcacagcgggtggaactcccaaaagtggggcgtacatgcaatccccccgaagctgttcccctgaactacgaccgacattcgtccggctacctacgccgcttagactcagatgctccaacgcctccacacccaggcccatggacaccgacactgccttacacaactccttggcgttgtcagaaatggtcgaagtgccccctaagtaattgtccttggaggaagtgggagcccccgaggcctccctcgctctccagatcggaagagcgtcgtgtagggaaagagtgtnnwnnwnngatctcaggtgtagatctcggtggtcgccgtatcatt

SEQ ID NO: 19 - DNA, 495 bp, synthetic construct
  source 1..495: mol_type = other DNA
aatgatacggcgaccaccgagatctacacctgagatcnnwnnwnnacactctttccctacacgacgctcttccgatctggagagcgagggaggcctcgggggctcccacttcctccaaggacaattacttagggggcacttcgaccatttctgacaacgccaaggagttgtgtaaggcagtgtcggtgtccatgggcctgggtgtggaggcgttggagcatctgagtcagcacgcccgacaagtacgccggttagtggtccgtcggccaggggaacagcttcggggggattgcatgtacgccccacttttgggagttccacccgctgtgcgtcccactccttgtgccccattggccgaatgcaaaggttctctgctagacgacagcgcaggcaagagcactgaagatactgctgagtagatcggaagagcgtcgtgtagggaaagagtgtnnwnnwnngatctcaggtgtagatctcggtggtcgccgtatcatt

SEQ ID NO: 20 - DNA, 30 bp, synthetic construct
  source 1..30: mol_type = other DNA
tgagatcccttgtagacactctttccctac

SEQ ID NO: 21 - DNA, 21 bp, synthetic construct
  source 1..21: mol_type = other DNA
tttgggagttccacccgctgt

SEQ ID NO: 22 - DNA, 24 bp, synthetic construct
  source 1..24: mol_type = other DNA
acaccgacactgccttacacaact

SEQ ID NO: 23 - DNA, 29 bp, synthetic construct
  source 1..29: mol_type = other DNA
aatgatacggcgaccaccgagatctacat

SEQ ID NO: 24 - DNA, 49 bp, synthetic construct
  source 1..49: mol_type = other DNA
catagcggtattacgcgagattacgatagccggacgaatgtcggtcgtt

SEQ ID NO: 25 - DNA, 50 bp, synthetic construct
  source 1..50: mol_type = other DNA
catagcggtattacgcgagattacgaacattcgtccggctacctacgccc

SEQ ID NO: 26 - DNA, 48 bp, synthetic construct
  source 1..48: mol_type = other DNA
catagcggtattacgcgagattacgatacgccggttagtggtccgtcc

SEQ ID NO: 27 - DNA, 50 bp, synthetic construct
  source 1..50: mol_type = other DNA
catagcggtattacgcgagattacgataaccggcgtacttgtcgggcgtc

SEQ ID NO: 28 - DNA, 26 bp, synthetic construct
  source 1..26: mol_type = other DNA
gtcggtcgtagttagatcggaagagc

SEQ ID NO: 29 - DNA, 26 bp, synthetic construct
  source 1..26: mol_type = other DNA
gtcggtcgtagttagatcggaagagc

SEQ ID NO: 30 - DNA, 26 bp, synthetic construct
  source 1..26: mol_type = other DNA
gtcggtcgtagttagatcggaagagc

SEQ ID NO: 31 - DNA, 26 bp, synthetic construct
  source 1..26: mol_type = other DNA
gtcggtcgtagttagatcggaagagc

SEQ ID NO: 32 - DNA, 26 bp, synthetic construct
  source 1..26: mol_type = other DNA
gtcggtcgtagttagatcggaagagc

SEQ ID NO: 33 - DNA, 26 bp, synthetic construct
  source 1..26: mol_type = other DNA
gtcggtcgtagttagatcggaagagc

SEQ ID NO: 34 - DNA, 26 bp, synthetic construct
  source 1..26: mol_type = other DNA
gtcggtcgtagttagatcggaagagc

SEQ ID NO: 35 - DNA, 26 bp, synthetic construct
  source 1..26: mol_type = other DNA
gtcggtcgtagttagatcggaagagc

SEQ ID NO: 36 - DNA, 28 bp, synthetic construct
  source 1..28: mol_type = other DNA
tacctacgccgcttaagatcggaagagc

SEQ ID NO: 37 - DNA, 28 bp, synthetic construct
  source 1..28: mol_type = other DNA
tacctacgccgcttaagatcggaagagc

SEQ ID NO: 38 - DNA, 28 bp, synthetic construct
  source 1..28: mol_type = other DNA
tacctacgccgcttaagatcggaagagc

SEQ ID NO: 39 - DNA, 28 bp, synthetic construct
  source 1..28: mol_type = other DNA
tacctacgccgcttaagatcggaagagc

SEQ ID NO: 40 - DNA, 28 bp, synthetic construct
  source 1..28: mol_type = other DNA
tacctacgccgcttaagatcggaagagc

SEQ ID NO: 41 - DNA, 28 bp, synthetic construct
  source 1..28: mol_type = other DNA
tacctacgccgcttaagatcggaagagc

SEQ ID NO: 42 - DNA, 28 bp, synthetic construct
  source 1..28: mol_type = other DNA
tacctacgccgcttaagatcggaagagc

SEQ ID NO: 43 - DNA, 28 bp, synthetic construct
  source 1..28: mol_type = other DNA
tacctacgccgcttaagatcggaagagc

SEQ ID NO: 44 - DNA, 28 bp, synthetic construct
  source 1..28: mol_type = other DNA
agtggtccgtcggcagatcggaagagcg

SEQ ID NO: 45 - DNA, 28 bp, synthetic construct
  source 1..28: mol_type = other DNA
agtggtccgtcggcagatcggaagagcg

SEQ ID NO: 46 - DNA, 28 bp, synthetic construct
  source 1..28: mol_type = other DNA
agtggtccgtcggcagatcggaagagcg

SEQ ID NO: 47 - DNA, 28 bp, synthetic construct
  source 1..28: mol_type = other DNA
agtggtccgtcggcagatcggaagagcg

SEQ ID NO: 48 - DNA, 28 bp, synthetic construct
  source 1..28: mol_type = other DNA
agtggtccgtcggcagatcggaagagcg

SEQ ID NO: 49 - DNA, 28 bp, synthetic construct
  source 1..28: mol_type = other DNA
agtggtccgtcggcagatcggaagagcg

SEQ ID NO: 50 - DNA, 28 bp, synthetic construct
  source 1..28: mol_type = other DNA
agtggtccgtcggcagatcggaagagcg

SEQ ID NO: 51 - DNA, 28 bp, synthetic construct
  source 1..28: mol_type = other DNA
agtggtccgtcggcagatcggaagagcg

SEQ ID NO: 52 - DNA, 26 bp, synthetic construct
  source 1..26: mol_type = other DNA
tgtcgggcgtgctagatcggaagagc

SEQ ID NO: 53 - DNA, 26 bp, synthetic construct
  source 1..26: mol_type = other DNA
tgtcgggcgtgctagatcggaagagc

SEQ ID NO: 54 - DNA, 26 bp, synthetic construct
  source 1..26: mol_type = other DNA
tgtcgggcgtgctagatcggaagagc

SEQ ID NO: 55 - DNA, 26 bp, synthetic construct
  source 1..26: mol_type = other DNA
tgtcgggcgtgctagatcggaagagc

SEQ ID NO: 56 - DNA, 26 bp, synthetic construct
  source 1..26: mol_type = other DNA
tgtcgggcgtgctagatcggaagagc

SEQ ID NO: 57 - DNA, 26 bp, synthetic construct
  source 1..26: mol_type = other DNA
tgtcgggcgtgctagatcggaagagc

SEQ ID NO: 58 - DNA, 26 bp, synthetic construct
  source 1..26: mol_type = other DNA
tgtcgggcgtgctagatcggaagagc

SEQ ID NO: 59 - DNA, 26 bp, synthetic construct
  source 1..26: mol_type = other DNA
tgtcgggcgtgctagatcggaagagc

SEQ ID NO: 60 - DNA, 30 bp, synthetic construct
  source 1..30: mol_type = other DNA
tagtggtccgtcggcagatcggaagagcgt

SEQ ID NO: 61 - DNA, 30 bp, synthetic construct
  source 1..30: mol_type = other DNA
cttgtcgggcgtgctagatcggaagagcgt

SEQ ID NO: 62 - DNA, 132 bp, synthetic construct
  source 1..132: mol_type = other DNA
aatgatacggcgaccaccgagatctacacctgagatcccttgtagacactctttccctacacgacgctcttccgatctagcacgcccgacaagtacgccggttagtggtccgtcggcagatcggaagagcca

SEQ ID NO: 63 - DNA, 132 bp, synthetic construct
  source 1..132: mol_type = other DNA
aatgatacggcgaccaccgagatctacacctgagatcccttgtagacactctttccctacacgacgctcttccgatctgccgacggaccactaaccggcgtacttgtcgggcgtgctagatcggaagagcca

SEQ ID NO: 64 - DNA, 39 bp, synthetic construct
  source 1..39: mol_type = other DNA
taagcggcgtaggtagccggacgaatgtcggtcgtagtt

SEQ ID NO: 65 - DNA, 39 bp, synthetic construct
  source 1..39: mol_type = other DNA
aactacgaccgacattcgtccggctacctacgccgctta

SEQ ID NO: 66 - DNA, 39 bp, synthetic construct
  source 1..39: mol_type = other DNA
agcacgcccgacaagtacgccggttagtggtccgtcggc

SEQ ID NO: 67 - DNA, 39 bp, synthetic construct
  source 1..39: mol_type = other DNA
gccgacggaccactaaccggcgtacttgtcgggcgtgct

SEQ ID NO: 68 - RNA, 100 nt, synthetic construct
  source 1..100: mol_type = other RNA
gttggagcatctgagtccaggttttagagctagaaatagcaagttaaaataaggctagtccgttatcaacttgaaaaagtggcaccgagtcggtgctttt

SEQ ID NO: 69 - RNA, 100 nt, synthetic construct
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 70 - RNA, 100 nt, synthetic construct
  source 1..100: mol_type = other RNA
ggggccactagggacaggatgttttagagctagaaatagcaagttaaaataaggctagtccgttatcaacttgaaaaagtggcaccgagtcggtgctttt

SEQ ID NO: 71 - RNA, 100 nt, synthetic construct
  source 1..100: mol_type = other RNA
gaaggctgagatcctggagggttttagagctagaaatagcaagttaaaataaggctagtccgttatcaacttgaaaaagtggcaccgagtcggtgctttt

SEQ ID NO: 72 - DNA, 78 bp, synthetic construct
  source 1..78: mol_type = other DNA
aatgatacggcgaccaccgagatctacacnnnnnnnnnnwnnwnnacactctttccctacacgacgctcttccgatct

SEQ ID NO: 73 - DNA, 14 bp, synthetic construct
  source 1..14: mol_type = other DNA
gatcggaagagcca

SEQ ID NO: 74 - DNA, 71 bp, synthetic construct
  source 1..71: mol_type = other DNA
caagcagaagacggcatacgagatnnnnnnnnggcagtcggtgatcatagcggtattacgcgagattacga

SEQ ID NO: 75 - DNA, 39 bp, synthetic construct
  source 1..39: mol_type = other DNA
tcgtaatctcgcgtaataccgctatgatcaccgactgcc

SEQ ID NO: 76 - DNA, 39 bp, synthetic construct
  source 1..39: mol_type = other DNA
ggcagtcggtgatcatagcggtattacgcgagattacga

SEQ ID NO: 77 - DNA, 34 bp, synthetic construct
  source 1..34: mol_type = other DNA
gtttaattgagttgtcatatgttaataacggtat

SEQ ID NO: 78 - DNA, 34 bp, synthetic construct
  source 1..34: mol_type = other DNA
ataccgttattaacatatgacaactcaattaaac

SEQ ID NO: 79 - DNA, 39 bp, synthetic construct; identical to SEQ ID NO: 64
  source 1..39: mol_type = other DNA

SEQ ID NO: 80 - DNA, 39 bp, synthetic construct; identical to SEQ ID NO: 65
  source 1..39: mol_type = other DNA

SEQ ID NO: 81 - DNA, 20 bp, synthetic construct
  source 1..20: mol_type = other DNA
gttggagcatctgagtccag

SEQ ID NO: 82 - DNA, 20 bp, synthetic construct
  source 1..20: mol_type = other DNA
ggggccactagggacaggat

SEQ ID NO: 83 - DNA, 20 bp, synthetic construct
  source 1..20: mol_type = other DNA
gaaggctgagatcctggagg

SEQ ID NO: 84 - DNA, 20 bp, synthetic construct
  source 1..20: mol_type = other DNA
cccgcaccttggcgcagcgg

SEQ ID NO: 85 - DNA, 20 bp, synthetic construct
  source 1..20: mol_type = other DNA
ctaacagttgcttttatcac

SEQ ID NO: 86 - DNA, 20 bp, synthetic construct
  source 1..20: mol_type = other DNA
gagtccgagcagaagaagaa

SEQ ID NO: 87 - DNA, 20 bp, synthetic construct
  source 1..20: mol_type = other DNA
ggaatcccttctgcagcacc

SEQ ID NO: 88 - DNA, 20 bp, synthetic construct
  source 1..20: mol_type = other DNA
gagcagggctggggagaagg

SEQ ID NO: 89 - DNA, 20 bp, synthetic construct
  source 1..20: mol_type = other DNA
acgagcaccactctgagata

SEQ ID NO: 90 - DNA, 20 bp, synthetic construct
  source 1..20: mol_type = other DNA
gtcatcttagtcattacctg

SEQ ID NO: 91 - DNA, 20 bp, synthetic construct
  source 1..20: mol_type = other DNA
cgtcatgagcagattaaacc

SEQ ID NO: 92 - DNA, 20 bp, synthetic construct
  source 1..20: mol_type = other DNA
ggccgagatgtctcgctccg

SEQ ID NO: 93 - DNA, 20 bp, synthetic construct
  source 1..20: mol_type = other DNA
cgctgaccgtgaacgataca

SEQ ID NO: 94 - DNA, 20 bp, synthetic construct
  source 1..20: mol_type = other DNA
cgtctgggcggtgctacaac

SEQ ID NO: 95 - DNA, 20 bp, synthetic construct
  source 1..20: mol_type = other DNA
tgtagcaccgcccagacgac

SEQ ID NO: 96 - DNA, 20 bp, synthetic construct
  source 1..20: mol_type = other DNA
gtctgggcggtgctacaact

SEQ ID NO: 97 - DNA, 20 bp, synthetic construct
  source 1..20: mol_type = other DNA
gagaaggcggcactctggtg

SEQ ID NO: 98 - DNA, 20 bp, synthetic construct
  source 1..20: mol_type = other DNA
ccccttcggtcaccacgagc

SEQ ID NO: 99 - DNA, 20 bp, synthetic construct
  source 1..20: mol_type = other DNA
cccttcggtcaccacgagca

SEQ ID NO: 100 - DNA, 20 bp, synthetic construct
  source 1..20: mol_type = other DNA
gtgtcacacaactgcccaac

SEQ ID NO: 101 - DNA, 20 bp, synthetic construct
  source 1..20: mol_type = other DNA
cgtgtcacacaactgcccaa

SEQ ID NO: 102 - DNA, 20 bp, synthetic construct
  source 1..20: mol_type = other DNA
acagagcaaagtggccgtcg

SEQ ID NO: 103 - DNA, 20 bp, synthetic construct
  source 1..20: mol_type = other DNA
agcctcccacatctctccta

SEQ ID NO: 104 - DNA, 20 bp, synthetic construct
  source 1..20: mol_type = other DNA
gaacggcatcccagccacga

SEQ ID NO: 105 - DNA, 20 bp, synthetic construct
  source 1..20: mol_type = other DNA
cccacatctctcctatggtc

SEQ ID NO: 106 - DNA, 20 bp, synthetic construct
  source 1..20: mol_type = other DNA
gcgctgagccctccaaaagg

SEQ ID NO: 107 - DNA, 20 bp, synthetic construct
  source 1..20: mol_type = other DNA
ccacatctctcctatggtct

SEQ ID NO: 108 - DNA, 20 bp, synthetic construct
  source 1..20: mol_type = other DNA
gcagcgctgagccctccaaa

SEQ ID NO: 109 - DNA, 20 bp, synthetic construct
  source 1..20: mol_type = other DNA
gaccagaggccggaatccag

SEQ ID NO: 110 - DNA, 20 bp, synthetic construct
  source 1..20: mol_type = other DNA
gtgcggcaacctacatgatg

SEQ ID NO: 111 - DNA, 20 bp, synthetic construct
  source 1..20: mol_type = other DNA
cctcactatccaaggactga

SEQ ID NO: 112 - DNA, 20 bp, synthetic construct
  source 1..20: mol_type = other DNA
caagtgaacctcactatcca

SEQ ID NO: 113 - DNA, 20 bp, synthetic construct
  source 1..20: mol_type = other DNA
gggactctacatctgcaagg

SEQ ID NO: 114 - DNA, 20 bp, synthetic construct
  source 1..20: mol_type = other DNA
cacgggactctacatctgca

SEQ ID NO: 115 - DNA, 20 bp, synthetic construct
  source 1..20: mol_type = other DNA
tgtgcggcaacctacatgat

SEQ ID NO: 116 - DNA, 20 bp, synthetic construct
  source 1..20: mol_type = other DNA
gatgtagagtcccgtgtcca

SEQ ID NO: 117 - DNA, 20 bp, synthetic construct
  source 1..20: mol_type = other DNA
ccgcacagacttcagtcacc

SEQ ID NO: 118 - DNA, 20 bp, synthetic construct
  source 1..20: mol_type = other DNA
tggcacaaatagctggccaa

SEQ ID NO: 119 - DNA, 20 bp, synthetic construct
  source 1..20: mol_type = other DNA
ggcacaaatagctggccaaa

SEQ ID NO: 120 - DNA, 20 bp, synthetic construct
  source 1..20: mol_type = other DNA
cggcttgtttctggacccgt

SEQ ID NO: 121 - DNA, 20 bp, synthetic construct
  source 1..20: mol_type = other DNA
caacgggtccagaaacaagc

SEQ ID NO: 122 - DNA, 20 bp, synthetic construct
  source 1..20: mol_type = other DNA
cttttctccaagacgggctg

SEQ ID NO: 123 - DNA, 20 bp, synthetic construct
  source 1..20: mol_type = other DNA
aggcaatgcctggatccgag

SEQ ID NO: 124 - DNA, 20 bp, synthetic construct
  source 1..20: mol_type = other DNA
tgcatcctgtcatttagctc

SEQ ID NO: 125 - DNA, 20 bp, synthetic construct
  source 1..20: mol_type = other DNA
gaaagcagcgaggcaatgcc

SEQ ID NO: 126 - DNA, 20 bp, synthetic construct
  source 1..20: mol_type = other DNA
gggactgctcacgttcatca

SEQ ID NO: 127 - DNA, 20 bp, synthetic construct
  source 1..20: mol_type = other DNA
ggattcatacctgctgatgt

SEQ ID NO: 128 - DNA, 20 bp, synthetic construct
  source 1..20: mol_type = other DNA
aaaagaggctgacggcaact

SEQ ID NO: 129 - DNA, 20 bp, synthetic construct
  source 1..20: mol_type = other DNA
aaaaagaggctgacggcaac

SEQ ID NO: 130 - DNA, 20 bp, synthetic construct
  source 1..20: mol_type = other DNA
actgccccggctggtcccaa

SEQ ID NO: 131 - DNA, 20 bp, synthetic construct
  source 1..20: mol_type = other DNA
cgatgccaaaaagaggctga

SEQ ID NO: 132 - DNA, 20 bp, synthetic construct
  source 1..20: mol_type = other DNA
gaaactctagccactcgtcc

SEQ ID NO: 133 - DNA, 20 bp, synthetic construct
  source 1..20: mol_type = other DNA
aaactctagccactcgtcct

SEQ ID NO: 134 - DNA, 20 bp, synthetic construct
  source 1..20: mol_type = other DNA
accctgatgggacgtacact

SEQ ID NO: 135 - DNA, 20 bp, synthetic construct
  source 1..20: mol_type = other DNA
taccctgatgggacgtacac

SEQ ID NO: 136 - DNA, 20 bp, synthetic construct
  source 1..20: mol_type = other DNA
caccacggcacaagtgaccc

SEQ ID NO: 137 - DNA, 20 bp, synthetic construct
  source 1..20: mol_type = other DNA
gctgaccgtgaacgatacag

SEQ ID NO: 138 - DNA, 20 bp, synthetic construct
  source 1..20: mol_type = other DNA
ctcccagtgtacgtcccatc

SEQ ID NO: 139 - DNA, 20 bp, synthetic construct
  source 1..20: mol_type = other DNA
tggggccactcgatccttga

SEQ ID NO: 140 - DNA, 20 bp, synthetic construct
  source 1..20: mol_type = other DNA
cgctgaccgtgaacgataca

SEQ ID NO: 141 - DNA, 20 bp, synthetic construct
  source 1..20: mol_type = other DNA
tcgctgaccgtgaacgatac

SEQ ID NO: 142 - DNA, 20 bp, synthetic construct
  source 1..20: mol_type = other DNA
gggtcgatctccaccacctg

SEQ ID NO: 143 - DNA, 20 bp, synthetic construct
  source 1..20: mol_type = other DNA
ggagtttagccagtccaact

SEQ ID NO: 144 - DNA, 20 bp, synthetic construct
  source 1..20: mol_type = other DNA
gagttggactggctaaactc

SEQ ID NO: 145 - DNA, 20 bp, synthetic construct
  source 1..20: mol_type = other DNA
caccaaggccatcgagagct

SEQ ID NO: 146 - DNA, 20 bp, synthetic construct
  source 1..20: mol_type = other DNA
atccacatcgaggctcctcg

SEQ ID NO: 147 - DNA, 20 bp, synthetic construct
  source 1..20: mol_type = other DNA
gagcccagaacttaacttcg

SEQ ID NO: 148 - DNA, 20 bp, synthetic construct
  source 1..20: mol_type = other DNA
catccacatcgaggctcctc

SEQ ID NO: 149 - DNA, 20 bp, synthetic construct
  source 1..20: mol_type = other DNA
ctacgccgacctcatcacca

SEQ ID NO: 150 - DNA, 20 bp, synthetic construct
  source 1..20: mol_type = other DNA
gctccctggacacccattcc

SEQ ID NO: 151 - DNA, 20 bp, synthetic construct
  source 1..20: mol_type = other DNA
tcccaaatcccagtgcaccc

SEQ ID NO: 152 - DNA, 20 bp, synthetic construct
  source 1..20: mol_type = other DNA
ttcgaagaccttctcacatc

SEQ ID NO: 153 - DNA, 20 bp, synthetic construct
  source 1..20: mol_type = other DNA
tcgaagaccttctcacatcc

SEQ ID NO: 154 - DNA, 20 bp, synthetic construct
  source 1..20: mol_type = other DNA
caagtggcccggatgtgaga

SEQ ID NO: 155 - DNA, 20 bp, synthetic construct
  source 1..20: mol_type = other DNA
gaaggtcttcgaagagccag

SEQ ID NO: 156 - DNA, 20 bp, synthetic construct
  source 1..20: mol_type = other DNA
actgtaccatctctctctgg

SEQ ID NO: 157 - DNA, 20 bp, synthetic construct
  source 1..20: mol_type = other DNA
ggaccatcttctggatgaga

SEQ ID NO: 158 - DNA, 20 bp, synthetic construct
  source 1..20: mol_type = other DNA
tctctcagctggtacacggc

SEQ ID NO: 159 - DNA, 20 bp, synthetic construct
  source 1..20: mol_type = other DNA
ctcgaccagcttgacatcac

SEQ ID NO: 160 - DNA, 20 bp, synthetic construct
  source 1..20: mol_type = other DNA
aagttcctgtgatgtcaagc

SEQ ID NO: 161 - DNA, 20 bp, synthetic construct
  source 1..20: mol_type = other DNA
ttcggaacccaatcactgac

SEQ ID NO: 162 - DNA, 20 bp, synthetic construct
  source 1..20: mol_type = other DNA
gattaaacccggccactttc

SEQ ID NO: 163 - DNA, 20 bp, synthetic construct
  source 1..20: mol_type = other DNA
acccggccactttcaggagg

SEQ ID NO: 164 - DNA, 20 bp, synthetic construct
  source 1..20: mol_type = other DNA
cgtcatgagcagattaaacc

SEQ ID NO: 165 - DNA, 20 bp, synthetic construct
  source 1..20: mol_type = other DNA
taaacccggccactttcagg

SEQ ID NO: 166 - DNA, 20 bp, synthetic construct
  source 1..20: mol_type = other DNA
gaacaaggtgttcccacccg

SEQ ID NO: 167 - DNA, 20 bp, synthetic construct
  source 1..20: mol_type = other DNA
cgggtgggaacaccttgttc

SEQ ID NO: 168 - DNA, 20 bp, synthetic construct
  source 1..20: mol_type = other DNA
tcaaacacagcgacctcggg

SEQ ID NO: 169 - DNA, 20 bp, synthetic construct
  source 1..20: mol_type = other DNA
cgtagaactggacttgacag

SEQ ID NO: 170 - DNA, 20 bp, synthetic construct
  source 1..20: mol_type = other DNA
atgacgagtggacccaggat

SEQ ID NO: 171 - DNA, 20 bp, synthetic construct
  source 1..20: mol_type = other DNA
gctgtcaagtccagttctac

SEQ ID NO: 172 - DNA, 20 bp, synthetic construct
  source 1..20: mol_type = other DNA
tgacgagtggacccaggata

SEQ ID NO: 173 - DNA, 20 bp, synthetic construct
  source 1..20: mol_type = other DNA
cttgacagcggaagtggttg

SEQ ID NO: 174 - DNA, 20 bp, synthetic construct
  source 1..20: mol_type = other DNA
accactatgacctgctacag

SEQ ID NO: 175 - DNA, 20 bp, synthetic construct
  source 1..20: mol_type = other DNA
cattttcaatagagaccccc

SEQ ID NO: 176 - DNA, 20 bp, synthetic construct
  source 1..20: mol_type = other DNA
gggtccacgacgtccatccc

SEQ ID NO: 177 - DNA, 20 bp, synthetic construct
  source 1..20: mol_type = other DNA
ggtccacgacgtccatccct

SEQ ID NO: 178 - DNA, 20 bp, synthetic construct
  source 1..20: mol_type = other DNA
gtccacgacgtccatccctg

SEQ ID NO: 179 - DNA, 20 bp, synthetic construct
  source 1..20: mol_type = other DNA
tccacgacgtccatccctgg

SEQ ID NO: 180 - DNA, 20 bp, synthetic construct
  source 1..20: mol_type = other DNA
ccaacatcgtggcctaccat

SEQ ID NO: 181 - DNA, 20 bp, synthetic construct
  source 1..20: mol_type = other DNA
cccatggtaggccacgatgt

SEQ ID NO: 182 - DNA, 20 bp, synthetic construct
  source 1..20: mol_type = other DNA
taggatcttcgtggctgtct

SEQ ID NO: 183 - DNA, 20 bp, synthetic construct
  source 1..20: mol_type = other DNA
accaggttgtagaagttgac

SEQ ID NO: 184 - DNA, 20 bp, synthetic construct
  source 1..20: mol_type = other DNA
aagttgacaggcagtgccat

SEQ ID NO: 185 - DNA, 20 bp, synthetic construct
  source 1..20: mol_type = other DNA
gcatccagcaacataagcgg

SEQ ID NO: 186 - DNA, 20 bp, synthetic construct
  source 1..20: mol_type = other DNA
taactttattgacccccagc

SEQ ID NO: 187 - DNA, 20 bp, synthetic construct
  source 1..20: mol_type = other DNA
caacccctcccaaagatgga

SEQ ID NO: 188 - DNA, 20 bp, synthetic construct
  source 1..20: mol_type = other DNA
ttctacaacctggtgatgtc

SEQ ID NO: 189 - DNA, 20 bp, synthetic construct
  source 1..20: mol_type = other DNA
gcctgtcaacttctacaacc

SEQ ID NO: 190 - DNA, 20 bp, synthetic construct
  source 1..20: mol_type = other DNA
aagtcaacttcaatgtcgga

SEQ ID NO: 191 - DNA, 20 bp, synthetic construct
  source 1..20: mol_type = other DNA
cgtgagtaaacctgaatctt

SEQ ID NO: 192 - DNA, 20 bp, synthetic construct
  source 1..20: mol_type = other DNA
acagcccaagatagttaagt

SEQ ID NO: 193 - DNA, 20 bp, synthetic construct
  source 1..20: mol_type = other DNA
attgtttagagctacccagc

SEQ ID NO: 194 - DNA, 20 bp, synthetic construct
  source 1..20: mol_type = other DNA
cttaccccacttaactatct

SEQ ID NO: 195 - DNA, 20 bp, synthetic construct
  source 1..20: mol_type = other DNA
cgaacatctcaagaaggtat

SEQ ID NO: 196 - DNA, 20 bp, synthetic construct
  source 1..20: mol_type = other DNA
ttaccccacttaactatctt

SEQ ID NO: 197 - DNA, 20 bp, synthetic construct
  source 1..20: mol_type = other DNA
ccaatccagccagaaagtac

SEQ ID NO: 198 - DNA, 20 bp, synthetic construct
  source 1..20: mol_type = other DNA
tgtgctagacatgaggtcta

SEQ ID NO: 199 - DNA, 20 bp, synthetic construct
  source 1..20: mol_type = other DNA
ggagaatgacgagtggaccc

SEQ ID NO: 200 - DNA, 20 bp, synthetic construct
  source 1..20: mol_type = other DNA
ggcgccctggccagtcgtct

SEQ ID NO: 201 - DNA, 20 bp, synthetic construct
  source 1..20: mol_type = other DNA
cagggttctggatatctgtg

SEQ ID NO: 202 - DNA, 20 bp, synthetic construct
  source 1..20: mol_type = other DNA
ggccacggagcgagacatct

SEQ ID NO: 203 - DNA, 20 bp, synthetic construct
  source 1..20: mol_type = other DNA
gggaaagacccagcatccgt

SEQ ID NO: 204 - DNA, 20 bp, synthetic construct
  source 1..20: mol_type = other DNA
ggcccagactgagcacgtga

SEQ ID NO: 205 - DNA, 20 bp, synthetic construct
  source 1..20: mol_type = other DNA
gtcatcttagtcattacctg

SEQ ID NO: 206 - DNA, 20 bp, synthetic construct
  source 1..20: mol_type = other DNA
ggaatcccttctgcagcacc

SEQ ID NO: 207 - DNA, 20 bp, synthetic construct
  source 1..20: mol_type = other DNA
gggtggggggagtttgctcc

SEQ ID NO: 208 - DNA, 20 bp, synthetic construct
  source 1..20: mol_type = other DNA
tggtaatgatggcttcaaca

SEQ ID NO: 209 - DNA, 20 bp, synthetic construct
  source 1..20: mol_type = other DNA
gaacacaaagcatagactgc

SEQ ID NO: 210 - DNA, 20 bp, synthetic construct
  source 1..20: mol_type = other DNA
gtgttcatctttggttttgt

SEQ ID NO: 211 - DNA, 20 bp, synthetic construct
  source 1..20: mol_type = other DNA
tgtccccgcacggagcccac

SEQ ID NO: 212 - DNA, 20 bp, synthetic construct
  source 1..20: mol_type = other DNA
gggggcctacaccttccaac

SEQ ID NO: 213 - DNA, 20 bp, synthetic construct
  source 1..20: mol_type = other DNA
tgtttggataggggtacacg

SEQ ID NO: 214 - DNA, 20 bp, synthetic construct
  source 1..20: mol_type = other DNA
agcccaagggaagtcaccgc

SEQ ID NO: 215 - DNA, 20 bp, synthetic construct
  source 1..20: mol_type = other DNA
gcgggccaacttcactctgc

SEQ ID NO: 216 - DNA, 20 bp, synthetic construct
  source 1..20: mol_type = other DNA
tcaagctttaccccaccata

SEQ ID NO: 217 - DNA, 20 bp, synthetic construct
  source 1..20: mol_type = other DNA
gttggagcatctgagtccag

SEQ ID NO: 218 - DNA, 20 bp, synthetic construct
  source 1..20: mol_type = other DNA
gagtccgagcagaagaagaa

SEQ ID NO: 219 - DNA, 20 bp, synthetic construct
  source 1..20: mol_type = other DNA
gaaggctgagatcctggagg

SEQ ID NO: 220 - DNA, 20 bp, synthetic construct
  source 1..20: mol_type = other DNA
gggaacccagcgagtgaaga

SEQ ID NO: 221 - DNA, 20 bp, synthetic construct
  source 1..20: mol_type = other DNA
gagccacattaaccggccct

SEQ ID NO: 222 - DNA, 20 bp, synthetic construct
  source 1..20: mol_type = other DNA
ggtgagggaggagagatgcc

SEQ ID NO: 223 - DNA, 20 bp, synthetic construct
  source 1..20: mol_type = other DNA
ggccgagatgtctcgctccg

SEQ ID NO: 224 - DNA, 20 bp, synthetic construct
  source 1..20: mol_type = other DNA
gaagttgacttactgaagaa

SEQ ID NO: 225 - DNA, 20 bp, synthetic construct
  source 1..20: mol_type = other DNA
gctactctctctttctggcc

SEQ ID NO: 226 - DNA, 20 bp, synthetic construct
  source 1..20: mol_type = other DNA
ggcagaaaccctggtggtcg

SEQ ID NO: 227 - DNA, 20 bp, synthetic construct
  source 1..20: mol_type = other DNA
ggatttcctcctcgaccacc

SEQ ID NO: 228 - DNA, 20 bp, synthetic construct
  source 1..20: mol_type = other DNA
gggtattattgatgctattc

SEQ ID NO: 229 - DNA, 20 bp, synthetic construct
  source 1..20: mol_type = other DNA
ggtacctatcgattgtcagg

SEQ ID NO: 230 - DNA, 20 bp, synthetic construct
  source 1..20: mol_type = other DNA
gacattaaagatagtcatct

SEQ ID NO: 231 - DNA, 20 bp, synthetic construct
  source 1..20: mol_type = other DNA
gtagagcggaggcaggaggc

SEQ ID NO: 232 - DNA, 20 bp, synthetic construct
  source 1..20: mol_type = other DNA
gaggttcacttgatttccac

SEQ ID NO: 233 - DNA, 20 bp, synthetic construct
  source 1..20: mol_type = other DNA
gtgcggcaacctacatgatg

SEQ ID NO: 234 - DNA, 20 bp, synthetic construct
  source 1..20: mol_type = other DNA
gcacaaggctcagctgaacc

SEQ ID NO: 235 - DNA, 20 bp, synthetic construct
  source 1..20: mol_type = other DNA
gataactacaccgaggaaat

SEQ ID NO: 236 - DNA, 20 bp, synthetic construct
  source 1..20: mol_type = other DNA
gccgtggcaaactggtactt

SEQ ID NO: 237 - DNA, 20 bp, synthetic construct
  source 1..20: mol_type = other DNA
gaagatgatggagtagatgg

SEQ ID NO: 238 - DNA, 20 bp, synthetic construct
  source 1..20: mol_type = other DNA
ggggcagctccggcgctcct

SEQ ID NO: 239 - DNA, 20 bp, synthetic construct
  source 1..20: mol_type = other DNA
gctgaccccgctgggcaggc

SEQ ID NO: 240 - DNA, 20 bp, synthetic construct
  source 1..20: mol_type = other DNA
gagggctcaccagaggtagg

SEQ ID NO: 241 - DNA, 20 bp, synthetic construct
  source 1..20: mol_type = other DNA
gctgtttctgcagccgcttt

SEQ ID NO: 242 - DNA, 20 bp, synthetic construct
  source 1..20: mol_type = other DNA
ggtcccggtggtgtgggccc

SEQ ID NO: 243 - DNA, 20 bp, synthetic construct
  source 1..20: mol_type = other DNA
ggtggtgtgggcccaggagg

SEQ ID NO: 244 - DNA, 20 bp, synthetic construct
  source 1..20: mol_type = other DNA
gcgtgacttccacatgagcg

SEQ ID NO: 245 - DNA, 20 bp, synthetic construct
  source 1..20: mol_type = other DNA
gtctgggcggtgctacaact

SEQ ID NO: 246 - DNA, 20 bp, synthetic construct
  source 1..20: mol_type = other DNA
gagcagggctggggagaagg

SEQ ID NO: 247 - DNA, 20 bp, synthetic construct
  source 1..20: mol_type = other DNA
ggaaacttggccactctatg

SEQ ID NO: 248 - DNA, 20 bp, synthetic construct
  source 1..20: mol_type = other DNA
ggcaccaactggatggatca

SEQ ID NO: 249 - DNA, 20 bp, synthetic construct
  source 1..20: mol_type = other DNA
gtctccctgatccatccagt

SEQ ID NO: 250 - DNA, 20 bp, synthetic construct
  source 1..20: mol_type = other DNA
gtttgcgactctgacagagc

SEQ ID NO: 251 - DNA, 20 bp, synthetic construct
  source 1..20: mol_type = other DNA
ggtttcaccgagacctcagt

SEQ ID NO: 252 - DNA, 20 bp, synthetic construct
  source 1..20: mol_type = other DNA
gatttctatgacctgtatgg

SEQ ID NO: 253 - DNA, 20 bp, synthetic construct
  source 1..20: mol_type = other DNA
gagaatcaaaatcggtgaat

SEQ ID NO: 254 - DNA, 20 bp, synthetic construct
  source 1..20: mol_type = other DNA
gacaccttcttccccagccc

SEQ ID NO: 255 - DNA, 20 bp, synthetic construct
  source 1..20: mol_type = other DNA
gctggtacacggcagggtca

SEQ ID NO: 256 - DNA, 20 bp, synthetic construct
  source 1..20: mol_type = other DNA
gaacaaggtgttcccacccg

SEQ ID NO: 257 - DNA, 20 bp, synthetic construct
  source 1..20: mol_type = other DNA
ggtgcacagtggggtcagca

SEQ ID NO: 258 - DNA, 20 bp, synthetic construct
  source 1..20: mol_type = other DNA
gacgtcgtcgatccacttag

SEQ ID NO: 259 - DNA, 20 bp, synthetic construct
  source 1..20: mol_type = other DNA
tcacccgtcaagacgtgttc

SEQ ID NO: 260 - DNA, 20 bp, synthetic construct
  source 1..20: mol_type = other DNA
tgtaccagaacgtgtccacg

SEQ ID NO: 261 - DNA, 20 bp, synthetic construct
  source 1..20: mol_type = other DNA
acggacactgtgatcatcgt

SEQ ID NO: 262 - DNA, 20 bp, synthetic construct
  source 1..20: mol_type = other DNA
ccaaccaggacgatcatacg

SEQ ID NO: 263 - DNA, 20 bp, synthetic construct
  source 1..20: mol_type = other DNA
tgtcactataggatcgcaag

SEQ ID NO: 264 - DNA, 20 bp, synthetic construct
  source 1..20: mol_type = other DNA
ccttgcaagacgacttactc

SEQ ID NO: 265 - DNA, 20 bp, synthetic construct
  source 1..20: mol_type = other DNA
gtgtccacaacgggttatct

SEQ ID NO: 266 - DNA, 20 bp, synthetic construct
  source 1..20: mol_type = other DNA
ggagacaactgctactccgt

SEQ ID NO: 267 - DNA, 20 bp, synthetic construct
  source 1..20: mol_type = other DNA
ttgtcctttccacgaactac

SEQ ID NO: 268 - DNA, 20 bp, synthetic construct
  source 1..20: mol_type = other DNA
gaagtacggggctaccactg

SEQ ID NO: 269 - DNA, 20 bp, synthetic construct
  source 1..20: mol_type = other DNA
acgagcaccactctgagata

SEQ ID NO: 270 - DNA, 20 bp, synthetic construct
  source 1..20: mol_type = other DNA
tgtcggagcctacaaacaaa

SEQ ID NO: 271 - DNA, 20 bp, synthetic construct
  source 1..20: mol_type = other DNA
atggacaccggtgtgattga

SEQ ID NO: 272 - DNA, 20 bp, synthetic construct
  source 1..20: mol_type = other DNA
gcaaccacgaaagccacctc

SEQ ID NO: 273 - DNA, 20 bp, synthetic construct
  source 1..20: mol_type = other DNA
gcttgtagtaggagtaatac

SEQ ID NO: 274 - DNA, 20 bp, synthetic construct
  source 1..20: mol_type = other DNA
tcatggagcttgaacacctc

SEQ ID NO: 275 - DNA, 20 bp, synthetic construct
  source 1..20: mol_type = other DNA
aaccagatcttgacctgcgt

SEQ ID NO: 276 - DNA, 20 bp, synthetic construct
  source 1..20: mol_type = other DNA
caggaaagctttaccaattc

SEQ ID NO: 277 - DNA, 20 bp, synthetic construct
  source 1..20: mol_type = other DNA
cggcacgacccggacaatat

SEQ ID NO: 278 - DNA, 20 bp, synthetic construct
  source 1..20: mol_type = other DNA
ggtggacaagcggcagatag

SEQ ID NO: 279 - DNA, 20 bp, synthetic construct
  source 1..20: mol_type = other DNA
ataggagaagatgatgtata

SEQ ID NO: 280 - DNA, 20 bp, synthetic construct
  source 1..20: mol_type = other DNA
gcatacagtgatttgatgaa

SEQ ID NO: 281 - DNA, 20 bp, synthetic construct
  source 1..20: mol_type = other DNA
gctgatgtagtcactcttga

SEQ ID NO: 282 - DNA, 20 bp, synthetic construct
  source 1..20: mol_type = other DNA
gcaaaactcaaccctacccc

SEQ ID NO: 283 - DNA, 20 bp, synthetic construct
  source 1..20: mol_type = other DNA
ctcgtctgataagacaacag

SEQ ID NO: 284 - DNA, 20 bp, synthetic construct
  source 1..20: mol_type = other DNA
cttgccccacagggcagtaa

SEQ ID NO: 285 - RNA, 100 nt, synthetic construct; identical to SEQ ID NO: 68
  source 1..100: mol_type = other RNA

SEQ ID NO: 286 - RNA, 100 nt, synthetic construct; identical to SEQ ID NO: 70
  source 1..100: mol_type = other RNA

SEQ ID NO: 287 - RNA, 100 nt, synthetic construct; identical to SEQ ID NO: 71
  source 1..100: mol_type = other RNA

SEQ ID NO: 288 - RNA, 100 nt, synthetic construct
  source 1..100: mol_type = other RNA
cccgcaccttggcgcagcgggttttagagctagaaatagcaagttaaaataaggctagtccgttatcaacttgaaaaagtggcaccgagtcggtgctttt

SEQ ID NO: 289 - RNA, 100 nt, synthetic construct
  source 1..100: mol_type = other RNA
ctaacagttgcttttatcacgttttagagctagaaatagcaagttaaaataaggctagtccgttatcaacttgaaaaagtggcaccgagtcggtgctttt

SEQ ID NO: 290 - RNA, 100 nt, synthetic construct; identical to SEQ ID NO: 69
  source 1..100: mol_type = other RNA

SEQ ID NO: 291 - RNA, 100 nt, synthetic construct
  source 1..100: mol_type = other RNA
ggaatcccttctgcagcaccgttttagagctagaaatagcaagttaaaataaggctagtccgttatcaacttgaaaaagtggcaccgagtcggtgctttt

SEQ ID NO: 292 - RNA, 100 nt, synthetic construct
  source 1..100: mol_type = other RNA
gagcagggctggggagaagggttttagagctagaaatagcaagttaaaataaggctagtccgttatcaacttgaaaaagtggcaccgagtcggtgctttt

SEQ ID NO: 293 - RNA, 100 nt, synthetic construct
  source 1..100: mol_type = other RNA
acgagcaccactctgagatagttttagagctagaaatagcaagttaaaataaggctagtccgttatcaacttgaaaaagtggcaccgagtcggtgctttt

SEQ ID NO: 294 - RNA, 100 nt, synthetic construct
  source 1..100: mol_type = other RNA
gtcatcttagtcattacctggttttagagctagaaatagcaagttaaaataaggctagtccgttatcaacttgaaaaagtggcaccgagtcggtgctttt

SEQ ID NO: 295 - RNA, 100 nt, synthetic construct
  source 1..100: mol_type = other RNA
cgtcatgagcagattaaaccgttttagagctagaaatagcaagttaaaataaggctagtccgttatcaacttgaaaaagtggcaccgagtcggtgctttt

SEQ ID NO: 296 - RNA, 100 nt, synthetic construct
  source 1..100: mol_type = other RNA
ggccgagatgtctcgctccggttttagagctagaaatagcaagttaaaataaggctagtccgttatcaacttgaaaaagtggcaccgagtcggtgctttt

SEQ ID NO: 297 - RNA, 100 nt, synthetic construct
  source 1..100: mol_type = other RNA
cgctgaccgtgaacgatacagttttagagctagaaatagcaagttaaaataaggctagtccgttatcaacttgaaaaagtggcaccgagtcggtgctttt

SEQ ID NO: 298 - RNA, 100 nt, synthetic construct
  source 1..100: mol_type = other RNA
cgtctgggcggtgctacaacgttttagagctagaaatagcaagttaaaataaggctagtccgttatcaacttgaaaaagtggcaccgagtcggtgctttt

SEQ ID NO: 299 - RNA, 100 nt, synthetic construct
  source 1..100: mol_type = other RNA
tgtagcaccgcccagacgacgttttagagctagaaatagcaagttaaaataaggctagtccgttatcaacttgaaaaagtggcaccgagtcggtgctttt

SEQ ID NO: 300 - RNA, 100 nt, synthetic construct
  source 1..100: mol_type = other RNA
gtctgggcggtgctacaactgttttagagctagaaatagcaagttaaaataaggctagtccgttatcaacttgaaaaagtggcaccgagtcggtgctttt

SEQ ID NO: 301 - RNA, 100 nt, synthetic construct
  source 1..100: mol_type = other RNA
gagaaggcggcactctggtggttttagagctagaaatagcaagttaaaataaggctagtccgttatcaacttgaaaaagtggcaccgagtcggtgctttt

SEQ ID NO: 302 - RNA, 100 nt, synthetic construct
  source 1..100: mol_type = other RNA
ccccttcggtcaccacgagcgttttagagctagaaatagcaagttaaaataaggctagtccgttatcaacttgaaaaagtggcaccgagtcggtgctttt

SEQ ID NO: 303 - RNA, 100 nt, synthetic construct
  source 1..100: mol_type = other RNA
cccttcggtcaccacgagcagttttagagctagaaatagcaagttaaaataaggctagtccgttatcaacttgaaaaagtggcaccgagtcggtgctttt

SEQ ID NO: 304 - RNA, 100 nt, synthetic construct
  source 1..100: mol_type = other RNA
gtgtcacacaactgcccaacgttttagagctagaaatagcaagttaaaataaggctagtccgttatcaacttgaaaaagtggcaccgagtcggtgctttt

SEQ ID NO: 305 - RNA, 100 nt, synthetic construct
  source 1..100: mol_type = other RNA
cgtgtcacacaactgcccaagttttagagctagaaatagcaagttaaaataaggctagtccgttatcaacttgaaaaagtggcaccgagtcggtgctttt

SEQ ID NO: 306 - RNA, 100 nt, synthetic construct
  source 1..100: mol_type = other RNA
acagagcaaagtggccgtcggttttagagctagaaatagcaagttaaaataaggctagtccgttatcaacttgaaaaagtggcaccgagtcggtgctttt

SEQ ID NO: 307 - RNA, 100 nt, synthetic construct
  source 1..100: mol_type = other RNA
agcctcccacatctctcctagttttagagctagaaatagcaagttaaaataaggctagtccgttatcaacttgaaaaagtggcaccgagtcggtgctttt

SEQ ID NO: 308 - RNA, 100 nt, synthetic construct
  source 1..100: mol_type = other RNA
gaacggcatcccagccacgagttttagagctagaaatagcaagttaaaataaggctagtccgttatcaacttgaaaaagtggcaccgagtcggtgctttt

SEQ ID NO: 309 - RNA, 100 nt, synthetic construct
  source 1..100: mol_type = other RNA
cccacatctctcctatggtcgttttagagctagaaatagcaagttaaaataaggctagtccgttatcaacttgaaaaagtggcaccgagtcggtgctttt

SEQ ID NO: 310 - RNA, 100 nt, synthetic construct
  source 1..100: mol_type = other RNA
gcgctgagccctccaaaagggttttagagctagaaatagcaagttaaaataaggctagtccgttatcaacttgaaaaagtggcaccgagtcggtgctttt

SEQ ID NO: 311 - RNA, 100 nt, synthetic construct
  source 1..100: mol_type = other RNA
ccacatctctcctatggtctgttttagagctagaaatagcaagttaaaataaggctagtccgttatcaacttgaaaaagtggcaccgagtcggtgctttt

SEQ ID NO: 312 - RNA, 100 nt, synthetic construct
  source 1..100: mol_type = other RNA
gcagcgctgagccctccaaagttttagagctagaaatagcaagttaaaataaggctagtccgttatcaacttgaaaaagtggcaccgagtcggtgctttt

SEQ ID NO: 313 - RNA, 100 nt, synthetic construct
  source 1..100: mol_type = other RNA
gaccagaggccggaatccaggttttagagctagaaatagcaagttaaaataaggctagtccgttatcaacttgaaaaagtggcaccgagtcggtgctttt

SEQ ID NO: 314 - RNA, 100 nt, synthetic construct
  source 1..100: mol_type = other RNA
gtgcggcaacctacatgatggttttagagctagaaatagcaagttaaaataaggctagtccgttatcaacttgaaaaagtggcaccgagtcggtgctttt

SEQ ID NO: 315 - RNA, 100 nt, synthetic construct
  source 1..100: mol_type = other RNA
cctcactatccaaggactgagttttagagctagaaatagcaagttaaaataaggctagtccgttatcaacttgaaaaagtggcaccgagtcggtgctttt

SEQ ID NO: 316 - RNA, 100 nt, synthetic construct
  source 1..100: mol_type = other RNA
caagtgaacctcactatccagttttagagctagaaatagcaagttaaaataaggctagtccgttatcaacttgaaaaagtggcaccgagtcggtgctttt

SEQ ID NO: 317 - RNA, 100 nt, synthetic construct
  source 1..100: mol_type = other RNA
gggactctacatctgcaagggttttagagctagaaatagcaagttaaaataaggctagtccgttatcaacttgaaaaagtggcaccgagtcggtgctttt

SEQ ID NO: 318 - RNA, 100 nt, synthetic construct
  source 1..100: mol_type = other RNA
cacgggactctacatctgcagttttagagctagaaatagcaagttaaaataaggctagtccgttatcaacttgaaaaagtggcaccgagtcggtgctttt

SEQ ID NO: 319 - RNA, 100 nt, synthetic construct
  source 1..100: mol_type = other RNA
tgtgcggcaacctacatgatgttttagagctagaaatagcaagttaaaataaggctagtccgttatcaacttgaaaaagtggcaccgagtcggtgctttt

SEQ ID NO: 320 - RNA, 100 nt, synthetic construct
  source 1..100: mol_type = other RNA
gatgtagagtcccgtgtccagttttagagctagaaatagcaagttaaaataaggctagtccgttatcaacttgaaaaagtggcaccgagtcggtgctttt

SEQ ID NO: 321 - RNA, 100 nt, synthetic construct
  source 1..100: mol_type = other RNA
ccgcacagacttcagtcaccgttttagagctagaaatagcaagttaaaataaggctagtccgttatcaacttgaaaaagtggcaccgagtcggtgctttt

SEQ ID NO: 322 - RNA, 100 nt, synthetic construct
  source 1..100: mol_type = other RNA
tggcacaaatagctggccaagttttagagctagaaatagcaagttaaaataaggctagtccgttatcaacttgaaaaagtggcaccgagtcggtgctttt

SEQ ID NO: 323 - RNA, 100 nt, synthetic construct
  source 1..100: mol_type = other RNA
ggcacaaatagctggccaaagttttagagctagaaatagcaagttaaaataaggctagtccgttatcaacttgaaaaagtggcaccgagtcggtgctttt

SEQ ID NO: 324 - RNA, 100 nt, synthetic construct
  source 1..100: mol_type = other RNA
cggcttgtttctggacccgtgttttagagctagaaatagcaagttaaaataaggctagtccgttatcaacttgaaaaagtggcaccgagtcggtgctttt

SEQ ID NO: 325 - RNA, 100 nt, synthetic construct
  source 1..100: mol_type = other RNA
caacgggtccagaaacaagcgttttagagctagaaatagcaagttaaaataaggctagtccgttatcaacttgaaaaagtggcaccgagtcggtgctttt

SEQ ID NO: 326 - RNA, 100 nt, synthetic construct
  source 1..100: mol_type = other RNA
cttttctccaagacgggctggttttagagctagaaatagcaagttaaaataaggctagtccgttatcaacttgaaaaagtggcaccgagtcggtgctttt

SEQ ID NO: 327 - RNA, 100 nt, synthetic construct
  source 1..100: mol_type = other RNA
aggcaatgcctggatccgaggttttagagctagaaatagcaagttaaaataaggctagtccgttatcaacttgaaaaagtggcaccgagtcggtgctttt

SEQ ID NO: 328 - RNA, 100 nt, synthetic construct
  source 1..100: mol_type = other RNA
tgcatcctgtcatttagctcgttttagagctagaaatagcaagttaaaataaggctagtccgttatcaacttgaaaaagtggcaccgagtcggtgctttt

SEQ ID NO: 329 - RNA, 100 nt, synthetic construct
  source 1..100: mol_type = other RNA
gaaagcagcgaggcaatgccgttttagagctagaaatagcaagttaaaataaggctagtccgttatcaacttgaaaaagtggcaccgagtcggtgctttt

SEQ ID NO: 330 - RNA, 100 nt, synthetic construct
  source 1..100: mol_type = other RNA
gggactgctcacgttcatcagttttagagctagaaatagcaagttaaaataaggctagtccgttatcaacttgaaaaagtggcaccgagtcggtgctttt

SEQ ID NO: 331 - RNA, 100 nt, synthetic construct
  source 1..100: mol_type = other RNA
ggattcatacctgctgatgtgttttagagctagaaatagcaagttaaaataaggctagtccgttatcaacttgaaaaagtggcaccgagtcggtgctttt

SEQ ID NO: 332 - RNA, 100 nt, synthetic construct
  source 1..100: mol_type = other RNA
aaaagaggctgacggcaactgttttagagctagaaatagcaagttaaaataaggctagtccgttatcaacttgaaaaagtggcaccgagtcggtgctttt

SEQ ID NO: 333 - RNA, 100 nt, synthetic construct
  source 1..100: mol_type = other RNA
aaaaagaggctgacggcaacgttttagagctagaaatagcaagttaaaataaggctagtccgttatcaacttgaaaaagtggcaccgagtcggtgctttt

SEQ ID NO: 334 - RNA, 100 nt, synthetic construct
  source 1..100: mol_type = other RNA
actgccccggctggtcccaagttttagagctagaaatagcaagttaaaataaggctagtccgttatcaacttgaaaaagtggcaccgagtcggtgctttt

SEQ ID NO: 335 - RNA, 100 nt, synthetic construct
  source 1..100: mol_type = other RNA
cgatgccaaaaagaggctgagttttagagctagaaatagcaagttaaaataaggctagtccgttatcaacttgaaaaagtggcaccgagtcggtgctttt

SEQ ID NO: 336 - RNA, 100 nt, synthetic construct
  source 1..100: mol_type = other RNA
gaaactctagccactcgtccgttttagagctagaaatagcaagttaaaataaggctagtccgttatcaacttgaaaaagtggcaccgagtcggtgctttt

SEQ ID NO: 337 - RNA, 100 nt, synthetic construct
  source 1..100: mol_type = other RNA
aaactctagccactcgtcctgttttagagctagaaatagcaagttaaaataaggctagtccgttatcaacttgaaaaagtggcaccgagtcggtgctttt

SEQ ID NO: 338 - RNA, 100 nt, synthetic construct
  source 1..100: mol_type = other RNA
accctgatgggacgtacactgttttagagctagaaatagcaagttaaaataaggctagtccgttatcaacttgaaaaagtggcaccgagtcggtgctttt

SEQ ID NO: 339 - RNA, 100 nt, synthetic construct
  source 1..100: mol_type = other RNA
taccctgatgggacgtacacgttttagagctagaaatagcaagttaaaataaggctagtccgttatcaacttgaaaaagtggcaccgagtcggtgctttt

SEQ ID NO: 340 - RNA, 100 nt, synthetic construct
  source 1..100: mol_type = other RNA
caccacggcacaagtgacccgttttagagctagaaatagcaagttaaaataaggctagtccgttatcaacttgaaaaagtggcaccgagtcggtgctttt

SEQ ID NO: 341 - RNA, 100 nt, synthetic construct
  source 1..100: mol_type = other RNA
gctgaccgtgaacgatacaggttttagagctagaaatagcaagttaaaataaggctagtccgttatcaacttgaaaaagtggcaccgagtcggtgctttt

SEQ ID NO: 342 - RNA, 100 nt, synthetic construct
  source 1..100: mol_type = other RNA
ctcccagtgtacgtcccatcgttttagagctagaaatagcaagttaaaataaggctagtccgttatcaacttgaaaaagtggcaccgagtcggtgctttt

SEQ ID NO: 343 - RNA, 100 nt, synthetic construct
  source 1..100: mol_type = other RNA
tggggccactcgatccttgagttttagagctagaaatagcaagttaaaataaggctagtccgttatcaacttgaaaaagtggcaccgagtcggtgctttt

SEQ ID NO: 344 - RNA, 100 nt, synthetic construct; identical to SEQ ID NO: 297
  source 1..100: mol_type = other RNA

SEQ ID NO: 345 - RNA, 100 nt, synthetic construct
  source 1..100: mol_type = other RNA
tcgctgaccgtgaacgatacgttttagagctagaaatagcaagttaaaataaggctagtccgttatcaacttgaaaaagtggcaccgagtcggtgctttt

SEQ ID NO: 346 - RNA, 100 nt, synthetic construct
  source 1..100: mol_type = other RNA
gggtcgatctccaccacctggttttagagctagaaatagcaagttaaaataaggctagtccgttatcaacttgaaaaagtggcaccgagtcggtgctttt

SEQ ID NO: 347 - RNA, 100 nt, synthetic construct
  source 1..100: mol_type = other RNA
ggagtttagccagtccaactgttttagagctagaaatagcaagttaaaataaggctagtccgttatcaacttgaaaaagtggcaccgagtcggtgctttt

SEQ ID NO: 348 - RNA, 100 nt, synthetic construct
  source 1..100: mol_type = other RNA
gagttggactggctaaactcgttttagagctagaaatagcaagttaaaataaggctagtccgttatcaacttgaaaaagtggcaccgagtcggtgctttt

SEQ ID NO: 349 - RNA, 100 nt, synthetic construct
  source 1..100: mol_type = other RNA
caccaaggccatcgagagctgttttagagctagaaatagcaagttaaaataaggctagtccgttatcaacttgaaaaagtggcaccgagtcggtgctttt

SEQ ID NO: 350 - RNA, 100 nt, synthetic construct
  source 1..100: mol_type = other RNA
atccacatcgaggctcctcggttttagagctagaaatagcaagttaaaataaggctagtccgttatcaacttgaaaaagtggcaccgagtcggtgctttt

SEQ ID NO: 351 - RNA, 100 nt, synthetic construct
  source 1..100: mol_type = other RNA
gagcccagaacttaacttcggttttagagctagaaatagcaagttaaaataaggctagtccgttatcaacttgaaaaagtggcaccgagtcggtgctttt

SEQ ID NO: 352 - RNA, 100 nt, synthetic construct
  source 1..100: mol_type = other RNA
catccacatcgaggctcctcgttttagagctagaaatagcaagttaaaataaggctagtccgttatcaacttgaaaaagtggcaccgagtcggtgctttt

SEQ ID NO: 353 - RNA, 100 nt, synthetic construct
  source 1..100: mol_type = other RNA
ctacgccgacctcatcaccagttttagagctagaaatagcaagttaaaataaggctagtccgttatcaacttgaaaaagtggcaccgagtcggtgctttt

SEQ ID NO: 354 - RNA, 100 nt, synthetic construct
  source 1..100: mol_type = other RNA
gctccctggacacccattccgttttagagctagaaatagcaagttaaaataaggctagtccgttatcaacttgaaaaagtggcaccgagtcggtgctttt

SEQ ID NO: 355 - RNA, 100 nt, synthetic construct
  source 1..100: mol_type = other RNA
tcccaaatcccagtgcacccgttttagagctagaaatagcaagttaaaataaggctagtccgttatcaacttgaaaaagtggcaccgagtcggtgctttt

SEQ ID NO: 356 - RNA, 100 nt, synthetic construct
  source 1..100: mol_type = other RNA
ttcgaagaccttctcacatcgttttagagctagaaatagcaagttaaaataaggctagtccgttatcaacttgaaaaagtggcaccgagtcggtgctttt

SEQ ID NO: 357 - RNA, 100 nt, synthetic construct
  source 1..100: mol_type = other RNA
tcgaagaccttctcacatccgttttagagctagaaatagcaagttaaaataaggctagtccgttatcaacttgaaaaagtggcaccgagtcggtgctttt

SEQ ID NO: 358 - RNA, 100 nt, synthetic construct
  source 1..100: mol_type = other RNA
caagtggcccggatgtgagagttttagagctagaaatagcaagttaaaataaggctagtccgttatcaacttgaaaaagtggcaccgagtcggtgctttt

SEQ ID NO: 359 - RNA, 100 nt, synthetic construct
  source 1..100: mol_type = other RNA
gaaggtcttcgaagagccaggttttagagctagaaatagcaagttaaaataaggctagtccgttatcaacttgaaaaagtggcaccgagtcggtgctttt

SEQ ID NO: 360 - RNA, 100 nt, synthetic construct
  source 1..100: mol_type = other RNA
actgtaccatctctctctgggttttagagctagaaatagcaagttaaaataaggctagtccgttatcaacttgaaaaagtggcaccgagtcggtgctttt

SEQ ID NO: 361 - RNA, 100 nt, synthetic construct
  source 1..100: mol_type = other RNA
ggaccatcttctggatgagagttttagagctagaaatagcaagttaaaataaggctagtccgttatcaacttgaaaaagtggcaccgagtcggtgctttt

SEQ ID NO: 362 - RNA, 100 nt, synthetic construct
  source 1..100: mol_type = other RNA
tctctcagctggtacacggcgttttagagctagaaatagcaagttaaaataaggctagtccgttatcaacttgaaaaagtggcaccgagtcggtgctttt

SEQ ID NO: 363 - RNA, 100 nt, synthetic construct
  source 1..100: mol_type = other RNA
ctcgaccagcttgacatcacgttttagagctagaaatagcaagttaaaataaggctagtccgttatcaacttgaaaaagtggcaccgagtcggtgctttt

SEQ ID NO: 364 - RNA, 100 nt, synthetic construct
  source 1..100: mol_type = other RNA
aagttcctgtgatgtcaagcgttttagagctagaaatagcaagttaaaataaggctagtccgttatcaacttgaaaaagtggcaccgagtcggtgctttt

SEQ ID NO: 365 - RNA, 100 nt, synthetic construct
  source 1..100: mol_type = other RNA
ttcggaacccaatcactgacgttttagagctagaaatagcaagttaaaataaggctagtccgttatcaacttgaaaaagtggcaccgagtcggtgctttt

SEQ ID NO: 366 - RNA, 100 nt, synthetic construct
  source 1..100: mol_type = other RNA
gattaaacccggccactttcgttttagagctagaaatagcaagttaaaataaggctagtccgttatcaacttgaaaaagtggcaccgagtcggtgctttt

SEQ ID NO: 367 - RNA, 100 nt, synthetic construct
  source 1..100: mol_type = other RNA
acccggccactttcaggagggttttagagctagaaatagcaagttaaaataaggctagtccgttatcaacttgaaaaagtggcaccgagtcggtgctttt

SEQ ID NO: 368 - RNA, 100 nt, synthetic construct; identical to SEQ ID NO: 295
  source 1..100: mol_type = other RNA

SEQ ID NO: 369 - RNA, 100 nt, synthetic construct
  source 1..100: mol_type = other RNA
taaacccggccactttcagggttttagagctagaaatagcaagttaaaataaggctagtccgttatcaacttgaaaaagtggcaccgagtcggtgctttt

SEQ ID NO: 370 - RNA, 100 nt, synthetic construct
  source 1..100: mol_type = other RNA
gaacaaggtgttcccacccggttttagagctagaaatagcaagttaaaataaggctagtccgttatcaacttgaaaaagtggcaccgagtcggtgctttt

SEQ ID NO: 371 - RNA, 100 nt, synthetic construct
  source 1..100: mol_type = other RNA
cgggtgggaacaccttgttcgttttagagctagaaatagcaagttaaaataaggctagtccgttatcaacttgaaaaagtggcaccgagtcggtgctttt

SEQ ID NO: 372 - RNA, 100 nt, synthetic construct
  source 1..100: mol_type = other RNA
tcaaacacagcgacctcggggttttagagctagaaatagcaagttaaaataaggctagtccgttatcaacttgaaaaagtggcaccgagtcggtgctttt

SEQ ID NO: 373 - RNA, 100 nt, synthetic construct
  source 1..100: mol_type = other RNA
cgtagaactggacttgacaggttttagagctagaaatagcaagttaaaataaggctagtccgttatcaacttgaaaaagtggcaccgagtcggtgctttt

SEQ ID NO: 374 - RNA, 100 nt, synthetic construct
  source 1..100: mol_type = other RNA
atgacgagtggacccaggatgttttagagctagaaatagcaagttaaaataaggctagtccgttatcaacttgaaaaagtggcaccgagtcggtgctttt

SEQ ID NO: 375 - RNA, 100 nt, synthetic construct
  source 1..100: mol_type = other RNA
gctgtcaagtccagttctacgttttagagctagaaatagcaagttaaaataaggctagtccgttatcaacttgaaaaagtggcaccgagtcggtgctttt

SEQ ID NO: 376 - RNA, 100 nt, synthetic construct
  source 1..100: mol_type = other RNA
tgacgagtggacccaggatagttttagagctagaaatagcaagttaaaataaggctagtccgttatcaacttgaaaaagtggcaccgagtcggtgctttt

SEQ ID NO: 377 - RNA, 100 nt, synthetic construct
  source 1..100: mol_type = other RNA
cttgacagcggaagtggttggttttagagctagaaatagcaagttaaaataaggctagtccgttatcaacttgaaaaagtggcaccgagtcggtgctttt

SEQ ID NO: 378 - RNA, 100 nt, synthetic construct
  source 1..100: mol_type = other RNA
accactatgacctgctacaggttttagagctagaaatagcaagttaaaataaggctagtccgttatcaacttgaaaaagtggcaccgagtcggtgctttt

SEQ ID NO: 379 - RNA, 100 nt, synthetic construct
  source 1..100: mol_type = other RNA
cattttcaatagagacccccgttttagagctagaaatagcaagttaaaataaggctagtccgttatcaacttgaaaaagtggcaccgagtcggtgctttt

SEQ ID NO: 380 - RNA, 100 nt, synthetic construct
  source 1..100: mol_type = other RNA
gggtccacgacgtccatcccgttttagagctagaaatagcaagttaaaataaggctagtccgttatcaacttgaaaaagtggcaccgagtcggtgctttt

SEQ ID NO: 381 - RNA, 100 nt, synthetic construct
  source 1..100: mol_type = other RNA
ggtccacgacgtccatccctgttttagagctagaaatagcaagttaaaataaggctagtccgttatcaacttgaaaaagtggcaccgagtcggtgctttt

SEQ ID NO: 382 - RNA, 100 nt, synthetic construct
  source 1..100: mol_type = other RNA
gtccacgacgtccatccctggttttagagctagaaatagcaagttaaaataaggctagtccgttatcaacttgaaaaagtggcaccgagtcggtgctttt

SEQ ID NO: 383 - RNA, 100 nt, synthetic construct
  source 1..100: mol_type = other RNA
tccacgacgtccatccctgggttttagagctagaaatagcaagttaaaataaggctagtccgttatcaacttgaaaaagtggcaccgagtcggtgctttt

SEQ ID NO: 384 - RNA, 100 nt, synthetic construct
  source 1..100: mol_type = other RNA
ccaacatcgtggcctaccatgttttagagctagaaatagcaagttaaaataaggctagtccgttatcaacttgaaaaagtggcaccgagtcggtgctttt

SEQ ID NO: 385 - RNA, 100 nt, synthetic construct
  source 1..100: mol_type = other RNA
cccatggtaggccacgatgtgttttagagctagaaatagcaagttaaaataaggctagtccgttatcaacttgaaaaagtggcaccgagtcggtgctttt

SEQ ID NO: 386 - RNA, 100 nt, synthetic construct
  source 1..100: mol_type = other RNA
taggatcttcgtggctgtctgttttagagctagaaatagcaagttaaaataaggctagtccgttatcaacttgaaaaagtggcaccgagtcggtgctttt

SEQ ID NO: 387 - RNA, 100 nt, synthetic construct
  source 1..100: mol_type = other RNA
accaggttgtagaagttgacgttttagagctagaaatagcaagttaaaataaggctagtccgttatcaacttgaaaaagtggcaccgagtcggtgctttt

SEQ ID NO: 388 - RNA, 100 nt, synthetic construct
  source 1..100: mol_type = other RNA
aagttgacaggcagtgccatgttttagagctagaaatagcaagttaaaataaggctagtccgttatcaacttgaaaaagtggcaccgagtcggtgctttt

SEQ ID NO: 389 - RNA, 100 nt, synthetic construct
  source 1..100: mol_type = other RNA
gcatccagcaacataagcgggttttagagctagaaatagcaagttaaaataaggctagtccgttatcaacttgaaaaagtggcaccgagtcggtgctttt

SEQ ID NO: 390 - RNA, 100 nt, synthetic construct
  source 1..100: mol_type = other RNA
taactttattgacccccagcgttttagagctagaaatagcaagttaaaataaggctagtccgttatcaacttgaaaaagtggcaccgagtcggtgctttt

SEQ ID NO: 391 - RNA, 100 nt, synthetic construct
  source 1..100: mol_type = other RNA
caacccctcccaaagatggagttttagagctagaaatagcaagttaaaataaggctagtccgttatcaacttgaaaaagtggcaccgagtcggtgctttt

SEQ ID NO: 392 - RNA, 100 nt, synthetic construct
  source 1..100: mol_type = other RNA
ttctacaacctggtgatgtcgttttagagctagaaatagcaagttaaaataaggctagtccgttatcaacttgaaaaagtggcaccgagtcggtgctttt

SEQ ID NO: 393 - RNA, 100 nt, synthetic construct
  source 1..100: mol_type = other RNA
gcctgtcaacttctacaaccgttttagagctagaaatagcaagttaaaataaggctagtccgttatcaacttgaaaaagtggcaccgagtcggtgctttt

SEQ ID NO: 394 - RNA, 100 nt, synthetic construct
  source 1..100: mol_type = other RNA
aagtcaacttcaatgtcggagttttagagctagaaatagcaagttaaaataaggctagtccgttatcaacttgaaaaagtggcaccgagtcggtgctttt

SEQ ID NO: 395 - RNA, 100 nt, synthetic construct
  source 1..100: mol_type = other RNA
cgtgagtaaacctgaatcttgttttagagctagaaatagcaagttaaaataaggctagtccgttatcaacttgaaaaagtggcaccgagtcggtgctttt

SEQ ID NO: 396 - RNA, 100 nt, synthetic construct
  source 1..100: mol_type = other RNA
acagcccaagatagttaagtgttttagagctagaaatagcaagttaaaataaggctagtccgttatcaacttgaaaaagtggcaccgagtcggtgctttt

SEQ ID NO: 397 - RNA, 100 nt, synthetic construct
  source 1..100: mol_type = other RNA
attgtttagagctacccagcgttttagagctagaaatagcaagttaaaataaggctagtccgttatcaacttgaaaaagtggcaccgagtcggtgctttt

SEQ ID NO: 398 - RNA, 100 nt, synthetic construct
  source 1..100: mol_type = other RNA
cttaccccacttaactatctgttttagagctagaaatagcaagttaaaataaggctagtccgttatcaacttgaaaaagtggcaccgagtcggtgctttt

SEQ ID NO: 399 - RNA, 100 nt, synthetic construct
  source 1..100: mol_type = other RNA
cgaacatctcaagaaggtatgttttagagctagaaatagcaagttaaaataaggctagtccgttatcaacttgaaaaagtggcaccgagtcggtgctttt

SEQ ID NO: 400 - RNA, 100 nt, synthetic construct
  source 1..100: mol_type = other RNA
ttaccccacttaactatcttgttttagagctagaaatagcaagttaaaataaggctagtccgttatcaacttgaaaaagtggcaccgagtcggtgctttt

SEQ ID NO: 401 - RNA, 100 nt, synthetic construct
  source 1..100: mol_type = other RNA
ccaatccagccagaaagtacgttttagagctagaaatagcaagttaaaataaggctagtccgttatcaacttgaaaaagtggcaccgagtcggtgctttt

SEQ ID NO: 402 - RNA, 100 nt, synthetic construct
  source 1..100: mol_type = other RNA
tgtgctagacatgaggtctagttttagagctagaaatagcaagttaaaataaggctagtccgttatcaacttgaaaaagtggcaccgagtcggtgctttt

SEQ ID NO: 403 - RNA, 100 nt, synthetic construct
  source 1..100: mol_type = other RNA
ggagaatgacgagtggacccgttttagagctagaaatagcaagttaaaataaggctagtccgttatcaacttgaaaaagtggcaccgagtcggtgctttt

SEQ ID NO: 404 - RNA, 100 nt, synthetic construct
  source 1..100: mol_type = other RNA
ggcgccctggccagtcgtctgttttagagctagaaatagcaagttaaaataaggctagtccgttatcaacttgaaaaagtggcaccgagtcggtgctttt

SEQ ID NO: 405 - RNA, 100 nt, synthetic construct
  source 1..100: mol_type = other RNA
cagggttctggatatctgtggttttagagctagaaatagcaagttaaaataaggctagtccgttatcaacttgaaaaagtggcaccgagtcggtgctttt

SEQ ID NO: 406 - RNA, 100 nt, synthetic construct
  source 1..100: mol_type = other RNA
ggccacggagcgagacatctgttttagagctagaaatagcaagttaaaataaggctagtccgttatcaacttgaaaaagtggcaccgagtcggtgctttt

SEQ ID NO: 407 - RNA, 100 nt, synthetic construct
  source 1..100: mol_type = other RNA
gggaaagacccagcatccgtgttttagagctagaaatagcaagttaaaataaggctagtccgttatcaacttgaaaaagtggcaccgagtcggtgctttt

SEQ ID NO: 408 - RNA, 100 nt, synthetic construct
  source 1..100: mol_type = other RNA
ggcccagactgagcacgtgagttttagagctagaaatagcaagttaaaataaggctagtccgttatcaacttgaaaaagtggcaccgagtcggtgctttt

SEQ ID NO: 409 - RNA, 100 nt, synthetic construct; identical to SEQ ID NO: 294
  source 1..100: mol_type = other RNA

SEQ ID NO: 410 - RNA, 100 nt, synthetic construct; identical to SEQ ID NO: 291
  source 1..100: mol_type = other RNA

SEQ ID NO: 411 - RNA, 100 nt, synthetic construct
  source 1..100: mol_type = other RNA
gggtggggggagtttgctccgttttagagctagaaatagcaagttaaaataaggctagtccgttatcaacttgaaaaagtggcaccgagtcggtgctttt

SEQ ID NO: 412 - RNA, 100 nt, synthetic construct
  source 1..100: mol_type = other RNA
tggtaatgatggcttcaacagttttagagctagaaatagcaagttaaaataaggctagtccgttatcaacttgaaaaagtggcaccgagtcggtgctttt

SEQ ID NO: 413 - RNA, 100 nt, synthetic construct
  source 1..100: mol_type = other RNA
gaacacaaagcatagactgcgttttagagctagaaatagcaagttaaaataaggctagtccgttatcaacttgaaaaagtggcaccgagtcggtgctttt

SEQ ID NO: 414 - RNA, 100 nt, synthetic construct
  source 1..100: mol_type = other RNA
gtgttcatctttggttttgtgttttagagctagaaatagcaagttaaaataaggctagtccgttatcaacttgaaaaagtggcaccgagtcggtgctttt

SEQ ID NO: 415 - RNA, 100 nt, synthetic construct
  source 1..100: mol_type = other RNA
tgtccccgcacggagcccacgttttagagctagaaatagcaagttaaaataaggctagtccgttatcaacttgaaaaagtggcaccgagtcggtgctttt

SEQ ID NO: 416 - RNA, 100 nt, synthetic construct
  source 1..100: mol_type = other RNA
gggggcctacaccttccaacgttttagagctagaaatagcaagttaaaataaggctagtccgttatcaacttgaaaaagtggcaccgagtcggtgctttt

SEQ ID NO: 417 - RNA, 100 nt, synthetic construct
  source 1..100: mol_type = other RNA
tgtttggataggggtacacggttttagagctagaaatagcaagttaaaataaggctagtccgttatcaacttgaaaaagtggcaccgagtcggtgctttt

SEQ ID NO: 418 - RNA, 100 nt, synthetic construct
  source 1..100: mol_type = other RNA
agcccaagggaagtcaccgcgttttagagctagaaatagcaagttaaaataaggctagtccgttatcaacttgaaaaagtggcaccgagtcggtgctttt

SEQ ID NO: 419 - RNA, 100 nt, synthetic construct
  source 1..100: mol_type = other RNA
gcgggccaacttcactctgcgttttagagctagaaatagcaagttaaaataaggctagtccgttatcaacttgaaaaagtggcaccgagtcggtgctttt

SEQ ID NO: 420 - RNA, 100 nt, synthetic construct
  source 1..100: mol_type = other RNA
tcaagctttaccccaccatagttttagagctagaaatagcaagttaaaataaggctagtccgttatcaacttgaaaaagtggcaccgagtcggtgctttt

SEQ ID NO: 421 - RNA, 100 nt, synthetic construct; identical to SEQ ID NO: 68
  source 1..100: mol_type = other RNA

SEQ ID NO: 422 - RNA, 100 nt, synthetic construct; identical to SEQ ID NO: 69
  source 1..100: mol_type = other RNA

SEQ ID NO: 423 - RNA, 100 nt, synthetic construct; identical to SEQ ID NO: 71
  source 1..100: mol_type = other RNA

SEQ ID NO: 424 - RNA, 100 nt, synthetic construct
  source 1..100: mol_type = other RNA
gggaacccagcgagtgaagagttttagagctagaaatagcaagttaaaataaggctagtccgttatcaacttgaaaaagtggcaccgagtcggtgctttt

SEQ ID NO: 425 - RNA, 100 nt, synthetic construct
  source 1..100: mol_type = other RNA
gagccacattaaccggccctgttttagagctagaaatagcaagttaaaataaggctagtccgttatcaacttgaaaaagtggcaccgagtcggtgctttt

SEQ ID NO: 426 - RNA, 100 nt, synthetic construct
  source 1..100: mol_type = other RNA
ggtgagggaggagagatgccgttttagagctagaaatagcaagttaaaataaggctagtccgttatcaacttgaaaaagtggcaccgagtcggtgctttt

SEQ ID NO: 427 - RNA, 100 nt, synthetic construct; identical to SEQ ID NO: 296
  source 1..100: mol_type = other RNA

SEQ ID NO: 428 - RNA, 100 nt, synthetic construct
  source 1..100: mol_type = other RNA
gaagttgacttactgaagaagttttagagctagaaatagcaagttaaaataaggctagtccgttatcaacttgaaaaagtggcaccgagtcggtgctttt

SEQ ID NO: 429 - RNA, 100 nt, synthetic construct
  source 1..100: mol_type = other RNA
gctactctctctttctggccgttttagagctagaaatagcaagttaaaataaggctagtccgttatcaacttgaaaaagtggcaccgagtcggtgctttt

SEQ ID NO: 430 - RNA, 100 nt, synthetic construct
  source 1..100: mol_type = other RNA
ggcagaaaccctggtggtcggttttagagctagaaatagcaagttaaaataaggctagtccgttatcaacttgaaaaagtggcaccgagtcggtgctttt

SEQ ID NO: 431 - RNA, 100 nt, synthetic construct
  source 1..100: mol_type = other RNA
ggatttcctcctcgaccaccgttttagagctagaaatagcaagttaaaataaggctagtccgttatcaacttgaaaaagtggcaccgagtcggtgctttt

SEQ ID NO: 432 - RNA, 100 nt, synthetic construct
  source 1..100: mol_type = other RNA
gggtattattgatgctattcgttttagagctagaaatagcaagttaaaataaggctagtccgttatcaacttgaaaaagtggcaccgagtcggtgctttt

SEQ ID NO: 433 - RNA, 100 nt, synthetic construct
  source 1..100: mol_type = other RNA
ggtacctatcgattgtcagggttttagagctagaaatagcaagttaaaataaggctagtccgttatcaacttgaaaaagtggcaccgagtcggtgctttt

SEQ ID NO: 434 - RNA, 100 nt, synthetic construct
  source 1..100: mol_type = other RNA
gacattaaagatagtcatctgttttagagctagaaatagcaagttaaaataaggctagtccgttatcaacttgaaaaagtggcaccgagtcggtgctttt

SEQ ID NO: 435 - RNA, 100 nt, synthetic construct
  source 1..100: mol_type = other RNA
gtagagcggaggcaggaggcgttttagagctagaaatagcaagttaaaataaggctagtccgttatcaacttgaaaaagtggcaccgagtcggtgctttt

SEQ ID NO: 436 - RNA, 100 nt, synthetic construct
  source 1..100: mol_type = other RNA
gaggttcacttgatttccacgttttagagctagaaatagcaagttaaaataaggctagtccgttatcaacttgaaaaagtggcaccgagtcggtgctttt

SEQ ID NO: 437 - RNA, 100 nt, synthetic construct; identical to SEQ ID NO: 314
  source 1..100: mol_type = other RNA

SEQ ID NO: 438 - RNA, 100 nt, synthetic construct
  source 1..100: mol_type = other RNA
gcacaaggctcagctgaaccgttttagagctagaaatagcaagttaaaataaggctagtccgttatcaacttgaaaaagtggcaccgagtcggtgctttt

SEQ ID NO: 439 - RNA, 100 nt, synthetic construct
  source 1..100: mol_type = other RNA
gataactacaccgaggaaatgttttagagctagaaatagcaagttaaaataaggctagtccgttatcaacttgaaaaagtggcaccgagtcggtgctttt

SEQ ID NO: 440 - RNA, 100 nt, synthetic construct
  source 1..100: mol_type = other RNA
gccgtggcaaactggtacttgttttagagctagaaatagcaagttaaaataaggctagtccgttatcaacttgaaaaagtggcaccgagtcggtgctttt

SEQ ID NO: 441 - RNA, 100 nt, synthetic construct
  source 1..100: mol_type = other RNA
gaagatgatggagtagatgggttttagagctagaaatagcaagttaaaataaggctagtccgttatcaacttgaaaaagtggcaccgagtcggtgctttt

SEQ ID NO: 442 - RNA, 100 nt, synthetic construct
  source 1..100: mol_type = other RNA
ggggcagctccggcgctcctgttttagagctagaaatagcaagttaaaataaggctagtccgttatcaacttgaaaaagtggcaccgagtcggtgctttt

SEQ ID NO: 443 - RNA, 100 nt, synthetic construct
  source 1..100: mol_type = other RNA
gctgaccccgctgggcaggcgttttagagctagaaatagcaagttaaaataaggctagtccgttatcaacttgaaaaagtggcaccgagtcggtgctttt

SEQ ID NO: 444 - RNA, 100 nt, synthetic construct
  source 1..100: mol_type = other RNA
gagggctcaccagaggtagggttttagagctagaaatagcaagttaaaataaggctagtccgttatcaacttgaaaaagtggcaccgagtcggtgctttt

SEQ ID NO: 445 - RNA, 100 nt, synthetic construct
  source 1..100: mol_type = other RNA
gctgtttctgcagccgctttgttttagagctagaaatagcaagttaaaataaggctagtccgttatcaacttgaaaaagtggcaccgagtcggtgctttt

SEQ ID NO: 446 - RNA, 100 nt, synthetic construct
  source 1..100: mol_type = other RNA
ggtcccggtggtgtgggcccgttttagagctagaaatagcaagttaaaataaggctagtccgttatcaacttgaaaaagtggcaccgagtcggtgctttt

SEQ ID NO: 447 - RNA, 100 nt, synthetic construct
  source 1..100: mol_type = other RNA
ggtggtgtgggcccaggagggttttagagctagaaatagcaagttaaaataaggctagtccgttatcaacttgaaaaagtggcaccgagtcggtgctttt

SEQ ID NO: 448 - RNA, 100 nt, synthetic construct
  source 1..100: mol_type = other RNA
gcgtgacttccacatgagcggttttagagctagaaatagcaagttaaaataaggctagtccgttatcaacttgaaaaagtggcaccgagtcggtgctttt

SEQ ID NO: 449 - RNA, 100 nt, synthetic construct; identical to SEQ ID NO: 300
  source 1..100: mol_type = other RNA

SEQ ID NO: 450 - RNA, 100 nt, synthetic construct; identical to SEQ ID NO: 292
  source 1..100: mol_type = other RNA

SEQ ID NO: 451 - RNA, 100 nt, synthetic construct
  source 1..100: mol_type = other RNA
ggaaacttggccactctatggttttagagctagaaatagcaagttaaaataaggctagtccgttatcaacttgaaaaagtggcaccgagtcggtgctttt

SEQ ID NO: 452 - RNA, 100 nt, synthetic construct
  source 1..100: mol_type = other RNA
ggcaccaactggatggatcagttttagagctagaaatagcaagttaaaataaggctagtccgttatcaacttgaaaaagtggcaccgagtcggtgctttt

SEQ ID NO: 453 - RNA, 100 nt, synthetic construct
  source 1..100: mol_type = other RNA
gtctccctgatccatccagtgttttagagctagaaatagcaagttaaaataaggctagtccgttatcaacttgaaaaagtggcaccgagtcggtgctttt

SEQ ID NO: 454 - RNA, 100 nt, synthetic construct
  source 1..100: mol_type = other RNA
gtttgcgactctgacagagcgttttagagctagaaatagcaagttaaaataaggctagtccgttatcaacttgaaaaagtggcaccgagtcggtgctttt

SEQ ID NO: 455 - RNA, 100 nt, synthetic construct
  source 1..100: mol_type = other RNA
ggtttcaccgagacctcagtgttttagagctagaaatagcaagttaaaataaggctagtccgttatcaacttgaaaaagtggcaccgagtcggtgctttt

SEQ ID NO: 456 - RNA, 100 nt, synthetic construct
  source 1..100: mol_type = other RNA
gatttctatgacctgtatgggttttagagctagaaatagcaagttaaaataaggctagtccgttatcaacttgaaaaagtggcaccgagtcggtgctttt

SEQ ID NO: 457 - RNA, 100 nt, synthetic construct
  source 1..100: mol_type = other RNA
gagaatcaaaatcggtgaatgttttagagctagaaatagcaagttaaaataaggctagtccgttatcaacttgaaaaagtggcaccgagtcggtgctttt

SEQ ID NO: 458 - RNA, 100 nt, synthetic construct
  source 1..100: mol_type = other RNA
gacaccttcttccccagcccgttttagagctagaaatagcaagttaaaataaggctagtccgttatcaacttgaaaaagtggcaccgagtcggtgctttt

SEQ ID NO: 459 - RNA, 100 nt, synthetic construct
  source 1..100: mol_type = other RNA
gctggtacacggcagggtcagttttagagctagaaatagcaagttaaaataaggctagtccgttatcaacttgaaaaagtggcaccgagtcggtgctttt

SEQ ID NO: 460 - RNA, 100 nt, synthetic construct; identical to SEQ ID NO: 370
  source 1..100: mol_type = other RNA

SEQ ID NO: 461 - RNA, 100 nt, synthetic construct
  source 1..100: mol_type = other RNA
ggtgcacagtggggtcagcagttttagagctagaaatagcaagttaaaataaggctagtccgttatcaacttgaaaaagtggcaccgagtcggtgctttt

SEQ ID NO: 462 - RNA, 100 nt, synthetic construct
  source 1..100: mol_type = other RNA
gacgtcgtcgatccacttaggttttagagctagaaatagcaagttaaaataaggctagtccgttatcaacttgaaaaagtggcaccgagtcggtgctttt

SEQ ID NO: 463 - RNA, 100 nt, synthetic construct
  source 1..100: mol_type = other RNA
tcacccgtcaagacgtgttcgttttagagctagaaatagcaagttaaaataaggctagtccgttatcaacttgaaaaagtggcaccgagtcggtgctttt

SEQ ID NO: 464 - RNA, 100 nt, synthetic construct
  source 1..100: mol_type = other RNA
tgtaccagaacgtgtccacggttttagagctagaaatagcaagttaaaataaggctagtccgttatcaacttgaaaaagtggcaccgagtcggtgctttt

SEQ ID NO: 465 - RNA, 100 nt, synthetic construct
  source 1..100: mol_type = other RNA
acggacactgtgatcatcgtgttttagagctagaaatagcaagttaaaataaggctagtccgttatcaacttgaaaaagtggcaccgagtcggtgctttt

SEQ ID NO: 466 - RNA, 100 nt, synthetic construct
  source 1..100: mol_type = other RNA
ccaaccaggacgatcatacggttttagagctagaaatagcaagttaaaataaggctagtccgttatcaacttgaaaaagtggcaccgagtcggtgctttt

SEQ ID NO: 467 - RNA, 100 nt, synthetic construct
  source 1..100: mol_type = other RNA
tgtcactataggatcgcaaggttttagagctagaaatagcaagttaaaataaggctagtccgttatcaacttgaaaaagtggcaccgagtcggtgctttt

SEQ ID NO: 468 - RNA, 100 nt, synthetic construct
  source 1..100: mol_type = other RNA
ccttgcaagacgacttactcgttttagagctagaaatagcaagttaaaataaggctagtccgttatcaacttgaaaaagtggcaccgagtcggtgctttt

SEQ ID NO: 469 - RNA, 100 nt, synthetic construct
  source 1..100: mol_type = other RNA
gtgtccacaacgggttatctgttttagagctagaaatagcaagttaaaataaggctagtccgttatcaacttgaaaaagtggcaccgagtcggtgctttt

SEQ ID NO: 470 - RNA, 100 nt, synthetic construct
  source 1..100: mol_type = other RNA
ggagacaactgctactccgtgttttagagctagaaatagcaagttaaaataaggctagtccgttatcaacttgaaaaagtggcaccgagtcggtgctttt

SEQ ID NO: 471 - RNA, 100 nt, synthetic construct
  source 1..100: mol_type = other RNA
ttgtcctttccacgaactacgttttagagctagaaatagcaagttaaaataaggctagtccgttatcaacttgaaaaagtggcaccgagtcggtgctttt

SEQ ID NO: 472 - RNA, 100 nt, synthetic construct
  source 1..100: mol_type = other RNA
gaagtacggggctaccactggttttagagctagaaatagcaagttaaaataaggctagtccgttatcaacttgaaaaagtggcaccgagtcggtgctttt

SEQ ID NO: 473 - RNA, 100 nt, synthetic construct; identical to SEQ ID NO: 293
  source 1..100: mol_type = other RNA

SEQ ID NO: 474 - RNA, 100 nt, synthetic construct
  source 1..100: mol_type = other RNA
tgtcggagcctacaaacaaagttttagagctagaaatagcaagttaaaataaggctagtccgttatcaacttgaaaaagtggcaccgagtcggtgctttt

SEQ ID NO: 475 - RNA, 100 nt, synthetic construct
  source 1..100: mol_type = other RNA
atggacaccggtgtgattgagttttagagctagaaatagcaagttaaaataaggctagtccgttatcaacttgaaaaagtggcaccgagtcggtgctttt

SEQ ID NO: 476 - RNA, 100 nt, synthetic construct
  source 1..100: mol_type = other RNA
gcaaccacgaaagccacctcgttttagagctagaaatagcaagttaaaataaggctagtccgttatcaacttgaaaaagtggcaccgagtcggtgctttt

SEQ ID NO: 477 - RNA, 100 nt, synthetic construct
  source 1..100: mol_type = other RNA
gcttgtagtaggagtaatacgttttagagctagaaatagcaagttaaaataaggctagtccgttatcaacttgaaaaagtggcaccgagtcggtgctttt

SEQ ID NO: 478 - RNA, 100 nt, synthetic construct
  source 1..100: mol_type = other RNA
tcatggagcttgaacacctcgttttagagctagaaatagcaagttaaaataaggctagtccgttatcaacttgaaaaagtggcaccgagtcggtgctttt

SEQ ID NO: 479 - RNA, 100 nt, synthetic construct
  source 1..100: mol_type = other RNA
aaccagatcttgacctgcgtgttttagagctagaaatagcaagttaaaataaggctagtccgttatcaacttgaaaaagtggcaccgagtcggtgctttt

SEQ ID NO: 480 - RNA, 100 nt, synthetic construct
  source 1..100: mol_type = other RNA
caggaaagctttaccaattcgttttagagctagaaatagcaagttaaaataaggctagtccgttatcaacttgaaaaagtggcaccgagtcggtgctttt

SEQ ID NO: 481 - RNA, 100 nt, synthetic construct
  source 1..100: mol_type = other RNA
cggcacgacccggacaatatgttttagagctagaaatagcaagttaaaataaggctagtccgttatcaacttgaaaaagtggcaccgagtcggtgctttt

SEQ ID NO: 482 - RNA, 100 nt, synthetic construct
  source 1..100: mol_type = other RNA
ggtggacaagcggcagataggttttagagctagaaatagcaagttaaaataaggctagtccgttatcaacttgaaaaagtggcaccgagtcggtgctttt

SEQ ID NO: 483 - RNA, 100 nt, synthetic construct
  source 1..100: mol_type = other RNA
ataggagaagatgatgtatagttttagagctagaaatagcaagttaaaataaggctagtccgttatcaacttgaaaaagtggcaccgagtcggtgctttt

SEQ ID NO: 484 - RNA, 100 nt, synthetic construct
  source 1..100: mol_type = other RNA
gcatacagtgatttgatgaagttttagagctagaaatagcaagttaaaataaggctagtccgttatcaacttgaaaaagtggcaccgagtcggtgctttt

SEQ ID NO: 485 - RNA, 100 nt, synthetic construct
  source 1..100: mol_type = other RNA
gctgatgtagtcactcttgagttttagagctagaaatagcaagttaaaataaggctagtccgttatcaacttgaaaaagtggcaccgagtcggtgctttt

SEQ ID NO: 486 - RNA, 100 nt, synthetic construct
  source 1..100: mol_type = other RNA
gcaaaactcaaccctaccccgttttagagctagaaatagcaagttaaaataaggctagtccgttatcaacttgaaaaagtggcaccgagtcggtgctttt

SEQ ID NO: 487 - RNA, 100 nt, synthetic construct
  source 1..100: mol_type = other RNA
ctcgtctgataagacaacaggttttagagctagaaatagcaagttaaaataaggctagtccgttatcaacttgaaaaagtggcaccgagtcggtgctttt

SEQ ID NO: 488 - RNA, 100 nt, synthetic construct
  source 1..100: mol_type = other RNA
cttgccccacagggcagtaagttttagagctagaaatagcaagttaaaataaggctagtccgttatcaacttgaaaaagtggcaccgagtcggtgctttt

SEQ ID NO: 489 - DNA, 48 bp, synthetic construct
  source 1..48: mol_type = other DNA
catagcggtattacgcgagattacgatagccggacgaatgtcggtcgt

SEQ ID NO: 490 - DNA, 49 bp, synthetic construct
  source 1..49: mol_type = other DNA
catagcggtattacgcgagattacgaacattcgtccggctacctacgcc

SEQ ID NO: 491 - DNA, 28 bp, synthetic construct
  source 1..28: mol_type = other DNA
aatgatacggcgaccaccgagatctaca

SEQ ID NO: 492 - DNA, 26 bp, synthetic construct
  source 1..26: mol_type = other DNA
gtcggtcgtagttagatcggaagagc

SEQ ID NO: 493 - DNA, 28 bp, synthetic construct
  source 1..28: mol_type = other DNA
tacctacgccgcttaagatcggaagagc

SEQ ID NO: 494 - DNA, 29 bp, synthetic construct
  source 1..29: mol_type = other DNA
aatgatacggcgaccaccgagatctacac

SEQ ID NO: 495 - DNA, 39 bp, synthetic construct; identical to SEQ ID NO: 75
  source 1..39: mol_type = other DNA

SEQ ID NO: 496 - DNA, 39 bp, synthetic construct; identical to SEQ ID NO: 76
  source 1..39: mol_type = other DNA

SEQ ID NO: 497 - DNA, 78 bp, synthetic construct
  source 1..78: mol_type = other DNA
aatgatacggcgaccaccgagatctacacatatgcgcnnwnnwnnacactctttccctacacgacgctcttccgatct

SEQ ID NO: 498 - DNA, 78 bp, synthetic construct
  source 1..78: mol_type = other DNA
aatgatacggcgaccaccgagatctacactggtacagnnwnnwnnacactctttccctacacgacgctcttccgatct

SEQ ID NO: 499 - DNA, 78 bp, synthetic construct
  source 1..78: mol_type = other DNA
aatgatacggcgaccaccgagatctacacaaccgttcnnwnnwnnacactctttccctacacgacgctcttccgatct

SEQ ID NO: 500 - DNA, 78 bp, synthetic construct
  source 1..78: mol_type = other DNA
aatgatacggcgaccaccgagatctacactaaccggtnnwnnwnnacactctttccctacacgacgctcttccgatct

SEQ ID NO: 501 - DNA, 78 bp, synthetic construct
  source 1..78: mol_type = other DNA
aatgatacggcgaccaccgagatctacacgaacatcgnnwnnwnnacactctttccctacacgacgctcttccgatct

SEQ ID NO: 502 - DNA, 78 bp, synthetic construct
  source 1..78: mol_type = other DNA
aatgatacggcgaccaccgagatctacacccttgtagnnwnnwnnacactctttccctacacgacgctcttccgatct

SEQ ID NO: 503 - DNA, 78 bp, synthetic construct
  source 1..78: mol_type = other DNA
aatgatacggcgaccaccgagatctacactcaggcttnnwnnwnnacactctttccctacacgacgctcttccgatct

SEQ ID NO: 504 - DNA, 78 bp, synthetic construct
  source 1..78: mol_type = other DNA
aatgatacggcgaccaccgagatctacacgttctcgtnnwnnwnnacactctttccctacacgacgctcttccgatct

SEQ ID NO: 505 - DNA, 78 bp, synthetic construct
  source 1..78: mol_type = other DNA
aatgatacggcgaccaccgagatctacacagaacgagnnwnnwnnacactctttccctacacgacgctcttccgatct

SEQ ID NO: 506 - DNA, 78 bp, synthetic construct
  source 1..78: mol_type = other DNA
aatgatacggcgaccaccgagatctacactgcttccannwnnwnnacactctttccctacacgacgctcttccgatct

SEQ ID NO: 507 - DNA, 78 bp, synthetic construct
  source 1..78: mol_type = other DNA
aatgatacggcgaccaccgagatctacaccttcgactnnwnnwnnacactctttccctacacgacgctcttccgatct

SEQ ID NO: 508 - DNA, 78 bp, synthetic construct
  source 1..78: mol_type = other DNA
aatgatacggcgaccaccgagatctacaccacctgttnnwnnwnnacactctttccctacacgacgctcttccgatct

SEQ ID NO: 509 - DNA, 78 bp, synthetic construct
  source 1..78: mol_type = other DNA
aatgatacggcgaccaccgagatctacacatcacacgnnwnnwnnacactctttccctacacgacgctcttccgatct

SEQ ID NO: 510 - DNA, 78 bp, synthetic construct
  source 1..78: mol_type = other DNA
aatgatacggcgaccaccgagatctacacccgtaagannwnnwnnacactctttccctacacgacgctcttccgatct

SEQ ID NO: 511 - DNA, 78 bp, synthetic construct
  source 1..78: mol_type = other DNA
aatgatacggcgaccaccgagatctacactacgccttnnwnnwnnacactctttccctacacgacgctcttccgatct

SEQ ID NO: 512 - DNA, 78 bp, synthetic construct
  source 1..78: mol_type = other DNA
aatgatacggcgaccaccgagatctacaccgacgttannwnnwnnacactctttccctacacgacgctcttccgatct

SEQ ID NO: 513 - DNA, 78 bp, synthetic construct
  source 1..78: mol_type = other DNA
aatgatacggcgaccaccgagatctacacatgcacgannwnnwnnacactctttccctacacgacgctcttccgatct

SEQ ID NO: 514 - DNA, 78 bp, synthetic construct
  source 1..78: mol_type = other DNA
aatgatacggcgaccaccgagatctacaccctgattgnnwnnwnnacactctttccctacacgacgctcttccgatct

SEQ ID NO: 515 - DNA, 78 bp, synthetic construct
  source 1..78: mol_type = other DNA
aatgatacggcgaccaccgagatctacacgtaggagtnnwnnwnnacactctttccctacacgacgctcttccgatct

SEQ ID NO: 516 - DNA, 78 bp, synthetic construct
  source 1..78: mol_type = other DNA
aatgatacggcgaccaccgagatctacacactaggagnnwnnwnnacactctttccctacacgacgctcttccgatct

SEQ ID NO: 517 - DNA, 78 bp, synthetic construct
  source 1..78: mol_type = other DNA
aatgatacggcgaccaccgagatctacaccactagctnnwnnwnnacactctttccctacacgacgctcttccgatct

SEQ ID NO: 518 - DNA, 78 bp, synthetic construct
  source 1..78: mol_type = other DNA
aatgatacggcgaccaccgagatctacacacgacttgnnwnnwnnacactctttccctacacgacgctcttccgatct

SEQ ID NO: 519 - DNA, 78 bp, synthetic construct
  source 1..78: mol_type = other DNA
aatgatacggcgaccaccgagatctacaccgtgtgtannwnnwnnacactctttccctacacgacgctcttccgatct

SEQ ID NO: 520 - DNA, 78 bp, synthetic construct
  source 1..78: mol_type = other DNA
aatgatacggcgaccaccgagatctacacgttgacctnnwnnwnnacactctttccctacacgacgctcttccgatct

SEQ ID NO: 521 - DNA, 78 bp, synthetic construct
  source 1..78: mol_type = other DNA
aatgatacggcgaccaccgagatctacacactccatcnnwnnwnnacactctttccctacacgacgctcttccgatct

SEQ ID NO: 522 - DNA, 78 bp, synthetic construct
  source 1..78: mol_type = other DNA
aatgatacggcgaccaccgagatctacaccaatgtggnnwnnwnnacactctttccctacacgacgctcttccgatct

SEQ ID NO: 523 - DNA, 78 bp, synthetic construct
  source 1..78: mol_type = other DNA
aatgatacggcgaccaccgagatctacacttgcagacnnwnnwnnacactctttccctacacgacgctcttccgatct

SEQ ID NO: 524 - DNA, 78 bp, synthetic construct
  source 1..78: mol_type = other DNA
aatgatacggcgaccaccgagatctacaccagtccaannwnnwnnacactctttccctacacgacgctcttccgatct

SEQ ID NO: 525 - DNA, 78 bp, synthetic construct
  source 1..78: mol_type = other DNA
aatgatacggcgaccaccgagatctacacacgttcagnnwnnwnnacactctttccctacacgacgctcttccgatct

SEQ ID NO: 526 - DNA, 78 bp, synthetic construct
  source 1..78: mol_type = other DNA
aatgatacggcgaccaccgagatctacacaacgtctgnnwnnwnnacactctttccctacacgacgctcttccgatct

SEQ ID NO: 527 - DNA, 78 bp, synthetic construct
  source 1..78: mol_type = other DNA
aatgatacggcgaccaccgagatctacactatcggtcnnwnnwnnacactctttccctacacgacgctcttccgatct

SEQ ID NO: 528 - DNA, 78 bp, synthetic construct
  source 1..78: mol_type = other DNA
aatgatacggcgaccaccgagatctacaccgctctatnnwnnwnnacactctttccctacacgacgctcttccgatct

SEQ ID NO: 529 - DNA, 78 bp, synthetic construct
  source 1..78: mol_type = other DNA
aatgatacggcgaccaccgagatctacacgattgctcnnwnnwnnacactctttccctacacgacgctcttccgatct

SEQ ID NO: 530 - DNA, 78 bp, synthetic construct
  source 1..78: mol_type = other DNA
aatgatacggcgaccaccgagatctacacgatgtgtgnnwnnwnnacactctttccctacacgacgctcttccgatct

SEQ ID NO: 531 - DNA, 78 bp, synthetic construct
  source 1..78: mol_type = other DNA
aatgatacggcgaccaccgagatctacaccgcaatctnnwnnwnnacactctttccctacacgacgctcttccgatct

SEQ ID NO: 532 - DNA, 78 bp, synthetic construct
  source 1..78: mol_type = other DNA
aatgatacggcgaccaccgagatctacactggtagctnnwnnwnnacactctttccctacacgacgctcttccgatct

SEQ ID NO: 533 - DNA, 78 bp, synthetic construct
  source 1..78: mol_type = other DNA
aatgatacggcgaccaccgagatctacacgataggctnnwnnwnnacactctttccctacacgacgctcttccgatct

SEQ ID NO: 534 - DNA, 78 bp, synthetic construct
  source 1..78: mol_type = other DNA
aatgatacggcgaccaccgagatctacacagtggatcnnwnnwnnacactctttccctacacgacgctcttccgatct

SEQ ID NO: 535 - DNA, 78 bp, synthetic construct
  source 1..78: mol_type = other DNA
aatgatacggcgaccaccgagatctacacttggacgtnnwnnwnnacactctttccctacacgacgctcttccgatct

SEQ ID NO: 536 - DNA, 78 bp, synthetic construct
  source 1..78: mol_type = other DNA
aatgatacggcgaccaccgagatctacacatgacgtcnnwnnwnnacactctttccctacacgacgctcttccgatct

SEQ ID NO: 537 - DNA, 78 bp, synthetic construct
  source 1..78: mol_type = other DNA
aatgatacggcgaccaccgagatctacacgaagttggnnwnnwnnacactctttccctacacgacgctcttccgatct

SEQ ID NO: 538 - DNA, 78 bp, synthetic construct
  source 1..78: mol_type = other DNA
aatgatacggcgaccaccgagatctacaccataccacnnwnnwnnacactctttccctacacgacgctcttccgatct

SEQ ID NO: 539 - DNA, 78 bp, synthetic construct
  source 1..78: mol_type = other DNA
aatgatacggcgaccaccgagatctacacctgttgacnnwnnwnnacactctttccctacacgacgctcttccgatct

SEQ ID NO: 540 - DNA, 78 bp, synthetic construct
  source 1..78: mol_type = other DNA
aatgatacggcgaccaccgagatctacactggcatgtnnwnnwnnacactctttccctacacgacgctcttccgatct

SEQ ID NO: 541 - DNA, 78 bp, synthetic construct
  source 1..78: mol_type = other DNA
aatgatacggcgaccaccgagatctacacatcgccatnnwnnwnnacactctttccctacacgacgctcttccgatct

SEQ ID NO: 542 - DNA, 78 bp, synthetic construct
  source 1..78: mol_type = other DNA
aatgatacggcgaccaccgagatctacacttgcgaagnnwnnwnnacactctttccctacacgacgctcttccgatct

SEQ ID NO: 543 - DNA, 78 bp, synthetic construct
  source 1..78: mol_type = other DNA
aatgatacggcgaccaccgagatctacacagttcgtcnnwnnwnnacactctttccctacacgacgctcttccgatct

SEQ ID NO: 544 - DNA, 78 bp, synthetic construct
  source 1..78: mol_type = other DNA
aatgatacggcgaccaccgagatctacacgagcagtannwnnwnnacactctttccctacacgacgctcttccgatct

SEQ ID NO: 545 - DNA, 78 bp, synthetic construct
  source 1..78: mol_type = other DNA
aatgatacggcgaccaccgagatctacacacagctcannwnnwnnacactctttccctacacgacgctcttccgatct

SEQ ID NO: 546 - DNA, 78 bp, synthetic construct
  source 1..78: mol_type = other DNA
aatgatacggcgaccaccgagatctacacgatcgagtnnwnnwnnacactctttccctacacgacgctcttccgatct

SEQ ID NO: 547 - DNA, 78 bp, synthetic construct
  source 1..78: mol_type = other DNA
aatgatacggcgaccaccgagatctacacagcgtgttnnwnnwnnacactctttccctacacgacgctcttccgatct

SEQ ID NO: 548 - DNA, 78 bp, synthetic construct
  source 1..78: mol_type = other DNA
aatgatacggcgaccaccgagatctacacgttacgcannwnnwnnacactctttccctacacgacgctcttccgatct

SEQ ID NO: 549 - DNA, 78 bp, synthetic construct
  source 1..78: mol_type = other DNA
aatgatacggcgaccaccgagatctacactgaagacgnnwnnwnnacactctttccctacacgacgctcttccgatct

SEQ ID NO: 550 - DNA, 78 bp, synthetic construct
  source 1..78: mol_type = other DNA
aatgatacggcgaccaccgagatctacacactgaggtnnwnnwnnacactctttccctacacgacgctcttccgatct

SEQ ID NO: 551 - DNA, 78 bp, synthetic construct
  source 1..78: mol_type = other DNA
aatgatacggcgaccaccgagatctacaccggttgttnnwnnwnnacactctttccctacacgacgctcttccgatct

SEQ ID NO: 552 - DNA, 78 bp, synthetic construct
  source 1..78: mol_type = other DNA
aatgatacggcgaccaccgagatctacacgttgttcgnnwnnwnnacactctttccctacacgacgctcttccgatct

SEQ ID NO: 553 - DNA, 78 bp, synthetic construct
  source 1..78: mol_type = other DNA
aatgatacggcgaccaccgagatctacacgaaggaagnnwnnwnnacactctttccctacacgacgctcttccgatct

SEQ ID NO: 554 - DNA, 78 bp, synthetic construct
  source 1..78: mol_type = other DNA
aatgatacggcgaccaccgagatctacacagcacttcnnwnnwnnacactctttccctacacgacgctcttccgatct

SEQ ID NO: 555 - DNA, 78 bp, synthetic construct
  source 1..78: mol_type = other DNA
aatgatacggcgaccaccgagatctacacgtcatcgannwnnwnnacactctttccctacacgacgctcttccgatct

SEQ ID NO: 556 - DNA, 78 bp, synthetic construct
  source 1..78: mol_type = other DNA
aatgatacggcgaccaccgagatctacactgtgactgnnwnnwnnacactctttccctacacgacgctcttccgatct

SEQ ID NO: 557 - DNA, 78 bp, synthetic construct
  source 1..78: mol_type = other DNA
aatgatacggcgaccaccgagatctacaccaacacctnnwnnwnnacactctttccctacacgacgctcttccgatct

SEQ ID NO: 558 - DNA, 78 bp, synthetic construct
  source 1..78: mol_type = other DNA
aatgatacggcgaccaccgagatctacacatgcctgtnnwnnwnnacactctttccctacacgacgctcttccgatct

SEQ ID NO: 559 - DNA, 78 bp, synthetic construct
  source 1..78: mol_type = other DNA
aatgatacggcgaccaccgagatctacaccatggctannwnnwnnacactctttccctacacgacgctcttccgatct

SEQ ID NO: 560 - DNA, 78 bp, synthetic construct
  source 1..78: mol_type = other DNA
aatgatacggcgaccaccgagatctacacgtgaagtgnnwnnwnnacactctttccctacacgacgctcttccgatct

SEQ ID NO: 561 - DNA, 78 bp, synthetic construct
  source 1..78: mol_type = other DNA
aatgatacggcgaccaccgagatctacaccgttgcaannwnnwnnacactctttccctacacgacgctcttccgatct

SEQ ID NO: 562 - DNA, 78 bp, synthetic construct
  source 1..78: mol_type = other DNA
aatgatacggcgaccaccgagatctacacatccggtannwnnwnnacactctttccctacacgacgctcttccgatct

SEQ ID NO: 563 - DNA, 78 bp, synthetic construct
  source 1..78: mol_type = other DNA
aatgatacggcgaccaccgagatctacacgcgtcattnnwnnwnnacactctttccctacacgacgctcttccgatct

SEQ ID NO: 564 - DNA, 78 bp, synthetic construct
  source 1..78: mol_type = other DNA
aatgatacggcgaccaccgagatctacacgcacaactnnwnnwnnacactctttccctacacgacgctcttccgatct

SEQ ID NO: 565 - DNA, 78 bp, synthetic construct
  source 1..78: mol_type = other DNA
aatgatacggcgaccaccgagatctacacgattaccgnnwnnwnnacactctttccctacacgacgctcttccgatct

SEQ ID NO: 566 - DNA, 78 bp, synthetic construct
  source 1..78: mol_type = other DNA
aatgatacggcgaccaccgagatctacacaccacgatnnwnnwnnacactctttccctacacgacgctcttccgatct

SEQ ID NO: 567 - DNA, 78 bp, synthetic construct
  source 1..78: mol_type = other DNA
aatgatacggcgaccaccgagatctacacgtcgaagannwnnwnnacactctttccctacacgacgctcttccgatct

SEQ ID NO: 568 - DNA, 78 bp, synthetic construct
  source 1..78: mol_type = other DNA
aatgatacggcgaccaccgagatctacacccttgatcnnwnnwnnacactctttccctacacgacgctcttccgatct

SEQ ID NO: 569 - DNA, 78 bp, synthetic construct
  source 1..78: mol_type = other DNA
aatgatacggcgaccaccgagatctacacaagcactgnnwnnwnnacactctttccctacacgacgctcttccgatct

SEQ ID NO: 570 - DNA, 78 bp, synthetic construct
  source 1..78: mol_type = other DNA
aatgatacggcgaccaccgagatctacacttcgttggnnwnnwnnacactctttccctacacgacgctcttccgatct

SEQ ID NO: 571 - DNA, 78 bp, synthetic construct
  source 1..78: mol_type = other DNA
aatgatacggcgaccaccgagatctacactcgctgttnnwnnwnnacactctttccctacacgacgctcttccgatct

SEQ ID NO: 572 - DNA, 78 bp, synthetic construct
  source 1..78: mol_type = other DNA
aatgatacggcgaccaccgagatctacacgaatccgannwnnwnnacactctttccctacacgacgctcttccgatct

SEQ ID NO: 573 - DNA, 78 bp, synthetic construct
  source 1..78: mol_type = other DNA
aatgatacggcgaccaccgagatctacacgtgccatannwnnwnnacactctttccctacacgacgctcttccgatct

SEQ ID NO: 574 - DNA, 78 bp, synthetic construct
  source 1..78: mol_type = other DNA
aatgatacggcgaccaccgagatctacaccttaggacnnwnnwnnacactctttccctacacgacgctcttccgatct

SEQ ID NO: 575 - DNA, 78 bp, synthetic construct
  source 1..78: mol_type = other DNA
aatgatacggcgaccaccgagatctacacaactgagcnnwnnwnnacactctttccctacacgacgctcttccgatct

SEQ ID NO: 576 - DNA, 78 bp, synthetic construct
  source 1..78: mol_type = other DNA
aatgatacggcgaccaccgagatctacacgacgatctnnwnnwnnacactctttccctacacgacgctcttccgatct

SEQ ID NO: 577 - DNA, 78 bp, synthetic construct
  source 1..78: mol_type = other DNA
aatgatacggcgaccaccgagatctacacatccagagnnwnnwnnacactctttccctacacgacgctcttccgatct

SEQ ID NO: 578 - DNA, 78 bp, synthetic construct
  source 1..78: mol_type = other DNA
aatgatacggcgaccaccgagatctacacagagtagcnnwnnwnnacactctttccctacacgacgctcttccgatct

SEQ ID NO: 579 - DNA, 78 bp, synthetic construct
  source 1..78: mol_type = other DNA
aatgatacggcgaccaccgagatctacactggactctnnwnnwnnacactctttccctacacgacgctcttccgatct

SEQ ID NO: 580 - DNA, 78 bp, synthetic construct
  source 1..78: mol_type = other DNA
aatgatacggcgaccaccgagatctacactacgctacnnwnnwnnacactctttccctacacgacgctcttccgatct

SEQ ID NO: 581 - DNA, 78 bp, synthetic construct
  source 1..78: mol_type = other DNA
aatgatacggcgaccaccgagatctacacgctatcctnnwnnwnnacactctttccctacacgacgctcttccgatct

SEQ ID NO: 582 - DNA, 78 bp, synthetic construct
  source 1..78: mol_type = other DNA
aatgatacggcgaccaccgagatctacacgcaagatcnnwnnwnnacactctttccctacacgacgctcttccgatct

SEQ ID NO: 583 - DNA, 78 bp, synthetic construct
  source 1..78: mol_type = other DNA
aatgatacggcgaccaccgagatctacacatcgatcgnnwnnwnnacactctttccctacacgacgctcttccgatct

SEQ ID NO: 584 - DNA, 78 bp, synthetic construct
  source 1..78: mol_type = other DNA
aatgatacggcgaccaccgagatctacaccggctaatnnwnnwnnacactctttccctacacgacgctcttccgatct

SEQ ID NO: 585 - DNA, 78 bp, synthetic construct
  source 1..78: mol_type = other DNA
aatgatacggcgaccaccgagatctacacacggaacannwnnwnnacactctttccctacacgacgctcttccgatct

SEQ ID NO: 586 - DNA, 78 bp, synthetic construct
  source 1..78: mol_type = other DNA
aatgatacggcgaccaccgagatctacaccgcatgatnnwnnwnnacactctttccctacacgacgctcttccgatct

SEQ ID NO: 587 - DNA, 78 bp, synthetic construct
  source 1..78: mol_type = other DNA
aatgatacggcgaccaccgagatctacacttccaaggnnwnnwnnacactctttccctacacgacgctcttccgatct

SEQ ID NO: 588 - DNA, 78 bp, synthetic construct
  source 1..78: mol_type = other DNA
aatgatacggcgaccaccgagatctacaccttgtcgannwnnwnnacactctttccctacacgacgctcttccgatct

SEQ ID NO: 589 - DNA, 78 bp, synthetic construct
  source 1..78: mol_type = other DNA
aatgatacggcgaccaccgagatctacacgagacgatnnwnnwnnacactctttccctacacgacgctcttccgatct

SEQ ID NO: 590 - DNA, 78 bp, synthetic construct
  source 1..78: mol_type = other DNA
aatgatacggcgaccaccgagatctacactgagctagnnwnnwnnacactctttccctacacgacgctcttccgatct

SEQ ID NO: 591 - DNA, 78 bp, synthetic construct
  source 1..78: mol_type = other DNA
aatgatacggcgaccaccgagatctacacactctcgannwnnwnnacactctttccctacacgacgctcttccgatct

SEQ ID NO: 592 - DNA, 78 bp, synthetic construct
  source 1..78: mol_type = other DNA
aatgatacggcgaccaccgagatctacacctgatcgtnnwnnwnnacactctttccctacacgacgctcttccgatct

SEQ ID NO: 593 - DNA, 78 bp, synthetic construct
  source 1..78: mol_type = other DNA
aatgatacggcgaccaccgagatctacaccgaccattnnwnnwnnacactctttccctacacgacgctcttccgatct

SEQ ID NO: 594 - DNA, 78 bp, synthetic construct
  source 1..78: mol_type = other DNA
aatgatacggcgaccaccgagatctacacgatagcgannwnnwnnacactctttccctacacgacgctcttccgatct

SEQ ID NO: 595 - DNA, 78 bp, synthetic construct
  source 1..78: mol_type = other DNA
aatgatacggcgaccaccgagatctacacaatggacgnnwnnwnnacactctttccctacacgacgctcttccgatct

SEQ ID NO: 596 - DNA, 78 bp, synthetic construct
  source 1..78: mol_type = other DNA
aatgatacggcgaccaccgagatctacaccgctagtannwnnwnnacactctttccctacacgacgctcttccgatct

SEQ ID NO: 597 - DNA, 78 bp, synthetic construct
  source 1..78: mol_type = other DNA
aatgatacggcgaccaccgagatctacactctctaggnnwnnwnnacactctttccctacacgacgctcttccgatct

SEQ ID NO: 598 - DNA, 78 bp, synthetic construct
  source 1..78: mol_type = other DNA
aatgatacggcgaccaccgagatctacacacattgcgnnwnnwnnacactctttccctacacgacgctcttccgatct

SEQ ID NO: 599 - DNA, 78 bp, synthetic construct
  source 1..78: mol_type = other DNA
aatgatacggcgaccaccgagatctacactgaggtgtnnwnnwnnacactctttccctacacgacgctcttccgatct

SEQ ID NO: 600 - DNA, 78 bp, synthetic construct
  source 1..78: mol_type = other DNA
aatgatacggcgaccaccgagatctacacaatgcctcnnwnnwnnacactctttccctacacgacgctcttccgatct

SEQ ID NO: 601 - DNA, 78 bp, synthetic construct
  source 1..78: mol_type = other DNA
aatgatacggcgaccaccgagatctacacctggagtannwnnwnnacactctttccctacacgacgctcttccgatct

SEQ ID NO: 602 - DNA, 78 bp, synthetic construct
  source 1..78: mol_type = other DNA
aatgatacggcgaccaccgagatctacacgtatgctgnnwnnwnnacactctttccctacacgacgctcttccgatct

SEQ ID NO: 603 - DNA, 78 bp, synthetic construct
  source 1..78: mol_type = other DNA
aatgatacggcgaccaccgagatctacactggagagtnnwnnwnnacactctttccctacacgacgctcttccgatct

SEQ ID NO: 604 - DNA, 78 bp, synthetic construct
  source 1..78: mol_type = other DNA
aatgatacggcgaccaccgagatctacaccgatagagnnwnnwnnacactctttccctacacgacgctcttccgatct

SEQ ID NO: 605 - DNA, 78 bp, synthetic construct
  source 1..78: mol_type = other DNA
aatgatacggcgaccaccgagatctacacctcattgcnnwnnwnnacactctttccctacacgacgctcttccgatct

SEQ ID NO: 606 - DNA, 78 bp, synthetic construct
  source 1..78: mol_type = other DNA
aatgatacggcgaccaccgagatctacacaccagcttnnwnnwnnacactctttccctacacgacgctcttccgatct

SEQ ID NO: 607 - DNA, 78 bp, synthetic construct
  source 1..78: mol_type = other DNA
aatgatacggcgaccaccgagatctacacgaatcgtgnnwnnwnnacactctttccctacacgacgctcttccgatct

SEQ ID NO: 608 - DNA, 78 bp, synthetic construct
  source 1..78: mol_type = other DNA
aatgatacggcgaccaccgagatctacacaggcttctnnwnnwnnacactctttccctacacgacgctcttccgatct

SEQ ID NO: 609 - DNA, 78 bp, synthetic construct
  source 1..78: mol_type = other DNA
aatgatacggcgaccaccgagatctacaccagttctgnnwnnwnnacactctttccctacacgacgctcttccgatct

SEQ ID NO: 610 - DNA, 78 bp, synthetic construct
  source 1..78: mol_type = other DNA
aatgatacggcgaccaccgagatctacacttggtgagnnwnnwnnacactctttccctacacgacgctcttccgatct

SEQ ID NO: 611 - DNA, 78 bp, synthetic construct
  source 1..78: mol_type = other DNA
aatgatacggcgaccaccgagatctacaccattcggtnnwnnwnnacactctttccctacacgacgctcttccgatct

SEQ ID NO: 612 - DNA, 78 bp, synthetic construct
  source 1..78: mol_type = other DNA
aatgatacggcgaccaccgagatctacactgtgaagcnnwnnwnnacactctttccctacacgacgctcttccgatct

SEQ ID NO: 613 - DNA, 78 bp, synthetic construct
  source 1..78: mol_type = other DNA
aatgatacggcgaccaccgagatctacactaagtggcnnwnnwnnacactctttccctacacgacgctcttccgatct

SEQ ID NO: 614 - DNA, 78 bp, synthetic construct
  source 1..78: mol_type = other DNA
aatgatacggcgaccaccgagatctacacacgtgatgnnwnnwnnacactctttccctacacgacgctcttccgatct

SEQ ID NO: 615 - DNA, 78 bp, synthetic construct
  source 1..78: mol_type = other DNA
aatgatacggcgaccaccgagatctacacgtagagcannwnnwnnacactctttccctacacgacgctcttccgatct

SEQ ID NO: 616 - DNA, 78 bp, synthetic construct
  source 1..78: mol_type = other DNA
aatgatacggcgaccaccgagatctacacgtcagttgnnwnnwnnacactctttccctacacgacgctcttccgatct

SEQ ID NO: 617 - DNA, 78 bp, synthetic construct
  source 1..78: mol_type = other DNA
aatgatacggcgaccaccgagatctacacattcgaggnnwnnwnnacactctttccctacacgacgctcttccgatct

SEQ ID NO: 618 - DNA, 78 bp, synthetic construct
  source 1..78: mol_type = other DNA
aatgatacggcgaccaccgagatctacacgatactggnnwnnwnnacactctttccctacacgacgctcttccgatct

SEQ ID NO: 619 - DNA, 78 bp, synthetic construct
  source 1..78: mol_type = other DNA
aatgatacggcgaccaccgagatctacacgccttgttnnwnnwnnacactctttccctacacgacgctcttccgatct

SEQ ID NO: 620 - DNA, 78 bp, synthetic construct
  source 1..78: mol_type = other DNA
aatgatacggcgaccaccgagatctacacttggtctcnnwnnwnnacactctttccctacacgacgctcttccgatct

SEQ ID NO: 621 - DNA, 78 bp, synthetic construct
  source 1..78: mol_type = other DNA
aatgatacggcgaccaccgagatctacacccgactatnnwnnwnnacactctttccctacacgacgctcttccgatct

SEQ ID NO: 622 - DNA, 78 bp, synthetic construct
  source 1..78: mol_type = other DNA
aatgatacggcgaccaccgagatctacacgtcctaagnnwnnwnnacactctttccctacacgacgctcttccgatct

SEQ ID NO: 623 - DNA, 78 bp, synthetic construct
  source 1..78: mol_type = other DNA
aatgatacggcgaccaccgagatctacacaccaatgcnnwnnwnnacactctttccctacacgacgctcttccgatct

SEQ ID NO: 624 - DNA, 78 bp, synthetic construct
  source 1..78: mol_type = other DNA
aatgatacggcgaccaccgagatctacacgatgcactnnwnnwnnacactctttccctacacgacgctcttccgatct

SEQ ID NO: 625 - DNA, 78 bp, synthetic construct
  source 1..78: mol_type = other DNA
aatgatacggcgaccaccgagatctacacgctggattnnwnnwnnacactctttccctacacgacgctcttccgatct

SEQ ID NO: 626 - DNA, 78 bp, synthetic construct
  source 1..78: mol_type = other DNA
aatgatacggcgaccaccgagatctacacatggttgcnnwnnwnnacactctttccctacacgacgctcttccgatct

SEQ ID NO: 627 - DNA, 78 bp, synthetic construct
  source 1..78: mol_type = other DNA
aatgatacggcgaccaccgagatctacaccagaatcgnnwnnwnnacactctttccctacacgacgctcttccgatct

SEQ ID NO: 628 - DNA, 78 bp, synthetic construct
  source 1..78: mol_type = other DNA
aatgatacggcgaccaccgagatctacacgaacgcttnnwnnwnnacactctttccctacacgacgctcttccgatct

SEQ ID NO: 629 - DNA, 78 bp, synthetic construct
  source 1..78: mol_type = other DNA
aatgatacggcgaccaccgagatctacactcgaaccannwnnwnnacactctttccctacacgacgctcttccgatct

SEQ ID NO: 630 - DNA, 78 bp, synthetic construct
  source 1..78: mol_type = other DNA
aatgatacggcgaccaccgagatctacacctatcgcannwnnwnnacactctttccctacacgacgctcttccgatct

SEQ ID NO: 631 - DNA, 78 bp, synthetic construct
  source 1..78: mol_type = other DNA
aatgatacggcgaccaccgagatctacactacggttgnnwnnwnnacactctttccctacacgacgctcttccgatct

SEQ ID NO: 632 - DNA, 78 bp, synthetic construct
  source 1..78: mol_type = other DNA
aatgatacggcgaccaccgagatctacacgagatgtcnnwnnwnnacactctttccctacacgacgctcttccgatct

SEQ ID NO: 633 - DNA, 78 bp, synthetic construct
  source 1..78: mol_type = other DNA
aatgatacggcgaccaccgagatctacaccttacagcnnwnnwnnacactctttccctacacgacgctcttccgatct

SEQ ID NO: 634 - DNA, 78 bp, synthetic construct
  source 1..78: mol_type = other DNA
aatgatacggcgaccaccgagatctacacaggaggaannwnnwnnacactctttccctacacgacgctcttccgatct

SEQ ID NO: 635 - DNA, 78 bp, synthetic construct
  source 1..78: mol_type = other DNA
aatgatacggcgaccaccgagatctacacgacgaatgnnwnnwnnacactctttccctacacgacgctcttccgatct

SEQ ID NO: 636 - DNA, 78 bp, synthetic construct
  source 1..78: mol_type = other DNA
aatgatacggcgaccaccgagatctacacgaagaggtnnwnnwnnacactctttccctacacgacgctcttccgatct

SEQ ID NO: 637 - DNA, 78 bp, synthetic construct
  source 1..78: mol_type = other DNA
aatgatacggcgaccaccgagatctacaccgtcaatgnnwnnwnnacactctttccctacacgacgctcttccgatct

SEQ ID NO: 638 - DNA, 78 bp, synthetic construct
  source 1..78: mol_type = other DNA
aatgatacggcgaccaccgagatctacactaccaggannwnnwnnacactctttccctacacgacgctcttccgatct

SEQ ID NO: 639 - DNA, 78 bp, synthetic construct
  source 1..78: mol_type = other DNA
aatgatacggcgaccaccgagatctacaccgtacgaannwnnwnnacactctttccctacacgacgctcttccgatct

SEQ ID NO: 640 - DNA, 78 bp, synthetic construct
  source 1..78: mol_type = other DNA
aatgatacggcgaccaccgagatctacacgacttaggnnwnnwnnacactctttccctacacgacgctcttccgatct

SEQ ID NO: 641 - DNA, 78 bp, synthetic construct
  source 1..78: mol_type = other DNA
aatgatacggcgaccaccgagatctacacagtgcagtnnwnnwnnacactctttccctacacgacgctcttccgatct

SEQ ID NO: 642 - DNA, 78 bp, synthetic construct
  source 1..78: mol_type = other DNA
aatgatacggcgaccaccgagatctacacttgatccgnnwnnwnnacactctttccctacacgacgctcttccgatct

SEQ ID NO: 643 - DNA, 78 bp, synthetic construct
  source 1..78: mol_type = other DNA
aatgatacggcgaccaccgagatctacactgccattcnnwnnwnnacactctttccctacacgacgctcttccgatct

SEQ ID NO: 644 - DNA, 78 bp, synthetic construct
  source 1..78: mol_type = other DNA
aatgatacggcgaccaccgagatctacaccttgctgtnnwnnwnnacactctttccctacacgacgctcttccgatct

SEQ ID NO: 645 - DNA, 78 bp, synthetic construct
  source 1..78: mol_type = other DNA
aatgatacggcgaccaccgagatctacaccctactgannwnnwnnacactctttccctacacgacgctcttccgatct

SEQ ID NO: 646 - DNA, 78 bp, synthetic construct
  source 1..78: mol_type = other DNA
aatgatacggcgaccaccgagatctacacccaagttgnnwnnwnnacactctttccctacacgacgctcttccgatct

SEQ ID NO: 647 - DNA, 78 bp, synthetic construct
  source 1..78: mol_type = other DNA
aatgatacggcgaccaccgagatctacactgatcggannwnnwnnacactctttccctacacgacgctcttccgatct

SEQ ID NO: 648 - DNA, 78 bp, synthetic construct
  source 1..78: mol_type = other DNA
aatgatacggcgaccaccgagatctacactagttgcgnnwnnwnnacactctttccctacacgacgctcttccgatct

SEQ ID NO: 649 - DNA, 78 bp, synthetic construct
  source 1..78: mol_type = other DNA
aatgatacggcgaccaccgagatctacacgtctgatcnnwnnwnnacactctttccctacacgacgctcttccgatct

SEQ ID NO: 650 - DNA, 78 bp, synthetic construct
  source 1..78: mol_type = other DNA
aatgatacggcgaccaccgagatctacaccgttatgcnnwnnwnnacactctttccctacacgacgctcttccgatct

SEQ ID NO: 651 - DNA, 78 bp, synthetic construct
  source 1..78: mol_type = other DNA
aatgatacggcgaccaccgagatctacacgctctgtannwnnwnnacactctttccctacacgacgctcttccgatct

SEQ ID NO: 652 - DNA, 78 bp, synthetic construct
  source 1..78: mol_type = other DNA
aatgatacggcgaccaccgagatctacacttaccgagnnwnnwnnacactctttccctacacgacgctcttccgatct

SEQ ID NO: 653 - DNA, 78 bp, synthetic construct
  source 1..78: mol_type = other DNA
aatgatacggcgaccaccgagatctacacgccataacnnwnnwnnacactctttccctacacgacgctcttccgatct

SEQ ID NO: 654 - DNA, 78 bp, synthetic construct
  source 1..78: mol_type = other DNA
aatgatacggcgaccaccgagatctacacctcagagtnnwnnwnnacactctttccctacacgacgctcttccgatct

SEQ ID NO: 655 - DNA, 78 bp, synthetic construct
  source 1..78: mol_type = other DNA
aatgatacggcgaccaccgagatctacaccgagactannwnnwnnacactctttccctacacgacgctcttccgatct

SEQ ID NO: 656 - DNA, 78 bp, synthetic construct
  source 1..78: mol_type = other DNA
aatgatacggcgaccaccgagatctacactgtgcgttnnwnnwnnacactctttccctacacgacgctcttccgatct

SEQ ID NO: 657 - DNA, 78 bp, synthetic construct
  source 1..78: mol_type = other DNA
aatgatacggcgaccaccgagatctacacttcaggagnnwnnwnnacactctttccctacacgacgctcttccgatct

SEQ ID NO: 658 - DNA, 78 bp, synthetic construct
  source 1..78: mol_type = other DNA
aatgatacggcgaccaccgagatctacacgactatgcnnwnnwnnacactctttccctacacgacgctcttccgatct

SEQ ID NO: 659 - DNA, 78 bp, synthetic construct
  source 1..78: mol_type = other DNA
aatgatacggcgaccaccgagatctacacaggttcgannwnnwnnacactctttccctacacgacgctcttccgatct

SEQ ID NO: 660 - DNA, 78 bp, synthetic construct
  source 1..78: mol_type = other DNA
aatgatacggcgaccaccgagatctacacagtctgtgnnwnnwnnacactctttccctacacgacgctcttccgatct

SEQ ID NO: 661 - DNA, 78 bp, synthetic construct
  source 1..78: mol_type = other DNA
aatgatacggcgaccaccgagatctacacacctaaggnnwnnwnnacactctttccctacacgacgctcttccgatct

SEQ ID NO: 662 - DNA, 78 bp, synthetic construct
  source 1..78: mol_type = other DNA
aatgatacggcgaccaccgagatctacactgcaggtannwnnwnnacactctttccctacacgacgctcttccgatct

SEQ ID NO: 663 - DNA, 78 bp, synthetic construct
  source 1..78: mol_type = other DNA
aatgatacggcgaccaccgagatctacacaaggacacnnwnnwnnacactctttccctacacgacgctcttccgatct

SEQ ID NO: 664 - DNA, 78 bp, synthetic construct
  source 1..78: mol_type = other DNA
aatgatacggcgaccaccgagatctacaccaacctagnnwnnwnnacactctttccctacacgacgctcttccgatct

SEQ ID NO: 665 - DNA, 78 bp, synthetic construct
  source 1..78: mol_type = other DNA
aatgatacggcgaccaccgagatctacacctgacacannwnnwnnacactctttccctacacgacgctcttccgatct

SEQ ID NO: 666 - DNA, 78 bp, synthetic construct
  source 1..78: mol_type = other DNA
aatgatacggcgaccaccgagatctacacactcgttgnnwnnwnnacactctttccctacacgacgctcttccgatct

SEQ ID NO: 667 - DNA, 78 bp, synthetic construct
  source 1..78: mol_type = other DNA
aatgatacggcgaccaccgagatctacacagctcctannwnnwnnacactctttccctacacgacgctcttccgatct

SEQ ID NO: 668 - DNA, 78 bp, synthetic construct
  source 1..78: mol_type = other DNA
aatgatacggcgaccaccgagatctacactacatcggnnwnnwnnacactctttccctacacgacgctcttccgatct

SEQ ID NO: 669 - DNA, 78 bp, synthetic construct
  source 1..78: mol_type = other DNA
aatgatacggcgaccaccgagatctacaccacaagtcnnwnnwnnacactctttccctacacgacgctcttccgatct

SEQ ID NO: 670 - DNA, 78 bp, synthetic construct
  source 1..78: mol_type = other DNA
aatgatacggcgaccaccgagatctacaccggattgannwnnwnnacactctttccctacacgacgctcttccgatct

SEQ ID NO: 671 - DNA, 78 bp, synthetic construct
  source 1..78: mol_type = other DNA
aatgatacggcgaccaccgagatctacacagtcgacannwnnwnnacactctttccctacacgacgctcttccgatct

SEQ ID NO: 672 - DNA, 78 bp, synthetic construct
  source 1..78: mol_type = other DNA
aatgatacggcgaccaccgagatctacacgtctccttnnwnnwnnacactctttccctacacgacgctcttccgatct

SEQ ID NO: 673 - DNA, 78 bp, synthetic construct
  source 1..78: mol_type = other DNA
aatgatacggcgaccaccgagatctacacgagatacgnnwnnwnnacactctttccctacacgacgctcttccgatct

SEQ ID NO: 674 - DNA, 78 bp, synthetic construct
  source 1..78: mol_type = other DNA
aatgatacggcgaccaccgagatctacacatcggtgtnnwnnwnnacactctttccctacacgacgctcttccgatct

SEQ ID NO: 675 - DNA, 78 bp, synthetic construct
  source 1..78: mol_type = other DNA
aatgatacggcgaccaccgagatctacactctcgcaannwnnwnnacactctttccctacacgacgctcttccgatct

SEQ ID NO: 676 - DNA, 78 bp, synthetic construct
  source 1..78: mol_type = other DNA
aatgatacggcgaccaccgagatctacactctaacgcnnwnnwnnacactctttccctacacgacgctcttccgatct

SEQ ID NO: 677 - DNA, 78 bp, synthetic construct
  source 1..78: mol_type = other DNA
aatgatacggcgaccaccgagatctacaccaatcgacnnwnnwnnacactctttccctacacgacgctcttccgatct

SEQ ID NO: 678 - DNA, 78 bp, synthetic construct
  source 1..78: mol_type = other DNA
aatgatacggcgaccaccgagatctacacgaggacttnnwnnwnnacactctttccctacacgacgctcttccgatct

SEQ ID NO: 679 - DNA, 78 bp, synthetic construct
  source 1..78: mol_type = other DNA
aatgatacggcgaccaccgagatctacactggagttgnnwnnwnnacactctttccctacacgacgctcttccgatct

SEQ ID NO: 680 - DNA, 78 bp, synthetic construct
  source 1..78: mol_type = other DNA
aatgatacggcgaccaccgagatctacacctaggcatnnwnnwnnacactctttccctacacgacgctcttccgatct

SEQ ID NO: 681 - DNA, 78 bp, synthetic construct
  source 1..78: mol_type = other DNA
aatgatacggcgaccaccgagatctacacctctactcnnwnnwnnacactctttccctacacgacgctcttccgatct

SEQ ID NO: 682 - DNA, 78 bp, synthetic construct
  source 1..78: mol_type = other DNA
aatgatacggcgaccaccgagatctacacagaagcgtnnwnnwnnacactctttccctacacgacgctcttccgatct

SEQ ID NO: 683 - DNA, 78 bp, synthetic construct
  source 1..78: mol_type = other DNA
aatgatacggcgaccaccgagatctacactcgaaggtnnwnnwnnacactctttccctacacgacgctcttccgatct

SEQ ID NO: 684 - DNA, 78 bp, synthetic construct
  source 1..78: mol_type = other DNA
aatgatacggcgaccaccgagatctacacgtcggtaannwnnwnnacactctttccctacacgacgctcttccgatct

SEQ ID NO: 685 - DNA, 78 bp, synthetic construct
  source 1..78: mol_type = other DNA
aatgatacggcgaccaccgagatctacacacgatgacnnwnnwnnacactctttccctacacgacgctcttccgatct

SEQ ID NO: 686 - DNA, 78 bp, synthetic construct
  source 1..78: mol_type = other DNA
aatgatacggcgaccaccgagatctacactccgtatgnnwnnwnnacactctttccctacacgacgctcttccgatct

SEQ ID NO: 687 - DNA, 78 bp, synthetic construct
  source 1..78: mol_type = other DNA
aatgatacggcgaccaccgagatctacacctaggtgannwnnwnnacactctttccctacacgacgctcttccgatct

SEQ ID NO: 688 - DNA, 78 bp, synthetic construct
  source 1..78: mol_type = other DNA
aatgatacggcgaccaccgagatctacaccattgcctnnwnnwnnacactctttccctacacgacgctcttccgatct

SEQ ID NO: 689 - DNA, 14 bp, synthetic construct
  source 1..14: mol_type = other DNA
gatcggaagagcca

SEQ ID NO: 690 - DNA, 71 bp, synthetic construct
  source 1..71: mol_type = other DNA
caagcagaagacggcatacgagatacgatcagggcagtcggtgatcatagcggtattacgcgagattacga

SEQ ID NO: 691 - DNA, 71 bp, synthetic construct
  source 1..71: mol_type = other DNA
caagcagaagacggcatacgagattcgagagtggcagtcggtgatcatagcggtattacgcgagattacga

SEQ ID NO: 692 - DNA, 71 bp, synthetic construct
  source 1..71: mol_type = other DNA
caagcagaagacggcatacgagatctagctcaggcagtcggtgatcatagcggtattacgcgagattacga

SEQ ID NO: 693 - DNA, 71 bp, synthetic construct
  source 1..71: mol_type = other DNA
caagcagaagacggcatacgagatatcgtctcggcagtcggtgatcatagcggtattacgcgagattacga

SEQ ID NO: 694 - DNA, 71 bp, synthetic construct
  source 1..71: mol_type = other DNA
caagcagaagacggcatacgagattcgacaagggcagtcggtgatcatagcggtattacgcgagattacga

SEQ ID NO: 695 - DNA, 71 bp, synthetic construct
  source 1..71: mol_type = other DNA
caagcagaagacggcatacgagatccttggaaggcagtcggtgatcatagcggtattacgcgagattacga

SEQ ID NO: 696 - DNA, 71 bp, synthetic construct
  source 1..71: mol_type = other DNA
caagcagaagacggcatacgagatatcatgcgggcagtcggtgatcatagcggtattacgcgagattacga

SEQ ID NO: 697 - DNA, 71 bp, synthetic construct
  source 1..71: mol_type = other DNA
caagcagaagacggcatacgagattgttccgtggcagtcggtgatcatagcggtattacgcgagattacga

SEQ ID NO: 698 - DNA, 71 bp, synthetic construct
  source 1..71: mol_type = other DNA
caagcagaagacggcatacgagatattagccgggcagtcggtgatcatagcggtattacgcgagattacga

SEQ ID NO: 699 - DNA, 71 bp, synthetic construct
  source 1..71: mol_type = other DNA
caagcagaagacggcatacgagatcgatcgatggcagtcggtgatcatagcggtattacgcgagattacga

SEQ ID NO: 700 - DNA, 71 bp, synthetic construct
  source 1..71: mol_type = other DNA
caagcagaagacggcatacgagatgatcttgcggcagtcggtgatcatagcggtattacgcgagattacga

SEQ ID NO: 701 - DNA, 71 bp, synthetic construct
  source 1..71: mol_type = other DNA
caagcagaagacggcatacgagataggatagcggcagtcggtgatcatagcggtattacgcgagattacga

SEQ ID NO: 702 - DNA, 71 bp, synthetic construct
  source 1..71: mol_type = other DNA
caagcagaagacggcatacgagatgtagcgtaggcagtcggtgatcatagcggtattacgcgagattacga

SEQ ID NO: 703 - DNA, 71 bp, synthetic construct
  source 1..71: mol_type = other DNA
caagcagaagacggcatacgagatagagtccaggcagtcggtgatcatagcggtattacgcgagattacga

SEQ ID NO: 704 - DNA, 71 bp, synthetic construct
  source 1..71: mol_type = other DNA
caagcagaagacggcatacgagatgctactctggcagtcggtgatcatagcggtattacgcgagattacga

SEQ ID NO: 705 - DNA, 71 bp, synthetic construct
  source 1..71: mol_type = other DNA
caagcagaagacggcatacgagatctctggatggcagtcggtgatcatagcggtattacgcgagattacga

SEQ ID NO: 706 - DNA, 71 bp, synthetic construct
  source 1..71: mol_type = other DNA
caagcagaagacggcatacgagatagatcgtcggcagtcggtgatcatagcggtattacgcgagattacga

SEQ ID NO: 707 - DNA, 71 bp, synthetic construct
  source 1..71: mol_type = other DNA
caagcagaagacggcatacgagatgctcagttggcagtcggtgatcatagcggtattacgcgagattacga

SEQ ID NO: 708 - DNA, 71 bp, synthetic construct
  source 1..71: mol_type = other DNA
caagcagaagacggcatacgagatgtcctaagggcagtcggtgatcatagcggtattacgcgagattacga

SEQ ID NO: 709 - DNA, 71 bp, synthetic construct
  source 1..71: mol_type = other DNA
caagcagaagacggcatacgagattatggcacggcagtcggtgatcatagcggtattacgcgagattacga

SEQ ID NO: 710 - DNA, 71 bp, synthetic construct
  source 1..71: mol_type = other DNA
caagcagaagacggcatacgagattcggattcggcagtcggtgatcatagcggtattacgcgagattacga

SEQ ID NO: 711 - DNA, 71 bp, synthetic construct
  source 1..71: mol_type = other DNA
caagcagaagacggcatacgagataacagcgaggcagtcggtgatcatagcggtattacgcgagattacga

SEQ ID NO: 712 - DNA, 71 bp, synthetic construct
  source 1..71: mol_type = other DNA
caagcagaagacggcatacgagatccaacgaaggcagtcggtgatcatagcggtattacgcgagattacga

SEQ ID NO: 713 - DNA, 71 bp, synthetic construct
  source 1..71: mol_type = other DNA
caagcagaagacggcatacgagatcagtgcttggcagtcggtgatcatagcggtattacgcgagattacga

SEQ ID NO: 714 - DNA, 71 bp, synthetic construct
  source 1..71: mol_type = other DNA
caagcagaagacggcatacgagatgatcaaggggcagtcggtgatcatagcggtattacgcgagattacga

SEQ ID NO: 715 - DNA, 71 bp, synthetic construct
  source 1..71: mol_type = other DNA
caagcagaagacggcatacgagattcttcgacggcagtcggtgatcatagcggtattacgcgagattacga

SEQ ID NO: 716 - DNA, 71 bp, synthetic construct
  source 1..71: mol_type = other DNA
caagcagaagacggcatacgagatatcgtggtggcagtcggtgatcatagcggtattacgcgagattacga

SEQ ID NO: 717 - DNA, 71 bp, synthetic construct
  source 1..71: mol_type = other DNA
caagcagaagacggcatacgagatcggtaatcggcagtcggtgatcatagcggtattacgcgagattacga

SEQ ID NO: 718 - DNA, 71 bp, synthetic construct
  source 1..71: mol_type = other DNA
caagcagaagacggcatacgagatagttgtgcggcagtcggtgatcatagcggtattacgcgagattacga

SEQ ID NO: 719 - DNA, 71 bp, synthetic construct
  source 1..71: mol_type = other DNA
caagcagaagacggcatacgagataatgacgcggcagtcggtgatcatagcggtattacgcgagattacga

SEQ ID NO: 720 - DNA, 71 bp, synthetic construct
  source 1..71: mol_type = other DNA
caagcagaagacggcatacgagattaccggatggcagtcggtgatcatagcggtattacgcgagattacga

SEQ ID NO: 721 - DNA, 71 bp, synthetic construct
  source 1..71: mol_type = other DNA
caagcagaagacggcatacgagatttgcaacgggcagtcggtgatcatagcggtattacgcgagattacga

SEQ ID NO: 722 - DNA, 71 bp, synthetic construct
  source 1..71: mol_type = other DNA
caagcagaagacggcatacgagatcacttcacggcagtcggtgatcatagcggtattacgcgagattacga

SEQ ID NO: 723 - DNA, 71 bp, synthetic construct
  source 1..71: mol_type = other DNA
caagcagaagacggcatacgagattagccatgggcagtcggtgatcatagcggtattacgcgagattacga

SEQ ID NO: 724 - DNA, 71 bp, synthetic construct
  source 1..71: mol_type = other DNA
caagcagaagacggcatacgagatacaggcatggcagtcggtgatcatagcggtattacgcgagattacga

SEQ ID NO: 725 - DNA, 71 bp, synthetic construct
  source 1..71: mol_type = other DNA
caagcagaagacggcatacgagataggtgttgggcagtcggtgatcatagcggtattacgcgagattacga

SEQ ID NO: 726 - DNA, 71 bp, synthetic construct
  source 1..71: mol_type = other DNA
caagcagaagacggcatacgagatcagtcacaggcagtcggtgatcatagcggtattacgcgagattacga

SEQ ID NO: 727 - DNA, 71 bp, synthetic construct
  source 1..71: mol_type = other DNA
caagcagaagacggcatacgagattcgatgacggcagtcggtgatcatagcggtattacgcgagattacga

SEQ ID NO: 728 - DNA, 71 bp, synthetic construct
  source 1..71: mol_type = other DNA
caagcagaagacggcatacgagatgaagtgctggcagtcggtgatcatagcggtattacgcgagattacga

SEQ ID NO: 729 - DNA, 71 bp, synthetic construct
  source 1..71: mol_type = other DNA
caagcagaagacggcatacgagatcttccttcggcagtcggtgatcatagcggtattacgcgagattacga

SEQ ID NO: 730 - DNA, 71 bp, synthetic construct
  source 1..71: mol_type = other DNA
caagcagaagacggcatacgagatcgaacaacggcagtcggtgatcatagcggtattacgcgagattacga

SEQ ID NO: 731 - DNA, 71 bp, synthetic construct
  source 1..71: mol_type = other DNA
caagcagaagacggcatacgagataacaaccgggcagtcggtgatcatagcggtattacgcgagattacga

SEQ ID NO: 732 - DNA, 71 bp, synthetic construct
  source 1..71: mol_type = other DNA
caagcagaagacggcatacgagatacctcagtggcagtcggtgatcatagcggtattacgcgagattacga

SEQ ID NO: 733 - DNA, 71 bp, synthetic construct
  source 1..71: mol_type = other DNA
caagcagaagacggcatacgagatcgtcttcaggcagtcggtgatcatagcggtattacgcgagattacga

SEQ ID NO: 734 - DNA, 71 bp, synthetic construct
  source 1..71: mol_type = other DNA
caagcagaagacggcatacgagattgcgtaacggcagtcggtgatcatagcggtattacgcgagattacga

SEQ ID NO: 735 - DNA, 71 bp, synthetic construct
  source 1..71: mol_type = other DNA
caagcagaagacggcatacgagataacacgctggcagtcggtgatcatagcggtattacgcgagattacga

SEQ ID NO: 736 - DNA, 71 bp, synthetic construct
  source 1..71: mol_type = other DNA
caagcagaagacggcatacgagatactcgatcggcagtcggtgatcatagcggtattacgcgagattacga

SEQ ID NO: 737 - DNA, 71 bp, synthetic construct
  source 1..71: mol_type = other DNA
caagcagaagacggcatacgagattgagctgtggcagtcggtgatcatagcggtattacgcgagattacga

SEQ ID NO: 738 - DNA, 71 bp, synthetic construct
  source 1..71: mol_type = other DNA
caagcagaagacggcatacgagattactgctcggcagtcggtgatcatagcggtattacgcgagattacga

SEQ ID NO: 739 - DNA, 71 bp, synthetic construct
  source 1..71: mol_type = other DNA
caagcagaagacggcatacgagatgacgaactggcagtcggtgatcatagcggtattacgcgagattacga

SEQ ID NO: 740 - DNA, 71 bp, synthetic construct
  source 1..71: mol_type = other DNA
caagcagaagacggcatacgagatcttcgcaaggcagtcggtgatcatagcggtattacgcgagattacga

SEQ ID NO: 741 - DNA, 71 bp, synthetic construct
  source 1..71: mol_type = other DNA
caagcagaagacggcatacgagatatggcgatggcagtcggtgatcatagcggtattacgcgagattacga

SEQ ID NO: 742 - DNA, 71 bp, synthetic construct
  source 1..71: mol_type = other DNA
caagcagaagacggcatacgagatacatgccaggcagtcggtgatcatagcggtattacgcgagattacga

SEQ ID NO: 743 - DNA, 71 bp, synthetic construct
  source 1..71: mol_type = other DNA
caagcagaagacggcatacgagatgtcaacagggcagtcggtgatcatagcggtattacgcgagattacga

SEQ ID NO: 744 - DNA, 71 bp, synthetic construct
  source 1..71: mol_type = other DNA
caagcagaagacggcatacgagatgtggtatgggcagtcggtgatcatagcggtattacgcgagattacga

SEQ ID NO: 745 - DNA, 71 bp, synthetic construct
  source 1..71: mol_type = other DNA
caagcagaagacggcatacgagatccaacttcggcagtcggtgatcatagcggtattacgcgagattacga

SEQ ID NO: 746 - DNA, 71 bp, synthetic construct
  source 1..71: mol_type = other DNA
caagcagaagacggcatacgagatgacgtcatggcagtcggtgatcatagcggtattacgcgagattacga

SEQ ID NO: 747 - DNA, 71 bp, synthetic construct
  source 1..71: mol_type = other DNA
caagcagaagacggcatacgagatacgtccaaggcagtcggtgatcatagcggtattacgcgagattacga

SEQ ID NO: 748 - DNA, 71 bp, synthetic construct
  source 1..71: mol_type = other DNA
caagcagaagacggcatacgagatgatccactggcagtcggtgatcatagcggtattacgcgagattacga

SEQ ID NO: 749 - DNA, 71 bp, synthetic construct
  source 1..71: mol_type = other DNA
caagcagaagacggcatacgagatagcctatcggcagtcggtgatcatagcggtattacgcgagattacga

SEQ ID NO: 750 - DNA, 71 bp, synthetic construct
  source 1..71: mol_type = other DNA
caagcagaagacggcatacgagatagctaccaggcagtcggtgatcatagcggtattacgcgagattacga

SEQ ID NO: 751 - DNA, 71 bp, synthetic construct
  source 1..71: mol_type = other DNA
caagcagaagacggcatacgagatagattgcgggcagtcggtgatcatagcggtattacgcgagattacga

SEQ ID NO: 752 - DNA, 71 bp, synthetic construct
  source 1..71: mol_type = other DNA
caagcagaagacggcatacgagatcacacatcggcagtcggtgatcatagcggtattacgcgagattacga

SEQ ID NO: 753 - DNA, 71 bp, synthetic construct
  source 1..71: mol_type = other DNA
caagcagaagacggcatacgagatgagcaatcggcagtcggtgatcatagcggtattacgcgagattacga

SEQ ID NO: 754 - DNA, 71 bp, synthetic construct
  source 1..71: mol_type = other DNA
caagcagaagacggcatacgagatatagagcgggcagtcggtgatcatagcggtattacgcgagattacga

SEQ ID NO: 755 - DNA, 71 bp, synthetic construct
  source 1..71: mol_type = other DNA
caagcagaagacggcatacgagatgaccgataggcagtcggtgatcatagcggtattacgcgagattacga

SEQ ID NO: 756 - DNA, 71 bp, synthetic construct
  source 1..71: mol_type = other DNA
caagcagaagacggcatacgagatcagacgttggcagtcggtgatcatagcggtattacgcgagattacga

SEQ ID NO: 757 - DNA, 71 bp, synthetic construct
  source 1..71: mol_type = other DNA
caagcagaagacggcatacgagatctgaacgtggcagtcggtgatcatagcggtattacgcgagattacga

SEQ ID NO: 758 - DNA, 71 bp, synthetic construct
  source 1..71: mol_type = other DNA
caagcagaagacggcatacgagatttggactgggcagtcggtgatcatagcggtattacgcgagattacga

SEQ ID NO: 759 - DNA, 71 bp, synthetic construct
  source 1..71: mol_type = other DNA
caagcagaagacggcatacgagatgtctgcaaggcagtcggtgatcatagcggtattacgcgagattacga

SEQ ID NO: 760 - DNA, 71 bp, synthetic construct
  source 1..71: mol_type = other DNA
caagcagaagacggcatacgagatccacattgggcagtcggtgatcatagcggtattacgcgagattacga

SEQ ID NO: 761 - DNA, 71 bp, synthetic construct
  source 1..71: mol_type = other DNA
caagcagaagacggcatacgagatgatggagtggcagtcggtgatcatagcggtattacgcgagattacga

SEQ ID NO: 762 - DNA, 71 bp, synthetic construct
  source 1..71: mol_type = other DNA
caagcagaagacggcatacgagataggtcaacggcagtcggtgatcatagcggtattacgcgagattacga

SEQ ID NO: 763 - DNA, 71 bp, synthetic construct
  source 1..71: mol_type = other DNA
caagcagaagacggcatacgagattacacacgggcagtcggtgatcatagcggtattacgcgagattacga

SEQ ID NO: 764 - DNA, 71 bp, synthetic construct
  source 1..71: mol_type = other DNA
caagcagaagacggcatacgagatcaagtcgtggcagtcggtgatcatagcggtattacgcgagattacga

SEQ ID NO: 765 - DNA, 71 bp, synthetic construct
  source 1..71: mol_type = other DNA
caagcagaagacggcatacgagatagctagtgggcagtcggtgatcatagcggtattacgcgagattacga

SEQ ID NO: 766 - DNA, 71 bp, synthetic construct
  source 1..71: mol_type = other DNA
caagcagaagacggcatacgagatctcctagtggcagtcggtgatcatagcggtattacgcgagattacga

SEQ ID NO: 767 - DNA, 71 bp, synthetic construct
  source 1..71: mol_type = other DNA
caagcagaagacggcatacgagatactcctacggcagtcggtgatcatagcggtattacgcgagattacga

SEQ ID NO: 768 - DNA, 71 bp, synthetic construct
  source 1..71: mol_type = other DNA
caagcagaagacggcatacgagatcaatcaggggcagtcggtgatcatagcggtattacgcgagattacga

SEQ ID NO: 769 - DNA, 71 bp, synthetic construct
  source 1..71: mol_type = other DNA
caagcagaagacggcatacgagattcgtgcatggcagtcggtgatcatagcggtattacgcgagattacga

SEQ ID NO: 770 - DNA, 71 bp, synthetic construct
  source 1..71: mol_type = other DNA
caagcagaagacggcatacgagattaacgtcgggcagtcggtgatcatagcggtattacgcgagattacga

SEQ ID NO: 771 - DNA, 71 bp, synthetic construct
  source 1..71: mol_type = other DNA
caagcagaagacggcatacgagataaggcgtaggcagtcggtgatcatagcggtattacgcgagattacga

SEQ ID NO: 772 - DNA, 71 bp, synthetic construct
  source 1..71: mol_type = other DNA
caagcagaagacggcatacgagattcttacggggcagtcggtgatcatagcggtattacgcgagattacga

SEQ ID NO: 773 - DNA, 71 bp, synthetic construct
  source 1..71: mol_type = other DNA
caagcagaagacggcatacgagatcgtgtgatggcagtcggtgatcatagcggtattacgcgagattacga

SEQ ID NO: 774 - DNA, 71 bp, synthetic construct
  source 1..71: mol_type = other DNA
caagcagaagacggcatacgagataacaggtgggcagtcggtgatcatagcggtattacgcgagattacga

SEQ ID NO: 775 - DNA, 71 bp, synthetic construct
  source 1..71: mol_type = other DNA
caagcagaagacggcatacgagatagtcgaagggcagtcggtgatcatagcggtattacgcgagattacga

SEQ ID NO: 776 - DNA, 71 bp, synthetic construct
  source 1..71: mol_type = other DNA
caagcagaagacggcatacgagattggaagcaggcagtcggtgatcatagcggtattacgcgagattacga

SEQ ID NO: 777 - DNA, 71 bp, synthetic construct
  source 1..71: mol_type = other DNA
caagcagaagacggcatacgagatctcgttctggcagtcggtgatcatagcggtattacgcgagattacga

SEQ ID NO: 778 - DNA, 71 bp, synthetic construct
  source 1..71: mol_type = other DNA
caagcagaagacggcatacgagatacgagaacggcagtcggtgatcatagcggtattacgcgagattacga

SEQ ID NO: 779 - DNA, 71 bp, synthetic construct
  source 1..71: mol_type = other DNA
caagcagaagacggcatacgagataagcctgaggcagtcggtgatcatagcggtattacgcgagattacga

SEQ ID NO: 780 - DNA, 71 bp, synthetic construct
  source 1..71: mol_type = other DNA
caagcagaagacggcatacgagatctacaaggggcagtcggtgatcatagcggtattacgcgagattacga

SEQ ID NO: 781 - DNA, 71 bp, synthetic construct
  source 1..71: mol_type = other DNA
caagcagaagacggcatacgagatcgatgttcggcagtcggtgatcatagcggtattacgcgagattacga

SEQ ID NO: 782 - DNA, 71 bp, synthetic construct
  source 1..71: mol_type = other DNA
caagcagaagacggcatacgagataccggttaggcagtcggtgatcatagcggtattacgcgagattacga

SEQ ID NO: 783 - DNA, 71 bp, synthetic construct
  source 1..71: mol_type = other DNA
caagcagaagacggcatacgagatgaacggttggcagtcggtgatcatagcggtattacgcgagattacga

SEQ ID NO: 784 - DNA, 71 bp, synthetic construct
  source 1..71: mol_type = other DNA
caagcagaagacggcatacgagatctgtaccaggcagtcggtgatcatagcggtattacgcgagattacga

SEQ ID NO: 785 - DNA, 71 bp, synthetic construct
  source 1..71: mol_type = other DNA
caagcagaagacggcatacgagatgcgcatatggcagtcggtgatcatagcggtattacgcgagattacga

SEQ ID NO: 786 - DNA, 71 bp, synthetic construct
  source 1..71: mol_type = other DNA
caagcagaagacggcatacgagattgataggcggcagtcggtgatcatagcggtattacgcgagattacga

SEQ ID NO: 787 - DNA, 71 bp, synthetic construct
  source 1..71: mol_type = other DNA
caagcagaagacggcatacgagatcatccaagggcagtcggtgatcatagcggtattacgcgagattacga

SEQ ID NO: 788 - DNA, 71 bp, synthetic construct
  source 1..71: mol_type = other DNA
caagcagaagacggcatacgagatgtgagactggcagtcggtgatcatagcggtattacgcgagattacga

SEQ ID NO: 789 - DNA, 71 bp, synthetic construct
  source 1..71: mol_type = other DNA
caagcagaagacggcatacgagatctgatgagggcagtcggtgatcatagcggtattacgcgagattacga

SEQ ID NO: 790 - DNA, 71 bp, synthetic construct
  source 1..71: mol_type = other DNA
caagcagaagacggcatacgagatacggtacaggcagtcggtgatcatagcggtattacgcgagattacga

SEQ ID NO: 791 - DNA, 71 bp, synthetic construct
  source 1..71: mol_type = other DNA
caagcagaagacggcatacgagatctcgacttggcagtcggtgatcatagcggtattacgcgagattacga

SEQ ID NO: 792 - DNA, 71 bp, synthetic construct
  source 1..71: mol_type = other DNA
caagcagaagacggcatacgagatacaacgtgggcagtcggtgatcatagcggtattacgcgagattacga

SEQ ID NO: 793 - DNA, 71 bp, synthetic construct
  source 1..71: mol_type = other DNA
caagcagaagacggcatacgagattgctgtgaggcagtcggtgatcatagcggtattacgcgagattacga

SEQ ID NO: 794 - DNA, 71 bp, synthetic construct
  source 1..71: mol_type = other DNA
caagcagaagacggcatacgagatccaagtagggcagtcggtgatcatagcggtattacgcgagattacga

SEQ ID NO: 795 - DNA, 71 bp, synthetic construct
  source 1..71: mol_type = other DNA
caagcagaagacggcatacgagataactgaggggcagtcggtgatcatagcggtattacgcgagattacga

SEQ ID NO: 796 - DNA, 71 bp, synthetic construct
  source 1..71: mol_type = other DNA
caagcagaagacggcatacgagataggtaggaggcagtcggtgatcatagcggtattacgcgagattacga

SEQ ID NO: 797 - DNA, 71 bp, synthetic construct
  source 1..71: mol_type = other DNA
caagcagaagacggcatacgagatttcgccatggcagtcggtgatcatagcggtattacgcgagattacga

SEQ ID NO: 798 - DNA, 71 bp, synthetic construct
  source 1..71: mol_type = other DNA
caagcagaagacggcatacgagatcaggtaagggcagtcggtgatcatagcggtattacgcgagattacga

SEQ ID NO: 799 - DNA, 71 bp, synthetic construct
  source 1..71: mol_type = other DNA
caagcagaagacggcatacgagatgtatcgagggcagtcggtgatcatagcggtattacgcgagattacga

SEQ ID NO: 800 - DNA, 71 bp, synthetic construct
  source 1..71: mol_type = other DNA
caagcagaagacggcatacgagatttcacggaggcagtcggtgatcatagcggtattacgcgagattacga

SEQ ID NO: 801 - DNA, 71 bp, synthetic construct
  source 1..71: mol_type = other DNA
caagcagaagacggcatacgagatgagctctaggcagtcggtgatcatagcggtattacgcgagattacga

SEQ ID NO: 802 - DNA, 71 bp, synthetic construct
  source 1..71: mol_type = other DNA
caagcagaagacggcatacgagatgtcagtcaggcagtcggtgatcatagcggtattacgcgagattacga

SEQ ID NO: 803 - DNA, 71 bp, synthetic construct
  source 1..71: mol_type = other DNA
caagcagaagacggcatacgagatcacgtctaggcagtcggtgatcatagcggtattacgcgagattacga

SEQ ID NO: 804 - DNA, 71 bp, synthetic construct
  source 1..71: mol_type = other DNA
caagcagaagacggcatacgagataattccggggcagtcggtgatcatagcggtattacgcgagattacga

SEQ ID NO: 805 - DNA, 71 bp, synthetic construct
  source 1..71: mol_type = other DNA
caagcagaagacggcatacgagattctaggagggcagtcggtgatcatagcggtattacgcgagattacga

SEQ ID NO: 806 - DNA, 71 bp, synthetic construct
  source 1..71: mol_type = other DNA
caagcagaagacggcatacgagatatccgttgggcagtcggtgatcatagcggtattacgcgagattacga

SEQ ID NO: 807 - DNA, 71 bp, synthetic construct
  source 1..71: mol_type = other DNA
caagcagaagacggcatacgagatgatagccaggcagtcggtgatcatagcggtattacgcgagattacga

SEQ ID NO: 808 - DNA, 71 bp, synthetic construct
  source 1..71: mol_type = other DNA
caagcagaagacggcatacgagattatgaccgggcagtcggtgatcatagcggtattacgcgagattacga

SEQ ID NO: 809 - DNA, 71 bp, synthetic construct
  source 1..71: mol_type = other DNA
caagcagaagacggcatacgagatcgattggaggcagtcggtgatcatagcggtattacgcgagattacga

SEQ ID NO: 810 - DNA, 71 bp, synthetic construct
  source 1..71: mol_type = other DNA
caagcagaagacggcatacgagatacaagctcggcagtcggtgatcatagcggtattacgcgagattacga

SEQ ID NO: 811 - DNA, 71 bp, synthetic construct
  source 1..71: mol_type = other DNA
caagcagaagacggcatacgagatgaaccttcggcagtcggtgatcatagcggtattacgcgagattacga

SEQ ID NO: 812 - DNA, 71 bp, synthetic construct
  source 1..71: mol_type = other DNA
caagcagaagacggcatacgagatagcgagatggcagtcggtgatcatagcggtattacgcgagattacga

SEQ ID NO: 813 - DNA, 71 bp, synthetic construct
  source 1..71: mol_type = other DNA
caagcagaagacggcatacgagatccgtaactggcagtcggtgatcatagcggtattacgcgagattacga

SEQ ID NO: 814 - DNA, 71 bp, synthetic construct
  source 1..71: mol_type = other DNA
caagcagaagacggcatacgagattcagacacggcagtcggtgatcatagcggtattacgcgagattacga

SEQ ID NO: 815 - DNA, 71 bp, synthetic construct
  source 1..71: mol_type = other DNA
caagcagaagacggcatacgagatcgaagtcaggcagtcggtgatcatagcggtattacgcgagattacga

SEQ ID NO: 816 - DNA, 71 bp, synthetic construct
  source 1..71: mol_type = other DNA
caagcagaagacggcatacgagatgtgatccaggcagtcggtgatcatagcggtattacgcgagattacga

SEQ ID NO: 817 - DNA, 71 bp, synthetic construct
  source 1..71: mol_type = other DNA
caagcagaagacggcatacgagatactggtgtggcagtcggtgatcatagcggtattacgcgagattacga

SEQ ID NO: 818 - DNA, 71 bp, synthetic construct
  source 1..71: mol_type = other DNA
caagcagaagacggcatacgagatctaacctgggcagtcggtgatcatagcggtattacgcgagattacga

SEQ ID NO: 819 - DNA, 71 bp, synthetic construct
  source 1..71: mol_type = other DNA
caagcagaagacggcatacgagatagccaactggcagtcggtgatcatagcggtattacgcgagattacga

SEQ ID NO: 820 - DNA, 71 bp, synthetic construct
  source 1..71: mol_type = other DNA
caagcagaagacggcatacgagatccagttgaggcagtcggtgatcatagcggtattacgcgagattacga

SEQ ID NO: 821 - DNA, 71 bp, synthetic construct
  source 1..71: mol_type = other DNA
caagcagaagacggcatacgagataagtgcagggcagtcggtgatcatagcggtattacgcgagattacga

SEQ ID NO: 822 - DNA, 71 bp, synthetic construct
  source 1..71: mol_type = other DNA
caagcagaagacggcatacgagataaccgtgtggcagtcggtgatcatagcggtattacgcgagattacga

SEQ ID NO: 823 - DNA, 71 bp, synthetic construct
  source 1..71: mol_type = other DNA
caagcagaagacggcatacgagatcgcgtattggcagtcggtgatcatagcggtattacgcgagattacga

SEQ ID NO: 824 - DNA, 71 bp, synthetic construct
  source 1..71: mol_type = other DNA
caagcagaagacggcatacgagatagttcgcaggcagtcggtgatcatagcggtattacgcgagattacga

SEQ ID NO: 825 - DNA, 71 bp, synthetic construct
  source 1..71: mol_type = other DNA
caagcagaagacggcatacgagattagtcagcggcagtcggtgatcatagcggtattacgcgagattacga

SEQ ID NO: 826 - DNA, 71 bp, synthetic construct
  source 1..71: mol_type = other DNA
caagcagaagacggcatacgagataacaccacggcagtcggtgatcatagcggtattacgcgagattacga

SEQ ID NO: 827 - DNA, 71 bp, synthetic construct
  source 1..71: mol_type = other DNA
caagcagaagacggcatacgagatgtaagcacggcagtcggtgatcatagcggtattacgcgagattacga

SEQ ID NO: 828 - DNA, 71 bp, synthetic construct
  source 1..71: mol_type = other DNA
caagcagaagacggcatacgagatgtccttgaggcagtcggtgatcatagcggtattacgcgagattacga

SEQ ID NO: 829 - DNA, 71 bp, synthetic construct
  source 1..71: mol_type = other DNA
caagcagaagacggcatacgagatcaggttcaggcagtcggtgatcatagcggtattacgcgagattacga

SEQ ID NO: 830 - DNA, 71 bp, synthetic construct
  source 1..71: mol_type = other DNA
caagcagaagacggcatacgagatccaacactggcagtcggtgatcatagcggtattacgcgagattacga

SEQ ID NO: 831 - DNA, 71 bp, synthetic construct
  source 1..71: mol_type = other DNA
caagcagaagacggcatacgagatgagagtacggcagtcggtgatcatagcggtattacgcgagattacga

SEQ ID NO: 832 - DNA, 71 bp, synthetic construct
  source 1..71: mol_type = other DNA
caagcagaagacggcatacgagatagatacggggcagtcggtgatcatagcggtattacgcgagattacga

SEQ ID NO: 833 - DNA, 71 bp, synthetic construct
  source 1..71: mol_type = other DNA
caagcagaagacggcatacgagatgttcttcgggcagtcggtgatcatagcggtattacgcgagattacga

SEQ ID NO: 834 - DNA, 71 bp, synthetic construct
  source 1..71: mol_type = other DNA
caagcagaagacggcatacgagatattccgctggcagtcggtgatcatagcggtattacgcgagattacga

SEQ ID NO: 835 - DNA, 71 bp, synthetic construct
  source 1..71: mol_type = other DNA
caagcagaagacggcatacgagataagctcacggcagtcggtgatcatagcggtattacgcgagattacga

SEQ ID NO: 836 - DNA, 71 bp, synthetic construct
  source 1..71: mol_type = other DNA
caagcagaagacggcatacgagattgatcacgggcagtcggtgatcatagcggtattacgcgagattacga

SEQ ID NO: 837 - DNA, 71 bp, synthetic construct
  source 1..71: mol_type = other DNA
caagcagaagacggcatacgagatcaatgcgaggcagtcggtgatcatagcggtattacgcgagattacga

SEQ ID NO: 838 - DNA, 71 bp, synthetic construct
  source 1..71: mol_type = other DNA
caagcagaagacggcatacgagatatgcgtcaggcagtcggtgatcatagcggtattacgcgagattacga

SEQ ID NO: 839 - DNA, 71 bp, synthetic construct
  source 1..71: mol_type = other DNA
caagcagaagacggcatacgagattacatcggggcagtcggtgatcatagcggtattacgcgagattacga

SEQ ID NO: 840 - DNA, 71 bp, synthetic construct
  source 1..71: mol_type = other DNA
caagcagaagacggcatacgagatactgcgaaggcagtcggtgatcatagcggtattacgcgagattacga

SEQ ID NO: 841 - DNA, 71 bp, synthetic construct
  source 1..71: mol_type = other DNA
caagcagaagacggcatacgagattctgtcgtggcagtcggtgatcatagcggtattacgcgagattacga

SEQ ID NO: 842 - DNA, 71 bp, synthetic construct
  source 1..71: mol_type = other DNA
caagcagaagacggcatacgagatctcaagctggcagtcggtgatcatagcggtattacgcgagattacga

SEQ ID NO: 843 - DNA, 71 bp, synthetic construct
  source 1..71: mol_type = other DNA
caagcagaagacggcatacgagataaccactcggcagtcggtgatcatagcggtattacgcgagattacga

SEQ ID NO: 844 - DNA, 71 bp, synthetic construct
  source 1..71: mol_type = other DNA
caagcagaagacggcatacgagatcttacagcggcagtcggtgatcatagcggtattacgcgagattacga

SEQ ID NO: 845 - DNA, 71 bp, synthetic construct
  source 1..71: mol_type = other DNA
caagcagaagacggcatacgagatagtcttggggcagtcggtgatcatagcggtattacgcgagattacga

SEQ ID NO: 846 - DNA, 71 bp, synthetic construct
  source 1..71: mol_type = other DNA
caagcagaagacggcatacgagatcacgcaatggcagtcggtgatcatagcggtattacgcgagattacga

SEQ ID NO: 847 - DNA, 71 bp, synthetic construct
  source 1..71: mol_type = other DNA
caagcagaagacggcatacgagatagcttcagggcagtcggtgatcatagcggtattacgcgagattacga

SEQ ID NO: 848 - DNA, 71 bp, synthetic construct
  source 1..71: mol_type = other DNA
caagcagaagacggcatacgagatcctcgttaggcagtcggtgatcatagcggtattacgcgagattacga

SEQ ID NO: 849 - DNA, 71 bp, synthetic construct
  source 1..71: mol_type = other DNA
caagcagaagacggcatacgagattgagacgaggcagtcggtgatcatagcggtattacgcgagattacga

SEQ ID NO: 850 - DNA, 71 bp, synthetic construct
  source 1..71: mol_type = other DNA
caagcagaagacggcatacgagatcacaggaaggcagtcggtgatcatagcggtattacgcgagattacga

SEQ ID NO: 851 - DNA, 71 bp, synthetic construct
  source 1..71: mol_type = other DNA
caagcagaagacggcatacgagatactcaacgggcagtcggtgatcatagcggtattacgcgagattacga

SEQ ID NO: 852 - DNA, 71 bp, synthetic construct
  source 1..71: mol_type = other DNA
caagcagaagacggcatacgagataagcgactggcagtcggtgatcatagcggtattacgcgagattacga

SEQ ID NO: 853 - DNA, 71 bp, synthetic construct
  source 1..71: mol_type = other DNA
caagcagaagacggcatacgagatcctacctaggcagtcggtgatcatagcggtattacgcgagattacga

SEQ ID NO: 854 - DNA, 71 bp, synthetic construct
  source 1..71: mol_type = other DNA
caagcagaagacggcatacgagatatctcctgggcagtcggtgatcatagcggtattacgcgagattacga

SEQ ID NO: 855 - DNA, 71 bp, synthetic construct
  source 1..71: mol_type = other DNA
caagcagaagacggcatacgagattcacgatgggcagtcggtgatcatagcggtattacgcgagattacga

SEQ ID NO: 856 - DNA, 71 bp, synthetic construct
  source 1..71: mol_type = other DNA
caagcagaagacggcatacgagatccacaacaggcagtcggtgatcatagcggtattacgcgagattacga

SEQ ID NO: 857 - DNA, 71 bp, synthetic construct
  source 1..71: mol_type = other DNA
caagcagaagacggcatacgagataggtctgtggcagtcggtgatcatagcggtattacgcgagattacga

SEQ ID NO: 858 - DNA, 71 bp, synthetic construct
  source 1..71: mol_type = other DNA
caagcagaagacggcatacgagatagaaggacggcagtcggtgatcatagcggtattacgcgagattacga

SEQ ID NO: 859 - DNA, 71 bp, synthetic construct
  source 1..71: mol_type = other DNA
caagcagaagacggcatacgagatgcgtatcaggcagtcggtgatcatagcggtattacgcgagattacga

SEQ ID NO: 860 - DNA, 71 bp, synthetic construct
  source 1..71: mol_type = other DNA
caagcagaagacggcatacgagatcaacacagggcagtcggtgatcatagcggtattacgcgagattacga

SEQ ID NO: 861 - DNA, 71 bp, synthetic construct
  source 1..71: mol_type = other DNA
caagcagaagacggcatacgagattccacgttggcagtcggtgatcatagcggtattacgcgagattacga

SEQ ID NO: 862 - DNA, 71 bp, synthetic construct
  source 1..71: mol_type = other DNA
caagcagaagacggcatacgagatatcgcaacggcagtcggtgatcatagcggtattacgcgagattacga

SEQ ID NO: 863 - DNA, 71 bp, synthetic construct
  source 1..71: mol_type = other DNA
caagcagaagacggcatacgagatacgtcgttggcagtcggtgatcatagcggtattacgcgagattacga

SEQ ID NO: 864 - DNA, 71 bp, synthetic construct
  source 1..71: mol_type = other DNA
caagcagaagacggcatacgagatcgaatacgggcagtcggtgatcatagcggtattacgcgagattacga

SEQ ID NO: 865 - DNA, 71 bp, synthetic construct
  source 1..71: mol_type = other DNA
caagcagaagacggcatacgagattgcttgctggcagtcggtgatcatagcggtattacgcgagattacga

SEQ ID NO: 866 - DNA, 71 bp, synthetic construct
  source 1..71: mol_type = other DNA
caagcagaagacggcatacgagatctcgaacaggcagtcggtgatcatagcggtattacgcgagattacga

SEQ ID NO: 867 - DNA, 71 bp, synthetic construct
  source 1..71: mol_type = other DNA
caagcagaagacggcatacgagatacatggagggcagtcggtgatcatagcggtattacgcgagattacga

SEQ ID NO: 868 - DNA, 71 bp, synthetic construct
  source 1..71: mol_type = other DNA
caagcagaagacggcatacgagatacaagacgggcagtcggtgatcatagcggtattacgcgagattacga

SEQ ID NO: 869 - DNA, 71 bp, synthetic construct
  source 1..71: mol_type = other DNA
caagcagaagacggcatacgagatcgccttatggcagtcggtgatcatagcggtattacgcgagattacga

SEQ ID NO: 870 - DNA, 71 bp, synthetic construct
  source 1..71: mol_type = other DNA
caagcagaagacggcatacgagatagcagacaggcagtcggtgatcatagcggtattacgcgagattacga

SEQ ID NO: 871 - DNA, 71 bp, synthetic construct
  source 1..71: mol_type = other DNA
caagcagaagacggcatacgagatgttaagcgggcagtcggtgatcatagcggtattacgcgagattacga

SEQ ID NO: 872 - DNA, 71 bp, synthetic construct
  source 1..71: mol_type = other DNA
caagcagaagacggcatacgagatcatggatcggcagtcggtgatcatagcggtattacgcgagattacga

SEQ ID NO: 873 - DNA, 71 bp, synthetic construct
  source 1..71: mol_type = other DNA
caagcagaagacggcatacgagatacagaggtggcagtcggtgatcatagcggtattacgcgagattacga

SEQ ID NO: 874 - DNA, 71 bp, synthetic construct
  source 1..71: mol_type = other DNA
caagcagaagacggcatacgagattaagtggcggcagtcggtgatcatagcggtattacgcgagattacga

SEQ ID NO: 875 - DNA, 71 bp, synthetic construct
  source 1..71: mol_type = other DNA
caagcagaagacggcatacgagatagtcaggtggcagtcggtgatcatagcggtattacgcgagattacga

SEQ ID NO: 876 - DNA, 71 bp, synthetic construct
  source 1..71: mol_type = other DNA
caagcagaagacggcatacgagatgccttaacggcagtcggtgatcatagcggtattacgcgagattacga

SEQ ID NO: 877 - DNA, 71 bp, synthetic construct
  source 1..71: mol_type = other DNA
caagcagaagacggcatacgagatgttggcatggcagtcggtgatcatagcggtattacgcgagattacga

SEQ ID NO: 878 - DNA, 71 bp, synthetic construct
  source 1..71: mol_type = other DNA
caagcagaagacggcatacgagatcaacctctggcagtcggtgatcatagcggtattacgcgagattacga

SEQ ID NO: 879 - DNA, 71 bp, synthetic construct
  source 1..71: mol_type = other DNA
caagcagaagacggcatacgagattggatggtggcagtcggtgatcatagcggtattacgcgagattacga

SEQ ID NO: 880 - DNA, 71 bp, synthetic construct
  source 1..71: mol_type = other DNA
caagcagaagacggcatacgagatctatccacggcagtcggtgatcatagcggtattacgcgagattacga

SEQ ID NO: 881 - DNA, 71 bp, synthetic construct
  source 1..71: mol_type = other DNA
caagcagaagacggcatacgagatgatctcagggcagtcggtgatcatagcggtattacgcgagattacga

SEQ ID NO: 882 - DNA, 24 bp, synthetic construct
  source 1..24: mol_type = other DNA
ttgtgactggtagcaggagcccat

SEQ ID NO: 883 - DNA, 25 bp, synthetic construct
  source 1..25: mol_type = other DNA
tctatcaggcttcagcagacccaga

SEQ ID NO: 884 - DNA, 52 bp, synthetic construct
  source 1..52: mol_type = other DNA
actagcgatcggtacctagcgccgaaacctattaccgcgacctagcgttgcg

SEQ ID NO: 885 - DNA, 52 bp, synthetic construct
  source 1..52: mol_type = other DNA
cgcaacgctaggtcgcggtaataggtttcggcgctaggtaccgatcgctagt

SEQ ID NO: 886 - DNA, 52 bp, synthetic construct; identical to SEQ ID NO: 884
  source 1..52: mol_type = other DNA

SEQ ID NO: 887 - DNA, 52 bp, synthetic construct; identical to SEQ ID NO: 885
  source 1..52: mol_type = other DNA

SEQ ID NO: 888 - DNA, 39 bp, synthetic construct; identical to SEQ ID NO: 64
  source 1..39: mol_type = other DNA

SEQ ID NO: 889 - DNA, 39 bp, synthetic construct; identical to SEQ ID NO: 65
  source 1..39: mol_type = other DNA

SEQ ID NO: 890 - DNA, 49 bp, synthetic construct; identical to SEQ ID NO: 24
  source 1..49: mol_type = other DNA

SEQ ID NO: 891 - DNA, 50 bp, synthetic construct; identical to SEQ ID NO: 25
  source 1..50: mol_type = other DNA

SEQ ID NO: 892 - DNA, 29 bp, synthetic construct
  source 1..29: mol_type = other DNA
aatgatacggcgaccaccgagatctacat

SEQ ID NO: 893 - DNA, 29 bp, synthetic construct
  source 1..29: mol_type = other DNA
aatgatacggcgaccaccgagatctacac

SEQ ID NO: 894 - DNA, 71 bp, synthetic construct; identical to SEQ ID NO: 74
  source 1..71: mol_type = other DNA

SEQ ID NO: 895 - DNA, 78 bp, synthetic construct; identical to SEQ ID NO: 72
  source 1..78: mol_type = other DNA

SEQ ID NO: 896 - DNA, 14 bp, synthetic construct
  source 1..14: mol_type = other DNA
gatcggaagagcca

SEQ ID NO: 897 - DNA, 39 bp, synthetic construct; identical to SEQ ID NO: 75
  source 1..39: mol_type = other DNA

SEQ ID NO: 898 - DNA, 39 bp, synthetic construct; identical to SEQ ID NO: 76
  source 1..39: mol_type = other DNA

SEQ ID NO: 899 - DNA, 50 bp, synthetic construct
  source 1..50: mol_type = other DNA
catagcggtattacgcgagattacgantagccggacgaatgtcggtcgtt

SEQ ID NO: 900 - DNA, 51 bp, synthetic construct
  source 1..51: mol_type = other DNA
catagcggtattacgcgagattacganntagccggacgaatgtcggtcgtt

SEQ ID NO: 901 - DNA, 52 bp, synthetic construct
  source 1..52: mol_type = other DNA
catagcggtattacgcgagattacgannntagccggacgaatgtcggtcgtt

SEQ ID NO: 902 - DNA, 53 bp, synthetic construct
  source 1..53: mol_type = other DNA
catagcggtattacgcgagattacgannnntagccggacgaatgtcggtcgtt

SEQ ID NO: 903 - DNA, 54 bp, synthetic construct
  source 1..54: mol_type = other DNA
catagcggtattacgcgagattacgannnnntagccggacgaatgtcggtcgtt

SEQ ID NO: 904 - DNA, 55 bp, synthetic construct
  source 1..55: mol_type = other DNA
catagcggtattacgcgagattacgannnnnntagccggacgaatgtcggtcgtt

SEQ ID NO: 905 - DNA, 51 bp, synthetic construct
  source 1..51: mol_type = other DNA
catagcggtattacgcgagattacganacattcgtccggctacctacgccc

SEQ ID NO: 906 - DNA, 52 bp, synthetic construct
  source 1..52: mol_type = other DNA
catagcggtattacgcgagattacgannacattcgtccggctacctacgccc

SEQ ID NO: 907 - DNA, 53 bp, synthetic construct
  source 1..53: mol_type = other DNA
catagcggtattacgcgagattacgannnacattcgtccggctacctacgccc

SEQ ID NO: 908 - DNA, 54 bp, synthetic construct
  source 1..54: mol_type = other DNA
catagcggtattacgcgagattacgannnnacattcgtccggctacctacgccc

SEQ ID NO: 909 - DNA, 55 bp, synthetic construct
  source 1..55: mol_type = other DNA
catagcggtattacgcgagattacgannnnnacattcgtccggctacctacgccc

SEQ ID NO: 910 - DNA, 56 bp, synthetic construct
  source 1..56: mol_type = other DNA
catagcggtattacgcgagattacgannnnnnacattcgtccggctacctacgccc

SEQ ID NO: 911 - RNA, 100 nt, synthetic construct; identical to SEQ ID NO: 288
  source 1..100: mol_type = other RNA

SEQ ID NO: 912 - RNA, 100 nt, synthetic construct; identical to SEQ ID NO: 71
  source 1..100: mol_type = other RNA

SEQ ID NO: 913 - RNA, 100 nt, synthetic construct; identical to SEQ ID NO: 69
  source 1..100: mol_type = other RNA

SEQ ID NO: 914 - RNA, 100 nt, synthetic construct; identical to SEQ ID NO: 291
  source 1..100: mol_type = other RNA

SEQ ID NO: 915 - DNA, 26 bp, synthetic construct
  source 1..26: mol_type = other DNA
gtcggtcgtagttagatcggaagagc

SEQ ID NO: 916 - DNA, 28 bp, synthetic construct
  source 1..28: mol_type = other DNA
tacctacgccgcttaagatcggaagagc